SEQUENCE LISTING (203 sequences)

SEQ ID NO: 1 - protein, 3 aa, Artificial Sequence
  Spacer motif
GGS

SEQ ID NO: 2 - protein, 3 aa, Artificial Sequence
  Spacer motif
GGT

SEQ ID NO: 3 - protein, 3 aa, Artificial Sequence
  Spacer motif
GGD

SEQ ID NO: 4 - protein, 3 aa, Artificial Sequence
  Spacer motif
GGE

SEQ ID NO: 5 - protein, 3 aa, Artificial Sequence
  Spacer motif
GGG

SEQ ID NO: 6 - protein, 3 aa, Artificial Sequence
  Spacer motif
AAS

SEQ ID NO: 7 - protein, 3 aa, Artificial Sequence
  Spacer motif
AAT

SEQ ID NO: 8 - protein, 3 aa, Artificial Sequence
  Spacer motif
AAD

SEQ ID NO: 9 - protein, 3 aa, Artificial Sequence
  Spacer motif
AAE

SEQ ID NO: 10 - protein, 3 aa, Artificial Sequence
  Spacer motif
AAA

SEQ ID NO: 11 - protein, 3 aa, Artificial Sequence
  Spacer motif
AGS

SEQ ID NO: 12 - protein, 3 aa, Artificial Sequence
  Spacer motif
AGT

SEQ ID NO: 13 - protein, 3 aa, Artificial Sequence
  Spacer motif
AGD

SEQ ID NO: 14 - protein, 3 aa, Artificial Sequence
  Spacer motif
AGE

SEQ ID NO: 15 - protein, 3 aa, Artificial Sequence
  Spacer motif
AGG

SEQ ID NO: 16 - protein, 6 aa, Artificial Sequence
  Spacer motif
GGSGGS

SEQ ID NO: 17 - protein, 9 aa, Artificial Sequence
  Spacer motif
GGSGGSGGS

SEQ ID NO: 18 - protein, 12 aa, Artificial Sequence
  Spacer motif
GGSGGSGGSGGS

SEQ ID NO: 19 - protein, 15 aa, Artificial Sequence
  Spacer motif
GGSGGSGGSGGSGGS

SEQ ID NO: 20 - protein, 6 aa, Artificial Sequence
  Spacer motif
GGTGGT

SEQ ID NO: 21 - protein, 9 aa, Artificial Sequence
  Spacer motif
GGTGGTGGT

SEQ ID NO: 22 - protein, 12 aa, Artificial Sequence
  Spacer motif
GGTGGTGGTGGT

SEQ ID NO: 23 - protein, 15 aa, Artificial Sequence
  Spacer motif
GGTGGTGGTGGTGGT

SEQ ID NO: 24 - protein, 3 aa, Artificial Sequence
  Glycan acceptor motif
NNT

SEQ ID NO: 25 - protein, 3 aa, Artificial Sequence
  Glycan acceptor motif
NNS

SEQ ID NO: 26 - protein, 3 aa, Artificial Sequence
  Glycan acceptor motif
NGT

SEQ ID NO: 27 - protein, 3 aa, Artificial Sequence
  Glycan acceptor motif
NGS

SEQ ID NO: 28 - protein, 3 aa, Artificial Sequence
  Glycan acceptor motif
NAT

SEQ ID NO: 29 - protein, 3 aa, Artificial Sequence
  Glycan acceptor motif
NAS

SEQ ID NO: 30 - protein, 3 aa, Artificial Sequence
  Glycan acceptor motif
NVT

SEQ ID NO: 31 - protein, 3 aa, Artificial Sequence
  Glycan acceptor motif
NVS

SEQ ID NO: 32 - protein, 3 aa, Artificial Sequence
  Glycan acceptor motif
NIT

SEQ ID NO: 33 - protein, 3 aa, Artificial Sequence
  Glycan acceptor motif
NIS

SEQ ID NO: 34 - protein, 6 aa, Artificial Sequence
  Synthesized GAABCP sequence
GGSNNT

SEQ ID NO: 35 - protein, 9 aa, Artificial Sequence
  Synthesized GAABCP sequence
GGSGGSNNT

SEQ ID NO: 36 - protein, 12 aa, Artificial Sequence
  Synthesized GAABCP sequence
GGSGGSGGSNNT

SEQ ID NO: 37 - protein, 15 aa, Artificial Sequence
  Synthesized GAABCP sequence
GGSGGSGGSGGSNNT

SEQ ID NO: 38 - protein, 18 aa, Artificial Sequence
  Synthesized GAABCP sequence
GGSGGSGGSGGSGGSNNT

SEQ ID NO: 39 - protein, 6 aa, Artificial Sequence
  Synthesized GAABCP sequence
GGSNNS

SEQ ID NO: 40 - protein, 9 aa, Artificial Sequence
  Synthesized GAABCP sequence
GGSGGSNNS

SEQ ID NO: 41 - protein, 12 aa, Artificial Sequence
  Synthesized GAABCP sequence
GGSGGSGGSNNS

SEQ ID NO: 42 - protein, 15 aa, Artificial Sequence
  Synthesized GAABCP sequence
GGSGGSGGSGGSNNS

SEQ ID NO: 43 - protein, 18 aa, Artificial Sequence
  Synthesized GAABCP sequence
GGSGGSGGSGGSGGSNNS

SEQ ID NO: 44 - protein, 6 aa, Artificial Sequence
  Synthesized GAABCP sequence
GGSNGT

SEQ ID NO: 45 - protein, 9 aa, Artificial Sequence
  Synthesized GAABCP sequence
GGSGGSNGT

SEQ ID NO: 46 - protein, 12 aa, Artificial Sequence
  Synthesized GAABCP sequence
GGSGGSGGSNGT

SEQ ID NO: 47 - protein, 15 aa, Artificial Sequence
  Synthesized GAABCP sequence
GGSGGSGGSGGSNGT

SEQ ID NO: 48 - protein, 18 aa, Artificial Sequence
  Synthesized GAABCP sequence
GGSGGSGGSGGSGGSNGT

SEQ ID NO: 49 - protein, 6 aa, Artificial Sequence
  Synthesized GAABCP sequence
GGSNAS

SEQ ID NO: 50 - protein, 9 aa, Artificial Sequence
  Synthesized GAABCP sequence
GGSGGSNAS

SEQ ID NO: 51 - protein, 12 aa, Artificial Sequence
  Synthesized GAABCP sequence
GGSGGSGGSNAS

SEQ ID NO: 52 - protein, 15 aa, Artificial Sequence
  Synthesized GAABCP sequence
GGSGGSGGSGGSNAS

SEQ ID NO: 53 - protein, 18 aa, Artificial Sequence
  Synthesized GAABCP sequence
GGSGGSGGSGGSGGSNAS

SEQ ID NO: 54 - protein, 45 aa, Artificial Sequence
  Synthesized GAABCP sequence
GGSGGSNNTGGSGGSNNTGGSGGSNNTGGSGGSNNTGGSGGSNNT

SEQ ID NO: 55 - protein, 90 aa, Artificial Sequence
  Synthesized GAABCP sequence
GGSGGSNNTGGSGGSNNTGGSGGSNNTGGSGGSNNTGGSGGSNNTGGSGGSNNTGGSGGSNNTGGSGGSNNTGGSGGSNNTGGSGGSNNT

SEQ ID NO: 56 - protein, 135 aa, Artificial Sequence
  Synthesized GAABCP sequence
GGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNT

SEQ ID NO: 57 - protein, 180 aa, Artificial Sequence
  Synthesized GAABCP sequence
GGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNT

SEQ ID NO: 58 - protein, 45 aa, Artificial Sequence
  Synthesized GAABCP sequence
GGSGGSNNSGGSGGSNNSGGSGGSNNSGGSGGSNNSGGSGGSNNS

SEQ ID NO: 59 - protein, 90 aa, Artificial Sequence
  Synthesized GAABCP sequence
GGSGGSNNSGGSGGSNNSGGSGGSNNSGGSGGSNNSGGSGGSNNSGGSGGSNNSGGSGGSNNSGGSGGSNNSGGSGGSNNSGGSGGSNNS

SEQ ID NO: 60 - protein, 135 aa, Artificial Sequence
  Synthesized GAABCP sequence
GGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNS

SEQ ID NO: 61 - protein, 180 aa, Artificial Sequence
  Synthesized GAABCP sequence
GGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNSGGSGGSNNS

SEQ ID NO: 62 - protein, 45 aa, Artificial Sequence
  Synthesized GAABCP sequence
GGSGGSNGTGGSGGSNGTGGSGGSNGTGGSGGSNGTGGSGGSNGT

SEQ ID NO: 63 - protein, 90 aa, Artificial Sequence
  Synthesized GAABCP sequence
GGSGGSNGTGGSGGSNGTGGSGGSNGTGGSGGSNGTGGSGGSNGTGGSGGSNGTGGSGGSNGTGGSGGSNGTGGSGGSNGTGGSGGSNGT

SEQ ID NO: 64 - protein, 135 aa, Artificial Sequence
  Synthesized GAABCP sequence
GGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGT

SEQ ID NO: 65 - protein, 180 aa, Artificial Sequence
  Synthesized GAABCP sequence
GGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGTGGSGGSNGT

SEQ ID NO: 66 - protein, 18 aa, Schizosaccharomyces pombe
MFLQNLFLGFLAVVCANA

SEQ ID NO: 67 - protein, 18 aa, Homo sapiens
MKWVTFISLLFLFSSAYS

SEQ ID NO: 68 - protein, 6 aa, Artificial Sequence
  Synthesize 6x His tag
HHHHHH

SEQ ID NO: 69 - protein, 8 aa, Artificial Sequence
  Synthesized Flag tag
AYKDDDDK

SEQ ID NO: 70 - protein, 10 aa, Artificial Sequence
  Synthesized Myc tag
EQKLISEEDK

SEQ ID NO: 71 - protein, 9 aa, Artificial Sequence
  Snthesized HA tag
YPYDVPDYA

SEQ ID NO: 72 - protein, 3 aa, Artificial Sequence
  Synthesized flexible peptide linker
GGS

SEQ ID NO: 73 - protein, 6 aa, Artificial Sequence
  Synthesized flexible peptide linker
GGSGGS

SEQ ID NO: 74 - protein, 9 aa, Artificial Sequence
  Synthesized flexible peptide linker
GGSGGSGGS

SEQ ID NO: 75 - protein, 12 aa, Artificial Sequence
  Synthesized flexible peptide linker
GGSGGSGGSGGS

SEQ ID NO: 76 - protein, 15 aa, Artificial Sequence
  Synthesized flexible peptide linker
GGSGGSGGSGGSGGS

SEQ ID NO: 77 - protein, 3 aa, Artificial Sequence
  Synthesized flexible peptide linker
GSG

SEQ ID NO: 78 - protein, 6 aa, Artificial Sequence
  Synthesized flexible peptide linker
GSGGSG

SEQ ID NO: 79 - protein, 9 aa, Artificial Sequence
  Synthesized flexible peptide linker
GSGGSGGSG

SEQ ID NO: 80 - protein, 12 aa, Artificial Sequence
  Synthesized flexible peptide linker
GSGGSGGSGGSG

SEQ ID NO: 81 - protein, 15 aa, Artificial Sequence
  Synthesized flexible peptide linker
GSGGSGGSGGSGGSG

SEQ ID NO: 82 - protein, 4 aa, Artificial Sequence
  Synthesized flexible peptide linker
GGGS

SEQ ID NO: 83 - protein, 8 aa, Artificial Sequence
  Synthesized flexible peptide linker
GGGSGGGS

SEQ ID NO: 84 - protein, 12 aa, Artificial Sequence
  Synthesized flexible peptide linker
GGGSGGGSGGGS

SEQ ID NO: 85 - protein, 16 aa, Artificial Sequence
  Synthesized flexible peptide linker
GGGSGGGSGGGSGGGS

SEQ ID NO: 86 - protein, 20 aa, Artificial Sequence
  Synthesized flexible peptide linker
GGGSGGGSGGGSGGGSGGGS

SEQ ID NO: 87 - protein, 4 aa, Artificial Sequence
  Synthesized flexible peptide linker
GGSG

SEQ ID NO: 88 - protein, 8 aa, Artificial Sequence
  Synthesized flexible peptide linker
GGSGGGSG

SEQ ID NO: 89 - protein, 12 aa, Artificial Sequence
  Synthesized flexible peptide linker
GGSGGGSGGGSG

SEQ ID NO: 90 - protein, 16 aa, Artificial Sequence
  Synthesized flexible peptide linker
GGSGGGSGGGSGGGSG

SEQ ID NO: 91 - protein, 20 aa, Artificial Sequence
  Synthesized flexible peptide linker
GGSGGGSGGGSGGGSGGGSG

SEQ ID NO: 92 - protein, 5 aa, Artificial Sequence
  Synthesized flexible peptide link
GGGGS

SEQ ID NO: 93 - protein, 10 aa, Artificial Sequence
  Synthesized flexible peptide linker
GGGGSGGGGS

SEQ ID NO: 94 - protein, 15 aa, Artificial Sequence
  Synthesized flexible peptide linker
GGGGSGGGGSGGGGS

SEQ ID NO: 95 - protein, 20 aa, Artificial Sequence
  Synthesized flexible peptide linker
GGGGSGGGGSGGGGSGGGGS

SEQ ID NO: 96 - protein, 25 aa, Artificial Sequence
  Synthesized flexible peptide linker
GGGGSGGGGSGGGGSGGGGSGGGGS

SEQ ID NO: 97 - protein, 204 aa, Homo sapiens
MAGPAYESPMRLMALELLLWHSALWTVEQATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 98 - protein, 174 aa, Artificial Sequence
  Modified human G-CSF
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 99 - protein, 243 aa, Artificial Sequence
  Modified human G-CSF (AQB-107)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTHHHHHH

SEQ ID NO: 100 - protein, 288 aa, Artificial Sequence
  Modified human G-CSF (ABQ-102)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 101 - protein, 333 aa, Artificial Sequence
  Modified human G-CSF (AQB-108)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 102 - protein, 378 aa, Artificial Sequence
  Modified human G-CSF (AQB-109)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 103 - protein, 31 aa, Homo sapiens
HAEGTFTSDVSSYLEGQAAKEFIAWLVKGRG

SEQ ID NO: 104 - protein, 31 aa, Artificial Sequence
  Modifed human GLP-1
HGEGTFTSDVSSYLEGQAAKEFIAWLVKGGG

SEQ ID NO: 105 - protein, 100 aa, Artificial Sequence
  Modified human GLP-1 (AQB-303)
MFLQNLFLGFLAVVCANAHGEGTFTSDVSSYLEGQAAKEFIAWLVKGGGGGSGGSNNTGGSGGSNNTGGSGGSNNTGGSGGSNNTGGSGGSNNTHHHHHH

SEQ ID NO: 106 - protein, 145 aa, Artificial Sequence
  Modified human GLP-1 (AQB-304)
MFLQNLFLGFLAVVCANAHGEGTFTSDVSSYLEGQAAKEFIAWLVKGGGGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 107 - protein, 190 aa, Artificial Sequence
  Modified human GLP-1 (AQB-301)
MFLQNLFLGFLAVVCANAHGEGTFTSDVSSYLEGQAAKEFIAWLVKGGGGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 108 - protein, 235 aa, Artificial Sequence
  Modified human GLP-1 (AQB-305)
MFLQNLFLGFLAVVCANAHGEGTFTSDVSSYLEGQAAKEFIAWLVKGGGGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 109 - protein, 185 aa, Homo sapiens
MPRLFFFHLLGVCLLLNEFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 110 - protein, 179 aa, Artificial Sequence
  Modified proRelaxin-2
MPRLFFFHLLGVCLLLNEFSRAVADSWMEEVIKLCGRELVRAQIAICGMSTWSSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSQLYSALANKCCHVGCTKRSLARFC

SEQ ID NO: 111 - protein, 185 aa, Artificial Sequence
  Modified proRelaxin-2
MPRLFFFHLLGVCLLLNEFSRAVADSWMEEVIKLCGRELVRAQIAICGMSTWSSLSQEDHHHHHHAPQTPRPVAEIVPSFINKDTETINMMSEFVANLPQELKLTLSEMQPALPQLQQHVPVLKDSSLLFEEFKKLIRNRQSEAADSSPSELKYLGLDTHSQLYSALANKCCHVGCTKRSLARFC

SEQ ID NO: 112 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 275 aa, Artificial Sequence
  Modified Fc fragment of IgG1 (AQB-203a)
MPRLFFFHLLGVCLLLNEFSRAVADSWMEEVIKLCGRELVRAQIAICGMSTWSSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSQLYSALANKCCHVGCTKRSLARFCGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTHHHHHH

SEQ ID NO: 114 - protein, 153 aa, Artificial Sequence
  Modified Fc fragment of IgG1 (AQB-236)
MPRLFFFHLLGVCLLLNEFSRAVAHHHHHHGGSGGSNNTGGSGGSNNTGGSGGSNNTGGSGGSNNTGGSGGSNNTGGGGSGGGGSDSWMEEVIKLCGRELVRAQIAICGMSTWSRRRRSVTGYGSRKKRQLYSALANKCCHVGCTKRSLARFC

SEQ ID NO: 115 - protein, 198 aa, Artificial Sequence
  Modified Fc fragment of IgG1 (AQB-237)
MPRLFFFHLLGVCLLLNEFSRAVAHHHHHHGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGGGSGGGGSDSWMEEVIKLCGRELVRAQIAICGMSTWSRRRRSVTGYGSRKKRQLYSALANKCCHVGCTKRSLARFC

SEQ ID NO: 116 - protein, 243 aa, Artificial Sequence
  Modified Fc fragment of IgG1 ((AQB-238)
MPRLFFFHLLGVCLLLNEFSRAVAHHHHHHGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGGGSGGGGSDSWMEEVIKLCGRELVRAQIAICGMSTWSRRRRSVTGYGSRKKRQLYSALANKCCHVGCTKRSLARFC

SEQ ID NO: 117 - protein, 288 aa, Artificial Sequence
  Modified Fc fragment of IgG1 (AQB-238a)
MPRLFFFHLLGVCLLLNEFSRAVAHHHHHHGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGGGSGGGGSDSWMEEVIKLCGRELVRAQIAICGMSTWSRRRRSVTGYGSRKKRQLYSALANKCCHVGCTKRSLARFC

SEQ ID NO: 118 - protein, 275 aa, Artificial Sequence
  Modified Fc fragment of IgG1 (AQB-210)
MPRLFFFHLLGVCLLLNEFSRAVAGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTDSWMEEVIKLCGRELVRAQIAICGMSTWSSLSQEDHHHHHHAPQTPRPVAEIVPSFINKDTETINMMSEFVANLPQELKLTLSEMQPALPQLQQHVPVLKDSSLLFEEFKKLIRNRQSEAADSSPSELKYLGLDTHSQLYSALANKCCHVGCTKRSLARFC

SEQ ID NO: 119 - protein, 179 aa, Artificial Sequence
  Modified Fc fragment of IgG1 (AQB-211)
MPRLFFFHLLGVCLLLNEFSRAVADSWMEEVIKLCGRELVRAQIAICGMSTWSHHHHHHGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSQLYSALANKCCHVGCTKRSLARFC

SEQ ID NO: 120 - protein, 275 aa, Artificial Sequence [sequence omitted]

SEQ ID NO: 121 - protein, 456 aa, Artificial Sequence [sequence omitted]

SEQ ID NO: 122 - protein, 369 aa, Artificial Sequence
  Modified Fc fragment of IgG1 (AQB-255)
MPRLFFFHLLGVCLLLNEFSRAVADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSNNTGGSGGSNNTGGSGGSNNTGGSGGSNNTGGSGGSNNTGGGGSGGGGSDSWMEEVIKLCGRELVRAQIAICGMSTWSGGGGSGGGGSQLYSALANKCCHVGCTKRSLARFC

SEQ ID NO: 123 - protein, 384 aa, Artificial Sequence [sequence omitted]

SEQ ID NO: 124 - protein, 18 aa, Artificial Sequence
  Synthesized spacer motif
GGSGGSGGSGGSGGSGGS

SEQ ID NO: 125 - protein, 21 aa, Artificial Sequence
  Synthesized spacer motif
GGSGGSGGSGGSGGSGGSGGS

SEQ ID NO: 126 - protein, 24 aa, Artificial Sequence
  Synthesized spacer motif
GGSGGSGGSGGSGGSGGSGGSGGS

SEQ ID NO: 127 - protein, 27 aa, Artificial Sequence
  Synthesized spacer motif
GGSGGSGGSGGSGGSGGSGGSGGSGGS

SEQ ID NO: 128 - protein, 30 aa, Artificial Sequence
  Synthesized spacer motif
GGSGGSGGSGGSGGSGGSGGSGGSGGSGGS

SEQ ID NO: 129 - protein, 18 aa, Artificial Sequence
  Synthesized spacer motif
GGTGGTGGTGGTGGTGGT

SEQ ID NO: 130 - protein, 20 aa, Artificial Sequence
  Synthesized spacer motif
GTGGTGGTGGTGGTGGTGGT

SEQ ID NO: 131 - protein, 24 aa, Artificial Sequence
  Synthesized spacer motif
GGTGGTGGTGGTGGTGGTGGTGGT

SEQ ID NO: 132 - protein, 27 aa, Artificial Sequence
  Synthesized spacer motif
GGTGGTGGTGGTGGTGGTGGTGGTGGT

SEQ ID NO: 133 - protein, 30 aa, Artificial Sequence
  Synthesized spacer motif
GGTGGTGGTGGTGGTGGTGGTGGTGGTGGT

SEQ ID NO: 134 - protein, 237 aa, Artificial Sequence
  Modified human G-CSF (AQB-114)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNT

SEQ ID NO: 135 - protein, 282 aa, Artificial Sequence
  Modified human G-CSF (AQB-115)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTGGSGGSNNTGGSGGSNNTGGSGGSNNTGGSGGSNNTGGSGGSNNT

SEQ ID NO: 136 - protein, 327 aa, Artificial Sequence
  Modified human G-CSF (AQB-116)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNT

SEQ ID NO: 137 - protein, 372 aa, Artificial Sequence
  Modified human G-CSF (AQB-117)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSNNT

SEQ ID NO: 138 - protein, 252 aa, Artificial Sequence
  Modified human G-CSF (AQB-118)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSNNTGGSNNTGGSNNTGGSNNTGGSNNTGGSNNTGGSNNTGGSNNTGGSNNTGGSNNT

SEQ ID NO: 139 - protein, 312 aa, Artificial Sequence
  Modified human G-CSF (AQB-119)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNT

SEQ ID NO: 140 - protein, 342 aa, Artificial Sequence
  Modified human G-CSF (AQB-120)
MFLQNLFLGFLAVVCANATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNT

SEQ ID NO: 141 - protein, 241 aa, Artificial Sequence
  Modified human GLP-1 (AQB-307)
MFLQNLFLGFLAVVCANAHGEGTFTSDVSSYLEGQAAKEFIAWLVKGGGGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 142 - protein, 301 aa, Artificial Sequence
  Modified human GLP-1 (AQB-308)
MFLQNLFLGFLAVVCANAHGEGTFTSDVSSYLEGQAAKEFIAWLVKGGGGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 143 - protein, 361 aa, Artificial Sequence
  Modified human GLP-1 (AQB-309)
MFLQNLFLGFLAVVCANAHGEGTFTSDVSSYLEGQAAKEFIAWLVKGGGGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 144 - protein, 286 aa, Artificial Sequence
  Modified human GLP-1 (AQB-310)
MFLQNLFLGFLAVVCANAHGEGTFTSDVSSYLEGQAAKEFIAWLVKGGGGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSHHHHHH

SEQ ID NO: 145 - protein, 331 aa, Artificial Sequence
  Modified human GLP-1 (AQB-311)
MFLQNLFLGFLAVVCANAHGEGTFTSDVSSYLEGQAAKEFIAWLVKGGGGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSHHHHHH

SEQ ID NO: 146 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 147 - protein, 192 aa, Artificial Sequence
  Modified human EPO
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

SEQ ID NO: 148 - protein, 324 aa, Artificial Sequence
  Modified human EPO (AQB-401)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSHHHHHH

SEQ ID NO: 149 - protein, 324 aa, Artificial Sequence
  Modified human EPO (AQB-402)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 150 - protein, 315 aa, Artificial Sequence
  Modified human EPO (AQB-403)
MFLQNLFLGFLAVVCANAGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDHHHHHH

SEQ ID NO: 151 - protein, 354 aa, Artificial Sequence
  Modified human EPO (AQB-404)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSHHHHHH

SEQ ID NO: 152 - protein, 384 aa, Artificial Sequence
  Modified human EPO (AQB-405)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSHHHHHH

SEQ ID NO: 153 - protein, 414 aa, Artificial Sequence
  Modified human EPO (AQB-406)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSHHHHHH

SEQ ID NO: 154 - protein, 264 aa, Artificial Sequence
  Modified human EPO (AQB-407)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSNNTGGSGGSGGSNNTGGSGGSGGSNNTGGSGGSGGSNNTGGSGGSGGSNNTGGSGGSHHHHHH

SEQ ID NO: 155 - protein, 384 aa, Artificial Sequence
  Modified human EPO (AQB-408)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSHHHHHH

SEQ ID NO: 156 - protein, 444 aa, Artificial Sequence
  Modified human EPO (AQB-409)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSHHHHHH

SEQ ID NO: 157 - protein, 39 aa, Homo sapiens
SYSMEHFRWGKPVGKKRRPVKVYPNGAEDESAEAFPLEF

SEQ ID NO: 158 - protein, 219 aa, Artificial Sequence
  Modified human ACTH (AQB-501)
MFLQNLFLGFLAVVCANASYSMEHFRWGKPVGKKRRPVKVYPNGAEDESAEAFPLEFGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSHHHHHH

SEQ ID NO: 159 - protein, 294 aa, Artificial Sequence
  Modified human ACTH (AQB-502)
MFLQNLFLGFLAVVCANASYSMEHFRWGKPVGKKRRPVKVYPNGAEDESAEAFPLEFGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSHHHHHH

SEQ ID NO: 160 - protein, 369 aa, Artificial Sequence
  Modified human ACTH (AQB-503)
MFLQNLFLGFLAVVCANASYSMEHFRWGKPVGKKRRPVKVYPNGAEDESAEAFPLEFGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSHHHHHH

SEQ ID NO: 161 - protein, 444 aa, Artificial Sequence
  Modified human ACTH (AQB-504)
MFLQNLFLGFLAVVCANASYSMEHFRWGKPVGKKRRPVKVYPNGAEDESAEAFPLEFGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSHHHHHH

SEQ ID NO: 162 - protein, 249 aa, Artificial Sequence
  Modified human ACTH (AQB-505)
MFLQNLFLGFLAVVCANASYSMEHFRWGKPVGKKRRPVKVYPNGAEDESAEAFPLEFGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSHHHHHH

SEQ ID NO: 163 - protein, 339 aa, Artificial Sequence
  Modified human ACTH (AQB-506)
MFLQNLFLGFLAVVCANASYSMEHFRWGKPVGKKRRPVKVYPNGAEDESAEAFPLEFGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSHHHHHH

SEQ ID NO: 164 - protein, 104 aa, Homo sapiens
SYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFM

SEQ ID NO: 165 - protein, 314 aa, Artificial Sequence
  Modified pPOMC (AQB-507)
MFLQNLFLGFLAVVCANASYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSHHHHHH

SEQ ID NO: 166 - protein, 359 aa, Artificial Sequence
  Modified pPOMC (AQB-508)
MFLQNLFLGFLAVVCANASYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSHHHHHH

SEQ ID NO: 167 - protein, 404 aa, Artificial Sequence
  Modified pPOMC (AQB-509)
MFLQNLFLGFLAVVCANASYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSHHHHHH

SEQ ID NO: 168 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 169 - protein, 500 aa, Artificial Sequence
  Modified human FIX (AQB-601)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSNITGGSGGSNITGGSGGSNITGGSGGSHHHHHH

SEQ ID NO: 170 - protein, 608 aa, Artificial Sequence
  Modified human FIX (AQB-602)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSHHHHHH

SEQ ID NO: 171 - protein, 653 aa, Artificial Sequence
  Modified human FIX (AQB-603)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 172 - protein, 698 aa, Artificial Sequence
  Modified human FIX (AQB-604)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSHHHHHH

SEQ ID NO: 173 - protein, 743 aa, Artificial Sequence
  Modified human FIX (AQB-605)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSHHHHHH

SEQ ID NO: 174 - protein, 533 aa, Artificial Sequence
  Modified human FIX (AQB-606)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSGGSNITGGSGGSGGSNITGGSGGSGGSNITGGSGGSGGSNITGGSGGSGGSNITGGSGGSHHHHHH

SEQ ID NO: 175 - protein, 593 aa, Artificial Sequence
  Modified human FIX (AQB-607)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 176 - protein, 713 aa, Artificial Sequence
  Modified human FIX (AQB-608)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSHHHHHH

SEQ ID NO: 177 - protein, 569 aa, Artificial Sequence
  Modified Fc fragment of IgG1 (AQB-701)
MFLQNLFLGFLAVVCANAQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKSGGSGGSNNTGGSGGSNNTGGSGGSNNTGGSGGSNNTGGSGGSNNTGGSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 178 - protein, 563 aa, Artificial Sequence
  Modified Fc fragment of IgG1 (AQB-702)
MFLQNLFLGFLAVVCANAQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKSGGSGGSNNTGGSGGSNNTGGSGGSNNTGGSGGSNNTGGSGGSNNTDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 179 - protein, 294 aa, Artificial Sequence
  Modified human erythropoietin sequence (AQB-401a)
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSHHHHHH

SEQ ID NO: 180 - protein, 159 aa, Artificial Sequence
  Modified human adrenocorticotropic hormone sequence (AQB-501a)
MFLQNLFLGFLAVVCANASYSMEHFRWGKPVGKKRRPVKVYPTGAEDESAEAFPLEFGGSGGSNNTGGSGGSNNTGGSGGSNNTGGSGGSNNTGGSGGSNNTGGSGGSNNTGGSGGSNNTGGSGGSNNTGGSGGSNNTGGSGGSNNTGGSGGSHHHHHH

SEQ ID NO: 181 - protein, 60 aa, Artificial Sequence
  Synthesized GAABCP sequence
GGSNNTGGSNNTGGSNNTGGSNNTGGSNNTGGSNNTGGSNNTGGSNNTGGSNNTGGSNNT

SEQ ID NO: 182 - protein, 120 aa, Artificial Sequence
  Synthesized GAABCP sequence
GGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNT

SEQ ID NO: 183 - protein, 150 aa, Artificial Sequence
  Synthesized GAABCP sequence
GGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNT

SEQ ID NO: 184 - protein, 300 aa, Artificial Sequence
  Synthesized GAABCP sequence
GGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNIT

SEQ ID NO: 185 - protein, 225 aa, Artificial Sequence
  Synthesized GAABCP sequence
GGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNT

SEQ ID NO: 186 - protein, 270 aa, Artificial Sequence
  Synthesized GAABCP sequence
GGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNT

SEQ ID NO: 187 - protein, 180 aa, Artificial Sequence
  Synthesized GAABCP sequence
GGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNTGGSGGSGGSGGSGGSNNT

SEQ ID NO: 188 - protein, 210 aa, Artificial Sequence
  Synthesized GAABCP sequence
GGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNTGGSGGSGGSGGSGGSGGSNNT

SEQ ID NO: 189 - protein, 135 aa, Artificial Sequence
  Synthesized GAABCP sequence
GGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNITGGSGGSNIT

SEQ ID NO: 190 - protein, 180 aa, Artificial Sequence
  Synthesized GAABCP sequence
GGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNIT

SEQ ID NO: 191 - protein, 225 aa, Artificial Sequence
  Synthesized GAABCP sequence
GGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNITGGSGGSGGSGGSNIT

SEQ ID NO: 192 - protein, 270 aa, Artificial Sequence
  Synthesized GAABCP sequence
GGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNITGGSGGSGGSGGSGGSNIT

SEQ ID NO: 193 - protein, 60 aa, Artificial Sequence
  Synthesized GAABCP sequence
GGSGGSGGSNITGGSGGSGGSNITGGSGGSGGSNITGGSGGSGGSNITGGSGGSGGSNIT

SEQ ID NO: 194 - protein, 120 aa, Artificial Sequence
  Synthesized GAABCP sequence
GGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNIT

SEQ ID NO: 195 - protein, 240 aa, Artificial Sequence
  Synthesized GAABCP sequence
GGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNITGGSGGSGGSNIT

SEQ ID NO: 196 - protein, 90 aa, Artificial Sequence
  Synthesized GAABCP sequence
GGSGGSNNTGGSGGSNNTGGSGGSNITGGSGGSNITGGSGGSNITGGSGGSNNTGGSGGSNNTGGSGGSNITGGSGGSNITGGSGGSNIT

SEQ ID NO: 197 - protein, 180 aa, Artificial Sequence
  Synthesized GAABCP sequence
GGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNT

SEQ ID NO: 198 - protein, 240 aa, Artificial Sequence
  Synthesized GAABCP sequence
GGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNTGGSGGSGGSNNT

SEQ ID NO: 199 - protein, 300 aa, Artificial Sequence
  Synthesized GAABCP sequence
GGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNT

SEQ ID NO: 200 - protein, 375 aa, Artificial Sequence
  Synthesized GAABCP sequence
GGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNTGGSGGSGGSGGSNNT

SEQ ID NO: 201 - protein, 60 aa, Artificial Sequence; identical to SEQ ID NO: 193
  Synthesized GAABCP sequence

SEQ ID NO: 202 - protein, 120 aa, Artificial Sequence; identical to SEQ ID NO: 194
  Synthesized GAABCP sequence

SEQ ID NO: 203 - protein, 240 aa, Artificial Sequence; identical to SEQ ID NO: 195
  Synthesized GAABCP sequence

(19) United States
(12) Patent Application Publication
(10) Pub. No.: US 2016/0296632 A1
(43) Pub. Date: Oct. 13, 2016

(54) ENGINEERED GLYCOPROTEINS AND USES THEREOF

(71) Applicant: AEQUUS BIOPHARMA, INC., Seattle, WA (US)

(72) Inventor: Stewart D. CHIPMAN, Bainbridge Island, WA (US)

(21) Appl. No.: 15/036,206

(22) PCT Filed: Nov. 13, 2014
(86) PCT No.: PCT/US2014/065552

§ 371 Date: May 12, 2016

Related U.S. Application Data
(60) Provisional application No. 61/903,886, filed on Nov. 13, 2013.

(51) Int. Cl. A61K 47/48; A61K 38/22; C12N 9/64; A61K 38/48; C12Q 1/56; A61K 38/19; A61K 38/18

(52) U.S. Cl. CPC A61K 47/48338; A61K 38/193; A61K 38/2221; A61K 38/1816; A61K 38/22; A61K 38/4846; C12Y 304/21022; C12Q 1/56; C12N 9/644

(57) ABSTRACT

The present invention provides for novel glycoproteins comprising therapeutically useful polypeptides, or fragments or variants thereof, conjugated to glycosylated amino acid block copolymer polypeptides. These novel glycoproteins possess improved half-life properties as compared to the unmodified therapeutically useful polypeptide. Furthermore, the invention encompasses methods for designing and synthesizing nucleic acid molecules encoding these novel glycoproteins, cloning recombinant expression vectors containing these nucleic acid molecules, making host cells transformed with these nucleic acid expression vectors, and recombinantly expressing, purifying and characterizing the novel glycoproteins of the invention. Additionally the present invention encompasses pharmaceutical compositions comprising these novel glycoproteins, as well as methods of treating, preventing or ameliorating disease, disorders or conditions using novel glycoproteins of the invention.

### CROSS REFERENCE TO RELATED APPLICATIONS

This application claims priority to U.S. Provisional Application No. 61/903,886, filed on Nov. 13, 2013, which application is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ABIO_002_01WO_ST25.txt. The text file is 278 KB, was created on Nov. 13, 2014, and is being submitted electronically via EFS-Web.

### FIELD OF THE INVENTION

Embodiments of the present invention relate to novel glycoprotein conjugate compositions with improved pharmacokinetic properties due to the conjugation of glycosylated amino acid block copolymer polypeptides to therapeutically useful polypeptides, fragments or variants thereof, as well as methods of their preparation and use.

### BACKGROUND OF THE INVENTION

Many polypeptides, and fragments or variants thereof, are useful as therapeutic agents in human and veterinary medicine. However, when utilized for these purposes, these therapeutically useful polypeptides frequently display a number of undesirable properties that can limit their utility, including susceptibility to proteolytic degradation, rapid clearance from the plasma compartment, immunogenic or allergenic properties, and the like. The relatively short intrinsic plasma half-life observed for many therapeutically useful polypeptides in turn often results in attenuated therapeutic efficacy. As a result, frequently administered doses are often required, which in turn may lead to problems with patient compliance and increasing treatment cost.

Several methods for increasing the half-life of therapeutically useful polypeptides have been described in the art. However the application of many of these methods to therapeutically useful polypeptides is burdened with complex methodology that adds to product development time and manufacturing costs, due to, e.g., 1) the need for post-expression modification steps, such as chemical conjugation with the need for subsequent re-purification to remove impurities, or 2) the need for extensive knowledge of the structure-activity relationships of the native polypeptide so that the half-life extending modifications do not interfere with biological activity. Typically, the added costs of these drug development and manufacturing requirements are off-set by increases in the cost of the drugs, thus obviating much of the cost-benefits to the patient gained by the prolonged activity of these agents. Accordingly, a great need exists for the development of novel methods of increasing the half-lives of a wide range of therapeutically useful polypeptides that does not compromise the overall therapeutic efficacy of these drugs, and does not require complex methods of manufacture or extended product development efforts.

The present invention provides a solution to this challenge, via a novel approach for increasing the plasma half-life of therapeutically useful polypeptides utilizing naturally occurring materials, and employing an efficient process that simplifies product development, and may lower manufacturing costs and complexity.

### SUMMARY OF THE INVENTION

Embodiments of the present invention provide for compositions of matter comprising therapeutically useful polypeptides, and fragments or variants thereof (hereinafter TUPs), conjugated to one or more amino acid extensions of variable sequence and/or length; wherein the amino acid extensions comprise a block copolymer arrangement of repeating glycan acceptor motifs separated by spacer motifs, (or “glycosylated amino acid block copolymer-based polypeptides” or “GAABCPs”). Accordingly, GAABCPs comprise at least two glycan acceptor motifs separated by a spacer motif, as described herein. In various embodiments, the present invention provides GAABCP-conjugated TUPs that possess plasma half-lives that are increased as compared to their corresponding unmodified TUPs.

In some embodiments, the size and composition of the GAABCP is designed to allow optimal control over the post-translational addition of glycan chains to the glycan acceptor motifs. Accordingly, in some embodiments, the size and composition of the GAABCP enables maximal and consistent occupancy of the GAABCP glycan acceptor sites, with appropriately formed glycan structures, when exposed to the appropriate glycan synthesis/transfer enzyme systems in vitro, ex vivo, and/or in vivo. Thus, in some embodiments, the present invention provides a GAABCP or a GAABCP-TUP comprising at least one glycan or glycan chain (e.g., an N-glycan chain). In various embodiments, the present invention provides a GAABCP or a GAABCP-TUP comprising a plurality of glycans or glycan chains (e.g., an N-glycan chain), e.g., at least about 5, 10, 15, 20 or more glycans or glycan chains, including all integers (e.g., 6, 7, 8, 9, etc.) in between. In some embodiments, the one or more glycan or glycan chain comprises at least one terminal sialic acid moiety. In some embodiments, at least 60% of all N-glycan chains are terminally sialyated.

In some embodiments, the size and composition of the GAABCP is adjusted based on the molecular weight of the TUP so that the resultant GAABCP-conjugated TUP will possess a half-life that is increased when compared to its corresponding unmodified TUP. For example, in some embodiments, the size of a GAABCP used for conjugating to a TUP that has a short amino acid sequence (e.g., a peptide) is adjusted to increase the size of the polypeptide conjugate to a suitable molecular weight. Similarly, in some embodiments, the size of a GAABCP used for conjugating to a TUP that has a long amino acid sequence may optionally be decreased to a suitable molecular weight. Such tunability allows the present invention to be easily applied to a wide range of TUPs of different molecular weights.

In some embodiments, the GAABCP-conjugated TUP is designed to have a molecular weight ranging from about about 50 kDa to about 300 kDa or more. In some such embodiments, the present invention provides a GAABCP-conjugated TUP that possesses a half-life that is increased when compared to its corresponding unmodified TUP.

In some embodiments, the present invention provides a GAABCP comprising at least about 45 amino acids. In some embodiments, the present invention provides a GAABCP comprising at least about 90 amino acids. In some embodiments, the present invention provides a GAABCP comprising at least about 135 amino acids. In some embodiments, the present invention provides a GAABCP comprising at least about 180 amino acids. In some embodiments, one or more of the GAABCPs are conjugated at the N-terminus or C-terminus of a TUP (e.g., FIGS. 1A & B), or both termini. In some embodiments, one or more GAABCPs are conjugated within the internal amino acid sequence of a TUP. In further embodiments, two or more TUPs are linked together by one or more GAABCPs, according to the present disclosure (e.g., FIG. 1C). In one such embodiment, the present invention provides a GAABCP-TUP comprising two antigen binding domains (e.g., a scFvs) linked via a GAABCP of the present invention. In one such embodiment, one of the antigen binding domains is capable of activating a T cell via its binding to an antigen on a T cell (e.g., via binding to CD3) and the other antigen binding domain is directed to a tumor associated antigen (TAA) (e.g., HER2, EGFR, CD19). In some embodiments, such “Glyco-BiTEs” are targeted to solid or liquid tumors via the TAA binding domain.

In some embodiments, one or more GAABCPs are conjugated to a TUP via genetic conjugation. In some embodiments, one or more GAABCPs are conjugated to a TUP via chemical conjugation. In some embodiments, a glycosylated GAABCP is synthesized by solid-phase synthesis, wherein at least one glycan acceptor motif comprised in the GAABCP comprises an amino acid (e.g., an asparagine residue) that is chemically attached to an N-glycan moiety, which has a specific, defined carbohydrate structure. In particular embodiments, a plurality of the glycan acceptor motifs present in a GAABCP comprise chemically attached N-glycan moieties with defined carbohydrate structure. In some embodiments such as these, at least some of the N-glycan moieties are terminally sialylated; and in one embodiment, the N-glycan moieties comprise a high level of terminal sialylation.

The GAABCP-TUPs may also optionally comprise one or more affinity tags to assist in purification and characterization of the GAABCP-TUP. Many such affinity tags are well known to those skilled in the art and may be positioned in any suitable location within the GAABCP-TUP sequence, as described herein. In certain embodiments, GAABCP-TUPs comprise an affinity tag encoded by any one of the sequences provided as SEQ ID NOS: 68-71, or as provided in Table 4.

Additionally, in some embodiments, GAABCP-TUPs may optionally comprise other features such as one or more linker domains (e.g., a flexible linker), as disclosed herein, separating a TUP from the GAABCP. In some embodiments, one or more GAABCPs are conjugated to a TUP via a linker domain comprising two or more amino acid residues selected from the group consisting of Gly (G) and/or Ser (S). In certain embodiments, one or more GAABCPs are conjugated to a TUP via a linker domain encoded by any one of the sequences provided as SEQ ID NOS: 72-96, or as provided in Table 4.

Furthermore, in some embodiments, TUPs may comprise a secretion signal sequence (or “signal peptide”) to ensure proper processing and secretion by the cellular secretory pathway during ex vivo or in vivo recombinant expression. Such signal sequences may be native to the TUP; or they may be non-native to the TUP, and thus derived from another polypeptide; or alternatively from the same TUP, but derived from different species. In certain embodiments, GAABCP-TUPs of the present invention comprise a non-native secretion signal peptide encoded by any one of the sequences provided as SEQ ID NO: 66-67, or as provided in Table 4.

Suitable glycan acceptor motifs for inclusion in the GAABCPs disclosed herein include N-glycan and O-glycan acceptor motifs. In some embodiments, for a given GAABCP, the glycan acceptor motif may vary in its specific amino acid sequence with each subsequent repeat (e.g., for N-glycan motifs, an NNT may be followed by a NNS, followed by an NGT, etc); or the glycan acceptor motif may be held constant within the repeats of the GAABCP (e.g., for N-glycan motifs, NNT followed by NNT, followed by NNT, etc.). Additionally, GAABCPs may optionally comprise only N-glycan motifs, only O-glycan motifs, or mixtures of both O- and N-glycan motifs.

In some particular embodiments, the glycan acceptor motifs comprised in a GAABCP of the present invention all comprise the primary structure NX4(T/S); wherein N, T, and S are asparagine, threonine, and serine, respectively; and X4 is any amino acid but proline; and (T/S) indicates that either a threonine residue or a serine residue may be present. In certain embodiments, X4 is selected from the group consisting of asparagine, glycine, alanine, valine, and isoleucine. In some non-limiting embodiments, GAABCPs comprise a glycan acceptor motif comprising an amino acid sequence selected from the group consisting of any one of SEQ ID NOS: 24-33, or as provided in Table 2.

GAABCPs may comprise any suitable spacer motif separating the repeating glycan motifs comprised therein. Suitable spacer motifs include any sequence that is permissive for the glycosylation of at least one glycan acceptor motif when appropriate conditions are provided. In some embodiments, a key feature of the spacer motif is that it separates adjacent glycan acceptor motifs, thus providing sufficient room for the efficient addition of successive glycan moieties. Accordingly, it is explicitly contemplated that each of the various GAABCPs of the present invention may employ any of a wide range of spacer motifs to achieve the goal of optimally spacing the glycan acceptor sites. In some embodiments, the spacer motif is from 1-150 amino acids in length. In particular embodiments, the spacer motif is at least about 6 amino acids, or at least about, 9, 12, 15, 18, 21, or more amino acids.

In certain embodiments, conjugation of a TUP with a GAABCP comprising spacer motifs that are longer (i.e., that comprise more amino amino acids in length) than the spacer motif in another similar GAABCP results in an increase in glycan occupancy as calculated according to the method described in Example 8. In certain embodiments, conjugation of a TUP with a GAABCP comprising a spacer motif that is longer than the spacer motif in another similar GAABCP results in an increase in plasma half-life. In certain embodiments, conjugation of a TUP with a GAABCP comprising spacer motifs that are at least 9 amino acids results in an increase in glycan occupance as compared to the same TUP conjugated with a similar GAABCP comprising spacer motifs that are at less than 9 amino acids in length.

In some embodiments, the spacer sequence is a non-native sequence with respect to the TUP to which the GAABCP is conjugated.

In some embodiments, for a given GAABCP, the spacer motif may vary in its specific amino acid sequence and length with each subsequent repeat (e.g., a GGS may be followed by a GGG, followed by a GGGGS, etc); or the spacer motif may be held constant within the repeats of the GAABCP (e.g., GGS followed by a GGS, followed by a GGS, etc). In particular embodiments, the GAABCPs of the present invention comprise spacer motifs comprising the primary structure (X1X2X3)ₙ; wherein X1 and X2 are selected from the groups consisting of glycine, alanine, valine, isoleucine, and leucine; X3 is selected from the group consisting of serine, threonine, asparagine, glutamine, glycine, alanine, valine, isoleucine, and leucine; and n is an integer between 1 and 50. Accordingly, certain non-limiting embodiments provide for GAABCPs comprising spacer motifs of the aforementioned primary structure wherein, e.g., X1 is glycine; X2 is glycine; X3 is serine; X1 and X2 are both glycine; or X1 and X2 are both glycine and X3 is serine. In one such certain embodiment, n is selected from 3 or at least 3. In some non-limiting embodiments, GAABCPs of the present invention comprise a spacer motif comprising an amino acid sequence selected from the group consisting of any one of SEQ ID NOS: 1-23 and 124-133, or as provided in Table 1.

In some embodiments, GAABCPs comprise between 2 and 100 glycan acceptor motifs (“glycan motif”), each glycan motif being separated from one another by a spacer motif, as disclosed herein. In one certain embodiment, all of the glycan motifs comprised in the GAABCP are N-glycan motifs.

In some particular embodiments, the present invention provides for GAABCPs comprising the primary amino acid sequence, [(X1X2X3)ₙ—NX4(T/S)]ₙ; wherein a is an integer between 2 and 100 (indicating the number of times the bracketed motif is repeated in the GAABCP); N, T, and S are asparagine, threonine, and serine, respectively; X4 is any amino acid but proline; X1 and X2 are selected from the groups consisting of glycine (G), alanine (A), valine (V), isoleucine (I), and leucine (L); X3 is selected from the group consisting of serine, threonine, asparagine, glutamine, glycine, alanine, valine, isoleucine, and leucine; and n is an integer between 1 and 50. Accordingly, in such embodiments, the (X1X2X3)ₙ portion of the structure represents an amino acid-based ‘spacer’ motif, while the NX4(T/S) comprises an N-glycan acceptor motif. In particular embodiments, GAABCPs comprise NX4(T/S)-type glycan acceptor motifs, wherein X4 is selected from the group consisting of asparagine, glycine, alanine, valine, and isoleucine.

In some non-limiting embodiments, GAABCPs of the present invention comprise a spacer motif comprising three repeats of the amino acid sequence Glycine-Glycine-Serine (e.g., SEQ ID NO: 17). In some non-limiting embodiments, GAABCPs comprise a glycan acceptor motif comprising the amino acid sequence Asparagine-Asparagine-Threonine (e.g., SEQ ID NO: 24). In some particular embodiments, GAABCPs comprise amino acid sequences containing a spacer motif of between 1 and 5 repeating units of GGS, and either one NNT glycan acceptor motif as shown in SEQ ID NOS: 34-38, or one NNS glycan acceptor motif as shown in SEQ ID NOS: 39-43, or one NGT glycan acceptor motif as shown in SEQ ID NOS: 44-48, or one NAS glycan acceptor motif as shown in SEQ ID NOS: 49-53. In still further embodiments, GAABCPs comprise 5, 10, 15, or 20 repeating units of amino acid sequences containing a spacer motif of two repeating units of GGS, and either one NNT glycan acceptor motif as shown in SEQ ID NOS: 54-57, or one NNS glycan acceptor motif as shown in SEQ ID NOS: 58-61, or one NGT glycan acceptor motif as shown in SEQ ID NOS: 62-65.

GAABCPs of the present invention are optionally utilized to increase the half-life of any TUP without previous detailed knowledge of the TUPs polypeptide structure/biological activity relationships (or SAR). In some embodiments, a TUP of the present invention is a biologically active polypeptide; and in other instances the TUP is not biologically active. Biological activity may be increased, decreased, or substantially unchanged following conjugation to a GAABCP, according to the present disclosure. In some embodiments, the TUP is of mammalian origin, such as e.g., of human origin. In some embodiments, the TUP is a native polypeptide, and in some embodiments it is a fragment or an amino acid sequence variant of the native polypeptide. Such variants may simply be a native TUP comprising a non-native secretion signal peptide, or such variants may be any other type of variant, e.g., as described in this specification.

In some embodiments, the TUP that is conjugated to the GAABCP, as described herein, is selected from a group consisting of a cytokine, a chemokine, a lymphokine, a ligand, a receptor, a hormone, an apoptosis-inducing polypeptide, an enzyme, an antibody, a proteolytic antibody fragment, an antigen specific antibody fragment, a coagulation factor, a complement protein, a growth factor, and fragments or variants thereof.

In some embodiments, the cytokine TUP is a growth factor selected from the group consisting of human growth hormone, granulocyte macrophage colony stimulating factor (GM-CSF), granulocyte colony stimulating factor (G-CSF), macrophage colony stimulating factor (M-CSF), platelet derived growth factor (PDGF), tumor necrosis factor (TNF), epidermal growth factor (EGF), vascular endothelial growth factor (VEGF), fibroblast growth factor, (FGF), nerve growth factor (NGF), and erythropoietin (EPO).

In some embodiments, the cytokine TUP is selected from the group the group consisting of beta-interferon, gamma-interferon interferon type I, interferon type II and interferon type III. Suitable cytokines further include, but are in no way limited to, cytokines selected from a group consisting of interferon gamma inducing factor I (IGIF), RANTES (regulated upon activation, normal T-cell expressed and presumably secreted), macrophage inflammatory protein-1-alpha (MIP-1-α), macrophage inflammatory protein-1-β (MIP-1-β), glucagon-like peptide 1 (GLP-1), Leishmania elongation initiating factor (LEIF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), and fragments or variants thereof.

In some embodiments, the hormone TUP is selected from the group consisting of insulin, insulin-like growth factor 1 (IGF-1), adrenocorticotropic hormone (ACTH), human growth hormone (hGH), glucagon, glucagon-like peptide 1 (GLP-1), glucagon-like peptide 2 (GLP-2), exendin-4, releasing hormone, thyroid stimulating hormone, melanocyte stimulating hormone, luteinizing hormone, follicle stimulating hormone, prolactin, antidiuretic hormone, oxytocin, calcitonin, thyroid hormone, parathyroid hormone, renin, relaxin-3, relaxin-2, etc., and fragments or variants thereof.

In some embodiments, the TUP is selected from the group consisting of a soluble gp-120 glycoprotein, a soluble gp-160 glycoprotein, an IL-1 receptor antagonist, a TNFalpha receptor antagonist, viral barrier to entry peptides (e.g., enfuvirtide, or the 18-amino acid peptide designated as CL58 that was derived from the CLDN1 intracellular and first transmembrane region that inhibits both de novo and established HCV infection in vitro, human apolipoprotein E peptide, hEP, which specifically blocked the entry of cell culture grown HCV (HCVcc) at submicromolar concentrations), peptides derived from human Fcγ receptor II (CD32) and other FcyR subtypes useful in the treatment of systemic lupus erythematosus or other autoimmune disorders, and fragments or variants thereof.

In some embodiments, the TUP is selected from the group consisting of an antibody, an antigen specific antibody fragment, a proteolytic antibody fragment or domain, a single chain antibody, a genetically or chemically optimized antibody, a proteolytic and antigen-binding fragment thereof, and fragments or variants thereof.

In some embodiments, the TUP is selected from the group consisting of a receptor, the extracellular domain of a receptor, membrane-associated proteins, selected from the group consisting of tumor necrosis factor (TNF) type I receptor, tumor necrosis factor (TNF) type II receptor, IL-1 receptor type II, and IL-4 receptor, CTLA-4, hTRAIL receptor, TACI, VEGFR1/2, Tie-2, lymphocyte function-associated antigen 3, FAS receptor, activin receptor type 2A, B-cell activating factor receptor, fibroblast growth factor receptors, other cytokine and immune cell regulating protein receptors, and fragments or variants thereof.

In certain embodiments, the GAABCP-TUP of the present invention comprises an amino acid sequence that is at least about 90%, 95%, 98%, 99%, or that is 100% identical to any one of the sequences provided as SEQ ID NOS: 97, 98, 103, 104, 146, 147, 157, 164, and 168, or as provided in Table 5.

In certain embodiments, the GAABCP-TUP of the present invention comprises an amino acid sequence that is at least about 90%, 95%, 98%, or 99% identical to any one of the sequences provided as SEQ ID NOS: 99-102, 105-108, and 113-123.

In certain embodiments, the GAABCP-TUP of the present invention comprises an amino acid sequence that identical to any one of the sequences provided as SEQ ID NOS: 99-102, 105-108, and 113-123.

In certain embodiments, the GAABCP-TUP of the present invention comprises or consists of an amino acid sequence that is at least about 90%, 95%, 98%, or 99% identical to any one of the sequences provided as SEQ ID NOS: 99-102, 105-108, and 113-123, 134-140, 141-145, 148-156, 158-163, 165-167, and 169-180.

In certain embodiments, the GAABCP-TUP of the present invention comprises or consists of an amino acid sequence that identical to any one of the sequences provided as SEQ ID NOS: 99-102, 105-108, and 113-123, 134-140, 141-145, 148-156, 158-163, 165-167, and 169-180.

Additionally, the present invention encompasses polynucleotide sequences encoding GAABCP-TUPs; vectors (e.g., plasmids) comprising GAABCP-TUP polynucleotide sequences; host cells (such as various mammalian host cells, including CHO cells, e.g., CHO DG44, CHO-S, CHO-BRI, CHOK1SV, etc.) comprising GAABCP-TUP vectors or polynucleotides; and GAABCP-TUP polypeptides and polypeptide sequences encoding GAABCP-TUPs.

The present invention also provides a method for increasing glycan occupancy on a polypeptide that is a glycoprotein or a glycoprotein conjugate comprising two or more glycan acceptor motifs separated by a spacer motif, said method comprising modifying a construct encoding the glycoprotein or the glycoprotein conjugate by increasing the length of the spacer sequence separating the glycan acceptor sites and expressing the modified glycoprotein or glycoprotein conjugate in a suitable expression system, thereby increasing glycan occupancy on the modified glycoprotein or glycoprotein conjugate as compared to its unmodified form. The present invention also provides a method of increasing the plasma half-life of a GAABCP-TUP, comprising increasing the glycan occupancy of the TUP according to such a method of increasing glycan occupancy.

In some embodiments, the polypeptide that is to be modified is the GAABCP-TUP as disclosed herein.

The present invention further includes a method of increasing the plasma half-life of a TUP, comprising conjugating a GAABCP to the TUP.

Also disclosed herein are methods for designing, producing, purifying, characterizing, and testing bioactivity and various pharmacokinetic properties (including plasma half-life) of the resultant GAABCP-TUPs. The invention also relates to pharmaceutical compositions comprising GAABCP-TUPs, as well as methods for utilizing GAABCP-TUPs as therapeutics for treating or preventing diseases, conditions, and injuries that are treatable with GAABCP-TUPs.

### BRIEF DESCRIPTION OF THE DRAWINGS

FIG. 1 shows cartoon representations of the polypeptide and carbohydrate components of 3 different non-limiting variants of a GAABCP-TUP, where the GAABCP is shown as being comprised of a glycosylation acceptor motif conjugated to between 1 and 99 repeats of a spacer motif and glycosylation acceptor motif. Carbohydrate structures on the glycosylation acceptor motif are shown as bi-antennate complex-type N-glycans utilizing the naming convention shown in the Figure legend. Panel A shows the TUP conjugated to the N-terminus of the GAABCP, panel B shows the TUP conjugated to the C-terminus of the GAABCP, and panel C shows one TUP conjugated to the N-terminus and one TUP conjugated to the C-terminus of the GAABCP.

FIG. 2 shows schematic representations of the polypeptide domain organization for 6 different GAABCP-TUPs, where each unique TUP domain is located N-terminal to the GAABCP domain. For AQB-102 (SEQ ID NO: 100) the TUP is a variant of human granulocyte-colony stimulating factor (modified hG-CSF; SEQ ID NO: 98) with a Pho1 secretion signal peptide (SEQ ID NO: 66). For AQB-304 (SEQ ID NO: 106) the TUP is a variant of human glucagon-like peptide (hGLP-1: SEQ ID NO: 104) with a Pho1 secretion signal peptide (SEQ ID NO: 66). For AQB-203a (SEQ ID NO: 113) the TUP is a variant of human prorelaxin-2 (SEQ ID NO: 110) with the B-chain/modified C-chain/A-chain organization shown with its native secretion signal peptide. For AQB-401a (SEQ ID NO: 179) the TUP is a variant of human erythropoietin (hEPO: SEQ ID NO: 147) with its native secretion signal peptide. For AQB-501a (SEQ ID NO: 180) the TUP is human adrenocorticotropic hormone (hACTH: SEQ ID NO: 157) with a Pho1 secretion signal peptide (SEQ ID NO: 66). For AQB-601 (SEQ ID NO: 169) the TUP is human coagulation factor IX (hFIX: SEQ ID NO: 168) with its native secretion signal peptide. All six TUPs are conjugated to the N-termini of the GAABCP sequence GGSGGSNNT repeated 10 times (SEQ ID NO: 55). All these GAABCP-TUPs have a 6×His-tag conjugated at the C-termini (SEQ ID NO: 68), with the presence or absence of an intervening flexible linker (SEQ ID NO: 73).

FIG. 3 shows schematic representations of the polypeptide domain organization for 7 different GAABCP-TUP constructs, where each unique GAABCP is located on the C-termini of a variant of hG-CSF (modified hG-CSF; SEQ ID NO: 98) with a Pho1 secretion signal peptide (SEQ ID NO: 66). For AQB-114 (SEQ ID NO: 134) the GAABCP is GGSGGSNNT repeated 5 times (SEQ ID NO: 54). For AQB-115 (SEQ ID NO: 135) the GAABCP is GGSGGSNNT repeated 10 times (SEQ ID NO: 55). For AQB-116 (SEQ ID NO: 136) the GAABCP is GGSGGSNNT repeated 15 times (SEQ ID NO: 56). For AQB-117 (SEQ ID NO: 137) the GAABCP is GGSGGSNNT repeated 20 times (SEQ ID NO: 57). For AQB-118 (SEQ ID NO: 138) the GAABCP is GGSNNT repeated 10 times (SEQ ID NO: 181). For AQB-119 (SEQ ID NO: 139) the GAABCP is GGSGGSGGSNNT repeated 10 times (SEQ ID NO: 182). For AQB-120 (SEQ ID NO: 140) the GAABCP is GGSGGSGGSGGSNNT repeated 10 times (SEQ ID NO: 183).

FIG. 4 shows schematic representations of the polypeptide domain organization for 7 different GAABCP-TUP constructs, where each unique GAABCP is located on the C-termini of a variant of hGLP-1 (modified hGLP-1; SEQ ID NO: 104) with a Pho1 secretion signal peptide (SEQ ID NO: 66). For AQB-303 (SEQ ID NO: 105) the GAABCP is GGSGGSNNT repeated 5 times (SEQ ID NO: 54). For AQB-304 (SEQ ID NO: 106) the GAABCP is GGSGGSNNT repeated 10 times (SEQ ID NO: 55). For AQB-301 (SEQ ID NO: 107) the GAABCP is GGSGGSNNT repeated 15 times (SEQ ID NO: 56). For AQB-305 (SEQ ID NO: 108) the GAABCP is GGSGGSNNT repeated 20 times (SEQ ID NO: 57). For AQB-309 (SEQ ID NO: 143) the GAABCP is GGSGGSGGSNIT repeated 25 times (SEQ ID NO: 184). For AQB-310 (SEQ ID NO: 144) the GAABCP is GGSGGSGGSGGSNIT repeated 15 times (SEQ ID NO: 191). For AQB-311 (SEQ ID NO: 145) the GAABCP is GGSGGSGGSGGSGGSNIT repeated 15 times (SEQ ID NO: 192). All these GAABCP-TUPs have a 6×His-tag conjugated at the C-termini (SEQ ID NO: 68), with the presence or absence of an intervening flexible linker (SEQ ID NO: 73).

FIG. 5 shows schematic representations of the polypeptide domain organization for 4 different GAABCP-TUP constructs, where each unique GAABCP is located on the N-termini of native hProRNL2 (SEQ ID NO: 109). For AQB-236 (SEQ ID NO: 114) the GAABCP is GGSGGSNNT repeated 5 times (SEQ ID NO: 54). For AQB-237 (SEQ ID NO: 115) the GAABCP is GGSGGSNNT repeated 10 times (SEQ ID NO: 55). For AQB-238 (SEQ ID NO: 116) the GAABCP is GGSGGSNNT repeated 15 times (SEQ ID NO: 56). For AQB-238a (SEQ ID NO: 117) the GAABCP is GGSGGSNNT repeated 20 times (SEQ ID NO: 57). All these GAABCP-TUPs have a 6×His-tag (SEQ ID NO: 68) conjugated to the N-terminus and a GGGGSGGGGS linker (SEQ ID NO: 93) conjugated to the C-terminus of the GAABCP domain, and the native human prorelaxin-2 signal peptide located at the N-terminus of the polypeptide.

FIG. 6 shows schematic representations of the polypeptide domain organization for 3 different GAABCP-TUP constructs, where the same GAABCP, GGSGGSNNT repeated 10 times (SEQ ID NO: 55), is conjugated at the N-terminus, the C-terminus and internal to the structure of variants of human prorelaxin-2. AQB-210 (SEQ ID NO: 118) has the GAABCP located on the N-termini of a variant of human prorelaxin-2 (SEQ ID NO: 111). AQB-211 (SEQ ID NO: 119) has the GAABCP located between the B and A-chains of native human prorelaxin-2 (SEQ ID NO: 109), in place of the native relaxin C chain, which is deleted. Directly N-terminal to the GAABCP is a 6×His tag (SEQ ID NO: 68) connecting the C terminus of the B chain to the GAABCP, and directly C-terminal to the GAABCP is a GGSGGS linker sequence (SEQ ID NO: 73) connecting the GAABCP to the N-terminus of the A chain. AQB-211a (SEQ ID NO: 120) has the GAABCP located on the C-termini of a variant of human prorelaxin-2 (SEQ ID NO: 111).

FIG. 7 shows schematic representations of the polypeptide domain organization for 5 different GAABCP-TUP constructs, where the GAABCP domain is conjugated to two distinct TUPS. The AQB-110 polypeptide (SEQ ID NO: 121) has the following domain structure from N- to C-terminus; Pho1 signal peptide (SEQ ID NO: 66), a variant of hG-CSF (SEQ ID NO: 98), GGSGGSNNT repeated 5 times (SEQ ID NO: 54), a (GGGGS)₂ linker (SEQ ID NO: 93), and the partial Fc domain of IgG1 (SEQ ID NO: 112). The AQB-255 polypeptide (SEQ ID NO: 122) has the following domain structure from N- to C-terminus; signal peptide from native human prorelaxin-2 (SEQ ID NO: 109), the partial Fc domain of IgG1 (SEQ ID NO: 112), GGSGGSNNT repeated 5 times (SEQ ID NO: 54), a (GGGGS)₂ linker (SEQ ID NO: 93), and native human prorelaxin-2 (SEQ ID NO: 109). The AQB-263 polypeptide (SEQ ID NO: 123) has the following domain structure from N- to C-terminus; signal peptide from native human prorelaxin-2 (SEQ ID NO: 109), GGSGGSNNT repeated 5 times (SEQ ID NO: 54), a (GGGGS)₂ linker (SEQ ID NO: 93), the partial Fc domain of IgG1 (SEQ ID NO: 112), and native human prorelaxin-2 (SEQ ID NO: 109). The AQB-701 polypeptide (SEQ ID NO: 177) has the following domain structure from N- to C-terminus; Pho1 signal peptide (SEQ ID NO: 66), modified sequence from anti-hCD19 scFv, GGSGGSNNT repeated 5 times (SEQ ID NO: 54), a (GGGGS)₂ linker (SEQ ID NO: 93), a modified sequence from anti-hCD3 scFv, and a 6×His-tag (SEQ ID NO: 68). The AQB-702 polypeptide (SEQ ID NO: 178) has the following domain structure from N- to C-terminus; Pho1 signal peptide (SEQ ID NO: 66), modified sequence from anti-hCD19 scFv, GGSGGSNNT repeated 5 times (SEQ ID NO: 54), modified sequence from anti-hCD3 scFv, and a 6×His-tag (SEQ ID NO: 68).

FIG. 8 shows schematic representations of the polypeptide domain organization for 7 different GAABCP-TUP constructs, where each unique GAABCP is located on the C-termini of a variant of human erythropoietin (modified hEPO; SEQ ID NO: 147) with its native secretion signal peptide. For AQB-401 (SEQ ID NO: 148) the GAABCP is GGSGGSGGSNNT repeated 10 times (SEQ ID NO: 182). For AQB-404 (SEQ ID NO: 151) the GAABCP is GGSGGSGGSGGSNNT repeated 10 times (SEQ ID NO: 183). For AQB-405 (SEQ ID NO: 152) the GAABCP is GGSGGSGGSGGSGGSNNT repeated 10 times (SEQ ID NO: 187). For AQB-406 (SEQ ID NO: 153) the GAABCP is GGSGGSGGSGGSGGSGGSNNT repeated 10 times (SEQ ID NO: 188. For AQB-407 (SEQ ID NO: 154) the GAABCP is GGSGGSGGSNNT repeated 5 times (SEQ ID NO: 182). For AQB-408 (SEQ ID NO: 155) the GAABCP is GGSGGSGGSNNT repeated 15 times (SEQ ID NO: 197). For AQB-409 (SEQ ID NO: 156) the GAABCP is GGSGGSGGSNNT repeated 20 times (SEQ ID NO: 198). All these GAABCP-TUPs have a 6×His-tag conjugated at the C-termini (SEQ ID NO: 68) with the presence of an intervening flexible linker (SEQ ID NO: 73).

FIG. 9 shows schematic representations of the polypeptide domain organization for 6 different GAABCP-TUP constructs, where each unique GAABCP is located on the C-termini of a variant of human adrenocorticotropic hormone (hACTH; SEQ ID NO: 157) with a Pho 1 secretion signal peptide (SEQ ID NO: 66). For AQB-501 (SEQ ID NO: 158) the GAABCP is GGSGGSGGSGGSNNT repeated 10 times (SEQ ID NO: 183). For AQB-502 (SEQ ID NO: 159) the GAABCP is GGSGGSGGSGGSNNT repeated 15 times (SEQ ID NO: 185). For AQB-503 (SEQ ID NO: 160) the GAABCP is GGSGGSGGSGGSNNT repeated 20 times (SEQ ID NO: 199). For AQB-504 (SEQ ID NO: 161) the GAABCP is GGSGGSGGSGGSNNT repeated 25 times (SEQ ID NO: 200). For AQB-505 (SEQ ID NO: 162) the GAABCP is GGSGGSGGSNNT repeated 15 times (SEQ ID NO: 197). For AQB-506 (SEQ ID NO: 163) the GAABCP is GGSGGSGGSGGSGGSNNT repeated 15 times (SEQ ID NO: 186). All these GAABCP-TUPs have a 6×His-tag conjugated at the C-termini (SEQ ID NO: 68) with the presence of an intervening flexible linker (SEQ ID NO: 73).

FIG. 10 shows schematic representations of the polypeptide domain organization for 3 different GAABCP-TUP constructs, where each unique GAABCP is located on the C-termini of a variant of human proopiomelanocortin [hPOMC(138-241); (SEQ ID NO: 164)] with a Pho 1 secretion signal peptide (SEQ ID NO: 66). For AQB-507 (SEQ ID NO: 165) the GAABCP is GGSGGSGGSNNT repeated 15 times (SEQ ID NO: 197). For AQB-508 (SEQ ID NO: 166) the GAABCP is GGSGGSGGSGGSNNT repeated 15 times (SEQ ID NO: 185). For AQB-509 (SEQ ID NO: 167) the GAABCP is GGSGGSGGSGGSGGSNNT repeated 15 times (SEQ ID NO: 186). All these GAABCP-TUPs have a 6×His-tag conjugated at the C-termini (SEQ ID NO: 68) with the presence of an intervening flexible linker (SEQ ID NO: 73).

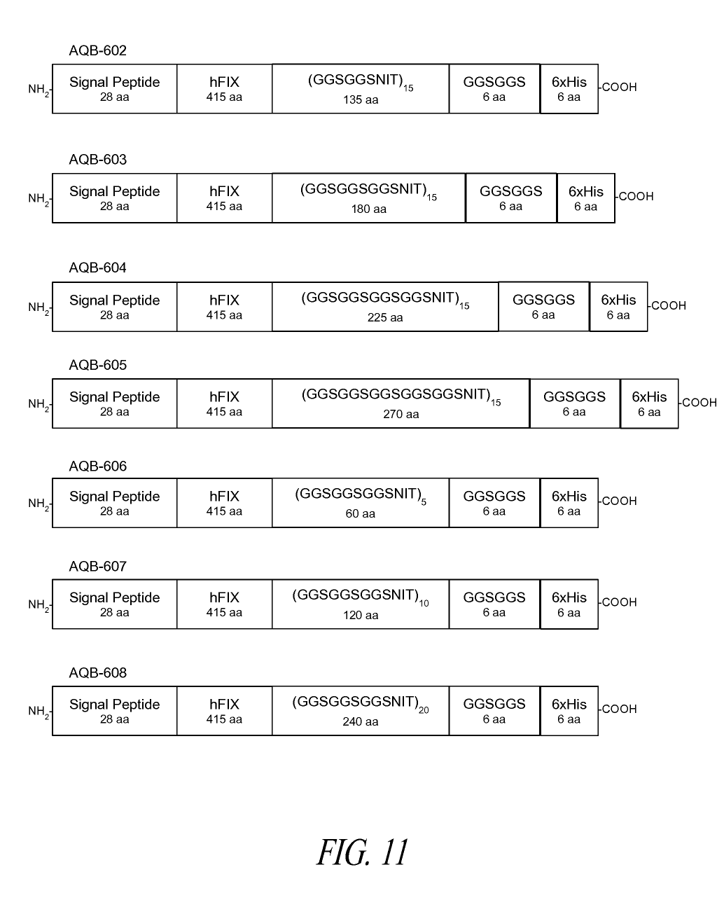

FIG. 11 shows schematic representations of the polypeptide domain organization for 7 different GAABCP-TUP constructs, where each unique GAABCP is located on the C-termini of human coagulation factor IX (hFIX; SEQ ID NO: 168) with its native secretion signal peptide. For AQB-602 (SEQ ID NO: 170) the GAABCP is GGSGGSNIT repeated 15 times (SEQ ID NO: 189). For AQB-603 (SEQ ID NO: 171) the GAABCP is GGSGGSGGSNIT repeated 15 times (SEQ ID NO: 190). For AQB-604 (SEQ ID NO: 172) the GAABCP is GGSGGSGGSGGSNIT repeated 15 times (SEQ ID NO: 191). For AQB-605 (SEQ ID NO: 173) the GAABCP is GGSGGSGGSGGSGGSNIT repeated 15 times (SEQ ID NO: 192). For AQB-606 (SEQ ID NO: 174) the GAABCP is GGSGGSGGSNIT repeated 5 times (SEQ ID NO: 201). For AQB-607 (SEQ ID NO: 175) the GAABCP is GGSGGSGGSNIT repeated 10 times (SEQ ID NO: 202). For AQB-608 (SEQ ID NO: 176) the GAABCP is GGSGGSGGSNIT repeated 20 times (SEQ ID NO: 203). All these GAABCP-TUPs have a 6×His-tag conjugated at the C-termini (SEQ ID NO: 68) with the presence of an intervening flexible linker (SEQ ID NO: 73).

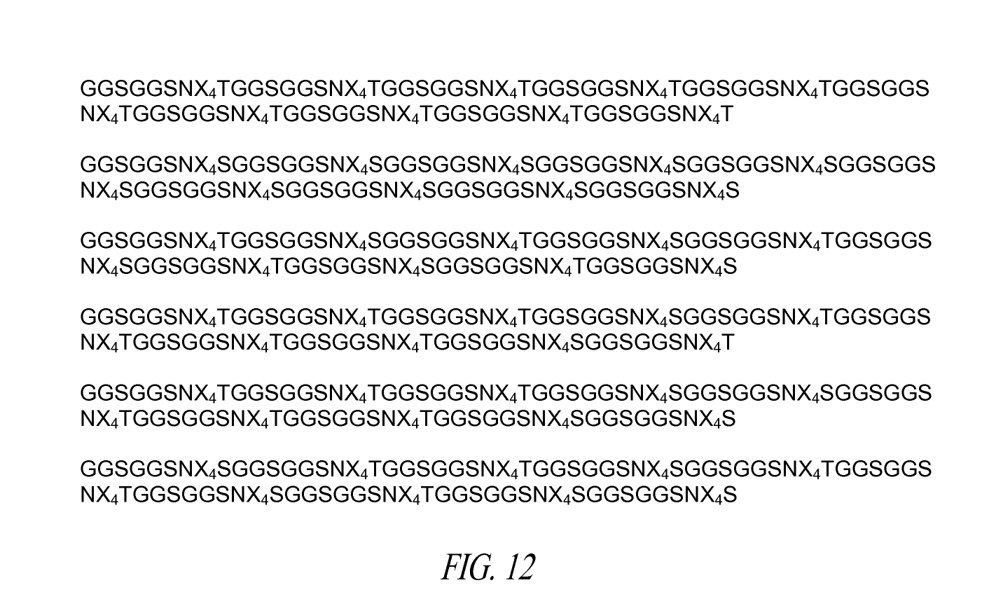

FIG. 12 shows schematic representations of 6 unique examples of GAABCPs, where the glycosylation acceptor motif amino acid sequence is either NX₄T or NX₄S, as defined herein, and the spacer motif amino acid sequence is held constant at GGSGGS (SEQ ID NO: 16) and the GAABCP repeat length at 10. The ratio of the NX₄T or NX₄S glycosylation acceptor motifs occur at differing frequencies in each construct.

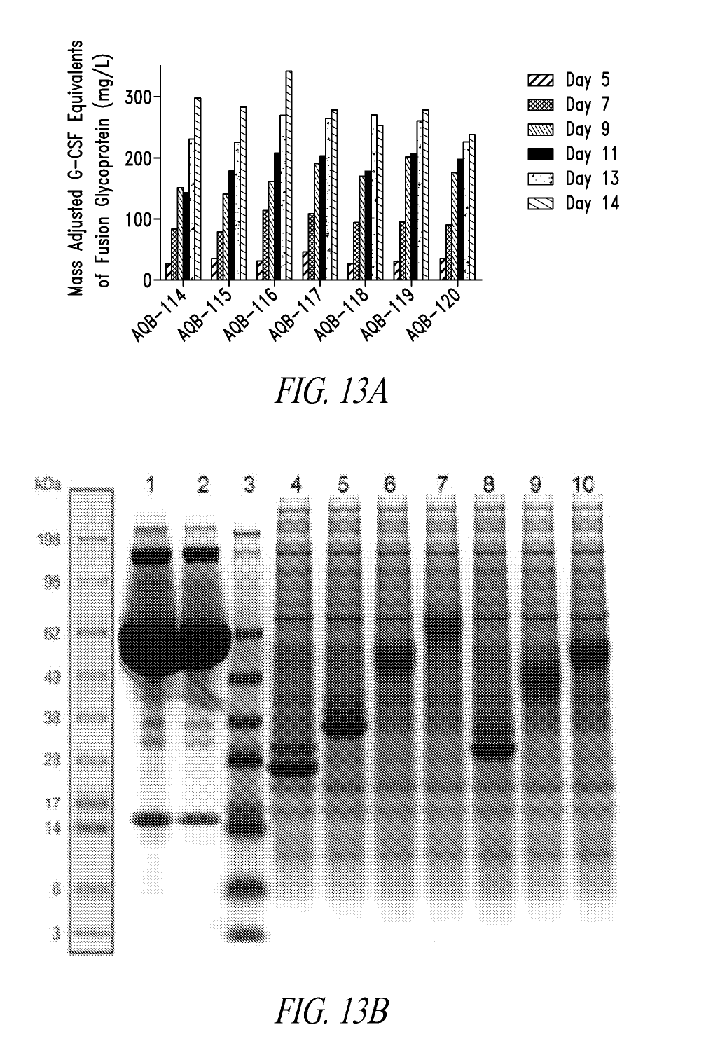

FIG. 13 shows SDS-PAGE and anti-TUP ELISA analysis of the recombinant expression of 7 variant GAABCP-TUPs in CHO cell pools. Synthetic genes for AQB-114 thru 120 (SEQ ID NOS: 134-140; also see FIG. 2 for domain structures) were cloned and stably expressed in CHOK1 SV GS-KO cell pools as described. These 7 individual cell pools were then cultured for 14 days, samples harvested on days 5, 7, 9, 11, 13 & 14, and the levels of fusion glycoprotein was quantitated using an anti-hG-CSF ELISA (R&D Systems) according to the manufacturer's instructions. Panel A shows the mass adjusted concentration of AQB-114 thru 120 fusion glycoproteins over time in culture. As expected the cultures show an accumulation of recombinant fusion glycoprotein over time. Panel B shows a SDS-PAGE analysis of the conditioned media at day 14. A 4-12% Bis-Tris Novex gel with MES buffer (Invitrogen) was run under reducing conditions and stained with InstantBlue (Expedeon). Lane 1 & 2: control antibody at 1 mg/mL. Lane 3: SeeBlue Plus 2 molecular weight standard (also to extreme left). Lane 4: AQB-114 d14 conditioned media. Lane 5: AQB-115 d14 conditioned media. Lane 6: AQB-116 d14 conditioned media. Lane 7: AQB-117 d14 conditioned media. Lane 8: AQB-118 d14 conditioned media. Lane 9: AQB-119 d14 conditioned media. Lane 10: AQB-120 d14 conditioned media.

FIG. 14 shows SDS-PAGE/Coomassie blue stain analysis of 7 purified variants of a GAABCP-TUPs with/without (+/−) digestion with PNGase F. Lanes 1 & 16: molecular weight standards. Lane 2: AQB-114 untreated. Lane 3: AQB-114 treated. Lane 4: AQB-115 untreated. Lane 5: AQB-115 treated. Lane 6: AQB-116 untreated. Lane 7: AQB-116 treated. Lane 8: AQB-117 untreated. Lane 9: AQB-117 treated. Lane 10: AQB-118 untreated. Lane 11: AQB-118 treated. Lane 12: AQB-119 untreated. Lane 13: AQB-119 treated. Lane 14: AQB-120 untreated. Lane 15: AQB-120 treated. Brackets highlight the position of the polypeptide conjugates.

FIG. 15 shows in vivo pharmacokinetic and pharmacodynamic analysis of AQB-114 thru 120 (SEQ ID NOS: 134-140; also see FIG. 2 for domain structures) in mice. Panel A: Mice were administered a single intravenous dose of equimolar amounts of the purified AQB-114 thru 120 fusion glycoproteins (see FIG. 2) plus hG-CSF (Neupogen; Amgen) and PEG-hG-CSF (Neulasta; Amgen). At the indicted times blood samples were harvested and the plasma concentration of the various test articles was quantitated using an hG-CSF ELISA (R&D Systems). The calculated pharmacokinetic parameters can be seen in Table 7. Panel B: Mice were administered a single subcutaneous dose of equimolar amounts of the purified AQB-115, 117, 119, 120 and PEG-hG-CSF (Neulasta; Amgen). At the indicated times blood samples were harvested and the absolute neutrophil counts (ANC) were determined using an Advia 120 an automated hematology analyzer (Siemens).

FIG. 16 shows SDS-PAGE and Western blot analysis of purified AQB-301, 304 and 305 fusion glycoproteins with/without (+/−) digestion with PNGase F to remove the N-glycan carbohydrate chains from the polypeptide. Panel A shows coomassie stained SDS-PAGE analysis. Lanes 1 & 25: molecular weight standards. Lanes 2 and 3: AQB-301−/+PNGase F treatment. Lanes 8 and 9: AQB-304−/+PNGase F treatment. Lanes 16 and 17: AQB-305−/+PNGase F treatment. Lanes 22 and 23: alpha1-anti trypsin control−/+PNGase F treatment. Lane 24: PNGase F control (note the migration of this band in all PNGase F treatment lanes). Panel B shows Western blot analysis of several GLP-1 based GAABCP-TUP probed with an anti-GLP-1 primary antibody (EMD Millipore) and a horseradish peroxidase conjugated secondary antibody (electrochemical detection was employed). Lanes 2 & 26: molecular weight standards. Lanes 3 and 4: AQB-301−/+PNGase F treatment. Lane 9 and 10: AQB-304−/+PNGase F treatment. Lanes 15 and 16: AQB-305−/+PNGase F treatment. Lane 25: hGLP-1 control. Brackets highlight the position of the polypeptide conjugates.

FIG. 17 shows western blot analysis of conditioned media from CHO-3E7 cell cultures transiently transfected with AQB-307 thru 311 fusion glycoprotein genes. Synthetic genes for AQB-307 thru 311 (SEQ ID NOS: 141-145; also see FIG. 3 for domain structures) were cloned and transiently expressed in CHO-3E7 cell cultures as described. These 5 individual cell pools were then cultured for 8 days, the culture media harvested, and the recombinant fusion glycoproteins were identified via Western blot analysis using and anti-GLP-1 antibody (EMD Millipore). Lane 1: molecular weight standards. Lane 2: hGLP-1 control. Lanes 3 & 4: replicates of AQB-307. Lanes 5 & 6: replicates of AQB-308. Lanes 7 & 8: replicates of AQB-309. Lanes 9 & 10: replicates of AQB-310. Lanes 11 & 12: replicates of AQB-311.

FIG. 18 shows western blot analysis of conditioned media from CHO-3E7 cell cultures transiently transfected with AQB-401 thru 409 fusion glycoprotein genes. Synthetic genes for AQB-401 thru 409 (SEQ ID NOS: 148-156; also see FIG. 7 for domain structures) were cloned and transiently expressed in CHO-3E7 cell cultures as described. These 9 individual cell pools were then cultured for 8 days, the culture media harvested, and the recombinant fusion glycoproteins were identified via Western blot analysis using and anti-hEPO-1 antibody (Santa Cruz Biotechnology). Lane 1: molecular weight standards. Lane 2: AQB-401. Lane 3: AQB-402. Lane 4: AQB-403. Lane 5: AQB-404. Lane 6: AQB-405. Lane 7: AQB-406. Lane 8: AQB-407. Lane 9: AQB-408. Lane 10: AQB-409.

FIG. 19 shows western blot analysis of recombinantly expressed GAABCP-TUPs. The genes for the AQB-210 and 211 polypeptides (SEQ ID NOS: 118 and 119) were recombinantly expressed in CHO-S cells, and then a sample of the conditioned media was electrophoresised, blotted and probed with an anti-His tag antibody (R&D Systems); the lower MW band corresponds to the expected MW of the naked polypeptide and the higher MW bands are N-glycosylated forms. His-tagged stds lane is BenchMark™ His-tagged Protein Standard (Life Technology, LC5606). His-tag control lane is recombinant Lipopolysaccharide Binding Protein (Life Technology, 10526-H08H-5).

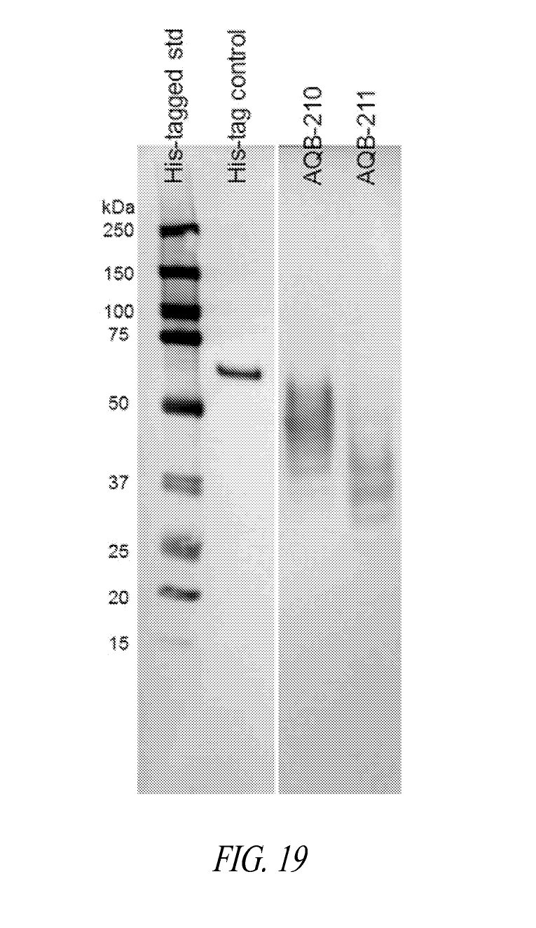

### DETAILED DESCRIPTION OF THE INVENTION

The practice of the present invention will employ, unless indicated specifically to the contrary, conventional methods of molecular biology, recombinant DNA techniques, protein expression, and protein/peptide/carbohydrate chemistry within the skill of the art, many of which are described below for the purpose of illustration. Such techniques are explained fully in the literature. See, e.g., Sambrook, et al., Molecular Cloning: A Laboratory Manual (3^{rd} Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications. The publications discussed above are provided solely for their disclosure before the filing date of the present application. Nothing herein is to be construed as an admission that the invention is not entitled to antedate such disclosure by virtue of prior invention.

### DEFINITIONS AND ABBREVIATIONS

Unless defined otherwise, all technical and scientific terms used herein have the same meaning as commonly understood by one of ordinary skill in the art to which this invention belongs. As used in the specification and appended claims, unless specified to the contrary, the following terms have the meaning indicated. With regard to this specification, any time a definition of a term as defined herein, differs from a definition given for that same term in an incorporated reference, the definition explicitly defined herein is the correct definition of the term.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

The term “adjacent” or “directly adjacent” when used herein with reference to the location of an inserted polypeptide extension, or a component of an inserted polypeptide extension, is meant to refer to a location that is not separated by any other amino acids. Thus e.g., but not to be limited by example, reference to a GAABCP that is directly adjacent to a His-tag, means that a junction exists between a terminal amino acid of the GAABCP and a terminal amino acid of the His-tag, and in this scenario, no other amino acids may separate the two adjacent components; conversely, “not directly adjacent” as used herein is meant to describe the scenario where at least one amino acid separates the two such components, and thus no such junction is formed.

As used herein, the term “amino acid” is intended to mean both naturally-occurring and non-naturally-occurring amino acids, as well as amino acid analogs and chemical mimetics. Naturally-occurring amino acids include the 20 (L)-amino acids utilized during protein biosynthesis as well as other amino acids formed during post-translational modification such as, for example, 4-hydroxyproline, hydroxylysine, desmosine, isodesmosine, homocysteine, citrulline and ornithine. Non-naturally-occurring amino acids include, for example, (D)-amino acids, norleucine, norvaline, p-fluorophenylalanine, ethionine and the like, which are known to a person skilled in the art. Amino acid analogs include modified forms of naturally and non-naturally-occurring amino acids. Such modifications can include, for example, substitution or replacement of chemical groups and moieties on the amino acid or by derivatization of the amino acid. Amino acid mimetics include, for example, organic structures that exhibit functionally similar properties such as charge and charge spacing characteristic of the reference amino acid. For example, an organic structure which mimics Arginine (Arg or R) would have a positive charge moiety located in similar three-dimensional molecular space and having the same degree of mobility as the s-amino group of the side chain of the naturally-occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

The term “antibody” as used herein is meant to have its normal scientific meaning, i.e., a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (ν), delta (δ), gamma (γ), sigma (ε), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. The term “antibody” includes antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory.

The term “block copolymer” is used herein to refer to a molecule (e.g. a polypeptide) comprising alternating elements (e.g., glycosylation motifs and spacer motifs). In various embodiments, the block copolymer of the present invention is a polypeptide comprising repeating amino acid sequences, forming glycan acceptor motifs, separated by amino acid sequences, forming spacer motifs.

In some embodiments, the term “block unit” is used to refer to a repeating unit that is present in a GAABCP. Thus, a block unit of the present invention is a polypeptide comprising at least one spacer motif fused to a glycan acceptor motif, wherein the block unit sequence is repeated throughout the GAABCP. In some embodiments, a block unit comprises multiple alternating glycan acceptor and spacer motifs, thus, a GAABCP can be a block unit in some embodiments. For example, in one such embodiment, but not to be limited in any way, a sequence such as follows: GGSGGSNNTGGSGGSNNT-GGSGGSNITGGSGGSNITGGSGGSNITGGSGGSNNTGGSGGSNNTGGSGGSNITGGSG GSNITGGSGGSNIT (SEQ ID NO: 196), comprises 2 block units each repeated once (the separate block units are shown via italics and underline), wherein each block unit itself is a GAABCP according to the present disclosure. Accordingly, in some embodiment the term GAABCP and block unit are used interchangeably, however, as is clear based upon the specification, a block unit may optionally comprise as few as one glycan acceptor motif and one spacer motif, whereas all GAABCPs comprise at least 2 glycan acceptor motifs separated by a spacer motif.

A “composition” can comprise an active agent, e.g., any GAABCP-TUP and/or any unmodified TUP, and a carrier, inert or active, e.g., a pharmaceutically acceptable carrier, diluent or excipient. In particular embodiments, the compositions are sterile, substantially free of endotoxins or non-toxic to recipients at the dosage or concentration employed.

Unless the context requires otherwise, throughout the present specification and claims, the word “comprise” and variations thereof, such as, “comprises” and “comprising” are to be construed in an open and inclusive sense, that is, as “including, but not limited to”.

By “consisting of” is meant including, and limited to, whatever follows the phrase “consisting of” Thus, the phrase “consisting of” indicates that the listed elements are required or mandatory and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “biological activity” or “bioactivity” refers to any response induced in an in vitro assay or in a cell, tissue, organ, or organism, (e.g., an animal, or a mammal, or a human) as the result of administering any compound, agent, polypeptide, conjugate, pharmaceutical composition, or any GAABCP or therapeutically useful polypeptide contemplated herein. Biological activity may refer to agonistic actions or antagonistic actions. The biological activity may be a beneficial effect; or the biological activity may not be beneficial, i.e. a toxicity. In some embodiments, biological activity will refer to the positive or negative effects that a drug or pharmaceutical composition (e.g. a pharmaceutical composition comprising a TUP and/or GAABCP-TUP, as herein described) has on a living subject, e.g., a mammal such as a human. Accordingly, the term “biologically active” is meant to describe any compound possessing biological activity, as herein described. Biological activity may be assessed by any appropriate means currently know to the skilled artisan. Such assays may be qualitative or quantitative. The skilled artisan will readily appreciate the need to employ different assays to assess the activity of different polypeptides; a task that is routine for the average researcher. Such assays are often easily implemented in a laboratory setting with little optimization requirements, and more often then not, commercial kits are available that provide simple, reliable, and reproducible readouts of biological activity for a wide range of polypeptides using various technologies common to most labs. When no such kits are available, ordinarily skilled researchers can easily design and optimize in-house bioactivity assays for target polypeptides without undue experimentation; as this is a routine aspect of the scientific process. Some non-limiting examples of such assays include, but are in no way limited to assays selected from the group consisting of: cell proliferation assays, cellular apoptosis assays, cellular chemotaxis or migration assays, receptor binding assays, receptor activation assays, enzyme assays, functional ELISAs, reporter gene assays, antiviral assays, etc. The term “similar biological activity” is intended herein to refer to any biological activity that is within one order of magnitude of the biological activity with which it is compared, and in some embodiments includes a biological activity that is about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, or 9.9 (including any integer or decimal point in between 1 and 10) fold higher than the biological activity with which it is compared (this can optionally be called a “minimal gain in biological activity”); or in other embodiments it includes a biological activity that is about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, or 9.9 (including any integer or decimal point in between 1 and 10) fold lower than the biological activity with which it is compared (This can optionally be called a “minimal loss of biological activity”). In some embodiments, the biological activity of two or more polypeptides (e.g, a GAABCP-TUP and the corresponding unmodified TUP) are compared and found to be similar. Similarity with regard to biological activity may be assessed by any appropriate readout; and in some embodiments similar activity means induction of similar quantities of any molecular species, e.g., a second messenger such as cAMP. In some embodiments, similar biological activity refers to a similar EC50 for a response, i.e. an EC50 within one order of magnitude of the EC50 with which it is compared. “Loss of biological activity” refers to the occasion wherein a GAABCP conjugated to a TUP renders the GAABCP-TUP not active with regard to the biological activity of the unmodified TUP. For example, without being limited by example, in one embodiment a unmodified granulocyte colony stimulating factor (G-CSF) polypeptide is considered to be therapeutically useful because it is able to induce cellular proliferation in cells of myeloid origin; and if the genetic conjugation of that unmodified G-CSF polypeptide with a GAABCP results in a GAABCP-G-CSF that cannot induce any detectable response in proliferation in the same myeloid cell line under the same conditions, then the modification will have resulted in loss of biological activity.

Reference throughout this specification to a “conjugate” or “conjugated to” or “conjugations” refers to any polypeptide, e.g. a TUP as described herein, which is bound to another substance, e.g., but not to be limited by example in any way, a TUP bound singularly, or in combination to a GAABCP, a flexible linker, a different polypeptide, another molecule of the identical polypeptide, or a non-proteinaceous conjugate. Conjugates may be formed in any suitable manner, such as, e.g., via genetic conjugation or posttranslational chemical conjugation. In some embodiments, the two or more substances bound together in the polypeptide conjugate comprise a TUP and a GAABCP. In some embodiments, the two or more substances bound together in the polypeptide conjugate comprise a GAABCP-TUP and at least a portion of another TUP, such as, e.g., one selected from the group consisting of a cytokine, a chemokine, a lymphokine, a ligand, a soluble receptor, a hormone, an apoptosis inducing polypeptide, a therapeutic enzyme, an antibody or antibody fragment, and a growth factor. In some embodiments the polypeptide conjugate comprises a polypeptide and a non-proteinaceous conjugate, e.g. a glycan, a non-proteinaceous linker, or a polymer, e.g. a polyethylene glycol (PEG) chain, or a derivative thereof, and in other embodiments the polypeptide conjugate comprises both proteinaceous and non-proteinaceous conjugates. “Genetic conjugation” as used herein refers to the in-frame fusion of two or more polynucleotide sequences encoding two or more polypeptides using recombinant DNA methodology, such that the resulting polypeptide fusion comprises both polypeptides in one continuous fusion polypeptide. “Chemical conjugation” as used herein refers to the chemical attachment of any two or more substances using chemical synthesis methodology. In some embodiments, chemical conjugation refers to the conjugation of a non-proteinaceous substance to a polypeptide. In some embodiments, chemical conjugation refers to the conjugation of two or more polypeptides, such that the resulting polypeptide fusion comprises both polypeptides in one continuous fusion polypeptide. In particular embodiments, chemical conjugation refers to the conjugation of a TUP to a GAABCP via chemical synthesis.

Reference to “domains” with regard to a GAABCP-TUP construct means a section of the sequence of a GAABCP-TUP that represents a functional or structural unit within the polypeptide or polynucleotide. Such domains include, but are not limited to sequences encoding TUPs, GAABCPs, linkers, affinity tags, secretion signal sequences, etc.

Reference to the term “e.g.” is intended to mean “e.g., but not limited to” and thus it should be understood that whatever follows is merely an example of a particular embodiment, but should in no way be construed as being a limiting example. Unless otherwise indicated, use of “e.g.” is intended to explicitly indicate that other embodiments have been contemplated and are encompassed by the present invention.

Reference throughout this specification to “embodiment” or “one embodiment” or “an embodiment” or “some embodiments” or “certain embodiments” means that a particular feature, structure or characteristic described in connection with the embodiment is included in at least one embodiment of the present invention. Thus, the appearances of the phrases “in one embodiment” or “in an embodiment” or “in certain embodiments” in various places throughout this specification are not necessarily all referring to the same embodiment. Furthermore, the particular features, structures, or characteristics may be combined in any suitable manner in one or more embodiments.

In some embodiments, the term “extended” or “extension” or “inserted” or “insertion” is intended to indicate purposeful positioning within the amino acid sequence of the TUP. In some embodiments, insertion or inserted may therefore relate to the construct design of the GAABCP-TUP gene, and not an accidental product of post-translation polypeptide processing or degradation. In some embodiments, “insertion” or “inserted” encompasses the replacement of at least a portion of the TUP sequence; in other embodiments no TUP sequence is replaced by the inserted GAABCP. All of these terms may be used interchangeably, and thus an insertion (or extension) may be made within the internal sequence of a TUP, or at one or both of its termini.

The term “Fc”, “Fc region”, or “FC portion”, etc. is used herein to refer to a portion of an antibody as defined herein that includes a polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and optionally, at least a portion of the flexible hinge N-terminal to these domains. For IgA and IgM Fc may include the J chain. For IgG, Fc comprises immunoglobulin domains Cgamma2 and Cgamma3 (Cγ2 and Cγ3) and optionally at least a portion of the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an antibody, antibody fragment, or Fc fusion. An Fc may be an antibody, Fc fusion, or a protein or protein domain that comprises Fc. In some embodiments, Fc refers to the region comprising a partial hinge sequence beginning with D221 (according to the EU index as in Kabat) and extending to the carboxy-terminus, thus further comprising a CH2 and CH3 domain. “Fc” also refers to Fc variants, as described herein, including, but not limited to aglycosylated Fc portions such as e.g., Fc portions comprising one or more mutation to or near the N-linked glycosylation motif beginning at N297 (according to the EU index as in Kabat) which leads to a reduction or block in the N-glycosylation of this site.

The term “flank” or “flanked” or “flanking” as used herein is meant to describe the surrounding of something; i.e. the phrase “the GAABCP is flanked by linkers” would mean that the GAABCP is surrounded by linkers, or more specifically, that linkers comprised in a polypeptide are positioned both N-terminally and C-terminally to a GAABCP domain.

The term “fragment” as used herein is meant to describe a portion of a native therapeutically useful polypeptide that is generated by any number of physiochemical means, including proteolytic fragments (e.g., generated by treatment of the native TUP with an enzyme or other suitable catalyst), chemical fragments (e.g., generated by treatment of the native TUP with chemical agents and altered physiochemical conditions), and expression of partial sequences of native polypeptides (i.e., generated by recombinant expression of a portion of the native TUP sequence).

The term “GAABCP” is used herein to describe the glycosylated amino acid block copolymer-based polypeptides of the present disclosure, and thus GAABCP is meant to have the meaning described throughout this specification. Loosely defined, GAABCPs are amino acid extensions of variable length; wherein the amino acid extensions comprise a block copolymer arrangement of repeating glycan acceptor motifs, as defined herein, separated by spacer motifs, as defined herein. Accordingly, GAABCPs comprise at least two such glycan acceptor motifs separated by such a spacer motif.

The term “GAABCP-TUP” refers generally to any GAABCP, as herein defined, conjugated to a TUP, as defined herein. In some embodiments, GAABCP TUPs are made by expressing a GAABCP-TUP expression vector in a suitable expression system. In some embodiments, a GAABCP and a TUP are made separately by expressing a GAABCP expression vector in a suitable expression system and separately expressing a TUP expression vector in a suitable expression system, then conjugating the TUP and the GAABCP together according to the present disclosure. In other embodiments, one or both of the GAABCP and the TUP are made synthetically, e.g., via solid-phase chemical synthesis, and then the GAABCP and TUP are conjugated according to the present disclosure. In one particular embodiment, a GAABCP is made via solid-phase synthesis. As is described throughout the specification, GAABCP-TUPs comprise glycan acceptor motifs which may optionally be glycosylated. Glycosylation of GAABCP-TUPs may occur in vitro, or in vivo. In certain embodiments, a GAABCP-TUP is glycosylated in vivo, as directed by a glycosyltransferase. In certain embodiments, a GAABCP-TUP is glycosylated in vitro, as directed by a glycosyltransferase. In certain other embodiments, a GAABCP-TUP is glycosylated in vitro, e.g., via a chemical reaction, an enzymatic glycosylation, or via a chemo-enzymatic conjugation. In some particular embodiments, a glycosylated GAABCP is produced via a solid-phase peptide synthesis method such as, e.g., but not limited in any way, using the methods disclosed in U.S. Pat. Nos. 7,943,763, 8,063,202, and 8,399,656, incorporated herein in their entirety. In one embodiment, such methods are utilized to synthesize a GAABCP comprising an N-glycan moiety conjugated to each of a plurality of glycan acceptor motifs comprised in a GAABCP according to the present disclosure, wherein the GAABCP is then able to be conjugated to a TUP via chemical conjugation or via chemo-enzymatic conjugation.

As used herein, the term “GAABCP-TUP expression vector” is meant to refer to any recombinant protein expression vector that comprises either a native or altered polynucleotide sequence encoding a GAABCP-TUP. Thus according to the present disclosure, the terms “GAABCP-relaxin expression vector” or “GAABCP-G-CSF expression vector” or “GAABCP-GLP-1 expression vector” should be understood to refer to recombinant protein expression vectors comprising nucleotide sequences encoding GAABCP-relaxin, GAABCP-G-CSF, and GAABCP-GLP-1, respectively, with the GAABCP being inserted in any suitable location with regard to the therapeutically useful polypeptide sequence so as to encode an in-frame glycoprotein conjugate comprising the TUP and the GAABCP as one continuous polypeptide sequence.

The term “glycan” or “saccharide” or “sugar” as used herein includes any carbohydrate molecule, including analogs of native carbohydrates. It may refer to a monosaccharide, or to chains of monosaccharides such as oligosaccharide or polysaccharide chains.

The term “glycan acceptor motif” or “glycosylation motif” or “glycan motif” as used herein is meant to describe any amino acid sequence capable of being glycosylated by a glycosyltransferase in vitro, ex vivo, or in vivo under proper conditions. The glycan acceptor motif may be a consensus motif, such as the mammalian N-glycosylation consensus motif N-X-(T/S), wherein X is any amino acid except for proline, and N, T, and S refer to asparagine (Asn), threonine (Thr), and serine (Ser), respectively; wherein (T/S) means that either a Thr (T) or a Ser (S) residue may be present. In some embodiments, N-glycosylation occurs when the oligosaccharyltransferase encounters an N-X-(T/S) motif comprised in a polypeptide and it catalyses the covalent attachment of a core oligosaccharide to the N residue of the motif. The glycan acceptor motif may also be a predicted or experimentally validated motif, such as, e.g., an O-glycan motif. Although no consensus motif for O-glycosylation is known, O-glycan preferences have been described which allow for the construction of optimal synthetic sequences to promote O-glycosylation (e.g., Gerken T A, et. al. (2006) J. Biol. Chem. 281(43):32403-16, incorporated herein). Furthermore, numerous O-glycan sites are known in the literature and are suitable for inclusion in a GAABCP (e.g., see the Net.O.Glyc server, which is an O-glycosylation prediction resource that also provides a large database of experimentally validated O-glycan sites. Julenius K., et. al. (2005) Glycobiology 15:153-164, incorporated herein). It is known, that O-glycan specificity can be promoted by the inclusion of approximately three appropriate amino acids flanking (i.e. both N- and C-terminal to) the glycan acceptor amino acid; as such, in some embodiments, O-glycan motifs contemplated for use in the present invention will comprise repeat motifs comprising at least 7 amino acids that comprise an O-glycan acceptor amino acid flanked by 3 appropriate amino acids on either side, e.g., as described in the Gerken and Julenius papers cited above.

The term “Glycan macroheterogeneity” refers to variations in the relative percentage of glycosylation acceptor motif site occupancy by glycan chains in a glycoprotein or glycoprotein conjugate.

The term “Glycan microheterogeneity” refers to variations in the structures of the glycan chains attached to the glycosylation acceptor motifs.

The term “Glycoprotein” is meant to describe any polypeptide bound to at least one glycan. In some embodiments, a glycoprotein is a polypeptide that has been glycosylated, as defined herein. In some embodiments, a glycoprotein is a polypeptide to which a glycan has been conjugated to, e.g. via chemical conjugation. In some embodiments, the glycoprotein is a glycoprotein conjugate, as defined herein. The term glycoprotein may also be used to describe a polypeptide that comprises at least one glycan acceptor motif, such that, when appropriate conditions are provided, the polypeptide is capable of being glycosylated.

The term “Glycoprotein conjugate” is used herein in reference to a polypeptide conjugated to a glycan or glycoprotein, as described herein. Most typically, the term glycoprotein conjugate is used herein in reference to a GAABCP-TUP (i.e., TUP as described herein, conjugated to a GAABCP, as disclosed herein). The glycoprotein conjugates referenced throughout this specification are therefore, by definition, non-naturally occurring proteins (e.g., fusions; for example, genetic or chemical fusions of two separate entities).

The term “glycosylation” refers to the process of adding a glycan to anything, including adding a glycan to an amino acid (natural or non-natural, as defined herein) and adding a glycan to another glycan, e.g., building an oligo- or polysaccharide chain. Encompassed in this is in vitro and in vivo glycosylation. “In vivo glycosylation” is intended to mean any attachment of a glycan moiety occurring in vivo, i.e. during post-translational processing in a glycosylating cell used for expression of the polypeptide, e.g. by way of N-linked and O-linked glycosylation. This would include glycosylation occurring in a cell in isolation, such as is commonly referred to as ex vivo, or a cell comprised in an organism or at least part of an organism. While the term “in vitro glycosylation” is intended to refer to any synthetic addition of a glycan to another molecule in vitro, this term typically is used herein to refer to the covalently linking a glycan to an amino acid, or to another glycan comprised in an oligosaccharide chain, via an enzymatic reaction catalyzed by a glycosyltransferase. In some embodiments, however, “in vitro glycosylation” encompasses non-glycosyltransferase catalyzed addition of a glycan to a glycan acceptor molecule, e.g., optionally using a cross-linking agent or by glycation. In other embodiments, the in vitro glycosylation will employ both the use of non-glycosylatranferase-mediated glycosylation and glycosyltransferase-mediated glycosylation. Any glycan that is suitable for conjugation to a glycan acceptor motif may be conjugated to a glycan acceptor motif by in vitro glycosylation, in accordance with the present invention. Such glycans include glycans that are synthesized (e.g., by a chemo-enzymatic process) and glycans that isolated from a biological source, e.g., from cells, tissues, culture supernatents, serum samples, eggs (e.g., chicken eggs), and any other suitable source. In some embodiments, a glycan moiety (e.g., an N-glycan moiety) with a specific carbohydrate structure is isolated from a suitable source or is synthesized using any suitable means known to the skilled artisan, (e.g., by a chemo-enzymatic process) and then the moiety is attached to a glycan acceptor motif (e.g., a glycan acceptor motif comprised in a GAABCP or a GAABCP-TUP as disclosed herein) using chemical reactions, via enzymatically catalyzed chemical reactions with naturally occurring glycotransferases, or via a combination of chemical and enzymatically catalyzed chemical reactions using glycosyltransferases. In some embodiments, an amino acid linked to a glycan moiety (e.g., an N-glycan moiety) with a specific carbohydrate structure is incorporated into a peptide (e.g., at least a portion of a GAABCP or an entire GAABCP, as disclosed herein) using solid-phase peptide synthesis methods know to those in the art such as, but not limited to the methods disclosed in U.S. Pat. Nos. 7,943,763, 8,063,202, and 8,399,656, incorporated herein in their entirety. In some particular embodiments, such methods are utilized to synthesize a GAABCP comprising an N-glycan moiety conjugated to each of a plurality of glycan acceptor motifs comprised in a GAABCP according to the present disclosure, wherein the GAABCP is then able to be conjugated to a TUP via chemical conjugation or via chemo-enzymatic conjugation.

The terms “half-life”, “serum half-life”, and “plasma half-life” are used interchangeably herein, and refer to the time it takes for the concentration of a molecule, which is in plasma to decrease to ½ of its original concentration. In one embodiment, this can refer to a molecule exposed to plasma in vitro, but preferably it refers to a molecule exposed to plasma in vivo (i.e. in the circulation of a subject), more preferably exposed to plasma in a mammal, e.g. a mouse, even more preferably exposed to plasma in a human). When measured in vivo, the plasma half-life of a GAABCP-TUP or its corresponding unmodified TUP is measured by taking blood samples at various time points after administration of the molecule, and determining the concentration of that molecule in each sample. Correlation of the plasma concentration with time allows calculation of the plasma half-life.

The term “half maximal effective concentration” or “EC50” or “potency” refers to the concentration of a therapeutically useful polypeptide (e.g., any GAABCP-TUP, or other TUP such as for example: a GLP-1 polypeptide, relaxin-2 polypeptide, G-CSF polypeptide, GAABCP-GLP-1, GAABCP-relaxin-2, GAABCP-G-CSF), as described, herein at which it induces a response halfway between the baseline and maximum after some specified exposure time in a given bioassay. The EC50 of a graded dose response curve therefore represents the concentration of a compound at which 50% of its maximal effect is observed. EC50 also represents the plasma concentration required for obtaining 50% of a maximum effect in vivo. Similarly, the “EC90” refers to the concentration of an agent or composition at which 90% of its maximal effect is observed. The “EC90” can be calculated from the “EC50” and the Hill slope, or it can be determined from the data directly, using routine knowledge in the art. In some embodiments, the EC50 of a GAABCP-TUP is less than about 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, a pharmaceutical composition will have an EC50 value of about 1 nM or less.

By a polypeptide having an amino acid sequence at least, for example, 95% “identical” to a query amino acid sequence (e.g., a amino acid sequence that can be compared to published sequences using the Basic Local Alignment Search Tool or BLAST algorithm; Johnson M, Zaretskaya I, Raytselis Y, Merezhuk Y, McGinnis S, Madden T L (2008) NCBI BLAST: a better web interface. Nucleic Acids Res. 36:W5-W9, incorporated herein) of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. Similarly, a “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The terms “in vitro”, “ex vivo”, and “in vivo” are intended herein to have their normal scientific meanings. Accordingly, e.g., “in vitro” is meant to refer to experiments or reactions that occur with isolated cellular components, such as, e.g., an enzymatic reaction performed in a test tube using an appropriate substrate, enzyme, donor, and optionally buffers/cofactors. “Ex vivo” is meant to refer to experiments or reactions carried out using functional organs or cells that have been removed from or propagated independently of an organism. “In vivo” is meant to refer to experiments or reactions that occur within a living organism in its normal intact state.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

A “longer plasma half-life” simply refers to an “increase” in the “plasma half-life” as defined herein. In some embodiments, longer plasma half-life refers to statistically significant increases in half-life. In some embodiments, longer plasma half-life refers to a half-life that is at least about several hours longer. In some embodiments, longer plasma half-life refers to a half-life that is at least about 2 hours longer, or 3, 4, 5, 6, 7, 8, 9, 10, 11, 12, 13, 14, 15, 16, 17, 18, 19, 20, 21, 22, 23 or (any integer or decimal in between) hours longer. In some embodiments, longer plasma half-life refers to a half-life that is at least about 1 day longer. In some embodiments, longer plasma half-life refers to a half-life that is at least about 2 days longer, or 3, 4, 5, 6, 7, 8, 9, 10, 11, 12, 13, 14, 15, 16, 17, 18, 19, 20, or 21, or (any integer or decimal in between) days longer. In some embodiments, longer plasma half-life refers to a half-life that is at least about 3 weeks longer, or more than 4 weeks longer. In some embodiments a longer plasma half-life is from 1-5 days longer, or from 2-7 days longer or from 1-2 weeks longer, or 2-3 weeks longer, or 3-4 weeks longer.

“Mammal” includes humans and both domestic animals such as laboratory animals and household pets, (e.g., cats, dogs, swine, cattle, sheep, goats, horses, and rabbits), and non-domestic animals such as wildlife and the like.

When a polypeptide or polynucleotide is said to be of “mammalian origin”, or “human origin”, this is explicitly meant to refer to a polypeptide that is a native mammalian or native human polypeptide or polynucleotide sequence, respectively; or a variant thereof, as described herein. Accordingly, for example, a polypeptide that is of mammalian origin may be a polypeptide that is naturally expressed in a mammal, or it may be a polypeptide that is derived from a polypeptide that is expressed in a mammal, thus retaining some desirable function that the native polypeptide possessed, but comprising a variant amino acid sequence.

The term “naked” as used herein refers to the unglycosylated state of a polypeptide or amino acid that is capable of being glycosylated when appropriate conditions are provided. Accordingly, a naked amino acid (e.g., a naked asparagine) is to be understood as an amino acid that is not glycosylated, as defined herein. Similarly, a naked polypeptide is a polypeptide that does not comprise any amino acids that are glycosylated, as defined herein.

The term “native sequence” or “native polypeptide” simply refers to any polynucleotide or polypeptide that is found in nature. In some embodiments, such a “native” sequence is a sequence that is identical to any reference sequence listed as any one of SEQ ID NOS: 97, 103, 109, 146, 157, 164, or 168, or as shown in Table 5), or any primary accepted reference sequence according to the UniProt Knowledgebase database (UniProt Consortium. Update on activities at the Universal Protein Resource (UniProt) in 2013. Nucleic Acids Res. 2013 41:D43-7, incorporated herein) or the UniGene database (Pontius J U, Wagner L, Schuler G D. UniGene: a unified view of the transcriptome. In: The NCBI Handbook. Bethesda (Md.): National Center for Biotechnology Information; 2003, incorporated herein).

In some embodiments, the term “non-native” refers to any sequence (e.g., polypeptide, polynucleotide) that differs from any native sequence as described herein. For example, in some embodiments, a non-native sequence is approximately 50%, 60%, 70%, 80%, 90%, or 95% identical to its corresponding native amino acid sequence, including all integers (e.g., 91%, 92%, 93%, 94%, 96%, 97%, 98%, 99%) and ranges above and in between (e.g., 50%-99%; 60%-99%; 70%-99%; 80%-99%; 90%-99%; 95%-99%). In some embodiments, the term “non-native” is used to refer to a sequence that is non-native to a particular TUP.

The term “N-glycosylation” is meant herein to have its normal, scientific meaning, e.g., the attachment of a sugar moiety to a nitrogen atom of an amino acid such as, e.g., an asparagine or arginine residue. Usually, the N-glycosylated oligosaccharide moiety has a common basic core structure composed of five monosaccharide residues, namely two N-acetylglucosamine residues and three mannose residues. The exact oligosaccharide structure will vary, in part, depending on the glycosylating organism in question and on the specific polypeptide. Depending on the host cell in question, the glycosylation is classified as a high mannose type, a complex type or a hybrid type. Complex N-linked oligosaccharides do not contain terminal mannose residues. They contain only terminal N-acetylglucosamine, galactose, and/or sialic acid residues. Hybrid oligosaccharides contain terminal mannose residues as well as terminal N-acetylglucosamine, galactose, and/or sialic acid residues. High mannose type glycans contain unsubstituted terminal mannose sugars. Accordingly, a glycan chain that is attached to a polypeptide via N-glycosylation is referred to as an “N-glycan” or “N-linked glycan”.

The term “O-glycosylation” is meant herein to have its normal, scientific meaning, e.g., the attachment of a sugar moiety (e.g., N-Acetylgalactosamine, N-Acetylglucosamine, fucose, glucose, mannose) to a hydroxyl group of an amino acid such as e.g., a serine, threonine, tyrosine, hydroxylysine, or a hydroxyproline residue. Accordingly, a glycan chain that is attached to a polypeptide via O-glycosylation is referred to herein as an “O-glycan” or “O-linked glycan”.

“Optional” or “optionally” means that the subsequently described event, or circumstances, may or may not occur, and that the description includes instances where said event or circumstance occurs and instances in which it does not.

“Pharmaceutical composition” refers to a formulation of a compound (e.g. a therapeutically useful polypeptide) and a medium generally accepted in the art for the delivery of the compound to an animal, e.g., humans. Such a medium may include any pharmaceutically acceptable carriers, diluents or excipients therefore.

“Pharmaceutically effective excipients” and “pharmaceutically effective carriers” are well-known to those of skill in the art, and methods for their preparation are also readily apparent to the skilled artisan. Such compositions, and methods for their preparation, may be found, e.g., in Remington's Pharmaceutical Sciences, 19th Edition (Mack Publishing Company, 1995, incorporated herein).

Reference herein to “pharmacokinetic properties” is meant to describe standard properties of a compound (e.g., a TUP or GAABCP-TUP) related to how the body of a subject affects the compound upon administration to the subject. Typically, such properties are referred to as ADME or LADME, and include Liberation, Absorption, Distribution, Metabolization, and Excretion. “Liberation” is the process of the release of a drug from a pharmaceutical formulation. “Absorption” is the process of a substance entering the blood circulation. “Distribution” is the dispersion or dissemination of substances throughout the fluids and tissues of the body. “Metabolization” is the irreversible transformation of the compound into derivative metabolites by the body. “Excretion” is the removal of the compounds from the body. The half-life of the compound is one such pharmacokinetic property, and is roughly related to the rate of metabolization and excretion of the compound.

The terms “polynucleotide”, “nucleotide”, “nucleotide sequence”, and “nucleic acid” are used interchangeably. They refer to a polymeric form of nucleotides of any length, either deoxyribonucleotides or ribonucleotides, or analogs thereof. Polynucleotides may have any three dimensional structure, and may perform any function known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may include non nucleotide components. A polynucleotide may be further modified after polymerization, such as by conjugation with a labeling component.

The terms “polypeptide” or “peptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example by disulfide bond formation, glycosylation, lipidation, acetylation, phosphorylation, hydroxylation, or any other manipulation, such as conjugation with a labeling component.

The terms “polypeptide domain” or “therapeutically useful polypeptide domain” or “unmodified therapeutically useful polypeptide domain” are used herein to refer to groups of amino acids within the primary structure of these polypeptides. The amino acid groupings may be assigned based on knowledge of the 2o or higher structure of the polypeptide, or based on the understanding of the structure-activity relationships of the polypeptide.

In certain embodiments, the “purity” of any given agent (e.g., a GAABCP-TUP or unmodified TUP) in a composition may be specifically defined. For instance, certain compositions of the present invention comprise an agent (e.g., a GAABCP-TUP) that is at least 80% pure, or an agent (e.g., a GAABCP-TUP) that is at least 85, 90, 91, 92, 93, 94, 95, 96, 97, 98, 99, or 100% pure, including all decimals in between, as measured, for example and by no means limiting, by SDS-PAGE, a well-known analytical method used frequently in biochemistry and analytical chemistry to separate polypeptides based on mass, or size exclusion chromatography-high pressure liquid chromatography (SEC-HPLC) with A280 detection, a well-known form of column chromatography used frequently in biochemistry and analytical chemistry to separate molecules based on mass.

As is discussed further in the detailed descriptions of the glycan acceptor motifs and the spacer motifs, it is important to note that whenever the term “repeat” or “repeating units” is used in reference to the number of units of the glycan acceptor motif/spacer motif comprised in a GAABCP, this term is meant to encompass exact repeats (i.e. identical repeating amino acid sequences encoding the glycan acceptor/spacer motifs) as well as different sequences encoding the glycan acceptor/spacer motifs. Thus the repeating elements of the block copolymer are not necessarily identical repeating amino acid sequences (although in some embodiments one or more exact sequences are repeated in a GAABCP), but they may instead, in some embodiments, simply be repeating glycan acceptor motifs separated by spacer motifs, as described herein.

The term “sialylation” is meant herein to have its normal, scientific meaning, e.g., the attachment of a sialic acid (N-acetylneuraminic acid) moiety (or chemical variants thereof) to a glycoprotein or glycoprotein conjugate in the terminal position of the glycan chain's chemical structure.

A “subject,” as used herein, includes any animal that exhibits a disease or symptom, or is at risk for exhibiting a disease or symptom, which can be treated with a GAABCP-TUP of the invention. Suitable subjects include laboratory animals (such as mouse, rat, rabbit, or guinea pig), farm animals, and domestic animals or pets (such as a cat or dog). Non-human primates and, preferably, human patients, are included.

“Substantially” or “essentially” means of ample or considerable amount, quantity, size; nearly totally or completely; for instance, 95% or greater of some given quantity.

The term “terminal sialyation” refers to sialic acid moieties that are present on the ends of oligosaccharide or polysaccharide chains, such as for example, linear or branched polysaccharides. In some embodiments, the GAABCP-TUPs of the present invention will have a high level of terminal sialylation (i.e. at least about 60% terminal sialyation), a moderate level of terminal sialylation (i.e., about 40-60% terminal sialyation), or a low level of terminal sialylation (i.e. less than about 40% terminal sialyation. Some preferred GAABCP-TUPs comprise at at least about 60% or more terminal sialyation. In some embodiments, the GAABCP of the present invention will have a high level of terminal sialyation that is at least about 60%, or at least about 61, 62, 63, 64, 65, 66, 67, 68, 69%, or greater. In certain preferred embodiments, the GAABCP-TUP of the present invention comprises a high level of terminal sialyation that is at least about 70% or about 71, 72, 73, 74, 75, 76, 77, 78, 79, 80, 81, 82, 83, 84, 85, 86, 87, 88, 89, 90, 91, 92, 93, 94, 95%, or greater terminal sialyation, including all decimals and ranges thereof. Accordingly, in some embodiments, GAABCP-TUPs comprise from about 60%-100% terminal sialyation, or from about 70%-100%, or from 70%-95%, 70%-90%, 70%-85%, 70%-80%, 70%-75%, 75%-100%, 75%-95%, 75%-90%, 75%-85%, 75%-80%, 80%-100%, 80%-95%, 80%-90%, 80%-85%, 85%-100%, 85%-95%, 85%-90%, 90%-100%, 90%-95%, or about 95%-100% terminal sialyation.

Reference to “terminus” or “termini”, as in the terms “N-terminus” and “C-terminus” are used herein according to their typical scientific meaning, namely the amino-terminus (or NH2-) and carboxy-terminus (or —COOH), respectively. Specifically, these terms are used in reference to a polypeptide. One skilled in the art will readily understand that each amino acid comprises a carboxyl group and an amine group, and the basis of a polypeptide chain is the peptide linkage that is formed between the amine group of one amino acid and the carboxyl group of another amino acid. Accordingly, all natural linear polypeptide chains comprise two terminal amino acids, one with a free amino group (i.e. the “N-terminus”) and one with a free carboxly group (i.e. the “C-terminus”). Accordingly, reference to the phrases “conjugated N-terminally”, “conjugated C-terminally”, and the like, are meant to simply refer to a terminal conjugation to the N- or C-terminus, respectively. As is clearly obvious to the skilled artisan, all polypeptide sequences listed herein proceed from left to right with the N-terminus being on the left of the sequence and the C-terminus being on the right.

“Therapeutic agent” refers to any compound that, when administered to a subject, (e.g., preferably a mammal, more preferably a human), in a therapeutically effective amount is capable of effecting treatment of a disease or condition as defined below.

“Therapeutically effective amount” or “Therapeutically effective dose” refers to an amount of a compound of the invention that, when administered to a subject, (e.g., preferably a mammal, more preferably a human), is sufficient to effect treatment, as defined below, of a disease or condition in the animal. The amount of a compound of the invention that constitutes a “therapeutically effective amount” will vary depending on the compound, the condition and its severity, the manner of administration, and the age of the animal to be treated, but can be determined routinely by one of ordinary skill in the art having regard to his own knowledge and to this disclosure.

Accordingly when a compound (e.g., a GAABCP-TUP of the present invention) is said to possess “therapeutic efficacy” this is intended to mean that the compound is capable of effecting treatment, as defined below, of a disease or condition in a subject; provided a “therapeutically effective amount” of the compound is administered under appropriate conditions.

The terms “therapeutically useful polypeptide” and “TUP” which are used interchangeably herein, refer to any polypeptide (or fragments and variants thereof), with a biological activity and/or other physiochemical properties that make it suitable for use as a therapeutic agent. Any “therapeutically useful polypeptide” is suitable for modification with a GAABCP, and modification of a “therapeutically useful polypeptide” with a GAABCP, according to the present disclosure, will result in a GAABCP-TUP. Further, a “therapeutically useful polypeptide” may be comprised of naturally occurring or non-naturally occurring amino acid sequences. These amino acid sequences may be unmodified or modified by any means of protein engineering including, but not limited to, genetic engineering, chemical or genetic conjugations, replacement of amino acid with one or more non-amino acid chemical analog, or combinations thereof. A “therapeutically useful polypeptide” may contain amino acid sequences that represent deletions, additions or alterations of single amino acids, or fragments, of the primary amino acid sequence found in the naturally occurring polypeptide such that the fragment polypeptide or variant polypeptide retains similar biological activity to the native form.

“Treating” or “treatment” as used herein covers the treatment of the disease or condition of interest in a subject, preferably a human, having the disease or condition of interest, and includes: (i) preventing or inhibiting the disease or condition from occurring in a subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady, injury or condition may not have a known causative agent (so that etiology has not yet been worked out), and it is, therefore, not yet recognized as an injury or disease but only as an undesirable condition or syndrome, wherein a more or less specific set of symptoms have been identified by clinicians.

The term “unmodified therapeutically useful polypeptide”, as used herein, refers to an amino acid sequence encoding a therapeutically useful polypeptide that does not contain a GAABCP.

The term “variant” or “variants thereof” refers to a polynucleotide or polypeptide differing from a reference nucleic acid or polypeptide, but retaining essential structural and functional properties thereof (e.g., in some embodiments similar biological activity). Generally, variants may be overall closely similar, and, in many regions, identical to the reference nucleic acid or polypeptide sequence. As used herein, “variant”, typically refers to a polynucleotide or polypeptide sequence encoding a TUP or a GAABCP-TUP, said sequence being different than the native sequence disclosed herein (e.g. any one of native vs. variant SEQ ID NOS: 97 v. 98-102 and 134-140, or 103 v. 104-108 and 141-145, or 146 v. 147-156, or 157 v. 158-163, or 164 v. 165-167, or 168 v. 169-176, or 109 v. 110, 111, 113-120 & 122-123), or any primary accepted reference native polypeptide sequence according to the UniProt Knowledgebase database (UniProt Consortium. Update on activities at the Universal Protein Resource (UniProt) in 2013. Nucleic Acids Res. 2013 41:D43-7.) or the UniGene database (Pontius J U, Wagner L, Schuler G D. UniGene: a unified view of the transcriptome. In: The NCBI Handbook. Bethesda (Md.): National Center for Biotechnology Information; 2003, incorporated herein). Accordingly, the term variant is also directed to proteins which comprise, or alternatively consist of, an amino acid sequence which is at least 50%, 55%, 60%, 65%, 70%, 75%, 80%, 85%, 90%, 95%, 96%, 97%, 98%, 99% or 100%, identical to, for example, the amino acid sequence of any TUP. Fragments of these polypeptides are also provided (e.g., those fragments described herein).

Reference to the phrase “when appropriate conditions are provided” with regard to the glycosylation of a GAABCP, refers to any condition that can promote or induce the glycosylation of at least one glycan acceptor motif comprised in a GAABCP. Such appropriate conditions may for example be in vitro glycosyltransferase assays comprising a GAABCP polypeptide, suitable donor sugar nucleotides or donor oligosaccharides, a glycosyltransferase or oligosaccharyltransferase, and optionally a suitable buffer system so as to promote efficient glycosylation. Additionally, appropriate conditions may include, e.g., ex vivo or in vivo glycosylation catalyzed by a suitable endogenous or exogenous glycosyltransferase or oligosaccharyltransferase.

### Overview

The present disclosure is based on novel glycoprotein conjugates comprising one or more glycosylated amino acid block copolymer polypeptides (GAABCP) conjugated to one or more therapeutically useful polypeptides (TUPs). Conjugation of a TUP and a GAABCP may be accomplished by any suitable means, (e.g., genetic or chemical conjugation), to form a novel GAABCP-TUP glycoprotein conjugate (or “GAABCP-TUP”), as disclosed herein.

In some instances, the TUP is a biologically active polypeptide and in other instances it is not. Bioactivity of biologically active TUPs may be increased, decreased, or substantially unchanged following conjugation to a GAABCP according to the present disclosure.

Accordingly, disclosed herein are GAABCP-TUPs, methods related to synthesizing and producing GAABCP-TUPs, and pharmaceutical compositions comprising GAABCP-TUPs. GAABCP-TUPs will be useful for treating and preventing numerous diseases and conditions. In some embodiments, the conjugation of the TUP to a block copolymer of the present invention increases the molecular weight of the TUP. In certain embodiments, the conjugation of the TUP to a block copolymer of the present invention increases the molecular weight of the TUP to at least about 50 kDa, or at least about 60, 70, 80, 100, 120, 140, 160, 180, 200, 250, 300 kDa or more. In some embodiments, the molecular weight increase reduces glomerular filtration. In some embodiments, the molecular weight increase results in an increase in the plasma half-life of the polypeptide conjugate.

In some embodiments, the conjugation of the TUP to a block copolymer of the present invention alters the isoelectric point (pI) of TUP. In one embodiment, the pI of the TUP is decreased by the conjugation of a block copolymer of the present invention. In certain embodiments, the conjugation of the TUP to a block copolymer of the present invention decreases the pI of the TUP by at least about 0.1, 0.2, 0.3, 0.5, 0.7, 1.0, 1.5, 2.0, 2.5, 3.0, 4.0 units, or more. In some embodiments, the reduced PI reduces immunogenicity. In some embodiments, the reduced immunogenicity is due to the inhibiting of an interaction between the polypeptide conjugate and antibodies or other components of the immune system. In some embodiments, the pI decrease results in an increase in the plasma half-life of the polypeptide conjugate.

In some embodiments, the conjugation of the TUP to a block copolymer of the present invention decreases the immunogenicity of the TUP. Immunogenicity testing can be performed, e.g., using the NetMHCIIpan algorithm (Karosiene E, Rasmussen M, Blicher T, Lund O, Buus S, Nielsen M (2013) NetMHCIIpan-3.0, a common pan-specific MHC class II prediction method including all three human MHC class II isotypes, HLA-DR, HLA-DP and HLA-DQ. Immunogenetics 65(9):655-65, incorporated herein), which calculates the potential binding affinity of proteins or peptides to MHCII complex. The higher the calculated binding affinity the higher is the risk to induce antibodies directed against the polypeptide of interest. In certain embodiments, the conjugation of the TUP to a block copolymer of the present invention decreases the immunogenicity of the TUP.

In still further embodiments, the conjugation of the TUP to a block copolymer of the present invention increases the molecular weight of the TUP decreases the isoelectric point (pI) of the TUP, and/or decreases the immunogenicity of the TUP.

### Increased Plasma Half-Life

In some embodiments, the present invention relates to glycoprotein conjugates that exhibit increased plasma half-lives. Increases in the plasma half-life of a GAABCP-TUP (compared to equimolar amounts of the corresponding unmodified TUP) need not be statistically significant, however in some instances they are. In some instances the half-life of a GAABCP-TUP may be increased about 2 fold (compared to equimolar amounts of the corresponding unmodified TUP). In some instances, the half-life of a GAABCP-TUP may be increased about 2, 3, 4, 5, 6, 7, 8, 9, 10, 25, 50, 100, 500, 1000, 5000, 10000 fold, or more (compared to equimolar amounts of the corresponding unmodified TUP). In some instances, the half-life of a GAABCP-TUP is about 1 day longer than an equimolar amount of the corresponding unmodified TUP, or about 2, 3, 4, 5, 6, 7, 8, 9, 10, 11, 12, 13, 14 days longer than an equimolar amount of the corresponding unmodified TUP. In some instances the half-life of a GAABCP-TUP is 2 weeks or more, e.g., 3, 4, 5, or more weeks longer than an equimolar amount of the corresponding unmodified TUP.

Plasma half-lives of the molecules (e.g., TUPs or GAABCP-TUPs, as described herein) may be tested in accordance with any of the standard techniques utilized for determining the in vivo pharmacokinetic properties of the molecule. For example, but not to be limited by example, in one method, predetermined equimolar doses of a GAABCP-TUP and its corresponding unmodified TUP are each administered to a separate animal as a single intravenous bolus injection, preferably a laboratory animal such as a rodent, e.g., mouse or rat; and then plasma is collected from the animals at predetermined time intervals and is analyzed, e.g., via an Enzyme-Linked Immunosorbent Assay (“ELISA”) specific to the TUP, to determine the molar equivalent plasma concentrations of the unmodified TUP and the GAABCP-TUP. The half-life of the administered molecule may then be calculated via a non-compartmental pharmacokinetic analysis algorithm (e.g., WINNonLin software version 4.1). In addition to the last time at which the unmodified TUP or GAABCP-TUP are measurable (4), this analysis may include observation or calculation of the following parameters: apparent terminal rate constant associated to the apparent terminal phase (λ_{z}, estimated by linear regression analysis of the logarithm of the plasma concentrations versus time in the mono-exponential terminal part of the curve), apparent terminal half-life (t_{1/2, z}, calculated according to the following equation: t_{1/2,z}=ln(2)/λ_{z}; AUC), area under the plasma concentration-time curve from time zero to infinity (AUC_{0-∞}), area under the plasma concentration-time curve per unit of dose (AUC/D), mean residence time calculated as the ratio between the area under the first moment curve (MRT), systemic clearance (CL, calculated as CL=D/AUC), and steady state volume of distribution (Vₛₛ, calculated as Vₛₛ=CL*MRT).

### Genetic Conjugation of GAABCPs to TUPs

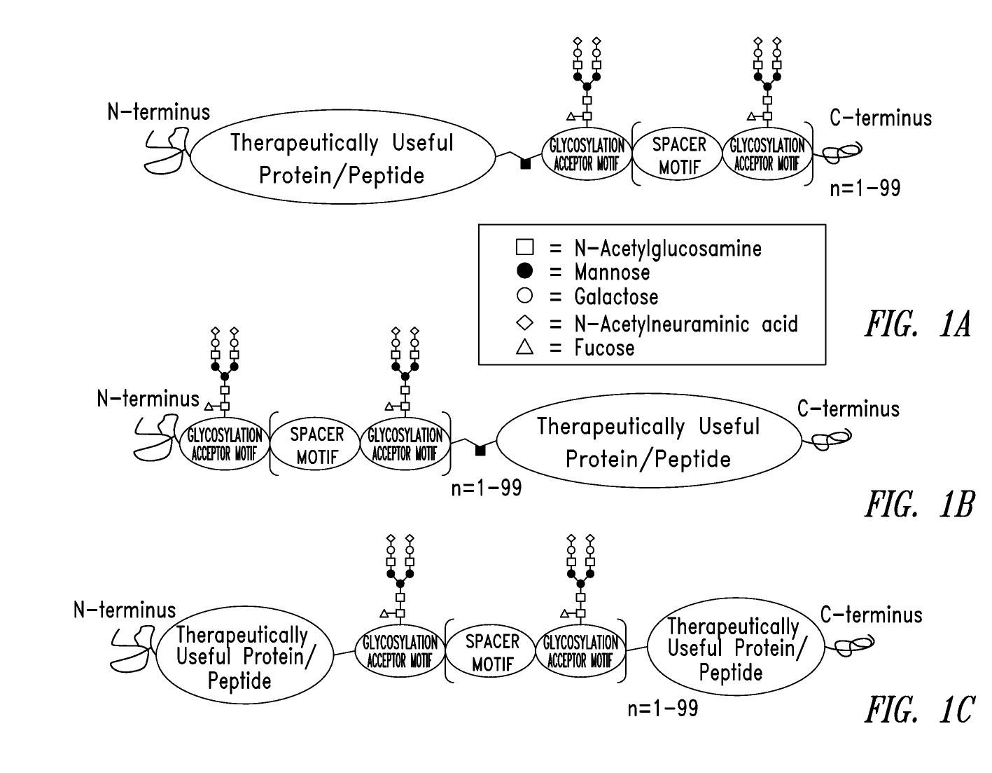

GAABCP-TUPs may comprise one of more GAABCPs conjugated to the N-terminus of the primary amino acid sequence of a TUP (FIG. 1B), the C-terminus of a TUP (FIG. 1a), or both the N- and C-termini of a TUP. Furthermore, GAABCP-TUPs may comprise one or more GAABCPs inserted within the internal amino acid sequence of a TUP. Still further, GAABCP-TUPs may comprise a combination of a GAABCPs conjugated at one or both termini of the TUP. Furthermore, GAABCP-TUPs may comprise one or more TUPs conjugated to one or more GAABCPs in any of the aforementioned combinations. For example, in one non-limiting embodiment, the present invention may comprise two TUPs linked together by a GAABCP (FIG. 1C), or linked together by an amino acid extension comprising a GAABCP and optionally any other optional element disclosed in the present specification. Thus any one or more TUPs, as described herein, conjugated to any one or more GAABCPs, as described herein, are encompassed in the present invention and are described by the term GAABCP-TUP.

When a GAABCP is said to be inserted “within the internal amino acid sequence” of a TUP, this is to be interpreted as being inserted anywhere except at the very most N- or C-terminal position of the primary amino acid sequence of the TUP. With the exception of one case (“signal peptide exception”), the presence of at least one amino acid from the unmodified TUP being both N- and C-terminal to the GAABCP is to be considered enough to fulfill the requirement of being within the internal amino acid sequence. The signal peptide exception relates to TUPs that comprise a secretion signal peptide (“signal peptide”). If the TUP comprises a signal peptide, it is to be understood that conjugation of a GAABCP to the C-terminus of the signal peptide and the N-terminus of the TUP sequence is not considered to be an internal insertion, but it is intended herein to be considered an N-terminal insertion. For many proteins, the location of the signal peptide cleavage site is known due to experimentation. However, in cases where experimental data confirming this site are lacking, the cleavage site can be easily predicted using a variety of free and commercially available software algorithms, such as, but not limited to, SignalP 4.1 Server (Petersen B, Nielsen H (2011) Nature Methods, 8:785-786, incorporated herein). Accordingly, reference to a “terminal insertion” or “non-internal insertion” or an insertion at a one or more “termini” is intended to refer to any insertion that is positioned at the actual N-, or C-termini of the TUP sequence, as well as an insertion described above as, in the signal peptide exception.

### Description of GAABCP Sequences

GAABCPs are polypeptides comprising an amino acid block copolymer sequence comprising two alternating amino acid elements: (1) glycan acceptor motifs and (2) spacer motifs, as described herein. Encompassed in the term “GAABCP” are both partially glycosylated and fully glycosylated GAABCPs, as well as naked GAABCPs, provided at least one glycan chain could potentially be added to the naked GAABCP by a glycosyltransferase when appropriate conditions a provided, as defined herein. Furthermore, the term “GAABCP” also encompasses any amino acid sequence, or related nucleotide sequences which encodes the amino acid sequence, of a GAABCP as described herein.

The GAABCPs of the present invention may comprise any number of repeating units of the aforementioned alternating amino acid elements, according to the limitations set forth herein. For the purpose of clarity, the number of repeats in a GAABCP is defined as the number of glycan acceptor motifs comprised in the GAABCP that are separated by spacer motifs. Thus, although a GAABCP may optionally comprise a spacer motif N-terminal to the first (i.e. most N-terminal) glycan acceptor motif, or C-terminal to the last (i.e. most C-terminal) glycan acceptor motif, such embodiments do not alter the number of repeats said to be in the GAABCP. So, for example, a GAABCP comprising the structure S-G-S-G-S-G-S, (wherein S represents a spacer motif and G represents a glycan acceptor motif) would be said to comprise 3 repeating units of a block copolymer (due to the presence of 3 glycan acceptors). Similarly, a GAABCP comprising the structure G-S-G-S-G or G-S-G-S-G-S also comprise 3 repeats.

In some instances, the number of repeating units of the GAABCP may be tailored for the specific TUP being modified. For example, but not to be limited in any way by theory, in some instances it may beneficial to conjugate a GAABCP with a large number of repeats to the TUP, while in other instances a smaller number of GAABCP repeats may be preferable. In some instances the number of the GAABCP repeats is determined by the molecular weight of the unmodified TUP. One skilled in the art can readily determine the appropriate number of GAABCP repeats needed to yield a GAABCP-TUP with the appropriate mass, such that, for example the resultant recombinant glycoprotein conjugate can avoid first pass clearance from the plasma compartment by the kidney due to excessively low molecular weight (Maack T (1975) Renal handling of low molecular weight proteins. Am. J. Med. 58(1):57-64; Binder U, Skerra A, Half-life Extension of Therapeutic Proteins via Genetic Fusion to Recombinant PEG Mimetics, in Therapeutic Proteins: Stratagies to Module Their Plasma Half-lives. 1^{st} Ed. 2012 Kontermann R., Ed., Wiley VCH Verlag, incorporated herein). Estimations of polypeptide mass can be calculated using algorithms such as the Compute pI/Mw tool on the ExPASy website (Gasteiger E., Hoogland C., Gattiker A., Duvaud S., Wilkins M. R., Appel R. D., Bairoch A.; Protein Identification and Analysis Tools on the ExPASy Server; (In) John M. Walker (ed): The Proteomics Protocols Handbook, Humana Press (2005), incorporated herein), and confirmed experimentally by analytical methods known to those skilled in the art (e.g., SDS-PAGE or SEC-HPLC with MALS detection). One skilled in the art can easily determine how many GAABCP repeats are required to yield a GAABCP-TUP with extended plasma half-life as taught by the present invention.

The GAABCP of the present invention may comprise from 2-100 repeats of the alternating glycan acceptor motifs/spacer sequences, as disclosed herein, or from 2-90, 2-80, 2-70, 2-65, 2-60, 2-55, 2-50, 5-90, 5-80, 5-70, 5-65, 5-60, 5-55, 5-50, 10-90, 10-80, 10-70, 10-65, 10-60, 10-55, 10-50, 15-90, 15-80, 15-70, 15-65, 15-60, 15-55, 15-50, 20-90, 20-80, 20-70, 20-65, 20-60, 20-55, 20-50, 25-90, 25-80, 25-70, 25-65, 25-60, 25-55, 25-50, 30-90, 30-80, 30-70, 30-65, 30-60, 30-55, 30-50, 40-90, 40-80, 40-70, 40-65, 40-60, 40-55, 40-50, 2-49, 2-48, 2-47, 2-46, 2-45, 2-44, 2-43, 2-42, 2-41, 2-40, 2-39, 2-38, 2-37, 2-36, 2-35, 2-34, 2-33, 2-32, 2-31, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-50, 3-49, 3-48, 3-47, 3-46, 3-45, 3-44, 3-43, 3-42, 3-41, 3-40, 3-39, 3-38, 3-37, 3-36, 3-35, 3-34, 3-33, 3-32, 3-31, 3-30, 3-29, 3-28, 3-27, 3-26, 3-25, 3-24, 3-23, 3-22, 3-21, 3-20, 3-19, 3-18, 3-17, 3-16, 3-15, 3-14, 3-13, 3-12, 3-11, 3-10, 3-9, 3-8, 3-7, 3-6, 3-5, 3-4, 4-50, 4-49, 4-48, 4-47, 4-46, 4-45, 4-44, 4-43, 4-42, 4-41, 4-40, 4-39, 4-38, 4-37, 4-36, 4-35, 4-34, 4-33, 4-32, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 4-7, 4-6, 4-5, 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 7-50, 7-49, 7-48, 7-47, 7-46, 7-45, 7-44, 7-43, 7-42, 7-41, 7-40, 7-39, 7-38, 7-37, 7-36, 7-35, 7-34, 7-33, 7-32, 7-31, 7-30, 7-29, 7-28, 7-27, 7-26, 7-25, 7-24, 7-23, 7-22, 7-21, 7-20, 7-19, 7-18, 7-17, 7-16, 7-15, 7-14, 7-13, 7-12, 7-11, 7-10, 7-9, 7-8, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 9-50, 9-49, 9-48, 9-47, 9-46, 9-45, 9-44, 9-43, 9-42, 9-41, 9-40, 9-39, 9-38, 9-37, 9-36, 9-35, 9-34, 9-33, 9-32, 9-31, 9-30, 9-29, 9-28, 9-27, 9-26, 9-25, 9-24, 9-23, 9-22, 9-21, 9-20, 9-19, 9-18, 9-17, 9-16, 9-15, 9-14, 9-13, 9-12, 9-11, 9-10, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 11-50, 11-49, 11-48, 11-47, 11-46, 11-45, 11-44, 11-43, 11-42, 11-41, 11-40, 11-39, 11-38, 11-37, 11-36, 11-35, 11-34, 11-33, 11-32, 11-31, 11-30, 11-29, 11-28, 11-27, 11-26, 11-25, 11-24, 11-23, 11-22, 11-21, 11-20, 11-19, 11-18, 11-17, 11-16, 11-15, 11-14, 11-13, 11-12, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 13-50, 13-49, 13-48, 13-47, 13-46, 13-45, 13-44, 13-43, 13-42, 13-41, 13-40, 13-39, 13-38, 13-37, 13-36, 13-35, 13-34, 13-33, 13-32, 13-31, 13-30, 13-29, 13-28, 13-27, 13-26, 13-25, 13-24, 13-23, 13-22, 13-21, 13-20, 13-19, 13-18, 13-17, 13-16, 13-15, 13-14, 14-50, 14-49, 14-48, 14-47, 14-46, 14-45, 14-44, 14-43, 14-42, 14-41, 14-40, 14-39, 14-38, 14-37, 14-36, 14-35, 14-34, 14-33, 14-32, 14-31, 14-30, 14-29, 14-28, 14-27, 14-26, 14-25, 14-24, 14-23, 14-22, 14-21, 14-20, 14-19, 14-18, 14-17, 14-16, 14-15, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 15-16, 16-50, 16-49, 16-48, 16-47, 16-46, 16-45, 16-44, 16-43, 16-42, 16-41, 16-40, 16-39, 16-38, 16-37, 16-36, 16-35, 16-34, 16-33, 16-32, 16-31, 16-30, 16-29, 16-28, 16-27, 16-26, 16-25, 16-24, 16-23, 16-22, 16-21, 16-20, 16-19, 16-18, 16-17, 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-32, 17-31, 17-30, 17-29, 17-28, 17-27, 17-26, 17-25, 17-24, 17-23, 17-22, 17-21, 17-20, 17-19, 17-18, 17-17, 17-16, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-29, 18-28, 18-27, 18-26, 18-25, 18-24, 18-23, 18-22, 18-21, 18-20, 18-19, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-29, 20-28, 20-27, 20-26, 20-25, 20-24, 20-23, 20-22, 20-21 repeats of the alternating glycan acceptor motifs/spacer sequences, as disclosed herein.

Thus the glycan acceptor motifs separated by the spacer sequence motifs, including the specific amino acids set forth herein, may in such embodiments be present in the GAABCP in the following number of units: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

In particular embodiments, the present invention provides a glycoprotein conjugate comprising a therapeutically useful polypeptide (TUP) conjugated to one or more GAABCPs, said GAABCPs comprising a polypeptide sequence encoding from 2-100 repeating glycan acceptor motifs separated by spacer motifs.

In further embodiments, the present invention provides a glycoprotein conjugate comprising a TUP conjugated to one or more GAABCPs, said GAABCPs consisting of a polypeptide sequence encoding from 5-50 repeating glycan acceptor motifs separated by spacer motifs.

### Description of Glycan Acceptor Motifs

Any amino acid sequence capable of directing the glycosyltransferase-mediated or chemical addition of one or more glycans to an amino acid comprised in a GAABCP is acceptable for use as a glycan acceptor motif comprised in a GAABCP. Accordingly, GAABCPs may in some instances comprise one or more glycan-acceptor amino acids selected from the group consisting of Asparagine (Asn or N), Arginine (Arg or R), Lysine (Lys or K), Proline (Pro or P), Serine (Ser or S), Threonine (Thr or T), Cysteine (Cys or C), Tyrosine (Tyr or Y), and Tryptophan (Trp or W). Certain GAABCPs comprise one or more glycan chains attached via N- or O-glycosylation to any of the aforementioned appropriate glycan acceptor amino acids. In some particular embodiments, a given GAABCP comprises only N-linked glycan acceptor motifs; only O-linked glycan acceptor motifs; or both O-linked and N-linked glycan acceptor motifs.

Accordingly, in some instances, GAABCPs of the present invention comprise repeating units of a peptide motif of the primary structure [(Spacer motif)-NX₄(T/S)]; wherein N, T, and S are asparagine, threonine, and serine, respectively; X₄ is any amino acid but proline; and (T/S) indicates that this residue may be either threonine or serine. In some such embodiments, X4 is selected from the group consisting of asparagine, glycine, alanine, valine, and isoleucine.

In other instances, the GAABCPs of the present invention comprise repeating units of a peptide motif of the primary structure [NX₄(T/S)-(Spacer motif)]; wherein N, T, and S are asparagine, threonine, and serine, respectively; X₄ is any amino acid but proline; and (T/S) indicates that this residue may be either threonine or serine. In some such embodiments, X4 is selected from the group consisting of asparagine, glycine, alanine, valine, and isoleucine.

Thus, certain embodiments provide for GAABCPs comprising a plurality of amino acid sequences encoding N-glycan acceptor motifs selected from the group consisting of NX₄T; NX₄S; or NX₄T and NX₄S, wherein the relative frequency of each sequence is in any number of combinations; and wherein said plurality of repeating glycan acceptor motifs are separated from one another by a spacer motif, as described herein.

In some embodiments, the present invention provides for a GAABCP-TUP comprising any one of the GAABCP sequences provided as SEQ ID NOS: 54-65. Thus, in one particular non-limiting embodiment, the present invention provides for GAABCPs comprising N-glycan motifs with sequences selected from the group consisting of the sequences provided as SEQ ID NOS: 24-33. In one particular non-limiting embodiment, the present invention provides for GAABCPs comprising repeats of the N-glycan motif NNT, said NNT repeats being separated from one another by spacer motifs, as disclosed herein (e.g., SEQ ID NOS: 34-38 & 54-57). In one particular non-limiting embodiment, the present invention provides for GAABCPs comprising repeats of the N-glycan motif NNS, said NNS repeats being separated from one another by spacer motifs, as disclosed herein (e.g., SEQ ID NOS: 39-43 & 58-61). In one particular non-limiting embodiment, the present invention provides for GAABCPs comprising repeats of the N-glycan motif NGT, said NGT repeats being separated from one another by spacer motifs, as disclosed herein (e.g., SEQ ID NOS: 44-48 & 62-65). In one particular non-limiting embodiment, the present invention provides for GAABCPs comprising repeats of the N-glycan motif NAS, said NAS repeats being separated from one another by spacer motifs, as disclosed herein (e.g., SEQ ID NOS: 49-53).

### Synthetic Production of Glycosylated GAABCPs

As is discussed further throughout this specification, GAABCPs comprise glycan acceptor motifs which may optionally comprise a glycan moiety. Glycan moieties may be attached to the GAABCP by any means.

In some instances, GAABCPs of the present invention are formed by the in vitro chemical addition of an N-glycan moiety with a specific, defined carbohydrate structure to the glycan acceptor motif. In this instance, the N-glycan moiety may in some embodiments be de novo synthesized using a chemo-enzymatic process and then chemically attached to the glycan acceptor motif present in the GAABCP-TUP (e.g., via an asparagine or cysteine residue) using conjugation chemistry well known to those skilled in the art. Alternatively the chemo-enzymatically synthesized N-glycan moiety can be further modified with an amino acid residue (e.g., an asparagine residue) attached at the proximal N-acetyl-beta-D-glucosamine residue of the oligosaccharide chain to form an N-glycan-Asn moiety. This N-glycan-Asn moiety can be further modified by methods known to those skilled in the art to enable its use in forming a GAABCP-polypeptide or GAABCP-TUP containing the chemo-enzymatically formed N-glycan using solid-phase peptide synthesis methods (such methods including but not being limited to the methods disclosed in U.S. Pat. Nos. 7,943,763, 8,063,202, and 8,399,656, incorporated herein in their entirety.

### Description of Spacer Motifs

The spacer sequence motifs (or “spacer motifs”) separating the glycan acceptor motifs in the GAABCPs of the present invention may comprise any suitable amino acid sequence. Suitable spacer motif amino acid sequences include those that physically separate the repeating glycan motifs of the GAABCP so as to promote or permit the glycosyltransferase-catalyzed attachment of at least one carbohydrate or carbohydrate chain to at least one of the glycan acceptor motifs when appropriate conditions are provided. The number of amino acids comprised in the spacer motifs may be constant throughout the GAABCP (e.g., a GAABCP comprising 3 or more glycan acceptor motifs, each glycan acceptor motif being spaced apart from the nearest glycan acceptor motif(s) by spacer motifs of an equal number of amino acids); or the spacer sequence size may vary (e.g., in a GAABCP comprising three or more glycan acceptor motifs, wherein not all of the motifs are equally spaced apart from the nearest glycan acceptor motif(s)). In one embodiment, at at least two of said spacer motifs have different amino acid sequence lengths. In one embodiment, all of said spacer motifs comprise an identical number of amino acids. In addition, for a given GAABCP, the spacer motif may vary in its specific amino acid sequence with each subsequent repeat, e.g., a GGS spacer motif may be followed by a GGG motif, followed by an GGGSS motif, etc); or the spacer motif may be held constant within the repeats of the GAABCP, e.g., GGS followed by a GGS, followed by a GGS. In one embodiment, at at least two of said spacer motifs have different amino acid sequence composition. In other embodiments, all of said spacer motifs comprise identical amino acid sequence compositions.

Spacer motifs may be located adjacent to the glycan acceptor motif, e.g., N- or C-terminal to the glycan acceptor motif, or spacer motifs can flank the glycan acceptor motif, i.e., spacer motifs may be located both N- and C-terminally to the glycan acceptor motif. The spacer motifs may comprise flexible amino acid sequences or rigid sequences.

In some embodiments, a GAABCP of the present invention comprises two or more glycan acceptor motifs separated by a spacer motif (e.g., a flexible spacer sequence) that comprises or consists of from 1 to about 150 amino acids, from 1 to about 100 amino acids, or from 1 to about 50 amino acids. In some embodiments, the spacer sequence comprises or consists of from about 6-100 amino acids, or from about 6-50, 6-25, 6-15, 9-50, 9-25, 9-15, 12-50, 12-25, 12-15 amino acids. In particular embodiments, the spacer sequence comprises or consists of about 150 amino acids, or about 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids.

In some embodiments, each spacer motif comprised in a GAABCP comprises or consists of greater than 3 amino acids, 6 amino acids, 9 amino acids, or 12 amino acids. In some embodiments, the spacer motif is at least about 6 amino acids, or at least about, 9, 12, 15, 18, 21 or more amino acids. In some embodiments, the spacer motif is no less than 6 amino acids in length. In some embodiments, the spacer motif is no less than 9 amino acids in length. In some embodiments, the spacer motif is no less than 12 amino acids in length.

In certain embodiments, it is preferable to increase the length of the spacer motif to include at least 6 amino acids or at least about 7, 8, 9, 10, 11, 12, 13, 14, 15, 16, 17, 18, 19, 20, 21, or more amino acids.

In some embodiments, the spacer sequence is a non-native sequence with respect to the TUP to which the GAABCP is conjugated.

In particular embodiments, the spacer motif separating the repeating glycan acceptor motifs comprises or consists of the primary structure (X₁X₂X₃)ₙ, wherein X₁ and X₂ are selected from the groups consisting of glycine (Gly or G), alanine (Ala or A), valine (Val or V), isoleucine (Ile or I), and leucine (Leu or L); X₃ is selected from the group consisting of serine (Ser or S), threonine (Thr or T), asparagine (Asp or N), glutamine (Gln or Q), G, A, V, I, and L; and n is an integer between 1 and and 5 (e.g., n may be 1, 2, 3, 4, or 5). In some embodiments, X1 is glycine. In some embodiments, X2 is glycine. In some embodiments, X3 is serine. In some embodiments, X1 and X2 are both glycine. In some embodiments, X1 and X2 are glycine and X3 is serine.

In other particular embodiments, the spacer motif separating the repeating glycan acceptor motifs comprises the primary structure (X1X2X3)ₙ, wherein X1 and X2 are selected from the groups consisting of glycine (Gly or G), alanine (Ala or A), valine (Val or V), isoleucine (Ile or I), and leucine (Leu or L); X3 is selected from the group consisting of serine (Ser or S), threonine (Thr or T), asparagine (Asp or N), glutamine (Gln or Q), G, A, V, I, and L; and n is an integer between 1 and 50 (e.g., n may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50. In certain embodiments, n is an integer greater than or equal to 3. In some embodiments, X1 is glycine, X2 is glycine, X3 is serine, X1 and X2 are both glycine, or X1 and X2 are glycine and X3 is serine. In certain embodiments, n is an integer greater than or equal to 2, 3, 4, 5, 6, or 7. In certain embodiments, n is 2 or at least 2.

In certain embodiments, but not to limit the scope of the invention, the spacer motif may comprise G and/or S residues, e.g., any one spacer motif selected from the group consisting of GGS (i.e. n=1) (SEQ ID NO: 1), GGSGGS (i.e. n=2) (SEQ ID NO: 16), GGSGGSGGS (i.e. n=3) (SEQ ID NO: 17), and other spacer motif sequences provided as SEQ ID NOS: 2-6, 11-15, 18-23, and 124-133, all of which are suitable for use in the present invention.

In some embodiments, but not to limit the scope of the invention, the spacer motif may consist of G and/or S residues, e.g., any one spacer motif selected from the group consisting of GGS (i.e. n=1) (SEQ ID NO: 1), GGSGGS (i.e. n=2) (SEQ ID NO: 16), GGSGGSGGS (i.e. n=3) (SEQ ID NO: 17), and other spacer motif sequences provided as SEQ ID NOS: 2-6, 11-15, 18-23, and 124-133, all of which are suitable for use in the present invention.

In other embodiments, but not to limit the scope of the invention, the spacer motif may comprise a sequence selected from the group consisting of any one of SEQ ID NOS: 7-10. As was described above, in certain embodiments, the glycan acceptor motifs disclosed herein are separated from one another by the spacer sequences disclosed herein. In some embodiments, the addition of the spacer motifs between the repeating glycan motifs results in maximal and consistent occupancy of the glycan acceptor sites when exposed to the appropriate glycan synthesis/transfer systems in vitro, ex vivo, or in vivo (i.e., when appropriate conditions are provided). In some embodiments, the addition of the spacer motifs between the repeating glycan motifs results in the post-translational addition of complex-type N-glycan structures that demonstrate a high-level of terminal sialylation of the carbohydrate chain (e.g., sialic acid occupancy on the termini of the carbohydrate chains greater than 60%) when exposed to the appropriate glycan synthesis/transfer systems in vitro, ex vivo, or in vivo (i.e., when appropriate conditions are provided).

According to the present disclosure, certain particular non-limiting embodiments of the present invention therefore provide GAABCPs comprising, or consisting of, from 2 to 50 repeats of any one GAABCP block unit polypeptide sequence selected from the group consisting of GGSNNT (SEQ ID NO: 34), GGSGGSNNT (SEQ ID NO: 35), GGSGGSGGSNNT (SEQ ID NO: 36), GGSGGSGGSGGSNNT (SEQ ID NO: 37), GGSGGSGGSGGSGGSNNT (SEQ ID NO: 38), GGSNNS (SEQ ID NO: 39), GGSGGSNNS (SEQ ID NO: 40), GGSGGSGGSNNS (SEQ ID NO: 41), GGSGGSGGSGGSNNS (SEQ ID NO: 42), GGSGGSGGSGGSGGSNNS (SEQ ID NO: 43), GGSNGT (SEQ ID NO: 44), GGSGGSNGT (SEQ ID NO: 45), GGSGGSGGSNGT (SEQ ID NO: 46), GGSGGSGGSGGSNGT (SEQ ID NO: 47), GGSGGSGGSGGSGGSNGT (SEQ ID NO: 48), GGSNAS (SEQ ID NO: 49), GGSGGSNAS (SEQ ID NO: 50), GGSGGSGGSNAS (SEQ ID NO: 51), GGSGGSGGSGGSNAS (SEQ ID NO: 52), and GGSGGSGGSGGSGGSNAS (SEQ ID NO: 53). Other particular non-limiting embodiments of the present invention provide GAABCPs comprising, or consisting of, from 2 to 100 repeats of any one GAABCP block unit polypeptide motif disclosed herein (such as e.g., any one of the GAABCP block unit sequences provided in Table 3 or any one of SEQ ID NOS: 34-53). In some embodiments, the present invention provides a GAABCP comprising 5, 10, 15, or 20 repeating units of said motif. Thus, GAABCPs comprising, or consisting of, these specific peptide motifs (i.e., these block units) may be present in the polypeptide in the following number of units: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 (thus including any subrange thereof), all of which are suitable for use in the present invention.

In some embodiments, the present invention provides a GAABCP comprising from 2 to 50 repeating glycan acceptor motifs, each glycan acceptor separated from its nearest neighboring glycan acceptor by a spacer sequence, wherein said GAABCP comprises two or more different GAABCP block unit sequences selected from any of those presented as SEQ ID NOS 34-65, or included in Table 3.

In some particular instances, GAABCPs of the present invention comprise or consist of at least 5, or exactly 5, repeats of any one of the GAABCP block unit sequences disclosed herein, e.g., (GGSGGSNNT)₅ (SEQ ID NO: 54), (GGSGGSNNS)₅ (SEQ ID NO: 58), and/or (GGSGGSNGT)₅ (SEQ ID NO: 62). In some particular instances, the GAABCPs comprise or consist of at least 10, or exactly 10, repeats of any one of the GAABCP block unit sequences disclosed herein, e.g., (GGSGGSNNT)₁₀ (SEQ ID NO: 55), (GGSGGSNNS)₁₀ (SEQ ID NO: 59), and/or (GGSGGSNGT)₁₀ (SEQ ID NO: 63). In still other instances, the present invention provides GAABCPs comprising or consisting of at least 15, or exactly 15, repeats of any one of the GAABCP block unit sequences disclosed herein, e.g., (GGSGGSNNT)₁₅ (SEQ ID NO: 56), (GGSGGSNNS)₁₅ (SEQ ID NO: 60), and/or (GGSGGSNGT)₁₅ (SEQ ID NO: 64). In further instances, GAABCPs comprising or consisting of at least 20, or exactly 20, repeats of any one of the GAABCP block unit sequences disclosed herein, e.g., (GGSGGSNNT)₂₀ (SEQ ID NO: 57), (GGSGGSNNS)₂₀ (SEQ ID NO: 61), and/or (GGSGGSNGT)₂₀ (SEQ ID NO: 65) are provided.

Description of Therapeutically Useful Polypeptides

The present invention provides for compositions of GAABCP-TUPs produced via the conjugation of one or more therapeutically useful polypeptides (TUP) with one or more GAABCP in the manner described herein. Any TUP is suitable for conjugation with the various GAABCPs described herein, according to the present disclosure.

In various embodiments, the present invention comprises the conjugation of a GAABCP to a TUP to increase the plasma half-life of the TUP. In some embodiments, the present invention provides a GAABCP-TUP comprising a GAABCP conjugated to a TUP, which TUP, when unconjugated, exhibits a plasma half-life of less than 48 hours, as determined according to any method described herein e.g., Example 7. In particular embodiments, the present invention provides a GAABCP-TUP comprising a GAABCP conjugated to a TUP, which TUP, when unconjugated, exhibits a plasma half-life of less than 24 hours, as determined according to any method described herein e.g., Example 7. In certain embodiments, the present invention provides a GAABCP-TUP comprising a GAABCP conjugated to a TUP, which TUP, when unconjugated, exhibits a plasma half-life of less than 12 hours, as determined according to any method described herein e.g., Example 7. A TUP may comprise a naturally expressed polypeptide sequence (i.e. a native sequence), or it may comprise a non-naturally expressed polypeptide sequence (i.e., a variant), or it may comprise a fragment of a naturally or non-naturally expressed polypeptide sequence. Non-native TUPs may comprise additions, deletions, and/or substitutions of amino acids, as compared to the corresponding native TUP amino acid sequence, provided that the resulting modified TUP has a desirable therapeutic use. In some instances, a non-native TUP comprises an amino acid sequence that is at least about 50%, 60%, 70%, 80%, 90%, or 95% identical to its corresponding native amino acid sequence, including all integers (e.g., 91%, 92%, 93%, 94%, 96%, 97%, 98%, 99%) and ranges above and in between (e.g., 50%-99%; 60%-99%; 70%-99%; 80%-99%; 90%-99%; 95%-99%). In some instances, a non-native TUP sequence is encoded by polynucleotide sequences that are at least about 50%, 60%, 70%, 80%, 90%, or 95% identical to its corresponding native polynucleotide sequence, including all integers (e.g., 91%, 92%, 93%, 94%, 96%, 97%, 98%, 99%) and ranges above and in between (e.g., 50%-99%; 60%-99%; 70%-99%; 80%-99%; 90%-99%; 95%-99%).

Native TUPs may be derived from any species. In some instances, the TUPs are of prokaryotic origin, and in other instances the TUPs are of eukaryotic origin. TUPs may also comprise hybrid or chimeric amino acid sequences deriving from more than one species. Accordingly, the present invention provides for TUPs that are in some instances of mammalian origin, e.g., of murine or human origin. In some particular embodiments, the TUPs of the present invention are native human polypeptides. The TUP may also be a fusion protein.

The TUP may comprise native or modified glycan acceptor sites, wherein, e.g., the modification results in the addition or deletion of one or more glycan acceptor motif. The TUP may comprise native or modified proteolytic cleavage sites, wherein, e.g., the modification results in the addition or deletion of one or more proteolytic cleavage site. The TUP may comprise native or modified post-translational modification sites, wherein, e.g., the modifications result in the addition or deletion of one or more site for post-translational modifications.

In particular, it is contemplated that the native TUPs encompassed by the present invention may include, but are not limited to, cytokines, chemokines, lymphokines, ligands, receptors, peptide hormones, apoptosis-inducing polypeptides, enzymes, antibodies, proteolytic antibody fragments or domains, antigen specific antibody fragments, coagulation factors, complement proteins, growth factors, and fragments or variants thereof.

Suitable cytokines include, but are in no way limited to, e.g., growth factors selected from a group consisting of human growth hormone, granulocyte macrophage colony stimulating factor (GM-CSF), granulocyte colony stimulating factor (G-CSF), macrophage colony stimulating factor (M-CSF), platelet derived growth factor (PDGF), tumor necrosis factor (TNF), epidermal growth factor (EGF), vascular endothelial growth factor (VEGF), fibroblast growth factor, (FGF), nerve growth factor (NGF), and erythropoietin (EPO). Suitable cytokines further include, but are in no way limited to, interferons such as, e.g., beta-interferon, gamma-interferon interferon type I, interferon type II and interferon type III. Suitable cytokines further include, but are in no way limited to, cytokines selected from a group consisting of interferon gamma inducing factor I (IGIF), RANTES (regulated upon activation, normal T-cell expressed and presumably secreted), macrophage inflammatory protein-1-alpha (MIP-1-α), macrophage inflammatory protein-1-β (MIP-1-β), glucagon-like peptide 1 (GLP-1), glucagon-like peptide 2 (GLP-2), Leishmania elongation initiating factor (LEIF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), and fragments or variants thereof.

Suitable peptide hormones include, but are in no way limited to, e.g., hormones selected from the group consisting of insulin, insulin-like growth factor 1 (IGF-1), adrenocorticotropic hormone (ACTH), human growth hormone (hGH), glucagon, glucagon-like peptide 1 (GLP-1), exendin-4, releasing hormone, thyroid stimulating hormone, melanocyte stimulating hormone, luteinizing hormone, follicle stimulating hormone, prolactin, antidiuretic hormone, oxytocin, calcitonin, thyroid hormone, parathyroid hormone, renin, relaxin-3, relaxin-2, etc, and fragments or variants thereof.

The present invention further provides for the conjugation of GAABCPs to TUPs selected from the group consisting of a soluble gp-120 glycoprotein, a soluble gp-160 glycoprotein, an IL-1 receptor antagonist, a TNFalpha receptor antagonist, viral barrier to entry peptides (e.g., enfuvirtide, or the 18-amino acid peptide designated as CL58 that was derived from the CLDN1 intracellular and first transmembrane region that inhibits both de novo and established HCV infection in vitro, or human apolipoprotein E peptide, hEP, which specifically blocked the entry of cell culture grown HCV (HCVcc) at submicromolar concentrations), peptides derived from human Fcγ receptor II (CD32) or other FcyR subtypes useful in the treatment of systemic lupus erythematosus or other autoimmune disorders, and human factor VII, VIII, IX, and other proteins in the blood coagulation cascade, and fragments or variants thereof. The present invention further provides for the addition of GAABCPs to TUPs selected from the group consisting of an antibody, an antigen specific antibody fragment, a proteolytic antibody fragment or domain, a single chain antibody, a genetically or chemically optimized antibody, or a proteolytic and antigen-binding fragment thereof, and fragments or variants thereof.

The present invention further provides for the addition of GAABCPs to TUPs selected from the group consisting of a receptor, the extracellular domain of a receptor, membrane-associated proteins, selected from the group consisting of tumor necrosis factor (TNF) type I receptor, tumor necrosis factor (TNF) type II receptor, IL-1 receptor type II, and IL-4 receptor, CTLA-4, hTRAIL receptor, TACI, VEGFR1/2, Tie-2, lymphocyte function-associated antigen 3, FAS receptor, activin receptor type 2A, B-cell activating factor receptor, fibroblast growth factor receptors, other cytokine and immune cell regulating protein receptors, and fragments or variants thereof.

G-CSF.

One non-limiting example of a TUP that is well suited for conjugation with a GAABCP according to the present disclosure is the human cytokine granulocyte colony-stimulating factor (hG-CSF; SEQ ID NO: 97). G-CSF is produced by monocytes and fibroblasts and it induces rapid proliferation and release of neutrophilic granulocytes from the marrow compartment to the bloodstream, thereby providing a therapeutic effect in fighting infection. As explained in U.S. Pat. No. 6,831,158, (incorporated herein by reference in its entirety) recombinant human (rh) G-CSF is generally used for treating various forms of leukopenia (a reduced level of white blood cells) and neutropenia (a reduced level of neutrophils). Leukopenia and neutropenia result in an increased susceptibility to various infections.

The plasma half-life of rhG-CSF is short, i.e., ˜2-4 hr (Kotto-Kome A C, et. al. (2004) Pharmacol. Res. 50(1):55-8, incorporated herein), thus requiring frequent administration to achieve desired responses. Accordingly, one utility of the present invention includes modification of G-CSF with a GAABCP according to the present invention.

Aside from G-CSF, per se, G-CSF analogs that are biologically functional or have G-CSF-like biological activity are also useful TUPs for modification with a GAABCP as disclosed herein. Methods of preparing rh-G-CSF are disclosed in U.S. Pat. No. 4,810,643, incorporated herein. Various G-CSF analogs, which contain a mutated form of the native G-CSF protein (SEQ ID NO: 98), are also reported in U.S. Pat. No. 4,810,643, incorporated herein. The polynucleotide encoding rhG-CSF and the amino acid structure of hG-CSF are both provided in U.S. Pat. No. 5,985,265, incorporated herein.

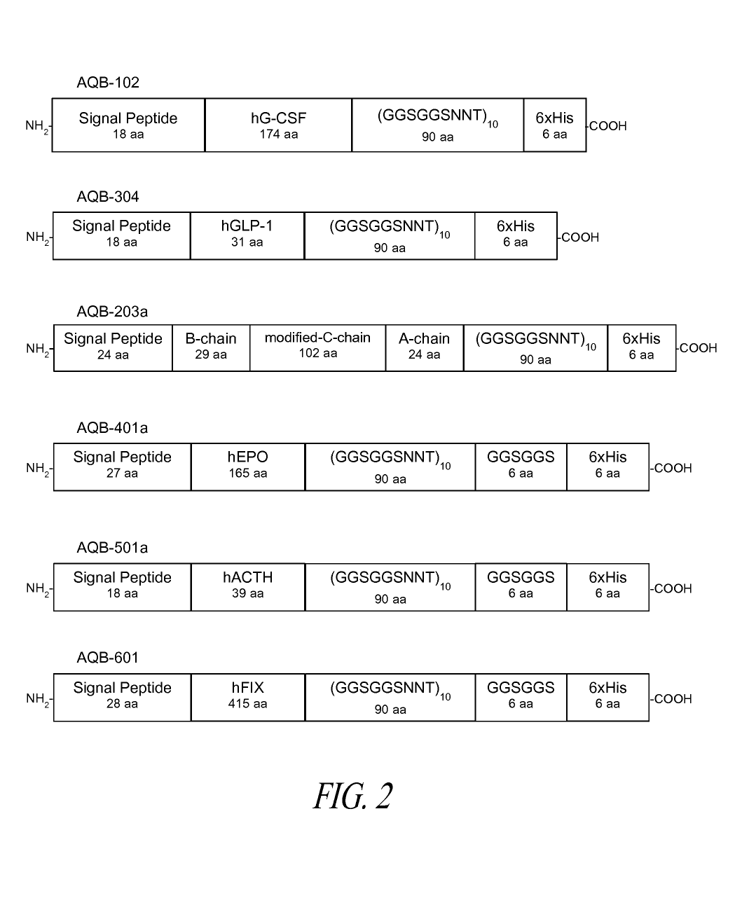

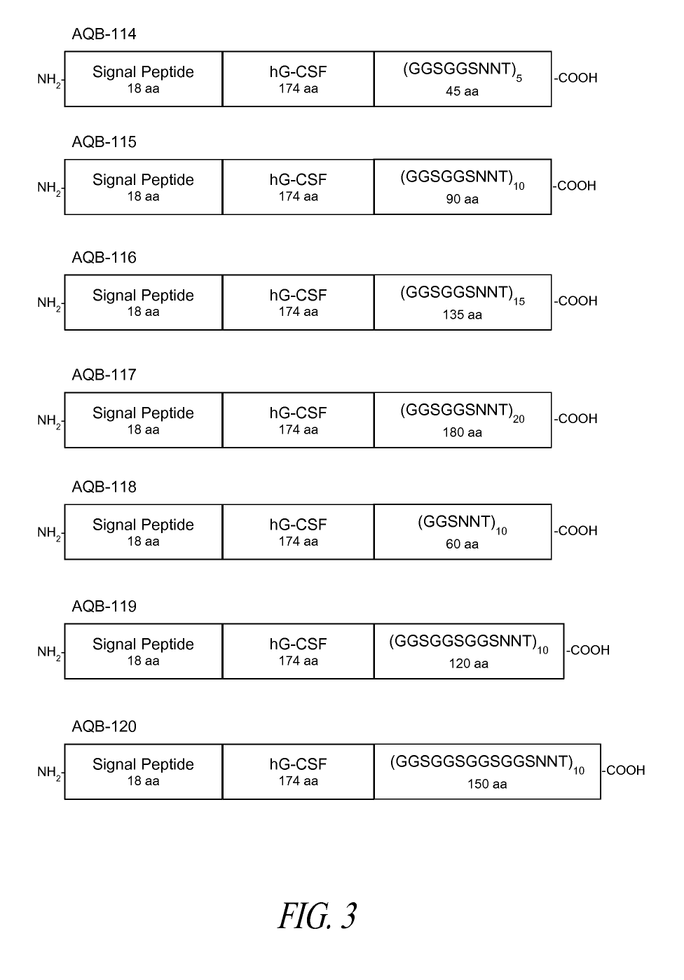

Accordingly, one example of the present invention provides for the genetic conjugation of the modified form of hG-CSF (SEQ ID NO: 98) to the N-terminus of a GAABCP with the amino acid sequence GGSGGSNNT repeated 10 times (SEQ ID NO: 55). This hG-CSF-based GAABCP-TUP contains a yeast Pho1 secretion signal peptide (SEQ ID NO: 66) N-terminal to the hG-CSF, and a C-terminal 6×His affinity tag (SEQ ID NO: 68) that creates a fully formed GAABCP-TUP according to the present disclosure (SEQ ID NO: 100). This example is presented as AQB-102 in FIG. 2, with a greater range of GAABCP variants shown in FIG. 3.

GLP-1.

Another non-limiting example of a TUP conjugated to a GAABCP is the human hormone glucagon-like peptide-1 (hGLP-1; SEQ ID NO: 103). Under physiological conditions, insulin secretion is controlled by several factors including two key incretin hormones, glucose-dependent insulinotropic polypeptide (GIP) and GLP-1. These hormones stimulate glucose-dependent insulin secretion and account for up to 70% of the insulin response after oral glucose intake, referred to as the ‘incretin effect’.

Clinical trials have demonstrated that exogenously administered GLP-1 normalized the insulin response in individuals with impaired glucose tolerance, and exhibited a blood-glucose lowering effect in patients with type II diabetes mellitus. However its rapid degradation to an inactive form by the enzyme dipeptidyl peptidase IV and other endogenous peptidases leads to the critical disadvantage of a short plasma half-life (t_{1/2}=1 to 2 min) following intravenous GLP-1 administration. Thus, native GLP-1 would need to be infused continually for effective therapy.

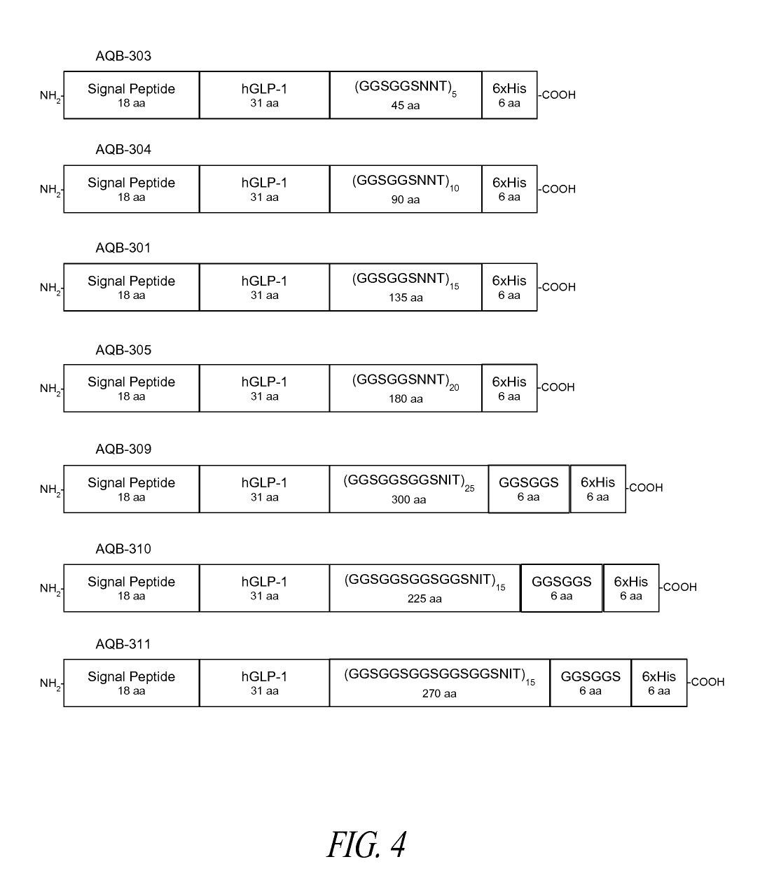

Accordingly, an additional example of the present invention provides for the genetic conjugation of a modified form of hGLP-1(7-37) (SEQ ID NO: 104) to the N-terminus of a GAABCP with the amino acid sequence GGSGGSNNT repeated 10 times (SEQ ID NO: 55). This modified GLP-1-based GAABCP-TUP contains the yeast Pho1 secretion signal peptide (SEQ ID NO: 66) N-terminal to the TUP plus a C-terminal 6×His affinity tag (SEQ ID NO: 68) that creates a fully formed GAABCP-TUP according to the present disclosure (SEQ ID NO: 106). This example is presented as AQB-304 in FIG. 2, with additional GAABCP variants shown in FIG. 4.

Relaxins.

Another non-limiting example of a TUP that may be conjugated with a GAABCP is the hormone human prorelaxin-2 (hRLN2; SEQ ID NO: 109), which is an endocrine and autocrine/paracrine peptide hormone of the insulin superfamily. Relaxin-2 signaling occurs in many different organs and cell types, and is initiated by the protein's binding to one of several class I (rhodopsin-like) G-protein-coupled receptor, principally its cognate receptor LGR7 (leucine-rich G protein-coupled receptor 7) also named RXFP1 (relaxin family peptide 1 receptor) (Kong R C, et al. (2010) Mol. Cell. Endocrinol. 320:1-15, incorporated herein). Binding of hRLN2 to its receptors can initiate signaling through several different transduction pathways.

hRLN2 has been demonstrated to have a wide range of potential clinically relevant applications including, but not limited to heart failure; fibrotic diseases, including heart, lungs, liver, skin (keloids), and kidneys; cancer; asthma; in the treatment of post-due date pregnancy (i.e., induce labor by hRLN2 treatment); and in the treatment of muscle diseases including acute injuries and chronic illnesses such as muscular dystrophy. However, a key limitation to the commercialization of hRLN2-based therapies is its short plasma half-life, which is less than 10 minutes after intravenous administration (Dschietzig T, Bartsch C, Baumann G, Stangl K. (2006) Relaxin-a pleiotropic hormone and its emerging role for experimental and clinical therapeutics. Pharmacol Ther. 112(1):38-56, incorporated herein). As a consequence, patients require continuous infusions of hRLN2 to achieve the sustained plasma levels required to achieve therapeutic effects.

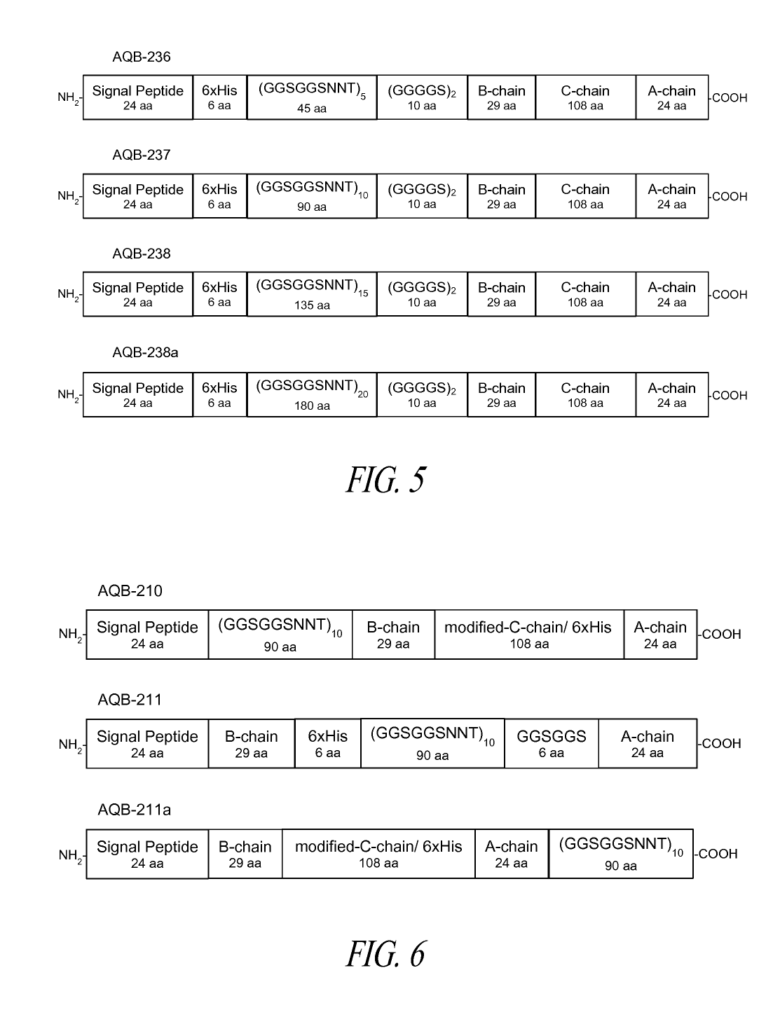

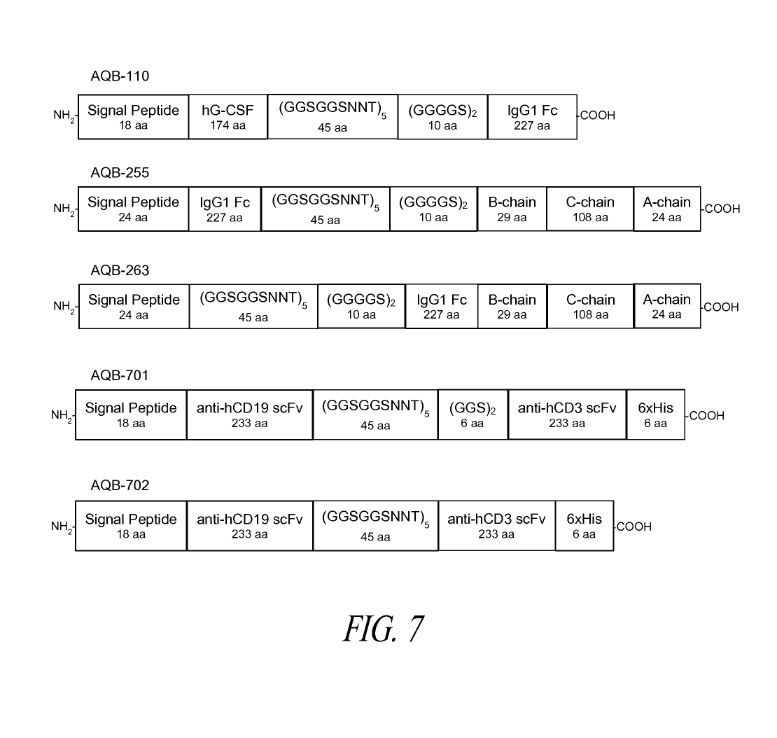

Accordingly, an additional example of the present invention provides for the genetic conjugation of native hRLN2 (SEQ ID NO: 109) to the C-terminus of GAABCP with the amino acid sequence GGSGGSNNT repeated 10 times (SEQ ID NO: 55) separated by a GGGGSGGGGS linker sequence (SEQ ID NO: 93). This modified hRLN2-based GAABCP-TUP also contains the native secretion signal peptide and a 6×His affinity tag (SEQ ID NO: 68) conjugated to the N-terminus of the GAABCP to create a fully formed GAABCP-TUP according to the present disclosure (SEQ ID NO: 115). This example is presented as AQB-237 in FIG. 5, where other GAABCP variants can also be seen in FIG. 5 and FIG. 6.

Aside from hRLN2, per se, other Relaxin-like polypeptides, and fragments or variants thereof, are known and are suitable TUPs for modification into a GAABCP-TUP according to the present disclosure. These include Relaxin 1, Relaxin 3, and the insulin-like peptide genes INSL3, INSL4, INSL5, and INSL6. Additionally, the present invention provides for the use of isoforms, splice variants, proteolytic fragments, combinations, and/or other variants of the relaxin-like polypeptide, such as, e.g., those described in U.S. Pat. No. 4,758,516, U.S. Pat. No. 4,871,670, U.S. Pat. No. 5,811,395, U.S. Pat. No. 5,759,807, WO 2013/004607, and WO 2009/055854, all of which are herein incorporated by reference.

EPO.

Another non-limiting example of a TUP that may be conjugated with a GAABCP is human erythropoietin (EPO; SEQ ID NO: 146). EPO, a member of the haematopoietic growth factor family, is synthesized mainly in the adult kidney and fetal liver in response to tissue hypoxia due to decreased blood oxygen availability. Recombinant EPO having the amino-acid sequence of naturally occurring EPO has been produced and approved to treat anemia associated with kidney functional failure, cancer, and other pathological conditions (Blackwell K, et al., The Oncologist 9 (suppl 5), pp 41-47 (2004)). In addition to its erythropoietic properties, EPO may also act on non-bone marrow cells such as neurons, suggesting other possible physiological/pathological functions of EPO in the central nervous system (CNS) and other organs/systems (Lappin T R, et al., Stem Cells 20, pp 485-492 (2002)). As EPO receptors have been found in many different organs, EPO may have multiple biological effects, such as acting as an anti-apoptotic agent.

The mean in vivo half-lives of rHuEPO alpha and rHuEPO beta administered intravenously or subcutaneously are only 8.5 and 17 hours respectively (Macdougall I C., Nephrol. Dial. Transplant. 16(suppl 3), pp 14-21 (2001); Joy M S., Ann. Pharmacother. 36, pp 1183-1192 (2002)). Patients therefore need an injection schedule of daily, twice weekly or three times per week, which imposes a burden on both patients and health care providers. Accordingly, one utility of the present invention includes modification of hEPO with a GAABCP according to the present invention.

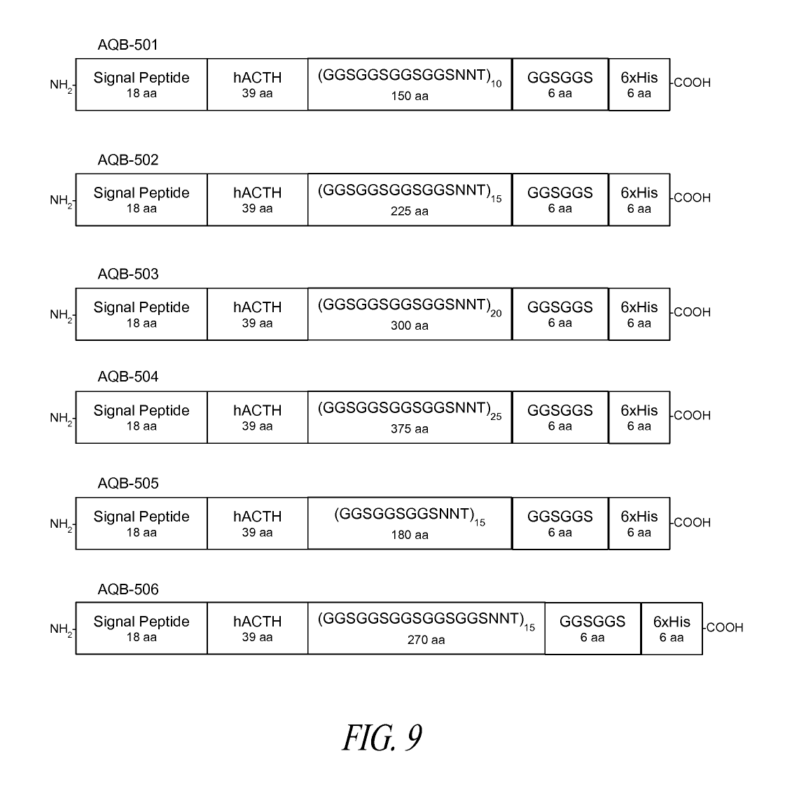

Aside from naturally occurring EPO, per se, EPO analogs that are biologically functional or have EPO-like biological activity such as the ability to cause bone marrow cells to increase production of reticulocytes and red blood cells are also useful TUPs for modification with a GAABCP as disclosed herein. Currently, three kinds of rHuEPO or rHuEPO analogs are commercially available, namely (i) rHuEPO alpha, (ii) rHuEPO beta, and (iii) darbepoetin alfa, also known as Novel Erythropoiesis Stimulating Protein (NESP) or Aranesp™. In certain embodiments, the present invention provides polypeptide conjugates comprising one or more GAABCP conjugated to any one of these or other EPO analogs known in the art. In particular embodiments, the present invention provides an EPO-GAABCP conjugate such as is shown in FIG. 2 as construct 401a. In certain embodiments, the present invention provides an EPO-GAABCP conjugate such as is shown in FIG. 8.

ACTH and POMC.

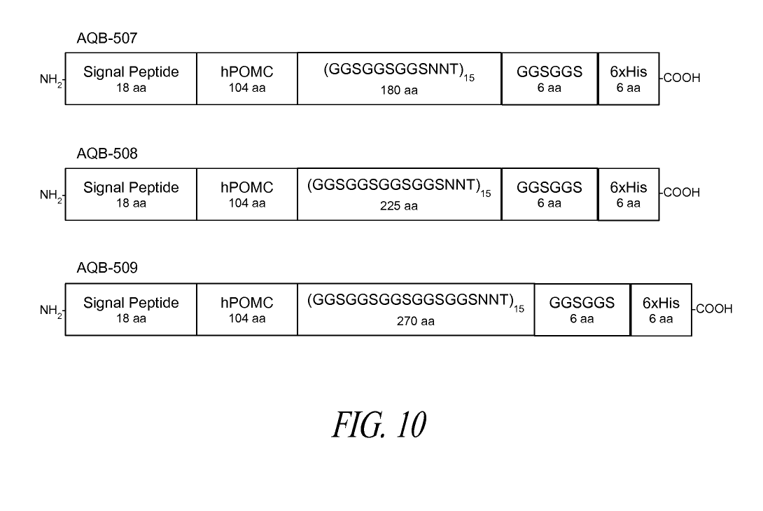

Another non-limiting example of a TUP conjugated to a GAABCP is the human polypeptide hormone adrenocorticotropic hormone (hACTH; SEQ ID NO: 157) and its precursor polypeptide pro-opiomelanocortin (hPOMC 138-241; SEQ ID NO: 164). See FIG. 2 construct 501a and FIGS. 9 & 10 for additional examples. ACTH is a 39 amino acid peptide hormone secreted by the anterior pituitary gland. ACTH is secreted from the anterior pituitary in response to corticotropin-releasing hormone (CRH) that is secreted from the hypothalamus. The release of ACTH stimulates the adrenal cortex with subsequent increased production of glucocorticosteroids and/or cortisol from the adrenal cortex. ACTH is synthesized from a precursor polypeptide pre-pro-opiomelanocortin (pre-POMC). The removal of the signal peptide during translation produces a 267 amino acid polypeptide POMC. POMC undergoes a series of post-translational modifications to yield various polypeptide fragments including and not limited to ACTH, β-lipotropin, γ-lipotropin, α, β, γ-Melanocyte Stimulating Hormone (MSH) and β-endorphin. POMC, ACTH and β-lipotropin are also secreted from the pituitary gland in response to the hormone corticotropin-releasing hormone (CRH). In some embodiments, the first 13 amino acids of ACTH1-39 are cleaved to form α-melanocyte-stimulating hormone (α-MSH). ACTH may be useful treating various diseases or conditions related to decreased ACTH production or secretion including, e.g., various types of hypocortisolism including Addison disease; primary adrenal insufficiency; secondary adrenal insufficiency; pituitary insufficiency; hypothalamic insufficiency. ACTH may be useful for treating renal disorders (8796416); migraines (20140294923); ALS (WO2011143152); shock conditions and respiratory and cardiocirculatory insufficiency (U.S. Pat. No. 5,380,710); exacerbations of Multiple Sclerosis, acute exacerbations of rheumatic disorders such as Rheumatoid Arthritis and Psoriatic Arthritis, acute episodes of Ulcerative Colitis, acute bouts of Systemic Lupus Erythematous, and Infantile Spasms (U.S. Pat. No. 5,413,797).

However, the serum half-life of ACTH is short approximately 15 minutes. Accordingly, in some embodiments, the present invention provides ACTH or an ACTH precursor (e.g., POMC) or related polypeptide (e.g., MSH, β-endorphin, etc) conjugated to a GAABCP according to the present invention. Further, in some embodiments, the present invention provides for the conjugation of a GAABCP to an ACTH analogue having ACTH activity (e.g., in vitro activation of an ACTH receptor or in vivo ability to stimulate glucocorticosteroids and/or cortisol production or secretion from the adrenal cortex).

Glyco-BiTEs.

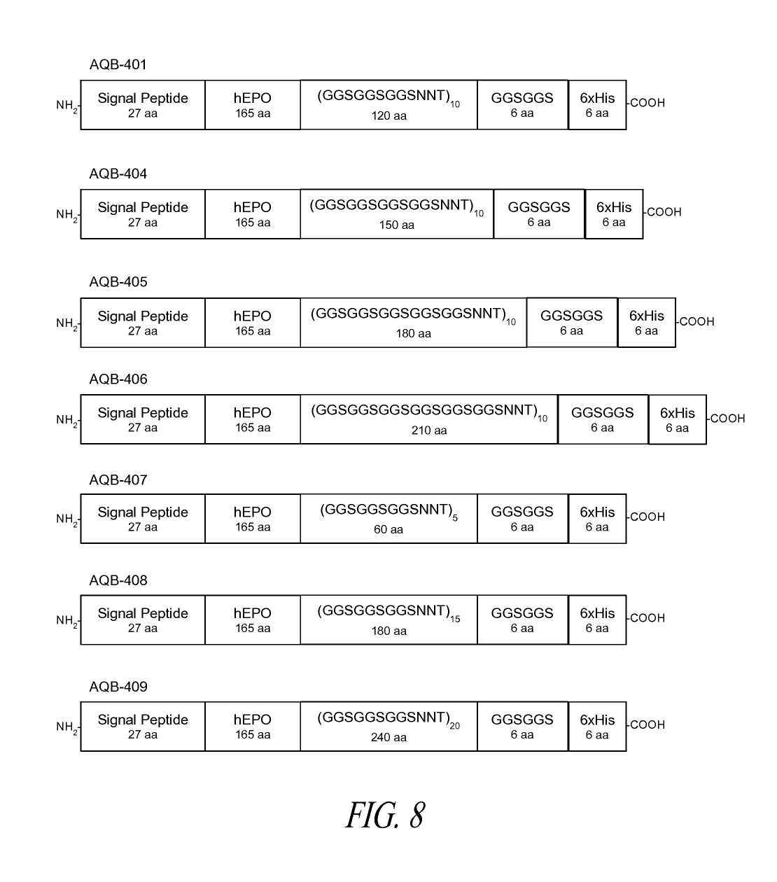

GAABCP conjugation also finds utility in the generation of long-acting bispecific antibodies such as, e.g., bispecific T-cell engagers (BiTE®s) (Sheridan C, Nat Biotechnol. 2012 (30):300-1)). A BiTE® is a bispecific fusion protein comprising two binding domains (e.g., single-chain variable fragments or “scFvs”), fused together, optionally via a linker sequence, wherein a first binding domain is directed against a tumor-associated antigen (TAA) and a second binding domain is directed against a trigger molecule on the effector cells, such as, e.g., CD3 on T cells (Kontermann, MABS 2012 (4) 182-197; Chames and Baty, MABS 2009 (1) 539-547; Moore et al. Blood 2011 (117) 4542-4551). In some embodiments, the present invention provides a glycoprotein conjugate comprising two binding domains suitable for inclusion in a BiTE linked together with a sequence comprising or consisting of a GAABCP of the present disclosure. Such GAABCP-BiTEs, or “Glyco-BiTEs” may be directed toward any suitable antigen. In particular embodiments, a Glyco-BiTE of the present invention targets a TAA specific for a solid tumor (e.g., HER2, EGFR). In particular embodiments, a Glyco-BiTE of the present invention targets a TAA specific for a liquid tumor (e.g., CD19). In some embodiments, addition of a GAABCP to a BiTE increases the half-life of the Glyco-BiTE as compared to the corresponding unconjugated BiTE. In some embodiments, the present invention further provides for the linking of any two binding domains (e.g., one or more scFvs of different human antibodies) with a GAABCP. In one embodiment, the present invention provides the hGlyco-BiTEs presented as SEQ ID NOS: 177 and 178). See FIG. 7 constructs 701 and 702 for additional examples.

### Description of Secretion Signal Peptides

GAABCP-TUPs may optionally comprise native secretion signal peptides, non-native secretion signal peptides, or mutated secretion signal peptides fused to their N-terminus to induce appropriate passage of the GAABCP-TUP through the secretory pathway (i.e. induction of secretion). In some instances, the secretion signal peptide comprised in the GAABCP-TUP is the native secretion signal peptide of the unmodified TUP. In some instances, a secretion signal peptide from another secreted protein is ligated to the N-terminus of the GAABCP-TUP, e.g., if the GAABCP-TUP is derived from a TUP that does not comprise a native secretion signal peptide. In still other instances, the secretion signal peptide that is naturally present at the N-terminus of the TUP is replaced by a secretion signal peptide from another secreted protein. Secretion signal peptides may be derived from any species, and thus may optionally be chimeric (e.g., a secretion signal peptide sequence derived from a protein of one species ligated to the N-terminus of a TUP derived from another species). One non-limiting example of a chimeric secretion signal peptide is the 18 amino acid secretion signal sequence from the Schizosaccharomycen pombe pho1+ acid phosphatase (Pho1) which has been utilized for the secretion of recombinantly expressed heterologous proteins (e.g, GFP and HPV 16 E7). Therefore, one embodiment of the present invention provides for the use of the Pho1 secretion signal peptide (SEQ ID NO: 66) with a GAABCP-TUP that is comprised of modified hG-CSF genetically conjugated to the N-terminus of (GGSGGSNNT)₁₀ (SEQ ID NO: 100). Another embodiment of the present invention provides for the use of the Pho1 secretion signal peptide (SEQ ID NO: 66) with a GAABCP-TUP that is comprised of modified hGLP-1 genetically conjugated to the N-terminus of (GGSGGSNNT)₁₀ (SEQ ID NO: 106). The human serum albumin signal peptide (SEQ ID NO: 67) has also been utilized as a signal peptide for recombinantly expressed proteins in mammalian systems (Kober L, Zehe C, Bode J (2013) Optimized signal peptides for the development of high expressing CHO cell lines. Biotechnol Bioeng. 110(4):1164-1173, incorporated herein). However, the skilled artisan can fully appreciate the interchangability of secretion signal peptides, and thus it is to be understood that such an embodiment also clearly encompasses the use of any other secretion signal peptides capable of inducing suitable secretion of a GAABCP-TUP.

### Description of Flexible Linkers

GAABCP-TUPs may also optionally comprise a sequence of amino acids that form an unstructured polypeptide domain in an aqueous solution (e.g., flexible linkers), to separate the various polypeptide domains of the GAABCP-TUPs. Examples of flexible linkers are well known to those skilled in the art (see e.g., Wriggers, Chakravarty, Jennings (2005) Biopolymers 80(6):736-46; George R A, Heringa J (2002) Protein Eng. 15:871-9; Argos, P (1990) J. Mol. Biol. 211:943-58, incorporated herein). Flexible linkers may comprise any suitable amino acid sequence. Typically, preferred flexible linkers will comprise an amino acid sequence that does not promote the formation of 2° or higher structures in the amino acid sequence, thereby potentially inhibiting biological activity of the resultant GAABCP-TUP. In certain embodiments, the flexible linker comprises at least one glycine, alanine, valine, isoleucine, leucine, serine, threonine, asparagine, and/or glutamine residue. In some preferred embodiments, the flexible linker comprises glycine and serine residues. Flexible linker sequences exhibiting a low immunogenicity score are preferred. Immunogenicity testing can be performed using the NetMHCIIpan algorithm (Karosiene E, Rasmussen M, Blicher T, Lund O, Buus S, Nielsen M (2013) NetMHCIIpan-3.0, a common pan-specific MHC class II prediction method including all three human MHC class II isotypes, HLA-DR, HLA-DP and HLA-DQ. Immunogenetics 65(9):655-65, incorporated herein), which calculates the potential binding affinity of proteins or peptides to MHCII complex. The higher the calculated binding affinity the higher is the risk to induce antibodies directed against the polypeptide of interest.

Non-limiting examples of acceptable flexible linkers include, e.g., the likes of the linker sequence motifs provided as SEQ ID NOS: 72-96, or pluralities thereof. In certain embodiments, the flexible linker comprises from 1-20 repeats of any one of the linker sequence motifs provided as SEQ ID NOS 72, 77, 82, 87 or 92. Thus, the motifs included in the specific flexible linkers set forth herein, may be present in the flexible linker the following number of times: 1, 2, 3, 4, 5, 6, 7, 8, 9, 10, 11, 12, 13, 14, 15, 16, 17, 18, 19 or 20. In particular embodiments, the linkers of the present invention comprise a sequence selected from the group consisting of (GGGS)₁₋₅ (e.g., SEQ ID NOS: 82-86); (GGSG)₁₋₅ (e.g., SEQ ID NOS: 87-91); (GGS)₁₋₅ (e.g., SEQ ID NOS: 72-76); (GSG)₁₋₅ (SEQ ID NOS: 77-81; (GGGGS)₁₋₅ (SEQ ID NOS: 92-96); (GGGSG)₁₋₅; and (GGSGG)₁₋₅, where the amino acids listed within the parentheses make up the optionally repeating flexible linker motif that is comprised in the flexible linker; and wherein the numbers outside of the parenthesis represent the number of repeats of said motif. Accordingly, in such embodiments, the number of repeating linker motifs ranges from 1-5 and thus includes 1, 2, 3, 4 or 5 repeats of the sequence in the parenthesis.

In some embodiments of the present invention, one or more flexible linkers connect a TUP to a GAABCP.

### Description of Affinity Tags

The GAABCP-TUP may also optionally comprise additional amino acid insertions such as one or more affinity tag (or “epitope tag”), to assist in its detection, purification, or solubilization. Many such affinity tags are known to those skilled in the art, and would be acceptable for use with a GAABCP-TUP (Lichty J J, Malecki J L, Agnew H D, Michelson-Horowitz D J, Tan S (2005) Comparison of affinity tags for protein purification. Protein Expr. Purif 41(1):98-105, incorporated herein). Non-limiting examples include any tag selected from the group consisting of: mono or polyhistidine sequences, e.g. 6×His (SEQ ID NO: 68), FLAG-tag (SEQ ID NO: 69), c-myc-tag (SEQ ID NO: 70), HA-tag (SEQ ID NO: 71), as well as V5, GFP (and variants thereof), immunoglobulin Fc portions (and variants thereof) (SEQ ID NO: 112), streptavidin, calmodulin, S-tag, SBP, CBP, softag 1, softag 3, Xpress, isopeptag, spytag, BCCP, GST, MBP, Nus, thioredocin, NANP, TC, and Ty. Such affinity tags may be conjugated to the GAABCP-TUP at any suitable location. In some aspects of the invention, the tags are fused to the termini of a GAABCP-TUP, e.g., the N-termini or the C-termini of the GAABCP-TUP, and in other aspects the tags are fused to the internal amino acid sequence of the GAABCP-TUP. As is common within the art, these tags may optionally be attached to the GAABCP-TUP via a cleavable sequence such as, e.g., a TEV protease recognition motif so as to allow for the optional removal of the tag via proteolytic digestion. Many similar tags are available in the art and they are all suitable for attaching an affinity tag to a GAABCP-TUP according to the specifications disclosed herein.

### 1° Structure of GAABCP-TUP Genes

GAABCP-TUPs are novel glycoprotein conjugates comprising one or more TUPs conjugated to one or more GAABCPs, as described herein, wherein a GAABCP may be conjugated to a TUP at any suitable location. A given GAABCP-TUP may comprise only one GAABCP, or it may comprise a plurality of GAABCPs. Additionally, GAABCPs may be ligated within the internal amino acid sequence of a TUP, and/or they may be ligated to one or both termini of a TUP.

In some embodiments, nucleotide sequences encoding GAABCPs are easily genetically conjugated to TUP-encoding nucleotide sequences using various genetic techniques common to the average molecular biologist. This can be done at low throughput (i.e., via traditional cloning techniques, e.g., restriction cloning, per cloning, etc), or this can be performed in a high throughput manner. For example, but not to be limited in any way, many high throughput cloning techniques have been described in the art, that enable the simultaneous construction of hundreds to thousands of fusion proteins (e.g, Festa, et. al. (2013) J. Proteomics 13(9):1381-99, incorporated herein). Furthermore, pharmaceutical and biotechnology companies routinely perform semi-high throughput and high throughput in vitro/in vivo pharmacokinetic screens that allow for reliable prediction of protein absorption, distribution, metabolism and excretion (e.g., Chaturvedi P R, et. al. (2001) Curr. Opin. Chem. Biol. 5(4):452-63, incorporated herein). Thus, without undue experimentation, the myriad of TUP-coding sequences readily available to the skilled artisan (e.g., as are freely available on the UniProt Knowledgebase database (UniProt Consortium (2013) Update on activities at the Universal Protein Resource (UniProt) in 2013. Nucleic Acids Res. 41:D43-7, incorporated herein) or the UniGene database (Pontius J U, Wagner L, Schuler G D. UniGene: a unified view of the transcriptome. In: The NCBI Handbook. Bethesda (Md.): National Center for Biotechnology Information; 2003, incorporated herein) are specifically contemplated for GAABCP modification according to the present invention.

N-/C-Terminal Ligation of TUP to GAABCP.

In some aspects, the present invention provides for the ligation of a GAABCP to one or both of the termini of a TUP (i.e., the N-terminus, C-terminus, or both the N- and the C-terminus). Without being limited by theory, it is believed that when a GAABCP is conjugated to the termini of a TUP, the vast majority of TUPs may be modified according to the present disclosure, to achieve sufficient increases in plasma half-life. The specific examples provided herein are therefore described to ensure that the invention is properly understood; however, it is important to stress that the present invention should not be construed as being limited to these examples. Indeed, quite the opposite is believed to be true, as the conjugation of one or more GAABCPs to increase half-life is contemplated as being appropriately applicable to any TUP that would benefit from an increased half-life.

Accordingly, one aspect of the present invention provides for GAABCP-TUPs comprising different TUPs conjugated by a peptide bond at its C-terminus to the N-terminus of a GAABCP. Non-limiting examples of such GAABCP-TUPs are illustrated in FIG. 2 as cartoon representations depicting the various domains from N-terminal to C-terminal, and the number of amino acids in the domain's polypeptide chains. In such embodiments the TUP may contain a native or non-native secretion signal peptide at its N-terminus. The resultant GAABCP-TUPs may also optionally comprise an affinity tag sequence located at the C-terminus, internal to the polypeptide, or at the N-terminus. FIG. 2 shows three embodiments each with a distinct TUP, modified hG-CSF in AQB-102 (SEQ ID NO: 100), modified hGLP-1 in AQB-304 (SEQ ID NO: 106), and modified prorelaxin-2 in AQB-203a (SEQ ID NO: 113), modified hEPO in AQB-401a (SEQ ID NO: 179), hACTH in AQB-501a (SEQ ID NO: 180) and hFIX in AQB 601 (SEQ ID NO: 168), that are conjugated to the N-terminus of the GAABCP comprising 10 repeating units of the peptide motif GGSGGSNNT (SEQ ID NO: 55). To facilitate purification of the GAABCP-TUP, a 6×His affinity tag is genetically conjugated to the C-terminus of the GAABCP domain of each construct (SEQ ID NO: 68) with or without the presence of an intervening flexible linker sequence (SEQ ID NO: 73).

The present invention also provides for GAABCP-TUPs comprising a given TUP conjugated by a peptide bond at its C-terminus to a GAABCP of differing repeat lengths. Non-limiting examples of such GAABCP-TUP constructs are illustrated in FIGS. 3, 4, 5, 8, 9 & 11 as cartoon representations depicting the various domains from N-terminal to C-terminal, and the number of amino acids in the domain's polypeptide chains. FIG. 3 shows four embodiments each with a Pho1 signal peptide conjugated to the N-terminus of a TUP comprised of hG-CSF, and each with distinct GAABCP consisting of either 5 (AQB-114; SEQ ID NO: 134), 10 (AQB-115; SEQ ID NO: 135), 15 (AQB-116; SEQ ID NO: 136) or 20 (AQB-117; SEQ ID NO: 137) repeating units of the peptide motif GGSGGSNNT. FIG. 4 shows four embodiments each with a Pho1 signal peptide (SEQ ID NO: 66) conjugated to the N-terminus of a TUP comprised of hGLP-1, and each with distinct GAABCP consisting of either 5 (AQB-303; SEQ ID NO: 105), 10 (AQB-304; SEQ ID NO: 106), 15 (AQB-301; SEQ ID NO: 107) or 20 (AQB-305; SEQ ID NO: 108) repeating units of the peptide motif GGSGGSNNT. A 6×His affinity tag, to facilitate purification of the GAABCP-TUP, is genetically conjugated internal to the C-chain of the polypeptide in each of these constructs (SEQ ID NO: 68).

The present invention further provides for GAABCP-TUPs comprising a flexible linker sequence, conjugated by a peptide bond, inserted between the GAABCP and the TUP. Said flexible linker sequence may be inserted between the GAABCP and any group of amino acids that form the domain structure of the TUP. In some embodiments, such a linker allows for GAABCP conjugation to a TUP without substantially interfering with the biological activity of the GAABCP-TUP. Such embodiments may also optionally comprise one or more affinity tags located at the C-terminus, the N-terminus, or internal to the GAABCP-TUP. The GAABCP-TUP may also comprise more than one flexible linker and/or affinity tag. In such instances, the affinity tags may be positioned adjacent to one another, or they may be separated from one another e.g., via a linker.

Non-limiting examples of GAABCP-TUPs with the GAABCP and TUP separated by flexible linkers are illustrated in FIG. 4 as cartoon representations depicting the various domains from N-terminal to C-terminal, and the number of amino acids in the domain's polypeptide chains. FIG. 5 shows four embodiments each with a native human relaxin-2 signal peptide conjugated to the N-terminus of a 6×His affinity tag (SEQ ID NO: 68), conjugated to the N-terminus of GAABCPs consisting of either 5 (AQB-236; SEQ ID NO: 114), 10 (AQB-237; SEQ ID NO: 115), 15 (AQB-238; SEQ ID NO: 116) or 20 (AQB-238a; SEQ ID NO: 117) repeating units of the peptide motif GGSGGSNNT, conjugated to the N-terminus of a GGGGSGGGGS linker domain (SEQ ID NO: 93), conjugated to the N-terminus of native human prorelaxin-2 (SEQ ID NO: 109). In such embodiments the TUP may contain a native or non-native secretion signal peptide at its N-terminus. The resultant GAABCP-TUP may also optionally comprise an affinity tag at the C-terminus, internal to the polypeptide, or at the N-terminus.

Internal Ligation of a GAABCP in the Sequence of a TUP.

The present invention also provides for GAABCP-TUPs comprising one or more GAABCPs conjugated within the internal amino acid sequence of a TUP. For example, in one embodiment, the present invention provides for GAABCP-TUPs comprising modified human prorelaxin-2 conjugated by a peptide bond to a GAABCP that is located within the amino acid sequence of the TUP. Non-limiting examples of GAABCP-TUPs with the GAABCP domain placed to either the N-terminus, C-terminus or internal to the TUP domain are illustrated in FIG. 6 as cartoon representations depicting the various domains from N-terminal to C-terminal, and the number of amino acids in the domain's polypeptide chains. In such embodiments the TUP may contain a native or non-native secretion signal peptide at its N-terminus. The resultant GAABCP-TUP may also optionally comprise an affinity tag sequence located at the C-terminus, internal to the polypeptide, or at the N-terminus. FIG. 5 shows four non-limiting embodiments of the invention each with a native relaxin-2 signal peptide at the N-terminus and containing a common form of GAABCP, comprising 10 repeating units of the peptide motif GGSGGSNNT (SEQ ID NO: 55), that is located either N-terminal to a variant of human prorelaxin-2 (AQB-210; SEQ ID NO: 118), internal to the amino acid sequence of a variant of human prorelaxin-2 (AQB-211; SEQ ID NO: 119), or C-terminal to a variant of human prorelaxin-2 (AQB-211a; SEQ ID NO: 120).

Conjugation of Multiple TUPs and/or GAABCPs.

The present invention also provides for GAABCP-TUPs comprising one or more GAABCPs conjugated with one or more TUPs, in either a linear or branched primary amino acid configuration. GAABCP-TUPs that exemplify this embodiment of the invention may be comprised of one or more GAABCPs and/or TUPs, these GAABCPs and TUPS may represent identical or differing primary amino acid sequences. The GAABCPs and TUPs may be separated by a linker sequence (e.g., a flexible linker), and may contain affinity tags, as described herein.

Non-limiting examples of such GAABCP-TUP constructs are illustrated in FIG. 7 as cartoon representations depicting the various domains from N-terminal to C-terminal, and the number of amino acids in the domain's polypeptide chains. In such embodiments, the TUP may contain a native or non-native secretion signal peptide at its N-terminus. The resultant GAABCP-TUP may also optionally comprise an affinity tag sequence located at the C-terminus, internal to the polypeptide, or at the N-terminus. FIG. 7 shows five embodiments of the invention each with an N-terminal signal peptide (AQB-110, 701 and 701: Pho1, i.e. SEQ ID NO: 66; AQB-255 and 263: native human pro relaxin-2) and a GAABCP comprising 5 repeating units of the peptide motif GGSGGSNNT (SEQ ID NO: 54), along with two TUPs that have varying orientations with respect to the GAABCP. In AQB-110 (SEQ ID NO: 121) a modified hG-CSF TUP is positioned N-terminal to the GAABCP, and a modified IgG1 Fc TUP (SEQ ID NO: 112) is located to the C-terminus of the GAABCP with an intervening GGGGSGGGGS linker domain (SEQ ID NO: 93). In AQB-255 (SEQ ID NO: 122) the modified IgG1 Fc TUP is N-terminal to the GAABCP, and a native relaxin-2 TUP is located to the C-terminus of the GAABCP with an intervening GGGGSGGGGS linker domain (SEQ ID NO: 93). In AQB-263 (SEQ ID NO: 123) both TUPS, a modified IgG1 Fc and a native relaxin-2 are located at the C-terminus of the GAABCP with an intervening GGGGSGGGGS flexible linker domain (SEQ ID NO: 93) separating the N-terminus of the modified IgG1 Fc from the C-terminus of the GAABCP; and wherein the C-terminus of the modified IgG1 Fc is conjugated directly to the N-terminus of the native relaxin-2 TUP. In AQB-701 (SEQ ID NO: 177) one TUP, an anti-hCD19 scFv sequence, is located to the N-terminus of the GAABCP, and the second TUP, an anti-CD3 scFv sequence, is located to the C-terminus of the GAABCP with an intervening GGSGGS flexible linker domain (SEQ ID NO: 73). In AQB-702 (SEQ ID NO: 178) the molecule is the same as AQB-701 but does not contain the flexible linker domain.

### GAABCP-TUP Gene Design

The present invention is also directed to a method of making GAABCP-TUPs. This method involves culturing a host cell comprising a novel gene encoding the GAABCP-TUP under conditions whereby the recombinantly inserted gene is expressed, thereby producing the recombinant GAABCP-TUP; and then purifying the recombinant GAABCP-TUP from the cell or culture medium, characterizing the glycoprotein conjugate using analytical assays known to those skilled in the art, and finally testing the GAABCP-TUP as desired, typically for bioactivity specific to the given TUP and various pharmacokinetic properties including plasma half-life.

The aforementioned novel gene is designed in silico to create a synthetic polynucleotide that encodes a polypeptide that is comprised of at least an N-terminally positioned mammalian secretion signal peptide domain; one or more TUP domains and one or more GAABCP domains (in any suitable order); and a stop codon positioned at the 5′ end of the gene. The novel gene can also optionally include polynucleotide fragments that encode flexible linker domains, an affinity tag domain, and/or 5′ and 3′ restriction sites, cloning sites and/or regulatory element domains. The order of the domains within the gene can be varied to optimize the expression of the gene and bioactivity and/or pharmacokinetic properties of the gene product, as herein described, except that the signal peptide domain is always at the 5′ end of the polynucleotide and the stop codon is always at the 3′ end of the polynucleotide. Optionally, the nucleotide sequence of the novel gene is then examined by computer algorithms, which are well known to those skilled in the art, in order to optimize the nucleotide sequence for expression in a particular cell line, e.g., CHO, NS0 or 293 cells. These algorithms substitute specific nucleotide codons encoding amino acids in the novel gene sequence for degenerate codon sequences that are optimally utilized in the particular mammalian cell line, while not altering the primary amino acid sequence. This process is known to those skilled in the art as ‘codon optimization’ (Fath S, Bauer, A S, Liss M, Spriestersbach A, et. al. (2011) Multiparameter RNA and Codon Optimization: A Standardized Tool to Assess and Enhance Autologous Mammalian Gene Expression. PLoS One 6(3):e17596, incorporated herein). Many programs/methods for codon optimization are commonly available to the skilled artisan, (e.g., but not to be limited in any way, Gene Composer™ (Lorimer D, Raymond A, Walchli J, Mixon M, et. al. (2009) Gene Composer: database software for protein construct design, codon engineering, and gene synthesis. BMC Biotechnology 9:36, incorporated herein). Alternatively, manual codon optimization may be performed by simply considering the codon usage frequency data in the host cell of choice. For example, but not to be limited in anyway, if the amino acid glutamic acid (Glu, E) is naturally encoded by the DNA nucleotides GAG in a particular position of the polynucleotide sequence, but in the cell line of choice the GAG codon is a rare and inefficiently utilized codon, whereas the codon GAA is frequently used to encode Glu, then the GAG sequence can be mutated to GAA, thus maintaining fidelity with regard to the encoded primary polypeptide sequence, but optimizing the codons used in the polynucleotide. Thus in similar embodiments, any codon in the polynucleotide sequence can be similarly optimized for recombinant protein expression provided the encoded amino acid sequence remains the same.

During this codon optimization process computer algorithms may also determine the optimal G/C content, eliminate certain nucleotide repeats, prevent the introduction of out of frame stop codons, eliminate Shine-Dalgarno sequences, eliminate Chi sequences, eliminate unwanted restriction sites, and reduce RNA secondary structure. Some such computer algorithms have been written so as to also engineer in a Kozak translation initiation sequence to optimize levels of mammalian protein expression for the novel gene. Finally the computer algorithm may design polynucleotide sequences flanking the 3′ and 5′ end of the novel gene that represent unique DNA restriction enzyme cleavage sites, to facilitate cloning the novel gene into a suitable recombinant protein expression vector.

### Gene Cloning and Expression Vectors

The GAABCP-TUPs of the present invention are in some embodiments made via standard recombinant techniques in molecular biology that are well known, (e.g., see, Joseph Sambrook, et al., Molecular Cloning: A Laboratory Manual, 2nd ed., 1.53 [Cold Spring Harbor Laboratory Press 1989], incorporated herein). The methodology is not limited to any particular cloning strategy. The skilled artisan may use any variety of cloning strategies to produce an expression vector containing a novel GAABCP-TUP gene that comprises a novel gene of the present invention.

In some instances, once the nucleotide sequence for the novel gene has been determined (optionally the ‘codon optimized’ sequence) the gene is synthesized de novo to form a cDNA that contains the novel gene plus additional nucleotide sequences containing unique restriction enzyme cleavage sites on the 5′ and 3′ ends of the gene. Several direct gene synthesis methods can be employed for this purpose, these methods are well known to those in the art (Montague M G, Lartigue C, Vashee S. (2012) Synthetic genomics: potential and limitations. Curr. Opin. Biotechnol. 23(5):659-665, incorporated herein). Polynucleotides representing the entire novel gene, plus the aforementioned additional nucleotide sequences, can be directly synthesized, or alternatively subfragments of the gene may be directly synthesized followed by ligation of these fragments using PCR primers to create the full-length gene. Following gene synthesis the nucleotide sequence of the novel gene with the flanking restriction sites, and optional regulatory elements, may be confirmed by direct gene sequencing of the cDNA that are well known to those skilled in the art (Pettersson E, Lundeberg J, Ahmadian A. (2009) Generations of sequencing technologies. Genomics 93:(2)105-111, incorporated herein). Once the cDNA sequence has been confirmed the cDNA is cloned into a recombinant protein expression vector using standard recombinant techniques in molecular biology. Expression vectors are typically selected to match the particular host cell used for expressing the recombinant protein in order to optimize the quantity and quality of recombinant protein expressed, e.g., the TOPO cloning vector pOptiVEC from Life Technologies is commonly used to express novel genes in the DG44 variant of CHO cells. Additional examples include the pTT5 clonging vector used to express genes in CHO-3E7 or CHO-BRI cells, and the pXC-17.4 cloning vector used to express gene in CHOK1 SV GS-KO cells. Expression vectors are typically engineered with nucleotide sequences that represent additional elements needed to optimize the expression of the novel gene in a particular host cell, including but not limited to, cloning sites to facilitate insertion of the cDNA containing the novel gene, a promoter/enhancer element to allow efficient, high-level gene expression, primer sites to allow sequencing of the cDNA insert, a polyadenylation signal to allow efficient transcription termination and polyadenylation of the novel gene's mRNA, and selection genes to allow for selection of transformants in bacterial and mammalian cells. The expression vector containing the novel gene is then transformed into competent bacteria, positive transformants are selected using one of the selectable markers in the expression vector, the bacterial cultures containing the expression vector are expanded, the bacterial cells are harvested, and the expanded amounts of the expression vector are purified under endotoxin-free conditions using methods that are well know to those in the art. As a final step the expression plasmid containing the novel gene is linearized by digestion with a specific DNA restriction enzyme.

In still other embodiments, a polynucleotide encoding the TUP is first cloned into an expression vector. Then, oligonucleotides that encode the repeating units of the GAABCP portion are cloned into the construct through a ligation or multimerization scheme, in which the oligonucleotides are ligated together to form a polynucleotide that encodes the GAABCP portion. In this manner, the oligonucleotides are added to the polynucleotide that encodes the TUP portion, thereby producing the chimeric DNA molecule containing the GAABCP-TUP gene within the expression vector. Then, a host cell capable of expressing the GAABCP-TUP gene may be transformed with the expression vector carrying the GAABCP-TUP gene, or the transformation may occur without the utilization of a carrier, such as an expression vector. Then, the transformed host cell is cultured under conditions suitable for expression of the GAABCP-TUP gene, resulting in the recombinant expression of the GAABCP-TUP gene.

This cloning process may also take place through “directional cloning”, which is well known in the art. Directional cloning refers to the insertion of a polynucleotide into a plasmid or vector in a specific and predefined orientation. Once cloned into a vector, a polynucleotide sequence can be lengthened at its 3′ end or other polynucleotides inserted at its 5′ and/or 3′ ends. Such a design provides an efficient and relatively easy way to create large polymers without having to perform multiple rounds of ligation. The vector preferably contains restriction sites upstream of a cloned polynucleotide, but downstream of regulatory elements required for expression to facilitate the insertion of the second polynucleotide. To facilitate directional cloning, “adapter oligonucleotides” may be ligated to the 5′ and 3′ ends of the chimeric DNA molecule encoding the protein conjugate. Preferably, the adapters contain restriction sites that are compatible with those present in the expression vector. The 3′ adapter oligonucleotide may also comprise a stop codon to designate the end of the encoding sequence to which it is ligated. The oligonucleotides encoding the polypeptide portion are preferably added in excess of the adapter oligonucleotides to increase the likelihood that a long polynucleotide is generated after ligation.

The linearized expression vector containing the novel GAABCP-TUP gene can be introduced into the host cell in accordance with techniques well known to those skilled in the art. These techniques include, but are not limited to; transformation using calcium phosphate co-precipitated with the linearized expression vector containing the novel gene, lipidic reagent cotransfection (i.e., Lipofectamine), electroporation, transduction by contacting the cells with a virus, or microinjection of the linearized expression vector containing the novel GAABCP-TUP gene directly into the cells.

The expression host cell will typically be a higher eukaryotic cell, such as a mammalian cell in order to facilitate the addition of appropriately formed O- or N-glycan carbohydrate chains. Alternatively, one skilled in the art will recognize that non-mammalian host cells can be engineered to contain the needed mammalian glycosylation enzyme systems to ensure the addition of appropriately formed N-glycan carbohydrate moieties (Jacobs P P, Callewaert N (2009) N-glycosylation engineering of biopharmaceutical expression systems. Curr. Mol. Med. 9(7):774-800, incorporated herein). Suitable host cells commonly utilized by those skilled in the art include, but are not limited to CHO cells and variants thereof, HEK293 cells and variants thereof, COS cells and variants thereof, and the like; however, these examples are not meant to be limiting the scope of the invention and other cell lines, or in vitro protein synthesis systems, capable of the post-translational addition of suitable formed O- or N-glycan carbohydrate chains to the recombinant protein expressed by the novel GAABCP-TUP gene may also be employed as a matter of choice.

A wide variety of host cell/expression vector combinations are employed in expressing the novel GAABCP-TUP gene of the present invention. Generally, expression vectors will include origins of replication and selectable markers permitting transformation of the host cell (e.g., dihydrofolate reductase or neomycin resistance for eukaryotic cell culture, or such as tetracycline or ampicillin resistance in E. coli, or the S. cerevisiae TRP1 gene), and a promoter derived from a highly-expressed gene to direct transcription of a downstream structural sequence. Such promoters can be derived from operons encoding glycolytic enzymes such as 3-phosphoglycerate kinase (PGK), A-factor, acid phosphatase, or heat shock proteins, among others. The heterologous structural sequence is assembled in appropriate phase with translation initiation and termination sequences, and in some embodiments, a secretion signal peptide capable of directing extracellular secretion of the novel GAABCP-TUP gene's translation product. Expression vectors may further comprise an origin of replication to ensure maintenance of the expression vector and to, if desirable, provide amplification within the host.

Mammalian expression vectors will typically comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements. Mammalian expression vectors contemplated for use in the invention, but by no means meant to limit the description of the invention, include vectors with inducible promoters, such as the dihydrofolate reductase promoters, any expression vector with a DHFR expression cassette or a DHFR/methotrexate co-amplification vector such as pED (Pstl, Sall, Sbal, Smai and EcoRI cloning sites, with the vector expressing both the cloned gene and DHFR; Kaufman R J, Sharp P A (1982) Amplification and expression of sequences cotransfected with a modular dihydrofolate reductase complimentary DNA gene. J. Mol. Biol. 159:601-621, incorporated herein). Alternatively, a glutamine synthetase/methionine sulfoximine co-amplification vector, such as pEE14 (Hindiii, Xbali, Smai, Sbal, EcoRI and Bell cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI, Sfii, Xhoi, Noti, Nhei, Hindiii, Nhei, Pvuii and Kpni cloning sites, constitutive RSV-LTR promoter, hygromycin selectable marker; Invitrogen), pCEP4 (BamHI, Sfii, Xhoi, Noti, Nhei, Hindiii, Nhei, Pvuii and Kpni cloning sites, constitutive hCMV immediate early gene promoter, hygromycin selectable marker; Invitrogen), pMEP4 (Kpni, Pvui, Nhei, Hindiii, Noti, Xhoi, Sfii, BamHI cloning sites, inducible metallothionein IIa gene promoter, hygromycin selectable marker, Invitrogen), pREPS (Bamm, Xhoi, Noti, Hindiii, Nhei and Kpni cloning sites, RSV-LTR promoter, histidinol selectable marker; Invitrogen), pREP9 (Kpni, Nhei, HindiII, Noti, Xhoi, Sfii, BamHI cloning sites, RSV-LTR promoter, G418 selectable marker; Invitrogen), and pEBVHis (RSV-LTR promoter, hygromycin selectable marker, N-terminal peptide purifiable via a variety of metal-chelating affinity chromatography resins and cleaved by enterokinase; Invitrogen).

Selectable mammalian expression vectors for use in the invention include, but are not limited to, pOPTIVec-TOPO cloning vector (pCMV, TOPO, G418, DHFR expression/methotrexate co-amplification), pTT5 (hCMV promoter, adenovirus tripartite leader, splice donor/acceptor, multiple cloning site, rabbit beta-globin polyadenylation site, Epstein-Barr virus origin of replication, bacterial origin of replication; CNRC), pRc/CMV (Hindiii, BstXI, Noti, Sbai and Apal cloning sites, G418 selection, Invitrogen), pRc/RSV (Hindii, Spei, BstXI, Noti, Xbai cloning sites, G418 selection, Invitrogen) and the like. Vaccinia virus mammalian expression vectors (see, for example, Kaufman R J, Current Protocols in Molecular Biology 16.12 (Frederick M. Ausubel, et al., eds. Wiley 1991, incorporated herein) that can be used in the present invention include, but are not limited to, pSC11 (Smai cloning site, TK- and -gal selection), pMJ601 (Sali, Smai, Afli, Nari, BspMII, BamHI, Apai, Nhei, Sacii, Kpni and Hindiii cloning sites; TK- and -gal selection), pTKgptF1S (EcoRI, Psti, Salii, Acci, Hindii, Sbai, BamHI and Hpa cloning sites, TK or XPRT selection) and the like.

In addition, an expression vector containing the novel GAABCP-TUP gene may include drug selection markers. Such markers aid in the selection or identification of host cells that contain high levels of the expression vector containing the novel GAABCP-TUP gene inserted in to their genome in a manner that resulted in high levels of recombinant protein expression for the novel GAABCP-TUP gene's translation product. For example, selection marker genes that confer resistance to neomycin, puromycin, hygromycin, methotrexate, dihydrofolate reductase (DHFR), guanine phosphoribosyl transferase (GPT), zeocin, and histidinol are useful selectable markers. Alternatively, enzymes such as herpes simplex virus thymidine kinase (tk) or chloramphenicol acetyltransferase (CAT) may be employed. Immunologic markers also can be employed. Any known selectable marker may be employed so long as it is capable of being expressed simultaneously with the nucleic acid encoding a gene product. Further examples of selectable markers are well known to one of skill in the art and include reporters such as enhanced green fluorescent protein (EGFP), beta-galactosidase (β-gal) or CAT. In addition, pools of transformed cells that have been selected using the above selection markers to express high levels of the expression vector with the novel GAABCP-TUP gene may also be selected for other properties of the host cell. These other selection criteria can be, but are not limited to, the controlling for growth rate, and/or down- or up-regulating the synthesis and/or expression of certain genes.

### Recombinant Expression and Host Cells

Consequently in the present invention, mammalian cells may be used as host cells and may be transformed by the expression vector containing the novel GAABCP-TUP gene, as defined herein. Examples of suitable cells include, but are not limited to, VERO cells, HELA cells such as ATCC No. CCL-2, CHO cell lines such as CHO-K1 (ATCC No. CCL-61), CHO DG44 (Invitrogen), CHO-S (Invitrogen), CHO-3E7 (National Research Council of Canada), CHO-BRI (National Research Council of Canada), and GS-CHO (LONZA), NS0 cells (ATCC No. CRL-2696), COS cells such as COS-7 cells and ATCC No. CRL-1650 cells, W138, BHK, HepG2, 3T3 such as ATCC No. CRL-6361, A549, PC12, K562 cells, 293 cells, Sf9 cells such as ATCC No. CRL-1711, and Cvl cells such as ATCC No. CCL-70. Preferred mammalian host cells for the present invention include CHO-based expression systems, and variants thereof. Host cells transfected with the expression vector containing the novel GAABCP-TUP gene can be cultured in a variety of formats to produce recombinant glycoprotein conjugates, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Host cells expressing the recombinant GAABCP-TUP glycoprotein conjugate of interest can be cultured in animal serum-free, chemically defined nutrient media (e.g., CD OptiCHO nutrient mixture). These nutrient media are known to be useful for supporting rapid growth and high cell densities for host cell culture, and can be modified as appropriate for supplementing certain limiting nutrients, activating promoters, selecting transformants or amplifying genes, so as to cause expression of the recombinant glycoprotein conjugate at high levels in the cell culture. Cell culture conditions needed for optimal host cell growth and maintenance, such as temperature, pH, dissolved oxygen, nutrient supplements, and the like, are those conditions previously used with the host cell selected for expression and will be apparent to the ordinarily skilled artisan. Host cell cultures expressing the recombinant GAABCP-TUP glycoprotein conjugate of interest are monitored over time in culture using in-process assays that are well known to those in the art, including viable cell density, % viability, glucose consumption, ammonia production, pH, dissolved oxygen content, quantity of recombinant protein of interest (by ELISA or other technique commonly used to measure the amount of a particular protein in solution), and quality of recombinant protein of interest (by SD S-PAGE and Western blot with a specific antibody to the unmodified TUP or GAABCP-TUP glycoprotein conjugate).

Host cells expressing the recombinant GAABCP-TUP of interest may typically be harvested by cooling the culture to 4° C., followed by low speed centrifugation to remove the cells, and removal of cellular debris by filtration thru a 0.22 um pore sized filtration device. Optionally one skilled in the art may recognize that the use of chemical protease and glycanase inhibitors that limit degradation of the expressed glycoprotein conjugate will be useful. Suitable protease inhibitors include, but are not limited to, leupeptin, pepstatin or aprotinin. Suitable glycosidase inhibitors include, but are not limited to, the selective human sialidase (NEU2) inhibitors, 2-deoxy-2,3-dehydro-N-acetylneuraminic acid (DANA or Neu5Ac2en) and Neu5AcN(3)9N(3)2en (Li Y, Cao H, Yu H, Chen Y, et. al. (2011) Identifying selective inhibitors against human cytosolic sialidase NEU2 by substrate specificity studies. Mol Biosyst. 7(4):1060-1072, incorporated herein). Genetic approaches to limited glycosidase activities can also be employed, including the use of RNA interference methodology (Ngantung F, Miller P, Brushett F, Tang G, Wang D. (2006) RNA interference of sialidase improves glycoprotein sialic acid content consistency. Biotechnol Bioeng. 95(1):106-119; Zhang M, Koskie K, Ross J, Kayser K, Caple M. (2010) Enhancing glycoprotein sialylation by targeted gene silencing in mammalian cells. Biotechnol Bioeng. 105(6):1094-1105, incorporated herein) and replacement of specific glycotransferase genes (Onitsuka M, Kim W, Ozaki H, Kawaguchi A, et. al. (2012) Enhancement of sialylation on humanized IgG-like bispecific antibody by overexpression of α2,6-sialyltransferase derived from Chinese hamster ovary cells. Appl. Microbiol. Biotechnol. 94(1):69-80, incorporated herein).

### GAABCP-TUP Purification and Characterization

Recombinant GAABCP-TUP-based glycoprotein conjugates may optionally be purified from the host proteins (e.g., native CHO proteins), cellular debris and other low molecule weight nutrients in the cell culture media via any one or more biophysical techniques commonly used by those skilled in the art. Typically, recombinant glycoprotein or glycoprotein conjugate purification entails combinations of individual purification procedures such as affinity purification (especially in the event an affinity tag is employed in the primary amino acid sequence of the GAABCP-TUP), lectin chromatography, salt fractionation, ion exchange chromatography, size exclusion chromatography, and hydrophobic interaction chromatography. One skilled in the art will recognize that which combination of methods is employed, and in what format, depends on the biophysical nature of the specific GAABCP-TUP, and the desired purity and yield for the glycoprotein conjugate purification process. These generalized purification methodologies can be utilized in a variety of formats, including high and low pressure column chromatography, and bulk resin interaction approaches. See, in general, Robert K. Scopes, Protein Purification: Principles and Practice (Charles R. Castor, ex., Springer-Verlag 1994) and Joseph Sambrook, Molecular Cloning: A Laboratory Manual, 2nd edition (Cold Spring Harbor Laboratory Press 1989, incorporated herein). Examples of multi-step purification separations are also described in Baron, et al. (1990) Crit. Rev. Biotechnol. 10:179-90 and Below, et al. (1994) J. Chromatogr. A. 679:67-83, incorporated herein.

The pH, composition, or volume of the starting host cell culture media containing the recombinant GAABCP-TUP may be adjusted prior to performing any subsequent glycoprotein conjugate purification steps. In addition, the first semi-purified GAABCP-TUP mixture, or any subsequent mixture thereof, may have its pH, composition, or volume adjusted to facilitate subsequent purification methods using techniques known to those of ordinary skill in the art. These methods include, but are not limited to, titration with and acid or base, dilution, tangential flow filtration, diafiltration and dialysis. These methods may also be employed to change the buffer composition of the final purified GAABCP-TUP glycoprotein conjugate in order to stabilize the glycoprotein conjugate against degradation, aggregation and/or precipitation.

The purified GAABCP-TUP glycoprotein conjugate may in some embodiments be at least 90% pure (as measured by size exclusion chromatography, high performance liquid chromatography, SEC-HPLC, sodium dodecyl sulfate polyacrylamide gel electrophoresis, SDS-PAGE, or capillary electrophoresis) or at least 95% pure, or at least 98% pure, or at least 99% or greater pure. Regardless of the exact numerical value of the purity of the GAABCP-TUP glycoprotein conjugate, the needed level of purity for the GAABCP-TUP required for subsequent bioactivity testing (if desired) and determination of pharmacokinetic properties is generally understood by those skilled in the art based on the particular TUP present. Those skilled in the art will understand that the GAABCP-TUP glycoprotein conjugate may be further characterized using standard protein and carbohydrate analytical methods.

Glycoprotein conjugate identity can be established using an anti-unmodified TUP antibody or anti-GAABCP-TUP antibody in a Western blot format. This analysis will typically be performed before and after digestion of the glycoprotein conjugate with glycosidases that remove all or part of the O- and/or N-linked carbohydrate chains. Identity may be further confirmed using N-terminal amino acid sequence analysis and further confirmed using amino acid analysis following acid hydrolysis. The protein may be quantitated in solution using an ELISA designed to detect the unmodified TUP, certain dye binding assays, e.g., Bradford assay, quantitating binding to a resin directed to an affinity tag contained in the GAABCP-TUP, or evaluating the A280 absorbance of the purified GAABCP-TUP glycoprotein conjugate in certain aqueous solutions compared to a protein standard of known concentration.

The carbohydrate composition of the GAABCP-TUP glycoprotein conjugate may also be further characterized using a suite of analytical methods known to those skilled in the art. These will typically include determination of total sialic acid content (N-acetylneuraminic acid [NANA] plus N-glycolylneuraminic acid [NGNA]; Rohrer J. (2000) Analyzing sialic acids using high-performance anion-exchange chromatography with pulsed amperometric detection. Anal. Biochem. 15; 283(1):3-9, incorporated herein), ratio of the sialic acid forms NANA/NGNA, and determination of the types and molecular weight of the O- and N-glycoforms by MALDI-TOF MS (Apte A, Meitei N. (2010) Bioinformatics in glycomics: glycan characterization with mass spectrometric data using SimGlycan. Methods Mol. Biol. 600:269-281; Gao H Y. (2009) Generation of asparagine-linked glycan structure databases and their use. J. Am. Soc. Mass. Spectrom. 20(9):1739-1742, incorporated herein) and RP-HPLC compared to known standards (Melmer M, Stangler T, Schiefermeier M, Brunner W, et. al. (2010) HILIC analysis of fluorescence-labeled N-glycans from recombinant biopharmaceuticals. Anal. Bioanal. Chem. 398(2):905-914, incorporated herein). These examples of protein and carbohydrate characterization are well known to those skilled in the art, but are not meant to be limiting in their scope. It is understood to those skilled in the art that biopharmaceuticals are highly complex and heterogeneous molecules. Protein glycosylation is an inherent source of this heterogeneity and is known to affect safety, efficacy, and serum half-life of therapeutic glycoproteins (Sold R J, Griebenow K (2010) Glycosylation of therapeutic proteins: An effective strategy to optimize efficacy. BioDrugs 24(1): 9-21, incorporated herein). In particular the importance of N-glycans that posess a high level of sialic acid occupancy in the terminal carbohydrate chain position on therapeutic protein plasma half-life has been well documented in the literature (Elliott S, Lorenzini T, Asher S, Aoki K, et. al. (2003) Enhancement of therapeutic protein in vivo activities through glycoengineering. Nature Biotechnol. 21:414-421; Hossler P. (2009) Optimal and consistent protein glycosylation in mammalian cell culture. Glycobiology 19:936-949; Hossler P. (2012) Protein glycosylation control in mammalian cell culture: past precedents and contemporary prospects. Adv. Biochem. Eng. Biotechnol. 127:187-219, incorporated herein). In addition sialic acid analysis is a regulatory requirement defined by the International Conference of Harmonization of Technical Requirement for Registration of Pharmacueticals for Human Use in their Q6B guidelines for characterisation of biopharmaceuticals. In particular, determination of relative levels of human and non-human type sialic acids (NeuAc/NANA and NeuGc/NGNA respectively) is important since some patients have high levels of anti-NeuGc antibodies which could lead to neutralization and rapid clearing of NeuGc-containing biopharmaceuticals (Varki, A. (2008) Sialic acids in human health and disease. Trends Mol. Med. 14(8):351-360, incorporated herein).

Any one of a variety of purification procedures may be performed on the host cell culture medium, or other material, comprising a semi-purified GAABCP-TUP or on any GAABCP-TUP mixtures resulting from any isolation steps including, but not limited to, affinity chromatography, ion exchange chromatography, hydrophobic interaction chromatography, gel filtration chromatography, high performance liquid chromatography (HPLC), reversed phase-HPLC (RP-HPLC), expanded bed adsorption, or any combination and/or repetition thereof and in any appropriate order. Equipment and other necessary materials used in performing the techniques described herein are commercially available. Pumps, fraction collectors, monitors, recorders, and entire systems are available from, for example, Applied Biosystems (Foster City, Calif.), Bio-Rad Laboratories, Inc. (Hercules, Calif.), and Amersham Biosciences, Inc. (Piscataway, N.J.). Chromatographic materials including, but not limited to, exchange matrix materials, media, and buffers are also available from such companies.

Equilibration, and other steps in the column chromatography processes described herein such as washing and elution, may be more rapidly accomplished using specialized equipment such as a pump. Commercially available pumps include, but are not limited to, HILOAD® Pump P-50, Peristaltic Pump P-1, Pump P-901, and Pump P-903 (Amersham Biosciences, Piscataway, N.J.). Examples of fraction collectors include RediFrac Fraction Collector, FRAC-100 and FRAC-200 Fraction Collectors, and SUPERFRAC® Fraction Collector (Amersham Biosciences, Piscataway, N.J.). Mixers are also available to form pH and linear concentration gradients. Commercially available mixers include Gradient Mixer GM-1 and In-Line Mixers (Amersham Biosciences, Piscataway, N.J.). The chromatographic process may be monitored using any commercially available monitor. Such monitors may be used to gather information like UV, pH, and conductivity. Examples of detectors include Monitor UV-1, UVICORD® S II, Monitor UVMII, Monitor UV-900, Monitor UPC-900, Monitor pH/C-900, and Conductivity Monitor (Amersham Biosciences, Piscataway, N.J.). Indeed, entire systems are commercially available including the various AKTA® systems from Amersham Biosciences (Piscataway, N.J.).

In some embodiments, the GAABCP-TUP-based glycoprotein conjugates produced by the methods herein may be misfolded, and thus lack, or have significantly reduced, biological activity. In such instances, the bioactivity of the protein may be restored by “disulfide refolding”. GAABCP-TUPs may be refolded using standard methods known in the art, such as those described in U.S. Pat. Nos. 4,511,502, 4,511,503, and 4,512,922, which are herein incorporated by reference. The GAABCP-TUPs may also be cofolded with other proteins to form heterodimers or heteromultimers.

Ion Exchange Chromatography.

In one embodiment, and as an optional, additional step, ion exchange chromatography may be performed on the GAABCP-TUP mixture. See generally Ion Exchange Chromatography: Principles and Methods (Cat. No. 18-1114-21, Amersham Biosciences (Piscataway, N.J.), incorporated herein). Commercially available ion exchange columns include HITRAP®, HIPREP®, and HILOAD® Columns (Amersham Biosciences, Piscataway, N.J.). Such columns utilize strong anion exchangers such as Q SEPHAROSE® Fast Flow, Q SEPHAROSE® High Performance, and Q SEPHAROSE® XL, strong cation exchangers such as SP SEPHAROSE® High Performance, SP SEPHAROSE® Fast Flow, and SP SEPHAROSE® XL; anion exchangers such as DEAE SEPHAROSE® Fast Flow; and weak cation exchangers such as CM SEPHAROSE® Fast Flow (Amersham Biosciences, Piscataway, N.J.). Anion or cation exchange column chromatography may be performed on the GAABCP-TUP at any stage of the purification process to isolate substantially purified GAABCP-TUP glycoprotein conjugate. The cation exchange chromatography step may be performed using any suitable cation exchange matrix. Useful cation exchange matrices include, but are not limited to, fibrous, porous, non-porous, microgranular, beaded, or cross-linked cation exchange matrix materials. Such cation exchange matrix materials include, but are not limited to, cellulose, agarose, dextran, polyacrylate, polyvinyl, polystyrene, silica, polyether, or composites of any of the foregoing.

The cation exchange matrix may be any suitable cation exchanger including strong and weak cation exchangers. Strong cation exchangers may remain ionized over a wide pH range and thus, may be capable of binding GAABCP-TUP glycoprotein conjugate over a wide pH range. Weak cation exchangers, however, may lose ionization as a function of pH. For example, a weak cation exchanger may lose charge when the pH drops below about pH 4 or pH 5. Suitable strong cation exchangers include, but are not limited to, charged functional groups such as sulfopropyl (SP), methyl sulfonate (S), or sulfoethyl (SE). The cation exchange matrix may be a strong cation exchanger, preferably having a GAABCP-TUP glycoprotein conjugate binding pH range of about 2.5 to about 6.0. Alternatively, the strong cation exchanger may have a GAABCP-TUP glycoprotein conjugate binding pH range of about pH 2.5 to about pH 5.5. The cation exchange matrix may be a strong cation exchanger having a GAABCP-TUP glycoprotein conjugate binding pH of about 3.0. Alternatively, the cation exchange matrix may be a strong cation exchanger, preferably having a GAABCP-TUP glycoprotein conjugate binding pH range of about 6.0 to about 8.0. The cation exchange matrix may be a strong cation exchanger preferably having a GAABCP-TUP glycoprotein conjugate binding pH range of about 8.0 to about 12.5. Alternatively, the strong cation exchanger may have a GAABCP-TUP glycoprotein conjugate binding pH range of about pH 8.0 to about pH 12.0.

Prior to loading the GAABCP-TUP glycoprotein conjugate, the cation exchange matrix may be equilibrated, for example, using several column volumes of a dilute, weak acid, e.g., four column volumes of 20 mM acetic acid, pH 3. Following equilibration, the GAABCP-TUP glycoprotein conjugate may be added and the column may be washed one to several times, prior to elution of substantially purified GAABCP-TUP glycoprotein conjugate also using a weak acid solution such as a weak acetic acid or phosphoric acid solution. For example, approximately 2-4 column volumes of 20 mM acetic acid, pH 3, may be used to wash the column. Additional washes using, e.g., 2-4 column volumes of 0.05 M sodium acetate, pH 5.5, or 0.05 M sodium acetate mixed with 0.1 M sodium chloride, pH 5.5, may also be used. Alternatively, using methods known in the art, the cation exchange matrix may be equilibrated using several column volumes of a dilute, weak base.

In other embodiments, substantially purified GAABCP-TUP glycoprotein conjugate may be eluted by contacting the cation exchanger matrix with a buffer having a sufficiently low pH or ionic strength to displace the GAABCP-TUP from the matrix. The pH of the elution buffer may range from about pH 2.5 to about pH 6.0. More specifically, the pH of the elution buffer may range from about pH 2.5 to about pH 5.5, about pH 2.5 to about pH 5.0. The elution buffer may have a pH of about 3.0. In addition, the quantity of elution buffer may vary widely and will generally be in the range of about 2 to about 10 column volumes.

Following adsorption of the GAABCP-TUP glycoprotein conjugate to the cation exchanger matrix, substantially purified GAABCP-TUP glycoprotein conjugate may be eluted by contacting the matrix with a buffer having a sufficiently high pH or ionic strength to displace the GAABCP-TUP glycoprotein conjugate from the matrix. Suitable buffers for use in high pH elution of substantially purified GAABCP-TUP glycoprotein conjugate may include, but not limited to, citrate, phosphate, formate, acetate, HEPES, and MES buffers ranging in concentration from at least about 5 mM to at least about 100 mM.

Reverse-Phase Chromatography.

RP-HPLC may be performed to purify proteins following suitable protocols that are known to those of ordinary skill in the art (see, e.g., Pearson et al. (1982) Anal. Biochem. 124:217-230; Rivier et al. (1983) J. Chrom. 268:112-119; Kunitani et al. (1986) J. Chrom. 359:391-402, incorporated herein). RP-HPLC may be performed on the GAABCP-TUP glycoprotein conjugate to isolate substantially purified GAABCP-TUP. In this regard, silica derivatized resins with alkyl functionalities with a wide variety of lengths, including, but not limited to, at least about C3 to at least about C30, at least about C3 to at least about C20, or at least about C3 to at least about C18, resins may be used. Alternatively, a polymeric resin may be used. For example, TosoHaas Amberchrome CG1000sd resin may be used, which is a styrene polymer resin. Cyano or polymeric resins with a wide variety of alkyl chain lengths may also be used. Furthermore, the RP-HPLC column may be washed with a solvent such as ethanol. The Source RP column is another example of a RP-HPLC column.

A suitable elution buffer containing an ion pairing agent and an organic modifier such as methanol, isopropanol, tetrahydrofuran, acetonitrile or ethanol, may be used to elute the GAABCP-TUP from the RP-HPLC column. The most commonly used ion pairing agents include, but are not limited to, acetic acid, formic acid, perchloric acid, phosphoric acid, trifluoroacetic acid, heptafluorobutyric acid, triethylamine, tetramethylammonium, tetrabutylammonium, and triethylammonium acetate. Elution may be performed using one or more gradients or isocratic conditions, with gradient conditions preferred to reduce the separation time and to decrease peak width. Another method involves the use of two gradients with different solvent concentration ranges. Examples of suitable elution buffers for use herein may include, but are not limited to, ammonium acetate and acetonitrile solutions.

Hydrophobic Interaction Chromatography (HIC) Purification Techniques.

Hydrophobic interaction chromatography (HIC) may be performed on the GAABCP-TUP (see generally, Hydrophobic Interaction Chromatography Handbook: Principles And Methods (Amersham Biosciences, Piscataway, N.J.) which is incorporated by reference herein). Suitable HIC matrices may include, but are not limited to, alkyl- or aryl-substituted matrices, such as butyl, hexyl-, octyl- or phenyl-substituted matrices including agarose, cross-linked agarose, sepharose, cellulose, silica, dextran, polystyrene, poly(methacrylate) matrices, and mixed mode resins, including but not limited to, a polyethyleneamine resin or a butyl- or phenylsubstituted poly(methacrylate) matrix. Commercially available sources for hydrophobic interaction column chromatography include, but are not limited to, HITRAP®, HIPREP®, and HILOAD® columns (Amersham Biosciences, Piscataway, N.J.).

Briefly, prior to loading, the HIC column may be equilibrated using standard buffers known to those of ordinary skill in the art, such as an acetic acid/sodium chloride solution or HEPES containing ammonium sulfate. Ammonium sulfate may be used as the buffer for loading the HIC column. After loading the GAABCP-TUP glycoprotein conjugate, the column may then washed using standard buffers and conditions to remove unwanted materials but retaining the GAABCP-TUP glycoprotein conjugate on the HIC column. The GAABCP-TUP glycoprotein conjugate may be eluted with about 3 to about 10 column volumes of a standard buffer, such as a HEPES buffer containing EDTA and lower ammonium sulfate concentration than the equilibrating buffer, or an acetic acid/sodium chloride buffer, among others. A decreasing linear salt gradient using, for example, a gradient of potassium phosphate, may also be used to elute the GAABCP-TUP glycoprotein conjugate. The eluant may then be concentrated, for example, by filtration such as diafiltration or ultrafiltration. Diafiltration may be utilized to remove the salt used to elute the GAABCP-TUP glycoprotein conjugate.

Affinity Purification Techniques.

Additionally, purification of the GAABCP-TUP glycoprotein conjugate can in some embodiments be enabled by the inclusion of an affinity tag. For example, GAABCP-TUP glycoprotein conjugate may be purified using Ni+2 affinity chromatography if a His-tag is employed, or by glutathione resin if a GST fusion is employed, or immobilized by anti-flag antibody if a flag-tag is used. For general guidance in suitable purification techniques, see Protein Purification: Principles and Practice, 3rd Ed., Scopes, Springer-Verlag NY (1994), the contents of which is herein incorporated by reference. The degree of purification necessary will vary depending on the screen or use of the GAABCP-TUP glycoprotein conjugate. In some instances no purification is necessary. For example in one embodiment, since the GAABCP-TUP glycoprotein conjugate is secreted, screening may take place directly from the media. As is well known in the art, some methods of selection do not involve purification of proteins.

Other Purification Techniques.

Purification methods also include electrophoretic, immunological, precipitation, dialysis, and chromatofocusing techniques. Ultrafiltration and diafiltration techniques, in conjunction with protein concentration, are also useful. Yet another isolation step using, for example, gel filtration (Gel Filtration: Principles and Methods, Amersham Biosciences, Piscataway N.J., which is incorporated by reference herein), hydroxyapatite chromatography (suitable matrices include, but are not limited to, HA Ultrogel, High Resolution (Calbiochem), CHT Ceramic Hydroxyapatite (BioRad), Bio-Gel HTP Hydroxyapatite (BioRad)), HPLC, expanded bed adsorption, ultrafiltration, diafiltration, lyophilization, and the like, may be performed on the first GAABCP-TUP glycoprotein conjugate mixture or any subsequent mixture thereof, to remove any excess salts and to replace the buffer with a suitable buffer for the next isolation step or even formulation of the final drug product.

The yield of GAABCP-TUP glycoprotein conjugate, including substantially purified GAABCP-TUP, may be monitored at each step described herein using techniques known to those of ordinary skill in the art. Such techniques may also be used to assess the yield of substantially purified GAABCP-TUP glycoprotein conjugate following the last isolation step. For example, the yield of GAABCP-TUP glycoprotein conjugate may be monitored using any of several reverse phase high pressure liquid chromatography columns, having a variety of alkyl chain lengths such as cyano RP-HPLC, C18RP-HPLC; as well as cation exchange HPLC and gel filtration HPLC.

In specific embodiments of the present invention, the yield of GAABCP-TUP glycoprotein conjugate after each purification step may be at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, or at least about 99.99%, of the GAABCP-TUP glycoprotein conjugate in the starting material for each purification step.

RP-HPLC material Vydac C4 (Vydac) consists of silica gel particles, the surfaces of which carry C4-alkyl chains. The separation of GAABCP-TUP glycoprotein conjugate from the proteinaceous impurities is based on differences in the strength of hydrophobic interactions. Elution is performed with an acetonitrile gradient in diluted trifluoroacetic acid. Preparative HPLC is performed using a stainless steel column (filled with 2.8 to 3.2 liter of Vydac C4 silica gel). The hydroxyapatite Ultrogel eluate is acidified by adding trifluoroacetic acid and loaded onto the Vydac C4 column. For washing and elution an acetonitrile gradient in diluted trifluoroacetic acid is used. Fractions are collected and immediately neutralized with phosphate buffer. The GAABCP-TUP glycoprotein conjugate fractions which are within the IPC limits are pooled.

DEAE Sepharose (Pharmacia) material consists of diethylaminoethyl (DEAE)-groups which are covalently bound to the surface of Sepharose beads. The binding of GAABCP-TUP glycoprotein conjugate to the DEAE groups is mediated by ionic interactions. Acetonitrile and trifluoroacetic acid pass through the column without being retained. After these substances have been washed off, trace impurities are removed by washing the column with acetate buffer at a low pH. Then the column is washed with neutral phosphate buffer and GAABCP-TUP glycoprotein conjugate is eluted with a buffer with increased ionic strength. The column is packed with DEAE Sepharose fast flow. The column volume is adjusted to assure a GAABCP-TUP glycoprotein conjugate load in the range of 3-10 mg GAABCP-TUP/mL gel. The column is washed with water and equilibration buffer (sodium/potassium phosphate). The pooled fractions of the HPLC eluate are loaded and the column is washed with equilibration buffer. Then the column is washed with washing buffer (sodium acetate buffer) followed by washing with equilibration buffer. Subsequently, GAABCP-TUP glycoprotein conjugate is eluted from the column with elution buffer (sodium chloride, sodium/potassium phosphate) and collected in a single fraction in accordance with the master elution profile. The eluate of the DEAE Sepharose column is adjusted to the specified conductivity. The resulting GAABCP-TUP glycoprotein conjugate is sterile filtered into suitable containers and stored at −70° C.

Additional methods that may be employed include, but are not limited to, steps to remove endotoxins. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin as a contaminant in a purified recombinant protein may, when administered at humans, produce adverse reactions including, fever, a lowering of the blood pressure, and activation of inflammation and coagulation, among other adverse physiological effects. Therefore, in pharmaceutical production, it is often desirable to remove most or all traces of endotoxin from drug products and/or drug containers, because even small amounts may cause adverse effects in humans. Accordingly, the term “endotoxin-free” or “substantially endotoxin-free” relates generally to compositions, solvents, and/or vessels that contain at most trace amounts (e.g., amounts having no clinically adverse physiological effects to a subject) of endotoxin, and preferably undetectable amounts of endotoxin.

Methods for reducing endotoxin levels are known to one of ordinary skill in the art and include, but are not limited to, purification techniques using silica supports, glass powder or hydroxyapatite, reverse-phase, specific lipopolysaccharide affinity matricides, size-exclusion, anion-exchange chromatography, hydrophobic interaction chromatography, a combination of these methods, and the like. Modifications or additional methods may be required to remove contaminants such as co-migrating proteins from the GAABCP-TUP glycoprotein conjugate of interest.

Endotoxins can be detected using routine techniques known in the art. For example, the Limulus Ameobocyte Lysate assay (Charles River Labs), which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). Alternate methods include, but are not limited to, a Kinetic LAL method that is turbidmetric and uses a 96 well format. To be substantially endotoxin-free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of protein. Typically, 1 ng lipopolysaccharide (LPS) corresponds to about 1-10 EU.

In certain embodiments, a composition has an endotoxin content of about or less than about 10 EU/mg of the GAABCP-TUP glycoprotein conjugate, about or less than about 9 EU/mg, about or less than about 8 EU/mg, about or less than about 7 EU/mg, about or less than about 6 EU/mg, about or less than about 5 EU/mg, about or less than about 4 EU/mg, about or less than about 3 EU/mg, about or less than about 2 EU/mg, about or less than about 1 EU/mg, about or less than about 0.1 EU/mg, or about or less than about 0.01 EU/mg of GAABCP-TUP glycoprotein conjugate. In certain embodiments, as noted above, a composition is at least about 95%, 96%, 97%, or 98% endotoxin-free, at least about 99% endotoxin-free, at least about 99.5% endotoxin-free, or at least about 99.99% endotoxin-free on a wt/wt protein basis.

Solutions of purified GAABCP-TUP intended for in vitro or in vivo bioactivity testing and determination of in vivo pharmacokinetic properties are also assessed for several other key properties, as is well known by those skilled in the art, including sterility (lack of pathogens), visual clarity (no visual precipitation), and neutral pH (approximately 7.0 to 7.5). Those skilled in the art are familiar with commonly utilized methodologies for determining these properties of the purified GAABCP-TUP glycoprotein conjugate.

### Pharmaceutical Compositions and Administration

As is well understood by one skilled in the art, one important utility of the present invention is the treatment of appropriate diseases, conditions, etc. with GAABCP-TUPs that possess beneficial increases in plasma half-life compared to the corresponding unmodified TUP. Accordingly, certain embodiments are provided herein, which relate to administering a pharmaceutically relevant dose of one or more GAABCP-TUPs to an appropriate subject prior to, concurrent with, or following the clinical manifestation, diagnosis, or induction of a GAABCP-TUP-treatable disease, condition, or injury.

Embodiments of the present invention provide for compositions (e.g., pharmaceutical compositions) comprising the GAABCP-TUPs of the present invention. Said pharmaceutical compositions may comprise, in addition to the GAABCP-TUP glycoprotein conjugate, a carrier. Broadly, the carrier may be a culture medium or a matrix (e.g., a purification matrix). In some embodiments, the carrier is a pharmaceutically acceptable carrier, in which case the composition is useful for preventing or treating disorders and/or diseases in a human or animal, most preferably in a mammal, or for diagnostic purposes. As an active ingredient of the composition, the GAABCP-TUP is preferably in a soluble form. In some embodiments, a composition comprises one or more pH buffering agents, i.e., buffers. Exemplary buffers include histidine (e.g., L-histidine, D-histidine), citrate buffers (e.g., sodium citrate, citric acid, mixtures thereof), and phosphate buffers (e.g., sodium phosphate, phosphate buffered saline (PBS)).

In some embodiments, the buffer is present at a concentration ranging from about 0.3 mM to about 100 mM, or about 0.3 mM to about 95 mM, or about 0.3 mM to about 90 mM, or about 0.3 mM to about 85 mM, or about 0.3 mM to about 80 mM, or about 0.3 mM to about 75 mM, or about 0.3 mM to about 70 mM, or about 0.3 mM to about 65 mM, or about 0.3 mM to about 60 mM, or about 0.3 mM to about 55 mM, or about 0.3 mM to about 50 mM, or about 0.3 mM to about 45 mM, or about 0.3 mM to about 40 mM, or about 0.3 mM to about 35 mM, or about 0.3 mM to about 30 mM, or about 0.3 mM to about 25 mM, or about 0.3 mM to about 20 mM, or about 0.3 mM to about 15 mM, or about 0.3 mM to about 10 mM, or about 0.3 mM to about 5 mM.

In some embodiments, the buffer is present at a concentration ranging from about 1 mM to about 100 mM, or about 1 mM to about 95 mM, or about 1 mM to about 90 mM, or about 1 mM to about 85 mM, or about 1 mM to about 80 mM, or about 1 mM to about 75 mM, or about 1 mM to about 70 mM, or about 1 mM to about 65 mM, or about 1 mM to about 60 mM, or about 1 mM to about 55 mM, or about 1 mM to about 50 mM, or about 1 mM to about 45 mM, or about 1 mM to about 40 mM, or about 1 mM to about 35 mM, or about 1 mM to about 30 mM, or about 1 mM to about 25 mM, or about 1 mM to about 20 mM, or about 1 mM to about 15 mM, or about 1 mM to about 10 mM, or about 1 mM to about 5 mM.

In some embodiments, the buffer is present at a concentration ranging from about 2 mM to about 100 mM, or about 2 mM to about 95 mM, or about 2 mM to about 90 mM, or about 2 mM to about 85 mM, or about 2 mM to about 80 mM, or about 2 mM to about 75 mM, or about 2 mM to about 70 mM, or about 2 mM to about 65 mM, or about 2 mM to about 60 mM, or about 2 mM to about 55 mM, or about 2 mM to about 50 mM, or about 2 mM to about 45 mM, or about 2 mM to about 40 mM, or about 2 mM to about 35 mM, or about 2 mM to about 30 mM, or about 2 mM to about 25 mM, or about 2 mM to about 20 mM, or about 2 mM to about 15 mM, or about 2 mM to about 10 mM, or about 2 mM to about 5 mM.

In some embodiments, the buffer is present at a concentration ranging from about 5 mM to about 100 mM, or about 5 mM to about 95 mM, or about 5 mM to about 90 mM, or about 5 mM to about 85 mM, or about 5 mM to about 80 mM, or about 5 mM to about 75 mM, or about 5 mM to about 70 mM, or about 5 mM to about 65 mM, or about 5 mM to about 60 mM, or about 5 mM to about 55 mM, or about 5 mM to about 50 mM, or about 5 mM to about 45 mM, or about 5 mM to about 40 mM, or about 5 mM to about 35 mM, or about 5 mM to about 30 mM, or about 5 mM to about 25 mM, or about 5 mM to about 20 mM, or about 5 mM to about 15 mM, or about 5 mM to about 10 mM.

In some embodiments, the buffer is present at a concentration ranging from about 10 mM to about 100 mM, or about 10 mM to about 95 mM, or about 10 mM to about 90 mM, or about 10 mM to about 85 mM, or about 10 mM to about 80 mM, or about 10 mM to about 75 mM, or about 10 mM to about 70 mM, or about 10 mM to about 65 mM, or about 10 mM to about 60 mM, or about 10 mM to about 55 mM, or about 10 mM to about 50 mM, or about 10 mM to about 45 mM, or about 10 mM to about 40 mM, or about 10 mM to about 35 mM, or about 10 mM to about 30 mM, or about 10 mM to about 25 mM, or about 10 mM to about 20 mM, or about 10 mM to about 15 mM.

In some embodiments, the buffer is present at a concentration ranging from about 15 mM to about 100 mM, or about 15 mM to about 95 mM, or about 15 mM to about 90 mM, or about 15 mM to about 85 mM, or about 15 mM to about 80 mM, or about 15 mM to about 75 mM, or about 15 mM to about 70 mM, or about 15 mM to about 65 mM, or about 15 mM to about 60 mM, or about 15 mM to about 55 mM, or about 15 mM to about 50 mM, or about 15 mM to about 45 mM, or about 15 mM to about 40 mM, or about 15 mM to about 35 mM, or about 15 mM to about 30 mM, or about 15 mM to about 25 mM, or about 15 mM to about 20 mM.

In some embodiments, the buffer is present at a concentration ranging from about 20 mM to about 100 mM, or about 20 mM to about 95 mM, or about 20 mM to about 90 mM, or about 20 mM to about 85 mM, or about 20 mM to about 80 mM, or about 20 mM to about 75 mM, or about 20 mM to about 70 mM, or about 20 mM to about 65 mM, or about 20 mM to about 60 mM, or about 20 mM to about 55 mM, or about 20 mM to about 50 mM, or about 20 mM to about 45 mM, or about 20 mM to about 40 mM, or about 20 mM to about 35 mM, or about 20 mM to about 30 mM, or about 20 mM to about 25 mM.

In some embodiments, the buffer is present at a concentration ranging from about 25 mM to about 100 mM, or about 25 mM to about 95 mM, or about 25 mM to about 90 mM, or about 25 mM to about 85 mM, or about 25 mM to about 80 mM, or about 25 mM to about 75 mM, or about 25 mM to about 70 mM, or about 25 mM to about 65 mM, or about 25 mM to about 60 mM, or about 25 mM to about 55 mM, or about 25 mM to about 50 mM, or about 25 mM to about 45 mM, or about 25 mM to about 40 mM, or about 25 mM to about 35 mM, or about 25 mM to about 30 mM.

In some embodiments, the buffer is present at a concentration ranging from about 30 mM to about 100 mM, or about 30 mM to about 95 mM, or about 30 mM to about 90 mM, or about 30 mM to about 85 mM, or about 30 mM to about 80 mM, or about 30 mM to about 75 mM, or about 30 mM to about 70 mM, or about 30 mM to about 65 mM, or about 30 mM to about 60 mM, or about 30 mM to about 55 mM, or about 30 mM to about 50 mM, or about 30 mM to about 45 mM, or about 30 mM to about 40 mM, or about 30 mM to about 35 mM.

In some embodiments, the buffer is present at a concentration ranging from about 35 mM to about 100 mM, or about 35 mM to about 95 mM, or about 35 mM to about 90 mM, or about 35 mM to about 85 mM, or about 35 mM to about 80 mM, or about 35 mM to about 75 mM, or about 35 mM to about 70 mM, or about 35 mM to about 65 mM, or about 35 mM to about 60 mM, or about 35 mM to about 55 mM, or about 35 mM to about 50 mM, or about 35 mM to about 45 mM, or about 35 mM to about 40 mM.

In some embodiments, the buffer is present at a concentration ranging from about 40 mM to about 100 mM, or about 40 mM to about 95 mM, or about 40 mM to about 90 mM, or about 40 mM to about 85 mM, or about 40 mM to about 80 mM, or about 40 mM to about 75 mM, or about 40 mM to about 70 mM, or about 40 mM to about 65 mM, or about 40 mM to about 60 mM, or about 40 mM to about 55 mM, or about 40 mM to about 50 mM, or about 40 mM to about 45 mM.

In some embodiments, the buffer is present at a concentration ranging from about 45 mM to about 100 mM, or about 45 mM to about 95 mM, or about 45 mM to about 90 mM, or about 45 mM to about 85 mM, or about 45 mM to about 80 mM, or about 45 mM to about 75 mM, or about 45 mM to about 70 mM, or about 45 mM to about 65 mM, or about 45 mM to about 60 mM, or about 45 mM to about 55 mM, or about 45 mM to about 50 mM.

In some embodiments, the buffer is present at a concentration ranging from about 50 mM to about 100 mM, or about 50 mM to about 95 mM, or about 50 mM to about 90 mM, or about 50 mM to about 85 mM, or about 50 mM to about 80 mM, or about 50 mM to about 75 mM, or about 50 mM to about 70 mM, or about 50 mM to about 65 mM, or about 50 mM to about 60 mM, or about 50 mM to about 55 mM.

In some embodiments, the buffer is present at a concentration ranging from about 55 mM to about 100 mM, or about 55 mM to about 95 mM, or about 55 mM to about 90 mM, or about 55 mM to about 85 mM, or about 55 mM to about 80 mM, or about 55 mM to about 75 mM, or about 55 mM to about 70 mM, or about 55 mM to about 65 mM, or about 55 mM to about 60 mM.

In some embodiments, the buffer is present at a concentration ranging from about 60 mM to about 100 mM, or about 60 mM to about 95 mM, or about 60 mM to about 90 mM, or about 60 mM to about 85 mM, or about 60 mM to about 80 mM, or about 60 mM to about 75 mM, or about 60 mM to about 70 mM, or about 60 mM to about 65 mM. In some embodiments, the buffer is present at a concentration ranging from about 65 mM to about 100 mM, or about 65 mM to about 95 mM, or about 65 mM to about 90 mM, or about 65 mM to about 85 mM, or about 65 mM to about 80 mM, or about 65 mM to about 75 mM, or about 65 mM to about 70 mM.

In some embodiments, the buffer is present at a concentration ranging from about 70 mM to about 100 mM, or about 70 mM to about 95 mM, or about 70 mM to about 90 mM, or about 70 mM to about 85 mM, or about 70 mM to about 80 mM, or about 70 mM to about 75 mM. In some embodiments, the buffer is present at a concentration ranging from about 75 mM to about 100 mM, or about 75 mM to about 95 mM, or about 75 mM to about 90 mM, or about 75 mM to about 85 mM, or about 75 mM to about 80 mM.

In some embodiments, the buffer is present at a concentration ranging from about 80 mM to about 100 mM, or about 80 mM to about 95 mM, or about 80 mM to about 90 mM, or about 80 mM to about 85 mM. In some embodiments, the buffer is present at a concentration ranging from about 85 mM to about 100 mM, or about 85 mM to about 95 mM, or about 85 mM to about 90 mM. In some embodiments, the buffer is present at a concentration ranging from about 90 mM to about 100 mM, or about 90 mM to about 95 mM, or about 95 mM to about 100 mM.

In some embodiments, the buffer is present at a concentration ranging from about 40-60, 41-60, 42-60, 43-60, 44-60, 45-60, 46-60, 47-60, 48-60, 49-60, 50-60, 51-60, 52-60, 53-60, 54-60, 55-60, 56-60, 57-60, 58-60, 59-60 mM, or about 40-59, 41-59, 42-59, 43-59, 44-59, 45-59, 46-59, 47-59, 48-59, 49-59, 50-59, 51-59, 52-59, 53-59, 54-59, 55-59, 56-59, 57-59, 58-59 mM, or about 40-58, 41-58, 42-58, 43-58, 44-58, 45-58, 46-58, 47-58, 48-58, 49-58, 50-58, 51-58, 52-58, 53-58, 54-58, 55-58, 56-58, 57-58 mM, or about 40-57, 41-57, 42-57, 43-57, 44-57, 45-57, 46-57, 47-57, 48-57, 49-57, 50-57, 51-57, 52-57, 53-57, 54-57, 55-57, 56-57 mM, or about 40-56, 41-56, 42-56, 43-56, 44-56, 45-56, 46-56, 47-56, 48-56, 49-56, 50-56, 51-56, 52-56, 53-56, 54-56, 55-56 mM, or about 40-55, 41-55, 42-55, 43-55, 44-55, 45-55, 46-55, 47-55, 48-55, 49-55, 50-55, 51-55, 52-55, 53-55, 54-55 mM, or about 40-54, 41-54, 42-54, 43-54, 44-54, 45-54, 46-54, 47-54, 48-54, 49-54, 50-54, 51-54, 52-54, 53-54 mM, or about 40-53, 41-53, 42-53, 43-53, 44-53, 45-53, 46-53, 47-53, 48-53, 49-53, 50-53, 51-53, 52-53 mM, or about 40-52, 41-52, 42-52, 43-52, 44-52, 45-52, 46-52, 47-52, 48-52, 49-52, 50-52, 51-52 mM, or about 40-51, 41-51, 42-51, 43-51, 44-51, 45-51, 46-51, 47-51, 48-51, 49-51, 50-51 mM, or about 40-50, 41-50, 42-50, 43-50, 44-50, 45-50, 46-50, 47-50, 48-50, 49-50 mM, or about 40-49, 41-49, 42-49, 43-49, 44-49, 45-49, 46-49, 47-49, 48-49 mM, or about 40-48, 41-48, 42-48, 43-48, 44-48, 45-48, 46-48, 47-48 mM, or about 40-47, 41-47, 42-47, 43-47, 44-47, 45-47, 46-47 mM, or about 40-46, 41-46, 42-46, 43-46, 44-46, 45-46 mM, or about 40-45, 41-45, 42-45, 43-45, 44-45 mM, or about 40-44, 41-44, 42-44, 43-44 mM, or about 40-43, 41-43, 42-43 mM, or about 40-42, 41-42 mM, or about 40-42 mM.

In some embodiments, the composition comprises a buffer at a concentration of about 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM, including all ranges in between.

In some embodiments, the presence of the buffer alters (e.g., improves, increases, decreases, reduces) one or more biochemical, physical, and/or pharmacokinetic properties of the GAABCP-TUP relative to a composition without the buffer or with a different buffer.

For instance, in certain embodiments, the GAABCP-TUP in the presence of the buffer has increased biological activity relative to a corresponding GAABCP-TUP in an otherwise identical or comparable composition without the buffer or with a different buffer. In some embodiments, the GAABCP-TUP in the buffer has at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater biological activity than a corresponding GAABCP-TUP in an otherwise identical or comparable composition without the buffer or with a different buffer.

In certain embodiments, the GAABCP-TUP in the presence of the buffer has increased “stability” (e.g., as measured by half-life), which is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a corresponding GAABCP-TUP in an otherwise identical or comparable composition without the buffer or with a different buffer.

In some embodiments, the “stability” of the GAABCP-TUP includes its “functional stability,” or the rate at which at least one biological activity of the GAABCP-TUP is reduced under a given set of conditions over time. In some embodiments, the biological activity of the GAABCP-TUP in the presence of the buffer is reduced at a rate that is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold slower or at least about 10%, 20%, 30%, 40%, 50%, 60%, 70%, 80%, 90%, 100%, 200%, 300%, 400%, or 500% slower than a corresponding GAABCP-TUP in an otherwise identical or comparable composition without the buffer or with a different buffer.

In certain embodiments, the “stability” of the GAABCP-TUP includes its “kinetic stability” or “thermal stability,” including its rate of unfolding, aggregation, or precipitation under a given set of conditions over time. In certain embodiments, the GAABCP-TUP in the presence of the buffer unfolds, aggregates, or precipitates at a rate that is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold slower or at least about 5%, 10%, 20%, 30%, 40%, 50%, 60%, 70%, 80%, 90%, 100%, 200%, 300%, 400%, or 500% slower than a corresponding GAABCP-TUP in an otherwise identical or comparable composition without the buffer or with a different buffer.

In certain embodiments, the GAABCP-TUP in the presence of the buffer unfolds, aggregates, or precipitates at a rate that is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold slower or at least about 5%, 10%, 20%, 30%, 40%, 50%, 60%, 70%, 80%, 90%, 100%, 200%, 300%, 400%, or 500% slower than a corresponding GAABCP-TUP when incubated at about 5° C., or at about room temperature (e.g., ˜20-25° C.), or at about 37° C. for about or at least about 3 hours, or about or at least about 3 days, or about or at least about 7 days in an otherwise identical or comparable composition without the buffer or with a different buffer.

In some embodiments, the GAABCP-TUP in the presence of the buffer has a melting temperature (Tm) that is at least about 5%, 10%, 20%, 30%, 40%, 50%, 60%, 70%, 80%, 90%, 100% greater than the melting temperature of a corresponding GAABCP-TUP in an otherwise identical or comparable composition without the buffer or with a different buffer. In some embodiments, the GAABCP-TUP in the presence of the buffer has a melting temperature (Tₘ) that is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C. higher than a corresponding GAABCP-TUP in an otherwise identical or comparable composition without the buffer or with a different buffer.

In some embodiments, the GAABCP-TUP has improved or increased homogeneity or monodispersion (e.g., ratio of monomers/oligomers, ratio of dimers/oligomers, ratio of monomers/dimers, ratio of dimers/monomers, ratio of interchain disulfide bond formation under reducing conditions, distribution of apparent molecular weights, including reduced high molecular weight and/or low molecular weight peaks as detected by either SDS-PAGE or HPLC analysis) in the presence of the buffer relative to a corresponding GAABCP-TUP in an otherwise identical or comparable composition without the buffer or with a different buffer. In some embodiments, the homogeneity or monodispersion of the GAABCP-TUP in the buffer is increased by at least about at least 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold or at least about 5%, 10%, 20%, 30%, 40%, 50%, 60%, 70%, 80%, 90%, 100%, 200%, 300%, 400%, or 500% relative to a corresponding GAABCP-TUP in an otherwise identical or comparable composition without the buffer or with a different buffer. In specific aspects, the buffer is a histidine buffer at a pH within the range of about pH 7.0 to about pH 7.5, or a citrate buffer at a pH within the range of about pH 7.5 to about pH 6.5, or a phosphate buffer at a pH within the range of about pH 7.5 to about pH 6.5.

In certain embodiments, the GAABCP-TUP composition in the presence of the phosphate, histidine or citrate buffer has decreased high molecular weight peak(s) by SE-HPLC analysis that is at least about 1.5, 2, 3, 4, 5, 6, 7, 8, 9, or 10-fold lower or at least about 5%, 10%, 20%, 30%, 40%, 50%, 60%, 70%, 80%, 90%, 100%, 200%, 300%, 400%, or 500% lower than a corresponding GAABCP-TUP when incubated at about 5° C., or at about room temperature (e.g., ˜20-25° C.), or at about 37° C. for about or at least about 3 hours, or about or at least about 3 days, or about or at least about 7 days in an otherwise identical or comparable composition without the buffer or with a different buffer. In some aspects, the GAABCP-TUP composition has a high molecular weight peak content by SE-HPLC analysis which is less than about 2% of the main peak after 2 days storage at 37° C. In some aspects, the GAABCP-TUP composition has a high molecular weight peak content which is less than about 1% of the main peak after 2 days storage at 37° C.

In certain embodiments, the GAABCP-TUP composition in the presence of the phosphate, histidine or citrate buffer has decreased low molecular weight peak(s) by SE-HPLC analysis that is at least about 1.5, 2, 3, 4, 5, 6, 7, 8, 9, or 10-fold lower or at least about 5%, 10%, 20%, 30%, 40%, 50%, 60%, 70%, 80%, 90%, 100%, 200%, 300%, 400%, or 500% lower than a corresponding GAABCP-TUP when incubated at about 5° C., or at about room temperature (e.g., ˜20-25° C.), or at about 37° C. for about or at least about 3 hours, or about or at least about 3 days, or about or at least about 7 days in an otherwise identical or comparable composition without the buffer or with a different buffer.

In certain embodiments, the GAABCP-TUP composition in the presence of the phosphate, histidine or citrate buffer has a decreased turbidity (A340) that is at least about 1.5, 2, 3, 4, 5, 6, 7, 8, 9, or 10-fold lower or at least about 5%, 10%, 20%, 30%, 40%, 50%, 60%, 70%, 80%, 90%, 100%, 200%, 300%, 400%, or 500% lower than a corresponding GAABCP-TUP when incubated at about 5° C., or at about room temperature (e.g., ˜20-25° C.), or at about 37° C. for about or at least about 3 hours, or about or at least about 3 days, or about or at least about 7 days in an otherwise identical or comparable composition without the buffer or with a different buffer. In some aspects, the GAABCP-TUP composition comprises a histidine buffer of about pH 7.0 to 7.5 and has a turbidity (A340) which is less than about 0.5 after 2 days storage at 37° C. In specific aspects, the GAABCP-TUP composition has a turbidity (A340) which is less than about 0.05 after 2 days storage at 37 C. In some aspects, the GAABCP-TUP composition comprises a citrate buffer of about pH 7.0 to 7.5 and has a turbidity (A340) which is less than about 0.5 after 2 days storage at 37° C. In specific aspects, the GAABCP-TUP composition has a turbidity (A340) which is less than about 0.05 after 2 days storage at 37° C. In some aspects, the GAABCP-TUP composition comprises a phosphate buffer of about pH 7.0 to 7.5 and has a turbidity (A340) which is less than about 0.5 after 2 days storage at 37° C. In specific aspects, the GAABCP-TUP composition has a turbidity (A340) which is less than about 0.05 after 2 days storage at 37 C.

In certain embodiments, the pH of the composition (e.g., in the presence of the buffering agent or buffer) is about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or about 8.0. In some embodiments, the pH of the composition ranges from about 6.0-6.1, 6.0-6.2, 6.0-6.3, 6.0-6.4, 6.0-6.5, 6.0-6.6, 6.0-6.7, 6.0-6.8, 6.0-6.9, 6.0-7.0, 6.0-7.1, 6.0-7.2, 6.0-7.3, 6.0-7.4, 6.0-7.5, 6.0-7.6, 6.0-7.7, 6.0-7.8, 6.0-7.9, 6.0-8.0, or from about 6.5-6.6, 6.5-6.7, 6.5-6.8, 6.5-6.9, 6.5-7.0, 6.5-7.1, 6.5-7.2, 6.5-7.3, 6.5-7.4, 6.5-7.5, 6.5-7.6, 6.5-7.7, 6.5-7.8, 6.5-7.9, 6.5-8.0, or from about 7.0-7.1, 7.0-7.2, 7.0-7.3, 7.0-7.4, 7.0-7.5, 7.0-7.6, 7.0-7.7, 7.0-7.8, 7.0-7.9, 7.0-8.0, or from about 7.2-7.3, 7.2-7.4, 7.2-7.5, 7.2-7.6, 7.2-7.7, 7.2-7.8, 7.2-7.9, 7.2-8.0, or from about 7.4-7.5, 7.4-7.6, 7.4-7.7, 7.4-7.8, 7.4-7.9, 7.4-8.0, or from about 7.5-7.6, 7.5-7.7, 7.5-7.8, 7.5-7.9, 7.5-8.0, or from about 7.6-7.7, 7.6-7.8, 7.6-7.9, or 7.6-8.0.

In some embodiments, the pH of the composition or buffer alters (e.g., improves, increases, decreases, reduces) one or more biochemical, physical, and/or pharmacokinetic properties of the GAABCP-TUP relative to a composition having a pH outside of the ranges above. In specific embodiments, the buffer is histidine and the pH of the composition ranges from about 7.0-7.5. In other embodiments, the buffer is a citrate buffer and the pH of the composition ranges from about 6.5-7.5. In other embodiments, the buffer is a sodium phosphate buffer and the pH of the composition ranges from about 7.0-7.5.

For instance, in certain embodiments, the GAABCP-TUP in a composition comprising (a) a histidine buffer and a pH of about 7.0-7.5, (b) a citrate buffer and a pH of about 6.5-7.5, or (c) a phosphate buffer and a pH of about 7.0-7.5 has increased biological activity relative to a comparable composition having a pH outside of said ranges in (a), (b), or (c) above. Exemplary activities include any of the activities described herein, such as, e.g., the induction of cAMP in THP-1 cells (ATCC # TIB-202), and other biological activities, including NO production, PI3K activation, etc. In some embodiments, the GAABCP-TUP has at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater biological activity than a corresponding GAABCP-TUP in a comparable composition having a pH outside of said ranges in (a), (b), or (c) above.

In certain embodiments, the GAABCP-TUP in a composition comprising (a) a histidine buffer and a pH of about 7.0-7.5, (b) a citrate buffer and a pH of about 6.5-7.5, or (c) a phosphate buffer and a pH of about 7.0-7.5 has increased “stability” (e.g., as measured by half-life), which is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a corresponding GAABCP-TUP in a comparable composition having a pH outside of said ranges in (a), (b), or (c) above.

In any of these therapeutic compositions and uses, the compositions can be formulated in pharmaceutically-acceptable, physiologically-acceptable, and/or pharmaceutical grade solutions for administration to a cell, subject, or an animal, either alone, or in combination with one or more other modalities of therapy. It will also be understood that, if desired, the compositions of the invention may be administered in combination with other agents as well, such as, e.g., other proteins or polypeptides or pharmaceutically-active agents. In this context “administered in combination” includes (1) part of the same unitary dosage form; and (2) administration separately, but as part of the same therapeutic treatment program or regimen, typically, but not necessarily, on the same day.

In some embodiments, the compositions comprise a mixture of 2 or more therapeutically useful polypeptides, e.g. 2 or more GAABCP-TUPs, or a mixture of one or more GAABCP-TUPs and one or more of the various unmodified TUPs described herein. In some aspects the compositions may comprise about 2 to about 50, or about 2 to about 25, or about 2 to about 15, or 1, 2, 3, 4, 5, 6, 7, 8, 9, 10, 11, 12, 13, 14, 15 or more GAABCP-TUPs described herein.

Therapeutic or pharmaceutical compositions comprising a therapeutic dose of a GAABCP-TUP include GAABCP-TUPs generated from any one or more homologues, orthologs, variants, fragments, modified polypeptides, naturally-occurring isoforms of the TUPs described herein (including, e.g., any of SEQ ID NOS: 97, 98, 103, 104, 109-112, 146, 147, 157, 164, or 168), and/or any of the GAABCP-TUPs described herein (including, e.g., any of SEQ ID NOS: 99-102, 105-108, 113-123, 134-140, 141-145, 148-156, 158-163, 165-167, or 169-180).

Generally, the composition comprises a pharmaceutically effective amount of the GAABCP-TUP which achieves the desired effect e.g., therapeutic or diagnostic. Pharmaceutically effective amounts can be estimated from cell culture assays. For example, a dose can be formulated in animal models to achieve a circulating concentration range that includes or encompasses a concentration point or range having the desired effect in an in vitro system (see, e. g., Molineux G, et. al. (1999) Exp. Hematol. 27:1724-1734, incorporated herein). This information can thus be used to accurately determine the doses in other mammals, including humans and animals. In general, dosage amounts range from about 1 ng/kg to about 10 mg/kg based on weight of the subject.

Toxicity and therapeutic efficacy of such compounds can be determined by standard pharmaceutical procedures in cell cultures or in experimental animals, (see, e.g., Vugmeyster Y, Xu X, Theil F, Khawli L A, Leach M W (2012) Pharmacokinetics and toxicology of therapeutic proteins: Advances and challenges. World J. Biol. Chem. 26; 3(4):73-92, incorporated herein). For example, the “LD50” (the dose lethal to 50% of the population) as well as the “ED50” (the dose therapeutically effective in 50% of the population) can be determined using methods known in the art. The dose ratio between toxic and therapeutic effects is the therapeutic index that can be expressed as the ratio between LD50 and ED50 compounds that exhibit high therapeutic indices. The data obtained from the cell culture and animal studies can be used in formulating a range of dosage of such compounds which lies preferably within a range of circulating concentrations that include the ED50 with little or no toxicity.

The compositions can be administered via any suitable route such as locally, orally, subcutaneously, systemically, intravenously, intramuscularly, mucosally, transdermally (e.g., via a patch). Accordingly, in addition to these general routes of administration, in some embodiments, the composition may be administered via a mode selected from the group consisting of: parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, and intranasal administration. The compositions may be encapsulated in liposomes, microparticles, microcapsules, nanoparticles and the like. Techniques for formulating and administering therapeutically useful polypeptides are also disclosed in Remington: The Science and Practice of Pharmacy (Alfonso R. Gennaro, et. al. eds. Philadelphia College of Pharmacy and Science 2000), incorporated herein in its entirety.

### Therapeutically Effective Doses of GAABCP-TUPs

The present invention relates to a versatile means of increasing the half-life of any therapeutically useful polypeptide. Any suitable polypeptide, as defined herein, that has a therapeutic use and that would benefit from an increased half-life is encompassed by the present invention. Accordingly, the present invention also provides for the use of GAABCP-TUPs for preparing a medicament for the treatment and/or prevention of any disorder, disease, or condition that is treatable or preventable with a GAABCP-TUP. Such breadth is merited due to the versatile applicability of the invention. Accordingly, some non-limiting examples of disorders/conditions that may be treated with the GAABCP-TUPs disclosed herein include heart and peripheral vascular disease and other circulatory system discorders, cancer, diabetes and other related metabolic disorders, asthma and allergies and other immune system disorders, alzheimers disease and parkinsons disease and other neurological disorders, digestive system disorders, rheumatoid and osteoarthritis and other musculo-skeletal system disorders, endocrine disorders, fibrotic diseases, infectious diseases, etc. These may be treated by administering an effective amount of a GAABCP-TUP to a subject via any suitable route of administration, including, but not limited to, those described herein. The present invention furthermore provides for the use of GAABCP-TUPs as a method for the treatment and/or prevention of disorders, diseases, and conditions, in particular those mentioned above. The present invention furthermore provides for the use of GAABCP-TUPs for use in a method for the treatment and/or prophylaxis of disorders, diseases, and conditions, in particular the disorders, diseases, and conditions mentioned above.

In order to fully illustrate the present invention and advantages thereof, the following specific examples are given, it being understood that the same are intended only as illustrative and in no way limitative.

### EXAMPLES

### Example 1

### Genetic Conjugation of a (GGSGGSNNT)₁₀ Polypeptide to the C-Terminus of Six Unique TUPs

Representative examples of the design of the domain structure of GAABCP-TUPs are shown in FIG. 2 where the specific GAABCP polypeptide, (GGSGGSNNT)₁₀ (SEQ ID NO: 55), is conjugated to the C-terminus of 6 unique TUPs. These examples are in not meant to limit the scope of the invention, but rather to exemplify a few specific examples of the application of the invention.

In AQB-102 (SEQ ID NO: 100) the GAABCP-TUP glycoprotein conjugate is comprised of the following domains from the N- to C-terminus; an 18 amino acid Pho1 secretion signal peptide, a modified form of human G-CSF, a GAABCP with the sequence GGSGGSNNT repeated 10 times (SEQ ID NO: 55), and a 6×His tag sequence, according to the present disclosure. In AQB-304 (SEQ ID NO: 106) the GAABCP-TUP glycoprotein conjugate is comprised of the following domains from the N- to C-terminus; an 18 amino acid Pho1 secretion signal peptide, a modified form of human GLP-1 (7-37), a GAABCP with the sequence GGSGGSNNT repeated 10 times (SEQ ID NO: 55), and a 6×His tag sequence. In AQB-203a (SEQ ID NO: 113) the GAABCP-TUP glycoprotein conjugate is comprised of the following domains from the N- to C-terminus; a 24 amino acid native secretion signal peptide, a modified form of human prorelaxin-2, a GAABCP with the sequence GGSGGSNNT repeated 10 times (SEQ ID NO: 55), and a 6×His tag sequence. In AQB-401a (SEQ ID NO: 179) the GAABCP-TUP glycoprotein conjugate is comprised of the following domains from the N- to C-terminus; a 27 amino acid native secretion signal peptide, a modified form of human erythropoietin, a GAABCP with the sequence GGSGGSNNT repeated 10 times (SEQ ID NO: 55), a flexible linker with the sequence GGSGGS (SEQ ID NO: 73), and a 6×His tag sequence. In AQB-501a (SEQ ID NO: 180) the GAABCP-TUP glycoprotein conjugate is comprised of the following domains from the N- to C-terminus; an 18 amino acid Pho1 secretion signal peptide, a modified form of human adrenocorticotropic hormone, a GAABCP with the sequence GGSGGSNNT repeated 10 times (SEQ ID NO: 55), a flexible linker with the sequence GGSGGS (SEQ ID NO: 73), and a 6×His tag sequence. In AQB-601 (SEQ ID NO: 169) the GAABCP-TUP glycoprotein conjugate is comprised of the following domains from the N- to C-terminus; a 28 amino acid native secretion signal peptide, human coagulation factor IX, a GAABCP with the sequence GGSGGSNNT repeated 10 times (SEQ ID NO: 55), a flexible linker with the sequence GGSGGS (SEQ ID NO: 73), and a 6×His tag sequence, according to the present disclosure.

### Example 2

### Genetic Conjugation of GAABCP Polypeptides of Varying Glycan Acceptor Repeat Number and Spacer Length to the C-Terminus of Modified Human Granulocyte-Colony Stimulating Factor (hG-CSF)

Representative examples of the design of the domain structure of GAABCP-TUPs are shown in FIG. 3 where the number of repeating units of the specific GAABCP polypeptide (GGSGGSNNT)ₙ (SEQ ID NO: 35) is varied such that n=5, 10, 15 & 20, or the GGS spacer sequence [defined as (X₁X₂X₃)ₙ where X₁=G, X₂=G and X₃═S] is varied such that n=1, 2, 3 & 4, and in both instances where the GAABCP polypeptide is located to the C-terminus of a modified form of hG-CSF. These examples demonstrate a few of the range of structural variants outlined in Tables 1 and 3 with respect to the repeating GAABCP amino acid sequence and the spacer motif amino acid sequence, respectively, conjugated to a specific TUP.

In AQB-114 (SEQ ID NO: 134) the GAABCP-TUP comprises the following domain from the N- to C-terminus; an 18 amino acid Pho1 secretion signal peptide, a modified form of hG-CSF, and a GAABCP with the sequence GGSGGSNNT repeated 5 times (SEQ ID NO: 54). AQB-115 (SEQ ID NO: 135), AQB-116 (SEQ ID NO: 136) and AQB-117 (SEQ ID NO: 137) are identical to AQB-114 except that the GGSGGSNNT (SEQ ID NO: 35) repeat lengths are 10, 15 & 20, respectively. AQB-118 (SEQ ID NO: 138), AQB-119 (SEQ ID NO: 139) and AQB-120 (SEQ ID NO: 140) are identical to AQB-115 except that the GGS spacer repeats are 1, 3 & 4 respectively.

### Example 3

### Recombinant Expression of GAABCP Polypeptides of Varying Glycan Acceptor Repeat Number and Spacer Length to the C-Terminus of Modified Human Granulocyte-Colony Stimulating Factor (hG-CSF)

Novel genes representing GAABCP-TUP fusion glycoproteins AQB-114 thru 120 (SEQ ID NO: 134-140) were synthesized de novo based on the sequence shown in the corresponding sequence listing using methods well known to those in the art (Fath S, Bauer A P, Liss M et al. (2011) Multiparameter RNA and codon optimization: a standardized tool to assess and enhance autologous mammalian gene expression. PLoS One 6(3):e17596, incorporated herein). Following gene synthesis, the encoding region of each gene was subcloned into the cloning vector pXC-17.4 using HindIII/EcoRI cloning sites and employing standard recombinant DNA cloning methods. These plasmids were then used to transform chemically competent E. coli cells using the heat shock method, and positive transformants were selected using LB plates containing the appropriate antibiotic for selection. These colonies of transformed E. coli cells were then grown up in shake flask culture and endotoxin-free plasmid DNA was prepared. DNA sequencing of the plasmid constructs was performed, using vector specific forward and reverse primers, to confirm the correct gene sequence. The plasmid inserts were also verified by restriction enzyme digest and the calculated sizes found to be within 15% of the expected values.

Each unique gene expression plasmid was then linearized by treatment with PvuI restriction enzyme, and cultures of CHOK1 SV GS-KO cells were then individually transfected with each expression vector using electroporation methodology. CHOK1 SV GS-KO cells that were positively transformed with the expression plasmid were selected using L-methionine sulfoximine in order to generate stable CHO cell pools for each recombinant fusion glycoprotein gene. Each cell pool was then allowed to recover its normal growth pattern using shake flask culture conditions well known to those in the art; cell # and % viability were checked daily.

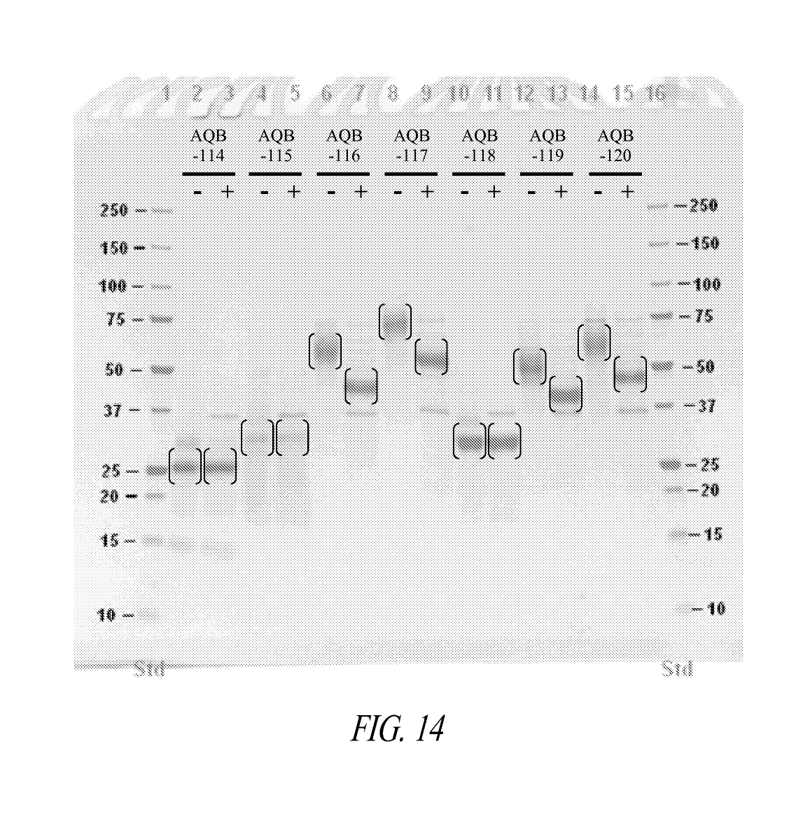

The CHO cell pools recombinantly expressing each of the seven fusion glycoproteins were then propagated to a volume of 1.2 L using shake flask culture format and a fed-batch overgrowth methodology (Davies S L, Lovelady C S, Grainger R K, Racher A J, et. al. (2013) Functional Heterogeneity and Heritability in CHO Cell Populations. Biotech. Bioeng. 110(1):260-274). On day 13 the media from each individual culture was harvested, then clarified by centrifugation and 0.22 um membrane filtration to obtain a clarified culture supernate to be used for purification of the recombinant fusion glycoproteins. FIG. 13A shows the expression levels of the recombinant fusion glycoprotein conjugates at various times over a 15 day culture period quantitated using a commercially available anti-TUP ELISA (hG-CSF Quantikine ELISA, R&D Systems) according to the manufacturer's instructions. These data suggest that the recombinant fusion glycoprotein is highly-expressed in the CHO cell pools and accumulates in the culture media over the 15 day period to levels of 250+ mg/L, which one skilled in the art would recognize as typical levels of recombinant protein expression. FIG. 13B shows the SDS-PAGE analysis of the day 13 cell pool conditioned media for each recombinant fusion glycoprotein (AQB-114 thru 120) to assess quality of product. In lanes 4 thru 10 is observed a major glycoprotein band in the CHO cell pool conditioned media corresponding to the molecular weight of each respective recombinant fusion glycoprotein, along with the many CHO host cell proteins.

### Example 4

### Purification and Characterization of GAABCP Polypeptides of Varying Glycan Acceptor Repeat Number and Spacer Length to the C-Terminus of Modified Human Granulocyte-Colony Stimulating Factor (hG-CSF)

Each fusion glycoprotein conditioned medium feedstock described in Example 3 was individually buffer exchanged into 50 mM Tris pH 8.0 using a tangential flow filtration system with a 10 kDa molecular weight cut-off membrane, then sterile filtered with a 0.22 um membrane. Anion exchange chromatography was performed on the conditioned supernates using a 25 mL Capto Q ion exchange chromatography column on an AKTA protein purification system (GE Healthcare). The column was equilibrated with 5 column volumes (CVs) of 50 mM Tris pH 8.0, each product individually loaded, followed by re-equilibration for 2 CVs of 50 mM Tris pH 8.0, and then washing with 50 mM Tris pH 8.0, 25 mM NaCl for 8 CV prior to elution. The products were eluted in a first phase with a linear 0 to 500 mM NaCl, 50 mM Tris-HCl pH 8.0 elution gradient over 10 CVs, and the second elution phase was a step to 50 mM Tris pH 8.0, 1M NaCl for 2 CVs. The eluted fractions were analyzed by SDS-PAGE and selected fractions containing the MW bands of interest were then pooled for size exclusion chromatography.

The pooled Capto Q fractions were then concentrated using a centrifugal filter unit with a 10 kDa MWCO filter to enable further purification using size exclusion chromatography on an AKTA protein purification system. These concentrated samples were injected on either a HiLoad 20/600 Superdex 75 pg for AQB-114, 115 and 118, or Superdex 200 pg for AQB-116, 117, 119 and 120. An isocratic elution in a PBS pH 7.4 mobile phase was performed at 2.7 mL/min and the eluate monitored by A280 and collected in 5 mL fractions. The fractions were supplemented with Tween-20 to give a final formulation of PBS pH 7.4, 0.05% (v/v) Tween-20. The eluted fractions were analyzed by SDS-PAGE and selected fractions containing the MW bands of interest were then pooled, and the concentration of the fusion glycoprotein estimated by A280 absorption. Based on this concentration data the sample was concentrated to approximately 1.0 mg fusion glycoprotein conjugate/mL using a centrifugal filter unit with a 10 kDa MWCO filter, and sterile filtered using a 0.22 um filter.

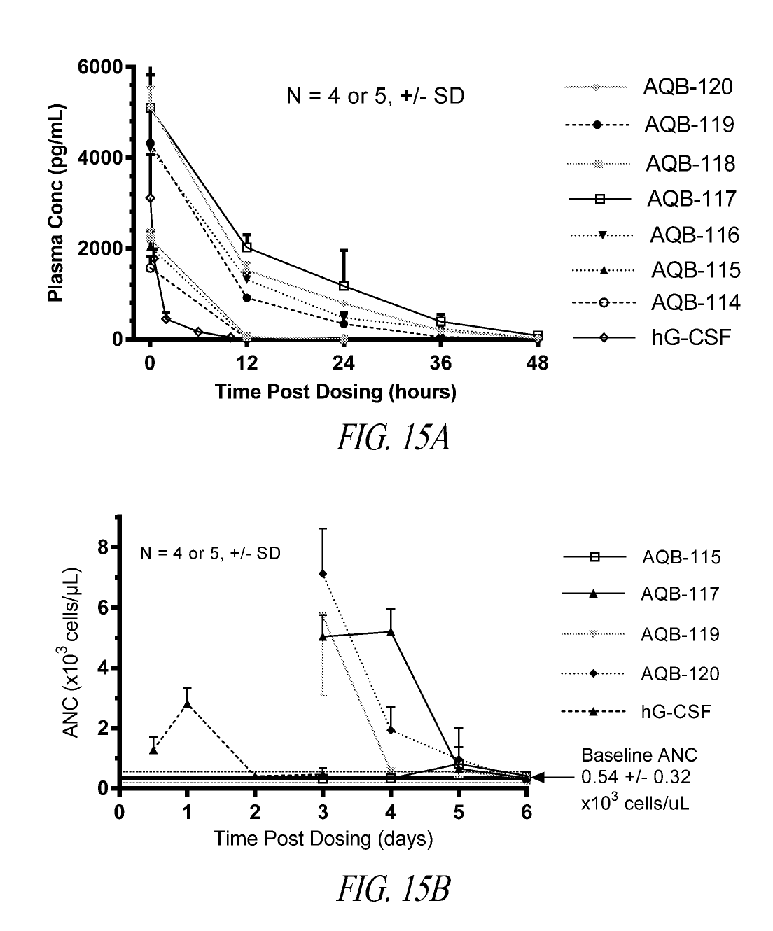

For each batch of purified fusion glycoprotein conjugate concentration was determined by anti-hG-CSF ELISA (as described previously) and amino acid analysis (see Cohen S, Strydom D. (1988) Amino acid analysis utilizing phenylisothiocyanate derivatives. Anal. Biochem. 174(1):1-16), identity by SDS-PAGE (see FIG. 14) before and after digestion with PNGase F to remove the N-glycan component (see Tarentino A, Plummer, T (1994) Enzymatic deglycosylation of asparagine-linked glycans: purification, properties, and specificity of oligosaccharide-cleaving enzymes from Flavobacterium meningosepticum. Methods in Enzymol. 230:44-57), aggregration status by size exclusion UPLC (see Chen X, Ge Y (2013) Ultrahigh pressure fast size exclusion chromatography for top-down proteomics. Proteomics. 13(17):2563-2566), carbohydrate content by HILIC-LC/MS analysis of the released N-glycans (see Wuhrer M, de Boer A R, Deelder A M (2009) Structural glycomics using hydrophilic interaction chromatography (HILIC) with mass spectrometry. Mass Spectrom. Rev. 28(2):192-206), released sialic acids by HPLC (see Rohrer J (2000) Analyzing sialic acids using high-performance anion-exchange chromatography with pulsed amperometric detection. Anal Biochem. 283(1):3-9), and endotoxin level using an end-point LAL assay (Charles River Labs) according to the manufacturer's instructions (all samples were <0.1 EU/mL). For the HILIC-LC/MS analysis average masses observed were matched to possible glycan structures using the GlycoMod software (Cooper C, Gasteiger E, Packer N. Predicting Glycan Composition from Experimental Mass Using GlycoMod (in) Handbook of Proteomic Methods (Conn P. M. ed.), pp. 225-232, Humana Press, Totowa, N.J. (2003), incorporated herein), with a tolerance of ±2.5 Da allowed. Together these data indicated that the fusion glycoproteins were 1) of well-defined concentration, 2) >90% purity, 3) primarily in a mono-molecular form (<2% aggregrated), 4) the polypeptide component was a single molecular species of the predicted molecular weight, 5) the carbohydrate component contained fucosylated complex-type N-glycans that were predominantly bi, tri and tetra-antennate forms, 6) the N-glycans were highly sialylated with >98% being the N-acetylneuraminic acid form, and 7) the batches contained low levels of endotoxins (<0.1 EU/mL).

### Example 5

### In Vitro Bioactivity Assessments of GAABCP Polypeptides of Varying Glycan Acceptor Repeat Number and Spacer Length to the C-Terminus of Modified Human Granulocyte-Colony Stimulating Factor (hG-CSF)

Murine NFS-60 cells (ATCC) were maintained in RPMI-1640 medium with 1% glutamine, 10% fetal bovine serum, 0.05 mM β-mercaptoethanol, and 62 ng/mL of M-CSF. For the proliferation assay, cells were washed twice with PBS, re-suspended in media devoid of growth factors, and 100 μLs of cell suspension with 5.0×10³ cells is dispensed per well in 96-well plates. A series of 2× concentrations of the AQB-114 thru 120 fusion glycoproteins, as well as molar equivalent of the comparator drug control (e.g., hG-CSF) were made over the desired testing range, and the concentrations were verified with an hG-CSF ELISA. Then, 100 μLs of the 2× solution was added to the cell culture. The cultures were incubated at 37° C. in a 5% CO₂, 100% humidity incubator for 48 hours. Cellular proliferation was then determined using the Rapid Cell Proliferation Kit (Calbiochem) according to the manufacturer's instructions, using background subtraction and comparison to an untreated control standard curve. Table 6 shows the bioactivity, expressed in pM, for each of the seven GAABCP-TUP fusion glycoproteins and the G-CSF comparator drug. These data indicate that genetic conjugation of the GAABCP domains to the C-terminus of a G-CSF TUP only resulted in a <3× loss of bioactivity compared to native G-CSF.

TABLE 6
In Vitro Bioactivity of AQB-114 thru 120 Plus Comparator
Drug in a Myeloid Cell Proliferation Assay.

| Test | G- | AQB- | AQB- | AQB- | AQB- | AQB- | AQB- | AQB- |
|---|---|---|---|---|---|---|---|---|
| Article | CSF | 114 | 115 | 116 | 117 | 118 | 119 | 120 |
| In Vitro | 2.6 | 2.0 | 5.6 | 4.9 | 7.3 | 4.3 | 2.8 | 4.7 |
| Bioactivity | | | | | | | | |
| (EC50, | | | | | | | | |
| in pM) | | | | | | | | |

### Example 6

### In Vivo Pharmacokinetic Assessment of GAABCP Polypeptides of Varying Glycan Acceptor Repeat Number and Spacer Length to the C-Terminus of Modified Human Granulocyte-Colony Stimulating Factor (hG-CSF)

In order to assess how GAABCP glycan repeat number (Group 1) and spacer motif lengths (Group 2) relate to plasma half-life, pharmacokinetic analysis on GAABCP-TUPs AQB-114 thru AQB-120 was performed.

Group 1.

AQB-114 thru AQB-117 each comprise the TUP hG-CSF C-terminally conjugated to a (GGSGGSNNT)ₙ-type GAABCP (SEQ ID NO: 35). In these constructs, the number of repeats ‘n’ is progressively increased as follows: for AQB-114, n=5; AQB-115, n=10; AQB-116, n=15; AQB-117, n=20.

Group 2.

AQB-118, AQB-115, AQB-119, and AQB-120 each comprise the TUP hG-CSF C-terminally conjugated to 10 repeats of a (GGS)ₙNNT-type GAABCP, wherein ‘n’ is progressively increased as follows: for AQB-118, n=1; AQB-115, n=2; AQB-119, n=3; AQB-120, n=4.

Pharmacokinetic analysis was performed by administering 180 ug of TUP equivalent mass/Kg of the AQB-114 thru 120 fusion glycoproteins, plus the hG-CSF comparator drug, as a single bolus intravenous injection via the lateral tail vein to 10-12 week old male C57/BL6 mice (5 mice per group). The concentrations of the 8 dosing solutions were verified with the anti-TUP ELISA. Blood samples were collected at the indicated times by venipuncture (from the abdominal aorta) into tubes containing EDTA at the indicated times. The samples were centrifuged for 10 minutes at a relative centrifugal force of 1000 g in a centrifuge set to maintain 4° C. The resultant plasma was collected and the concentration of the AQB-114 thru 120 fusion glycoproteins, and comparator drug, was determined by anti-TUP ELISA.

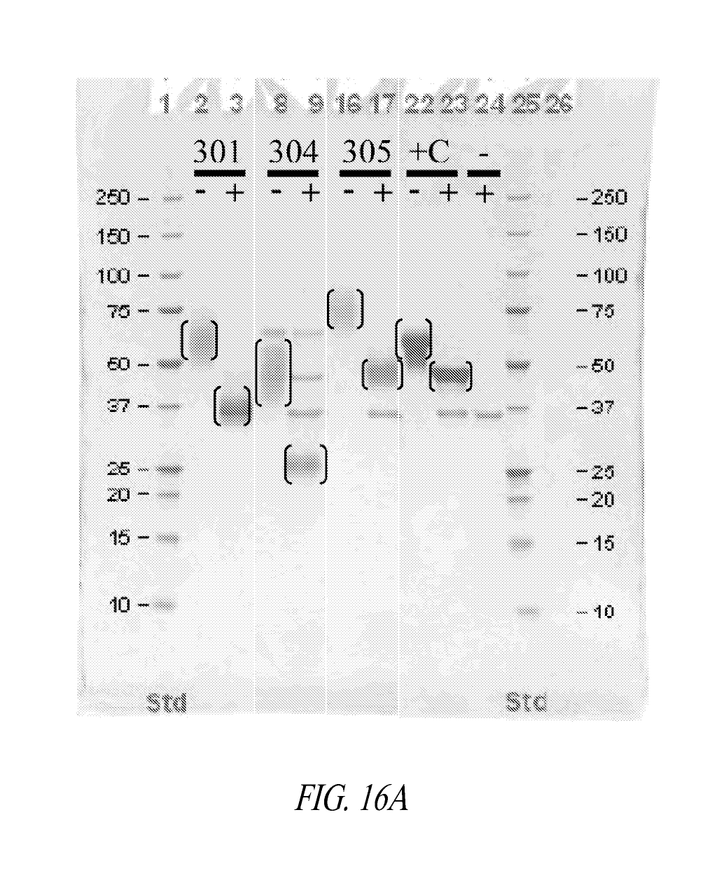

FIG. 15A shows that the Group 1 fusion glycoproteins with GAABCPs comprising 15 (AQB-116) and 20 (AQB-117) repeats of the GGSGGSNNT (SEQ ID NO: 35) block sequence possessed extended plasma residence times as compared to the native hG-CSF protein. Similarly, Group 2 fusion glycoproteins with GAABCs comprising 10 mer NNT repeats with spacer motifs containing the amino acid sequence GGSGGSGGS (SEQ ID NO: 17) (AQB-119) or GGSGGSGGSGGS (SEQ ID NO: 18) (AQB-120) possessed extended plasma residence times compared to the native hG-CSF protein. In contrast the fusion glycoproteins with GGSGGSNNT repeats of 5 (SEQ ID NO: 54) (AQB-114) and 10 (SEQ ID NO: 55) (AQB-115) or 10 mer NNT repeats with spacer motifs containing the amino acid sequence GGS (AQB-118) or GGSGGS (AQB-115) did not posess extended plasma residence times compared to the native G-CSF protein. Non-compartmental pharmacokinetic parameters were calculated using WinNonLin software (Pharsight), and the data is shown in Table 7. These data confirm the conclusions from the graphical data that GAABCP-TUP plasma half-life can be prolonged by either increasing the number of glycan acceptor motifs present in the GAABCP, or by increasing the spacing between the glycan acceptor motifs.

TABLE 7
In Vivo Pharmacokinetic Parameters for AQB-114 thru 120 Plus Comparator
Drugs Following a Single IV Bolus Injection in Mice.

| | Neupogen | AQB- | AQB- | AQB- | AQB- | AQB- | AQB- | AQB- |
|---|---|---|---|---|---|---|---|---|
| | (hG-CSF) | 114 | 115 | 116 | 117 | 118 | 119 | 120 |
| AUC_{(0-∞)} | 4350 | 4340 | 5930 | 45300 | 70300 | 7480 | 35500 | 55400 |
| (ng · h/mL) | | | | | | | | |
| Cl_{(0-∞)} | 0.345 | 0.430 | 0.447 | 0.0927 | 0.0712 | 0.303 | 0.107 | 0.0800 |
| (mL/min/kg) | | | | | | | | |
| Vd_{ss(0-∞)} | 49.9 | 72.0 | 78.5 | 60.6 | 55.5 | 59.9 | 54.1 | 51.8 |
| (mL/kg) | | | | | | | | |
| MRT_{(0-∞)} | 2.41 | 2.79 | 2.93 | 10.9 | 13.0 | 3.29 | 8.42 | 10.8 |
| (h) | | | | | | | | |
| t½ | 2.30 | 2.34 | 2.46 | 5.73 | 6.20 | 2.33 | 3.56 | 4.91 |
| (h) | | | | | | | | |

### Example 7

### In Vivo Pharmacodynamic Assessment of GAABCP Polypeptides of Varying Glycan Acceptor Repeat Number and Spacer Length to the C-Terminus of Modified Human Granulocyte-Colony Stimulating Factor (hG-CSF)

Pharmacodynamic analysis was performed by administering 0.5 mg of TUP equivalent mass/Kg of the AQB-115, 117, 119 and 120 fusion glycoproteins, plus the hG-CSF comparator drug, as a bolus subcutaneous injection in the dorsal region of 10-12 week old male C57/BL6 mice (5 mice per group). The concentrations of the 5 dosing solutions were verified with the anti-TUP ELISA. Blood samples were collected at the start of the experiment to establish the baseline count of the absolute neutrophil count (ANC) in the blood, and then at the indicated times after the start of the experiment. The blood samples were collected in EDTA treated tubes via the abdominal aorta and processed for a standard hematology panel on an ADVIA 12 clinical chemistry analyzer (Siemens Healthcare). FIG. 15B shows that the fusion glycoproteins with GGSGGSNNT repeats of 20 (SEQ ID NO: 57) (AQB-117), or 10 mer NNT repeats with spacer motifs containing the amino acid sequence GGSGGSGGS (SEQ ID NO: 17) (AQB-119) or GGSGGSGGSGGS (SEQ ID NO: 18) (AQB-120) possessed ANC levels that were both greater in magnitude and longer in duration that those observed for the native hG-CSF protein. In contrast, the fusion glycoprotein with GGSGGSNNT repeats of 10 (SEQ ID NO: 55) (AQB-115) possessed ANC levels that were neither greater in magnitude nor longer in duration that those observed for the native G-CSF protein.

### Example 8

### Relationship Between GAABCP Spacer Motif Amino Acid Length and N-Glycan Macroheterogeneity, and GAABCP-TUP Plasma Half-Life

GAABCP glycan occupancy, e.g., the percentage of glycan acceptor motifs in a given GAABCP that are bound by a glycan, is an important factor in prolonging the plasma half-life of a TUP via GAABCP conjugation. Accordingly, one goal of the present invention is to engineer GAABCP's that comprise high degrees of glycan occupancy. It is well known to those skilled in the art that the N-glycans must contain high levels of terminal sialic acid moieties on the bi-, tri or tetra-antennate complex type N-glycan in order to prevent first pass clearance of the GAABCP-TUP by the asialoglycoprotein receptor system in the hepatic circulation.

For a GAABCP with a given number of glycan acceptor motifs, the glycan macroheterogeneity is defined as the percent of potential glycan acceptor motifs that are occupied by an N-glycan chain. The greater the percentage of glycan acceptor motifs that are occupied by N-glycan chains the lower the macroheterogeneity of the GAABCP domain. Lower GAABCP macroheterogeneity means a larger number of N-glycan chains have been added as a post-translational modification for a GAABCP-TUP fusion glycoprotein of a given number of repeating sequences, which in turn results in a prolonged plasma half-life for the GAABCP-TUP compared to one with high macroheterogeneity.

The number of N-glycan units on a GAABCP-TUP can be estimated by applying the following formula:

[(Average MWapp of Intact Fusion Glycoprotein)−(Average MWapp of PNGase F treated Fusion Glycoprotein)]/(Mean MW of the Population of the N-glycan Chains)=Number of N-glycan Units

The average apparent molecular weight (MWapp) of the intact fusion glycoprotein and the average MWapp of the PNGase F treated fusion glycoprotein (which removes all of the N-glycan units) can be determined using SDS-PAGE, and the mean molecular weight (MW) of the N-glycan chains can be determined by HILIC-MS analysis of the released N-glycan chains.

To determine the effect spacer size might have on glycan occupancy, we generated the series of GAABCP-TUPs represented by AQB-118, 115, 119 & 120 (FIG. 3) (SEQ ID NOS: 138, 135, 139 & 140, respectively), each of which contain the TUP hG-CSF conjugated to a GAABCP that comprises 10 glycan acceptor motifs of the sequence NNT, each glycan acceptor separated from its nearest neighboring glycan acceptor by a spacer motif with the sequence GGS repeated 1, 2, 3 or 4 times respectively. For each one of these four GAABCP-TUPs it has been shown that the mean molecular weight of the population of N-glycan chains was 2,200 Daltons (data not shown).

Fusion glycoprotein variants AQB-118, 115, 119 & 120 (also see FIG. 2 for domain structures) were purified according to Example 4 and then they were treated with PNGase F (New England Biolabs) according to the manufacture's recommendations to remove the N-glycans. Treated and untreated samples were then analyzed by 4-12% Bis-Tris Novex SDS-PAGE with MES buffer (Invitrogen) run under reducing conditions and stained with Coomassie blue.

In FIG. 14, SDS-PAGE data shows that for AQB-119, which comprises a (GGS)₃ spacer sequence (lane 12 compared to 13) and AQB-120, which comprises a (GGS)₄ spacer (lane 14 compared to 15) a molecular weight differential is observed between the PNGase F treated and untreated samples, thus, demonstrating the presence of N-glycans. For AQB-115, which comprises a (GGS)₂ spacer sequence (lane 4 compared to 5) and AQB-118, which comprises a (GGS)₁ spacer (lane 10 compared to 11), no significant differentials were detectable.

Based upon the observed differentials, we applied the above formula to determine the number of N-glycan units on each fusion glycoprotein. For this series of GAABCP-TUPs, as the size of the spacer motif increased from 3 or 6 amino acids (AQB-118 or AQB-115) to 9 or 12 amino acids (AQB-119 or AQB-120) the percent N-glycan occupancy went from <5% to 30-35%. Data in Example 6/FIG. 15A/Table 7 shows that this observed increases in % N-glycan occupancy (due to longer spacer motif length) strongly correlated to an increase in plasma half life for AQB-119 and 120 as compared to AQB-118 and 115.

These data demonstrated that increasing the GAABCP spacer motif in this series of GAABCP-TUPs from 3 or 6 amino acids to 9 or 12 amino acids resulted in higher % of N-glycan occupancy and prolonged plasma half-life. Thus, taken together with the other examples presented herein, this data supports that increases in glycan occupancy can be achieved by increasing the spacer sequence between the repeating glycan acceptor motifs; and such increases in glycan occupancy correlate with increased plasma half-life.

### Example 9

### Genetic Conjugation of GAABCP Polypeptides of Varying Glycan Acceptor Repeat Number, Glycan Acceptor Motif Sequence, and Spacer Length to the C-Terminus of Modified Human Glugagon-Like Peptide-1 (hGLP-1)

Representative examples of the design of the domain structure of GAABCP-TUPs are shown in FIG. 4 where the number of repeating units of the specific GAABCP polypeptide (GGSGGSNNT)ₙ is varied such that n=5, 10, 15, 20 and 25, the glycan acceptor motif amino acid sequence is NNT or NIT, or the GGS spacer sequence [defined as (X₁X₂X₃)ₙ where X₁=G, X₂=G and X₃═S] is varied such that n=2, 3, 4 & 4, and in all instances where the GAABCP polypeptide is located to the C-terminus of a modified form of hGLP-1. These examples demonstrate a few of the range of structural variants outlined in Tables 1 thru 3 with respect to the repeating GAABCP amino acid sequence, the spacer motif amino acid sequence and the glycan acceptor motif, respectively, conjugated to a specific TUP.

In AQB-303 (SEQ ID NO: 105) the GAABCP-TUP comprises the following domains from the N- to C-terminus; an 18 amino acid Pho1 secretion signal peptide, a modified form of hGLP-1, a GAABCP with the sequence GGSGGSNNT repeated 5 times (SEQ ID NO: 54), and a 6×His tag sequence, according to the present disclosure. AQB-304 (SEQ ID NO: 106), AQB-301 (SEQ ID NO: 107) and AQB-305 (SEQ ID NO: 108) are identical to AQB-303 except that the GGSGGSNNT (SEQ ID NO: 35) repeat lengths are 10, 15 & 20, respectively. AQB-309 (SEQ ID NO: 143), AQB-310 (SEQ ID NO: 144) and AQB-311 (SEQ ID NO: 145) utilize the glycan acceptor motif amino acid sequence NIT. AQB-303 thru 305 (SEQ ID NOS: 105-108), AQB-309 (SEQ ID NO: 143), AQB-310 (SEQ ID NO: 144) and AQB-311 (SEQ ID NO: 145) have GGS spacer repeats are 2, 3, 4 & 5 respectively.

### Example 10

### Recombinant Expression of GAABCP Polypeptides of Varying Glycan Acceptor Repeat Number to the C-Terminus of Modified Human Glugagon-Like Peptide-1 (hGLP-1)

Novel genes representing GAABCP-TUP fusion glycoproteins AQB-301, 304 & 305 (SEQ ID NOS: 106-108) were synthesized de novo based on the sequence shown in the corresponding sequence listing using methods well known to those in the art (Fath S, Bauer A P, Liss M et al. (2011) Multiparameter RNA and codon optimization: a standardized tool to assess and enhance autologous mammalian gene expression. PLoS One 6(3):e17596, incorporated herein). Following gene synthesis, the encoding region of each gene was subcloned into the cloning vector pTT5 using EcoR1/BamH1 cloning sites and employing standard molecular biology methods. These plasmids were then used to transform chemically competent E. coli cells using the heat shock method, and positive transformants were selected using LB plates containing the appropriate antibiotic for selection. These colonies of transformed E. coli cells were then grown up in shake flask culture and endotoxin-free plasmid DNA was prepared. DNA sequencing of the plasmid constructs was performed, using vector specific forward and reverse primers, to confirm the correct gene sequence. The plasmids were also verified by restriction enzyme digest and the calculated sizes found to be within 15% of the expected values.

Each unique gene expression plasmid was then linearized by treatment with PstI restriction enzyme, and individual 1 L cultures of CHO-3E7 cells were then transiently transfected with each expression vector using fully deacetylated linear polyethylenimine methodology (see U.S. Pat. No. 6,013,240, European Patent 0,770,140). The 1 L cultures of transiently transfected CHO-3E7 cells recombinantly expressing each of the seven fusion glycoproteins were then grown for 8 days using shake flask culture conditions and a fed-batch methodology well known to those in the art (see Raymond C, Tom R, Perret S, Moussouami P, et. a. (2011) A simplified polyethylenimine-mediated transfection process for large-scale and high-throughput applications. Methods. 55(1):44-51); cell # and % viability were checked daily. On the day of harvest the media from each individual culture was removed, and then clarified by centrifugation and 0.22 um membrane filtration to obtain a culture supernate feedstock to be used for purification of the recombinant fusion glycoproteins.

### Example 11

### Purification and Characterization of GAABCP Polypeptides of Varying Glycan Acceptor Repeat Number to the C-Terminus of Modified Human Glugagon-Like Peptide-1 (hGLP-1)

Immobilized metal affinity chromatography (IMAC) was performed on the individual culture supernate feedstocks using 60 mL Fractogel chelate columns charged with Ni⁺⁺ ions (EMD Millipore) on an AKTA protein purification system (GE Healthcare). Columns were loaded with the feedstock followed by washing with 10 mM imidazole for 6 CV, followed by elution with a linear 10 to 300 mM imidazole gradient over 10 CVs with fractionation. The eluted fractions were analyzed by SDS-PAGE and selected fractions containing the MW bands of interest were then pooled for size exclusion chromatography.

The pooled IMAC fractions were then concentrated using a centrifugal filter unit with a 10 kDa MWCO filter to enable further purification via size exclusion chromatography on an AKTA protein purification system. Each individual concentrated sample was applied to a Superdex 200 26/600 column running a PBS pH 7.4 mobile phase, and the column was developed with isocratic elution at 2.7 mL/min and 5 mL fractions were collected. The fractions were supplemented with Tween-20 to give a final formulation of PBS pH 7.4, 0.05% (v/v) Tween-20. The eluded fractions were analyzed by SDS-PAGE and selected fractions containing the MW bands of interest were then pooled, and the fusion glycoprotein concentrations estimated by A280 absorption. Based on this concentration data the sample was concentrated to approximately 1.0 mg fusion glycoprotein conjugate/mL using a centrifugal filter unit with a 10 kDa MWCO filter, and sterile filtered using a 0.22 um filter.

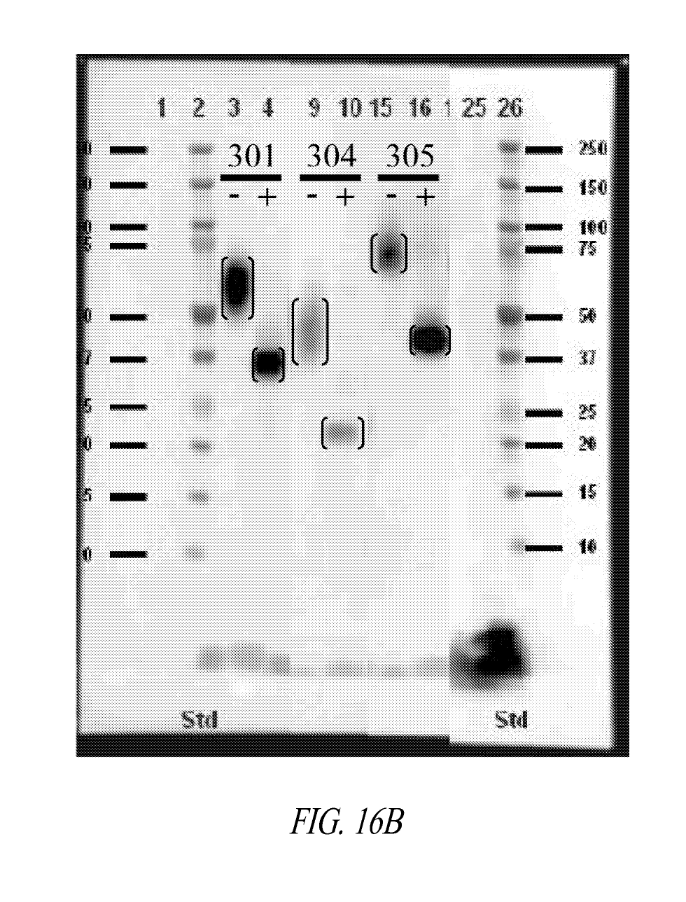

For each batch of purified fusion glycoprotein conjugate concentration was determined by anti-hGLP-1 ELISA (EMD Millipore) and amino acid analysis (see Cohen S, Strydom D. (1988) Amino acid analysis utilizing phenylisothiocyanate derivatives. Anal Biochem. 174(1):1-16), identity by SDS-PAGE (FIG. 16A) and Western blot with an anti-hGLP-1 (7-39) antibody (EMD Millipore; FIG. 16B) before and after digestion with PNGase F to remove the N-glycan component (see Tarentino A, Plummer, T (1994) Enzymatic deglycosylation of asparagine-linked glycans: purification, properties, and specificity of oligosaccharide-cleaving enzymes from Flavobacterium meningosepticum. Methods in Enzymol. 230:44-57), N-terminal sequencing for confirmation of an intact N-terminus, aggregration status by size exclusion UPLC (see Chen X, Ge Y (2013) Ultrahigh pressure fast size exclusion chromatography for top-down proteomics. Proteomics. 13(17):2563-2566), carbohydrate content by HILIC-LC/MS analysis of the released N-glycans (see Wuhrer M, de Boer A R, Deelder A M (2009) Structural glycomics using hydrophilic interaction chromatography (HILIC) with mass spectrometry. Mass Spectrom. Rev. 28(2):192-206), released sialic acid quantitation by HPLC (see Rohrer J (2000) Analyzing sialic acids using high-performance anion-exchange chromatography with pulsed amperometric detection. Anal Biochem. 283(1):3-9), and endotoxin level using an end-point LAL assay (Charles River Labs) according to the manufacturer's instructions (all samples were <0.1 EU/mL). For the HILIC-LC/MS analysis average masses observed may be matched to possible glycan structures using the GlycoMod software (Cooper C, Gasteiger E, Packer N. Predicting Glycan Composition from Experimental Mass Using GlycoMod (in) Handbook of Proteomic Methods (Conn P. M. ed.), pp. 225-232, Humana Press, Totowa, N.J. (2003), incorporated herein), with a tolerance of ±2.5 Da allowed. N-terminal sequencing is performed on the glycoprotein conjugates using an automated Edman degradation methodology on an ABI Procise™ Protein Sequencing System running a Spheri-5 PTH C18 (5 um, 220×2.1 mm) column. Together these data indicated that the AQB-301, AQB-304 & AQB-305 fusion glycoproteins were 1) of well-defined concentration, 2) >90% purity, 3) primarily in a mono-molecular form (<2% aggregrated), 4) the polypeptide component was a single molecular species of the predicted molecular weight, 5) the carbohydrate component contained fucosylated complex-type N-glycans that were predominantly bi, tri and tetra-antennate forms, 6) the N-glycans were highly sialylated with >98% being the N-acetylneuraminic acid form, and 7) the batches contained low levels of endotoxins.

### Example 12

### Recombinant Expression of GAABCP Polypeptides of Varying Glycan Acceptor Repeat Number and and Varying Spacer Motif Size Conjugated to the C-Terminus of Modified Human Glugagon-Like Peptide-1 (hGLP-1)

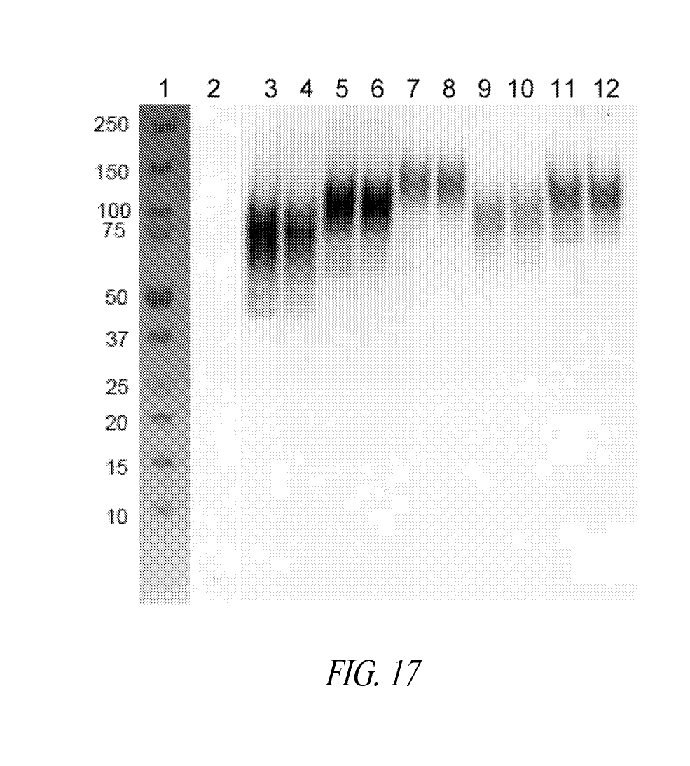

Novel genes representing GAABCP-TUP fusion glycoproteins AQB-307 thru 311 (SEQ ID NOS: 141-145) were cloned and expressed essentially as described in Example 10. For each batch of clarified conditioned media the molecular weight of the immunoreactive bands, corresponding to the recombinant fusion glycoprotein, was determined by Western blot with an anti-hGLP-1 antibody (FIG. 17). These data indicated that each fusion glycoprotein had an expected distribution of immunoreactive molecular weight forms, and that the average molecular weight for each correlated with both the number of GGSGGSGGSNIT repeats (15, lane 3 & 4; 20, lanes 5 & 6, or 25, lanes 7 &8) and the length of the GGS spacer (9 amino acids, lane 3 & 4; 12 amino acids, lanes 9 & 10, and 15 amino acids, lane 11 & 12).

### Example 13

### Recombinant Expression of GAABCP Polypeptides of Varying Glycan Acceptor Repeat Number/Sequence and Varying Spacer Motif Size Conjugated to the C- or N-Terminus of Modified Human Erythropoietin (hEPO)

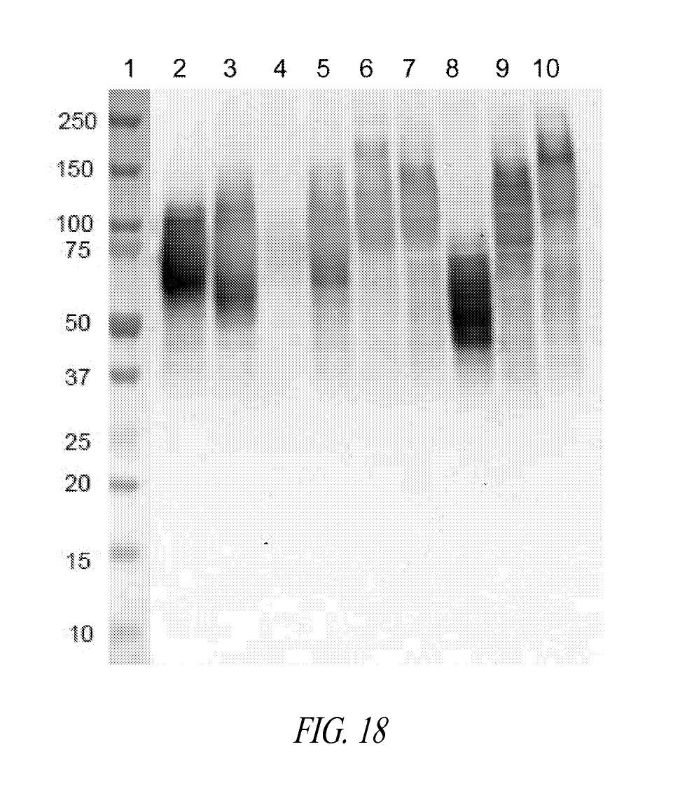

Novel genes representing GAABCP-TUP fusion glycoproteins AQB-401 thru 409 (SEQ ID NOS: 148-156) were cloned and expressed essentially as described in Example 9. For each batch of clarified conditioned media the molecular weight of the immunoreactive bands, corresponding to the recombinant fusion glycoprotein, was determined by Western blot with an anti-hEPO antibody (FIG. 18). These data indicated that each fusion glycoprotein had an expected distribution of immunoreactive molecular weight forms, and further that the average molecular weight for each fusion glycoprotein correlated with both 1) the number of GGSGGSGGSNNT (SEQ ID NO: 36) repeats (5, Lane 8, 10, lane 2; 15, lane 9, or 20, lane 10), and 2) the length of the GGS spacer (9 amino acids, lane 2; 12 amino acids, lane 5; 15 amino acids, lane 6; or 18 amino acids, lane 7). Whether the glycan acceptor motif sequence was comprised of NNT (lane 2) or NIT (lane 3) did not appear to make a significant difference in the distribution of immunoreactive bands. Genetic conjugation of the (GGSGGSGGSNNT)₁₀ (SEQ ID NO: 182) sequence to the N-terminus of the hEPO sequence (AQB-403, lane 3) appeared to diminish the expression of immunoreactive bands compared to conjugation to the C-terminus (AQB-401, lane 2).

### Example 14

### Genetic Conjugation of a (GGSGGSNNT)5 Polypeptide to Multiple TUPs

Representative examples of the design of the domain structure of GAABCP-TUPs are shown in FIG. 7, where the specific GAABCP polypeptide (GGSGGSNNT)₅ (SEQ ID NO: 54) is conjugated to two distinct TUPs, with or without intervening linker sequences. These examples are in not meant to limit the scope of the invention, but rather to exemplify a few specific examples of this specific application of the invention.

In AQB-110 (SEQ ID NO: 121) the GAABCP-TUP comprises a GAABCP with the sequence GGSGGSNNT repeated 5 times (SEQ ID NO: 54), with modified hG-CSF (SEQ ID NO: 98) conjugated to the N-terminal of the GAABCP and a portion of the IgG1 Fc domain (SEQ ID NO: 112) conjugated to the C-terminal with an intervening GGGGSGGGGS (SEQ ID NO: 93) linker sequence. This GAABCP-TUP further comprises an 18 amino acid Pho1 secretion signal peptide located at the N-terminus of the preprotein.

In AQB-255 (SEQ ID NO: 122) the GAABCP-TUP comprises from N-terminus to C-terminus, a native human prorelaxin-2 secretion signal peptide, a portion of the IgG1 Fc domain (SEQ ID NO: 112), a GAABCP with the sequence GGSGGSNNT repeated 5 times (SEQ ID NO: 54), a GGGGSGGGGS (SEQ ID NO: 93) linker sequence, and native human prorelaxin-2 (SEQ ID NO: 109).

In AQB-263 (SEQ ID NO: 123) the GAABCP-TUP comprises from N-terminus to C-terminus, a native human prorelaxin-2 secretion signal peptide, a GAABCP with the sequence GGSGGSNNT repeated 5 times (SEQ ID NO: 54), a GGGGSGGGGS (SEQ ID NO: 93) linker sequence, a portion of the IgG1 Fc domain (SEQ ID NO: 112), and a native human prorelaxin-2 (SEQ ID NO: 109).

In AQB-701 (SEQ ID NO: 177) the GAABCP-TUP comprises from N-terminus to C-terminus, an 18 amino acid Pho1 secretion signal peptide, a sequence encoding a single chain variable region fragment of an anti-CD19 antibody, a GAABCP with the sequence GGSGGSNNT repeated 5 times (SEQ ID NO: 54), a GGSGGS (SEQ ID NO: 73) linker sequence, a sequence encoding a single chain variable region fragment of an anti-CD3 antibody, and a 6×His tag.

In AQB-702 (SEQ ID NO: 178) the GAABCP-TUP comprises from N-terminus to C-terminus, an 18 amino acid Pho1 secretion signal peptide, a sequence encoding a single chain variable region fragment of an anti-CD19 antibody, a GAABCP with the sequence GGSGGSNNT repeated 5 times (SEQ ID NO: 54), a sequence encoding a single chain variable region fragment of an anti-CD3 antibody, and a 6×His tag.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### SEQUENCE TABLES

TABLE 1
SPACER MOTIFS

| | Type/ | | SEQ |
|---|---|---|---|
| Name | species | Amino acid Sequences | ID NO: |
| SPACER | Protein/ | GGS | 1 |
| MOTIF | Artificial | | |
| SPACER | Protein/ | GGT | 2 |
| MOTIF | Artificial | | |
| SPACER | Protein/ | GGD | 3 |
| MOTIF | Artificial | | |
| SPACER | Protein/ | GGE | 4 |
| MOTIF | Artificial | | |
| SPACER | Protein/ | GGG | 5 |
| MOTIF | Artificial | | |
| SPACER | Protein/ | AAS | 6 |
| MOTIF | Artificial | | |
| SPACER | Protein/ | AAT | 7 |
| MOTIF | Artificial | | |
| SPACER | Protein/ | AAD | 8 |
| MOTIF | Artificial | | |
| SPACER | Protein/ | AAE | 9 |
| MOTIF | Artificial | | |
| SPACER | Protein/ | AAA | 10 |
| MOTIF | Artificial | | |
| SPACER | Protein/ | AGS | 11 |
| MOTIF | Artificial | | |
| SPACER | Protein/ | AGT | 12 |
| MOTIF | Artificial | | |
| SPACER | Protein/ | AGD | 13 |
| MOTIF | Artificial | | |
| SPACER | Protein/ | AGE | 14 |
| MOTIF | Artificial | | |
| SPACER | Protein/ | AGG | 15 |
| MOTIF | Artificial | | |
| SPACER | Protein/ | GGSGGS | 16 |
| MOTIF | Artificial | | |
| SPACER | Protein/ | GGSGGSGGS | 17 |
| MOTIF | Artificial | | |
| SPACER | Protein/ | GGSGGSGGSGGS | 18 |
| MOTIF | Artificial | | |
| SPACER | Protein/ | GGSGGSGGSGGSGGS | 19 |
| MOTIF | Artificial | | |
| SPACER | Protein/ | GGSGGSGGSGGSGGSGGS | 124 |
| MOTIF | Artificial | | |
| SPACER | Protein/ | GGSGGSGGSGGSGGSGGSGGS | 125 |
| MOTIF | Artificial | | |
| SPACER | Protein/ | GGSGGSGGSGGSGGSGGSGGSGGS | 126 |
| MOTIF | Artificial | | |
| SPACER | Protein/ | GGSGGSGGSGGSGGSGGSGGSGGS | 127 |
| MOTIF | Artificial | GGS | |
| SPACER | Protein/ | GGSGGSGGSGGSGGSGGSGGSGGS | 128 |
| MOTIF | Artificial | GGSGGS | |
| SPACER | Protein/ | GGTGGT | 20 |
| MOTIF | Artificial | | |
| SPACER | Protein/ | GGTGGTGGT | 21 |
| MOTIF | Artificial | | |
| SPACER | Protein/ | GGTGGTGGTGGT | 22 |
| MOTIF | Artificial | | |
| SPACER | Protein/ | GGTGGTGGTGGTGGT | 23 |
| MOTIF | Artificial | | |
| SPACER | Protein/ | GGTGGTGGTGGTGGTGGT | 129 |
| MOTIF | Artificial | | |
| SPACER | Protein/ | GGTGGTGGTGGTGGTGGTGGT | 130 |
| MOTIF | Artificial | | |
| SPACER | Protein/ | GGTGGTGGTGGTGGTGGTGGTGGT | 131 |
| MOTIF | Artificial | | |
| SPACER | Protein/ | GGTGGTGGTGGTGGTGGTGGTGGT | 132 |
| MOTIF | Artificial | GGT | |
| SPACER | Protein/ | GGTGGTGGTGGTGGTGGTGGTGGT | 133 |
| MOTIF | Artificial | GGTGGT | |

TABLE 2
GLYCAN ACCEPTOR MOTIFS

| | Type/ | | SEQ |
|---|---|---|---|
| Name | species | Amino acid Sequences | ID NO: |
| GYCAN | Protein/ | NNT | 24 |
| ACCEPTOR | Artificial | | |
| MOTIF | | | |
| GYCAN | Protein/ | NNS | 25 |
| ACCEPTOR | Artificial | | |
| MOTIF | | | |
| GYCAN | Protein/ | NGT | 26 |
| ACCEPTOR | Artificial | | |
| MOTIF | | | |
| GYCAN | Protein/ | NGS | 27 |
| ACCEPTOR | Artificial | | |
| MOTIF | | | |
| GYCAN | Protein/ | NAT | 28 |
| ACCEPTOR | Artificial | | |
| MOTIF | | | |
| GYCAN | Protein/ | NAS | 29 |
| ACCEPTOR | Artificial | | |
| MOTIF | | | |
| GYCAN | Protein/ | NVT | 30 |
| ACCEPTOR | Artificial | | |
| MOTIF | | | |
| GYCAN | Protein/ | NVS | 31 |
| ACCEPTOR | Artificial | | |
| MOTIF | | | |
| GYCAN | Protein/ | NIT | 32 |
| ACCEPTOR | Artificial | | |
| MOTIF | | | |
| GYCAN | Protein/ | NIS | 33 |
| ACCEPTOR | Artificial | | |
| MOTIF | | | |

TABLE 3
GAABCP BLOCK UNIT SEQUENCES AND GAABCP SEQUENCES

| | | | SEQ |
|---|---|---|---|
| Name | Type/species | Amino acid Sequences | ID NO: |
| GAABCP | Protein/ | GGSNNT | 34 |
| | Artificial | | |
| GAABCP | Protein/ | GGSGGSNNT | 35 |
| | Artificial | | |
| GAABCP | Protein/ | GGSGGSGGSNNT | 36 |
| | Artificial | | |
| GAABCP | Protein/ | GGSGGSGGSGGSNNT | 37 |
| | Artificial | | |
| GAABCP | Protein/ | GGSGGSGGSGGSGGSNNT | 38 |
| | Artificial | | |
| GAABCP | Protein/ | GGSGGSGGSGGSGGSGGSNNT | 181 |
| | Artificial | | |
| GAABCP | Protein/ | GGSGGSGGSGGSGGSGGSGGSNNT | 182 |
| | Artificial | | |
| GAABCP | Protein/ | GGSNNS | 39 |
| | Artificial | | |
| GAABCP | Protein/ | GGSGGSNNS | 40 |
| | Artificial | | |
| GAABCP | Protein/ | GGSGGSGGSNNS | 41 |
| | Artificial | | |
| GAABCP | Protein/ | GGSGGSGGSGGSNNS | 42 |
| | Artificial | | |
| GAABCP | Protein/ | GGSGGSGGSGGSGGSNNS | 43 |
| | Artificial | | |
| GAABCP | Protein/ | GGSGGSGGSGGSGGSGGSNNS | 183 |
| | Artificial | | |
| GAABCP | Protein/ | GGSGGSGGSGGSGGSGGSGGSNNS | 184 |
| | Artificial | | |
| GAABCP | Protein/ | GGSNGT | 44 |
| | Artificial | | |
| GAABCP | Protein/ | GGSGGSNGT | 45 |
| | Artificial | | |
| GAABCP | Protein/ | GGSGGSGGSNGT | 46 |
| | Artificial | | |
| GAABCP | Protein/ | GGSGGSGGSGGSNGT | 47 |
| | Artificial | | |
| GAABCP | Protein/ | GGSGGSGGSGGSGGSNGT | 48 |
| | Artificial | | |
| GAABCP | Protein/ | GGSGGSGGSGGSGGSGGSNGT | 185 |
| | Artificial | | |
| GAABCP | Protein/ | GGSGGSGGSGGSGGSGGSGGSNGT | 186 |
| | Artificial | | |
| GAABCP | Protein/ | GGSNAS | 49 |
| | Artificial | | |
| GAABCP | Protein/ | GGSGGSNAS | 50 |
| | Artificial | | |
| GAABCP | Protein/ | GGSGGSGGSNAS | 51 |
| | Artificial | | |
| GAABCP | Protein/ | GGSGGSGGSGGSNAS | 52 |
| | Artificial | | |
| GAABCP | Protein/ | GGSGGSGGSGGSGGSNAS | 53 |
| | Artificial | | |
| GAABCP | Protein/ | GGSGGSGGSGGSGGSGGSNAS | 187 |
| | Artificial | | |
| GAABCP | Protein/ | GGSGGSGGSGGSGGSGGSGGSNAS | 188 |
| | Artificial | | |
| GAABCP | Protein/ | GGSGGSNNTGGSGGSNNTGGSGGSNNTGGSGGSNNTGGSGGSNNT | 54 |
| | Artificial | | |
| GAABCP | Protein/ | GGSGGSNNTGGSGGSNNTGGSGGSNNTGGSGGSNNTGGSGGSNNTG | 55 |
| | Artificial | GSGGSNNTGGSGGSNNTGGSGGSNNTGGSGGSNNTGGSGGSNNT | |
| GAABCP | Protein/ | GGSGGSNNTGGSGGSNNTGGSGGSNNTGGSGGSNNTGGSGGSNNTG | 56 |
| | Artificial | GSGGSNNTGGSGGSNNTGGSGGSNNTGGSGGSNNTGGSGGSNNTGG | |
| | | SGGSNNTGGSGGSNNTGGSGGSNNTGGSGGSNNTGGSGGSNNT | |
| GAABCP | Protein/ | GGSGGSNNTGGSGGSNNTGGSGGSNNTGGSGGSNNTGGSGGSNNTG | 57 |
| | Artificial | GSGGSNNTGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTGGSGGSNNTGGSGGSNNTGGSGGSNNTGGSGGSNNT | |
| GAABCP | Protein/ | GGSGGSNNSGGSGGSNNSGGSGGSNNSGGSGGSNNSGGSGGSNNS | 58 |
| | Artificial | | |
| GAABCP | Protein/ | GGSGGSNNSGGSGGSNNSGGSGGSNNSGGSGGSNNSGGSGGSNNSG | 59 |
| | Artificial | GSGGSNNSGGSGGSNNSGGSGGSNNSGGSGGSNNSGGSGGSNNS | |
| GAABCP | Protein/ | GGSGGSNNSGGSGGSNNSGGSGGSNNSGGSGGSNNSGGSGGSNNSG | 60 |
| | Artificial | GSGGSNNSGGSGGSNNSGGSGGSNNSGGSGGSNNSGGSGGSNNSGG | |
| | | SGGSNNSGGSGGSNNSGGSGGSNNSGGSGGSNNSGGSGGSNNS | |
| GAABCP | Protein/ | GGSGGSNNSGGSGGSNNSGGSGGSNNSGGSGGSNNSGGSGGSNNSG | 61 |
| | Artificial | GSGGSNNSGGSGGSNNSGGSGGSNNSGGSGGSNNSGGSGGSNNSGG | |
| | | SGGSNNSGGSGGSNNSGGSGGSNNSGGSGGSNNSGGSGGSNNSGGS | |
| | | GGSNNSGGSGGSNNSGGSGGSNNSGGSGGSNNSGGSGGSNNS | |
| GAABCP | Protein/ | GGSGGSNGTGGSGGSNGTGGSGGSNGTGGSGGSNGTGGSGGSNGT | 62 |
| | Artificial | | |
| GAABCP | Protein/ | GGSGGSNGTGGSGGSNGTGGSGGSNGTGGSGGSNGTGGSGGSNGTG | 63 |
| | Artificial | GSGGSNGTGGSGGSNGTGGSGGSNGTGGSGGSNGTGGSGGSNGT | |
| GAABCP | Protein/ | GGSGGSNGTGGSGGSNGTGGSGGSNGTGGSGGSNGTGGSGGSNGTG | 64 |
| | Artificial | GSGGSNGTGGSGGSNGTGGSGGSNGTGGSGGSNGTGGSGGSNGTGG | |
| | | SGGSNGTGGSGGSNGTGGSGGSNGTGGSGGSNGTGGSGGSNGT | |
| GAABCP | Protein/ | GGSGGSNGTGGSGGSNGTGGSGGSNGTGGSGGSNGTGGSGGSNGTG | 65 |
| | Artificial | GSGGSNGTGGSGGSNGTGGSGGSNGTGGSGGSNGTGGSGGSNGTGG | |
| | | SGGSNGTGGSGGSNGTGGSGGSNGTGGSGGSNGTGGSGGSNGTGGS | |
| | | GGSNGTGGSGGSNGTGGSGGSNGTGGSGGSNGTGGSGGSNGT | |
| GAABCP | Protein/ | GGSNNTGGSNNTGGSNNTGGSNNTGGSNNTGGSNNTGGSNNTGGSN | 181 |
| | Artificial | NTGGSNNTGGSNNT | |
| GAABCP | Protein/ | GGSGGSGGSNNTGGSGGSGGSNNTGGSGGSGGSNNTGGSGGSGGSN | 182 |
| | Artificial | NTGGSGGSGGSNNTGGSGGSGGSNNTGGSGGSGGSNNTGGSGGSGG | |
| | | SNNTGGSGGSGGSNNTGGSGGSGGSNNT | |
| GAABCP | Protein/ | GGSGGSGGSGGSNNTGGSGGSGGSGGSNNTGGSGGSGGSGGSNNTG | 183 |
| | Artificial | GSGGSGGSGGSNNTGGSGGSGGSGGSNNTGGSGGSGGSGGSNNTGG | |
| | | SGGSGGSGGSNNTGGSGGSGGSGGSNNTGGSGGSGGSGGSNNTGGS | |
| | | GGSGGSGGSNNT | |
| GAABCP | Protein/ | GGSGGSGGSNITGGSGGSGGSNITGGSGGSGGSNITGGSGGSGGSN | 184 |
| | Artificial | ITGGSGGSGGSNITGGSGGSGGSNITGGSGGSGGSNITGGSGGSGG | |
| | | SNITGGSGGSGGSNITGGSGGSGGSNITGGSGGSGGSNITGGSGGS | |
| | | GGSNITGGSGGSGGSNITGGSGGSGGSNITGGSGGSGGSNITGGSG | |
| | | GSGGSNITGGSGGSGGSNITGGSGGSGGSNITGGSGGSGGSNITGG | |
| | | SGGSGGSNITGGSGGSGGSNITGGSGGSGGSNITGGSGGSGGSNIT | |
| | | GGSGGSGGSNITGGSGGSGGSNIT | |
| GAABCP | Protein/ | GGSGGSGGSGGSNNTGGSGGSGGSGGSNNTGGSGGSGGSGGSNNTG | 185 |
| | Artificial | GSGGSGGSGGSNNTGGSGGSGGSGGSNNTGGSGGSGGSGGSNNTGG | |
| | | SGGSGGSGGSNNTGGSGGSGGSGGSNNTGGSGGSGGSGGSNNTGGS | |
| | | GGSGGSGGSNNTGGSGGSGGSGGSNNTGGSGGSGGSGGSNNTGGSG | |
| | | GSGGSGGSNNTGGSGGSGGSGGSNNTGGSGGSGGSGGSNNT | |
| GAABCP | Protein/ | GGSGGSGGSGGSGGSNNTGGSGGSGGSGGSGGSNNTGGSGGSGGSG | 186 |
| | Artificial | GSGGSNNTGGSGGSGGSGGSGGSNNTGGSGGSGGSGGSGGSNNTGG | |
| | | SGGSGGSGGSGGSNNTGGSGGSGGSGGSGGSNNTGGSGGSGGSGGS | |
| | | GGSNNTGGSGGSGGSGGSGGSNNTGGSGGSGGSGGSGGSNNTGGSG | |
| | | GSGGSGGSGGSNNTGGSGGSGGSGGSGGSNNTGGSGGSGGSGGSGG | |
| | | SNNTGGSGGSGGSGGSGGSNNTGGSGGSGGSGGSGGSNNT | |
| GAABCP | Protein/ | GGSGGSGGSGGSGGSNNTGGSGGSGGSGGSGGSNNTGGSGGSGGSG | 187 |
| | Artificial | GSGGSNNTGGSGGSGGSGGSGGSNNTGGSGGSGGSGGSGGSNNTGG | |
| | | SGGSGGSGGSGGSNNTGGSGGSGGSGGSGGSNNTGGSGGSGGSGGS | |
| | | GGSNNTGGSGGSGGSGGSGGSNNTGGSGGSGGSGGSGGSNNT | |
| GAABCP | Protein/ | GGSGGSGGSGGSGGSGGSNNTGGSGGSGGSGGSGGSGGSNNTGGSG | 188 |
| | Artificial | GSGGSGGSGGSGGSNNTGGSGGSGGSGGSGGSGGSNNTGGSGGSGG | |
| | | SGGSGGSGGSNNTGGSGGSGGSGGSGGSGGSNNTGGSGGSGGSGGS | |
| | | GGSGGSNNTGGSGGSGGSGGSGGSGGSNNTGGSGGSGGSGGSGGSG | |
| | | GSNNTGGSGGSGGSGGSGGSGGSNNT | |
| GAABCP | Protein/ | GGSGGSNITGGSGGSNITGGSGGSNITGGSGGSNITGGSGGSNITG | 189 |
| | Artificial | GSGGSNITGGSGGSNITGGSGGSNITGGSGGSNITGGSGGSNITGG | |
| | | SGGSNITGGSGGSNITGGSGGSNITGGSGGSNITGGSGGSNIT | |
| GAABCP | Protein/ | GGSGGSGGSNITGGSGGSGGSNITGGSGGSGGSNITGGSGGSGGSN | 190 |
| | Artificial | ITGGSGGSGGSNITGGSGGSGGSNITGGSGGSGGSNITGGSGGSGG | |
| | | SNITGGSGGSGGSNITGGSGGSGGSNITGGSGGSGGSNITGGSGGS | |
| | | GGSNITGGSGGSGGSNITGGSGGSGGSNITGGSGGSGGSNIT | |
| GAABCP | Protein/ | GGSGGSGGSGGSNITGGSGGSGGSGGSNITGGSGGSGGSGGSNITG | 191 |
| | Artificial | GSGGSGGSGGSNITGGSGGSGGSGGSNITGGSGGSGGSGGSNITGG | |
| | | SGGSGGSGGSNITGGSGGSGGSGGSNITGGSGGSGGSGGSNITGGS | |
| | | GGSGGSGGSNITGGSGGSGGSGGSNITGGSGGSGGSGGSNITGGSG | |
| | | GSGGSGGSNITGGSGGSGGSGGSNITGGSGGSGGSGGSNIT | |
| GAABCP | Protein/ | GGSGGSGGSGGSGGSNITGGSGGSGGSGGSGGSNITGGSGGSGGSG | 192 |
| | Artificial | GSGGSNITGGSGGSGGSGGSGGSNITGGSGGSGGSGGSGGSNITGG | |
| | | SGGSGGSGGSGGSNITGGSGGSGGSGGSGGSNITGGSGGSGGSGGS | |
| | | GGSNITGGSGGSGGSGGSGGSNITGGSGGSGGSGGSGGSNITGGSG | |
| | | GSGGSGGSGGSNITGGSGGSGGSGGSGGSNITGGSGGSGGSGGSGG | |
| | | SNITGGSGGSGGSGGSGGSNITGGSGGSGGSGGSGGSNIT | |
| GAABCP | Protein/ | GGSGGSGGSNITGGSGGSGGSNITGGSGGSGGSNITGGSGGSGGSN | 193 |
| | Artificial | ITGGSGGSGGSNIT | |
| GAABCP | Protein/ | GGSGGSGGSNITGGSGGSGGSNITGGSGGSGGSNITGGSGGSGGSN | 194 |
| | Artificial | ITGGSGGSGGSNITGGSGGSGGSNITGGSGGSGGSNITGGSGGSGG | |
| | | SNITGGSGGSGGSNITGGSGGSGGSNIT | |
| GAABCP | Protein/ | GGSGGSGGSNITGGSGGSGGSNITGGSGGSGGSNITGGSGGSGGSN | 195 |
| | Artificial | ITGGSGGSGGSNITGGSGGSGGSNITGGSGGSGGSNITGGSGGSGG | |
| | | SNITGGSGGSGGSNITGGSGGSGGSNITGGSGGSGGSNITGGSGGS | |
| | | GGSNITGGSGGSGGSNITGGSGGSGGSNITGGSGGSGGSNITGGSG | |
| | | GSGGSNITGGSGGSGGSNITGGSGGSGGSNITGGSGGSGGSNITGG | |
| | | SGGSGGSNIT | |
| GAABCP | Protein/ | GGSGGSNNTGGSGGSNNTGGSGGSNITGGSGGSNITGGSGGSNITG | 196 |
| | Artificial | GSGGSNNTGGSGGSNNTGGSGGSNITGGSGGSNITGGSGGSNIT | |
| GAABCP | Protein/ | GGSGGSGGSNNTGGSGGSGGSNNTGGSGGSGGSNNTGGSGGSGGSN | 197 |
| | Artificial | NTGGSGGSGGSNNTGGSGGSGGSNNTGGSGGSGGSNNTGGSGGSGG | |
| | | SNNTGGSGGSGGSNNTGGSGGSGGSNNTGGSGGSGGSNNTGGSGGS | |
| | | GGSNNTGGSGGSGGSNNTGGSGGSGGSNNTGGSGGSGGSNNT | |
| GAABCP | Protein/ | GGSGGSGGSNNTGGSGGSGGSNNTGGSGGSGGSNNTGGSGGSGGSN | 198 |
| | Artificial | NTGGSGGSGGSNNTGGSGGSGGSNNTGGSGGSGGSNNTGGSGGSGG | |
| | | SNNTGGSGGSGGSNNTGGSGGSGGSNNTGGSGGSGGSNNTGGSGGS | |
| | | GGSNNTGGSGGSGGSNNTGGSGGSGGSNNTGGSGGSGGSNNTGGSG | |
| | | GSGGSNNTGGSGGSGGSNNTGGSGGSGGSNNTGGSGGSGGSNNTGG | |
| | | SGGSGGSNNT | |
| GAABCP | Protein/ | GGSGGSGGSGGSNNTGGSGGSGGSGGSNNTGGSGGSGGSGGSNNTG | 199 |
| | Artificial | GSGGSGGSGGSNNTGGSGGSGGSGGSNNTGGSGGSGGSGGSNNTGG | |
| | | SGGSGGSGGSNNTGGSGGSGGSGGSNNTGGSGGSGGSGGSNNTGGS | |
| | | GGSGGSGGSNNTGGSGGSGGSGGSNNTGGSGGSGGSGGSNNTGGSG | |
| | | GSGGSGGSNNTGGSGGSGGSGGSNNTGGSGGSGGSGGSNNTGGSGG | |
| | | SGGSGGSNNTGGSGGSGGSGGSNNTGGSGGSGGSGGSNNTGGSGGS | |
| | | GGSGGSNNTGGSGGSGGSGGSNNT | |
| GAABCP | Protein/ | GGSGGSGGSGGSNNTGGSGGSGGSGGSNNTGGSGGSGGSGGSNNTG | 200 |
| | Artificial | GSGGSGGSGGSNNTGGSGGSGGSGGSNNTGGSGGSGGSGGSNNTGG | |
| | | SGGSGGSGGSNNTGGSGGSGGSGGSNNTGGSGGSGGSGGSNNTGGS | |
| | | GGSGGSGGSNNTGGSGGSGGSGGSNNTGGSGGSGGSGGSNNTGGSG | |
| | | GSGGSGGSNNTGGSGGSGGSGGSNNTGGSGGSGGSGGSNNTGGSGG | |
| | | SGGSGGSNNTGGSGGSGGSGGSNNTGGSGGSGGSGGSNNTGGSGGS | |
| | | GGSGGSNNTGGSGGSGGSGGSNNTGGSGGSGGSGGSNNTGGSGGSG | |
| | | GSGGSNNTGGSGGSGGSGGSNNTGGSGGSGGSGGSNNTGGSGGSGG | |
| | | SGGSNNT | |
| GAABCP | Protein/ | GGSGGSGGSNITGGSGGSGGSNITGGSGGSGGSNITGGSGGSGGSN | 201 |
| | Artificial | ITGGSGGSGGSNIT | |
| GAABCP | Protein/ | GGSGGSGGSNITGGSGGSGGSNITGGSGGSGGSNITGGSGGSGGSN | 202 |
| | Artificial | ITGGSGGSGGSNITGGSGGSGGSNITGGSGGSGGSNITGGSGGSGG | |
| | | SNITGGSGGSGGSNITGGSGGSGGSNIT | |
| GAABCP | Protein/ | GGSGGSGGSNITGGSGGSGGSNITGGSGGSGGSNITGGSGGSGGSN | 203 |
| | Artificial | ITGGSGGSGGSNITGGSGGSGGSNITGGSGGSGGSNITGGSGGSGG | |
| | | SNITGGSGGSGGSNITGGSGGSGGSNITGGSGGSGGSNITGGSGGS | |
| | | GGSNITGGSGGSGGSNITGGSGGSGGSNITGGSGGSGGSNITGGSG | |
| | | GSGGSNITGGSGGSGGSNITGGSGGSGGSNITGGSGGSGGSNITGG | |
| | | SGGSGGSNIT | |

TABLE 4
SECRETION SIGNAL PEPTIDES, AFFINITY TAGS,
AND FLEXIBLE LINKER SEQUENCES

| Name | Type/species | Amino acid Sequences | SEQ ID NO: |
|---|---|---|---|
| Pho1 signal | Protein/S. pombe | MFLQNLFLGFLAVVCANA | 66 |
| peptide | | | |
| HSA signal | Protein/H. sapians | MKWVTFISLLFLFSSAYS | 67 |
| peptide | | | |
| 6x His Tag | Protein/Artificial | HHHHHH | 68 |
| Flag-tag | Protein/Artificial | AYKDDDDK | 69 |
| Myc-tag | Protein/Artificial | EQKLISEEDK | 70 |
| HA-tag | Protein/Artificial | YPYDVPDYA | 71 |
| Flexible linker | Protein/Artificial | GGS | 72 |
| Flexible linker | Protein/Artificial | GGSGGS | 73 |
| Flexible linker | Protein/Artificial | GGSGGSGGS | 74 |
| Flexible linker | Protein/Artificial | GGSGGSGGSGGS | 75 |
| Flexible linker | Protein/Artificial | GGSGGSGGSGGSGGS | 76 |
| Flexible linker | Protein/Artificial | GSG | 77 |
| Flexible linker | Protein/Artificial | GSGGSG | 78 |
| Flexible linker | Protein/Artificial | GSGGSGGSG | 79 |
| Flexible linker | Protein/Artificial | GSGGSGGSGGSG | 80 |
| Flexible linker | Protein/Artificial | GSGGSGGSGGSGGSG | 81 |
| Flexible linker | Protein/Artificial | GGGS | 82 |
| Flexible linker | Protein/Artificial | GGGSGGGS | 83 |
| Flexible linker | Protein/Artificial | GGGSGGGSGGGS | 84 |
| Flexible linker | Protein/Artificial | GGGSGGGSGGGSGGGS | 85 |
| Flexible linker | Protein/Artificial | GGGSGGGSGGGSGGGSGGGS | 86 |
| Flexible linker | Protein/Artificial | GGSG | 87 |
| Flexible linker | Protein/Artificial | GGSGGGSG | 88 |
| Flexible linker | Protein/Artificial | GGSGGGSGGGSG | 89 |
| Flexible linker | Protein/Artificial | GGSGGGSGGGSGGGSG | 90 |
| Flexible linker | Protein/Artificial | GGSGGGSGGGSGGGSGGGSG | 91 |
| Flexible linker | Protein/Artificial | GGGGS | 92 |
| Flexible linker | Protein/Artificial | GGGGSGGGGS | 93 |
| Flexible linker | Protein/Artificial | GGGGSGGGGSGGGGS | 94 |
| Flexible linker | Protein/Artificial | GGGGSGGGGSGGGGSGGGGS | 95 |
| Flexible linker | Protein/Artificial | GGGGSGGGGSGGGGSGGGGSGGGGS | 96 |

TABLE 5
NATIVE AND UNMODIFIED TUPS, AND CORRESPONDING GAABCP CONLIGATED TUPS

| | Type/ | | SEQ |
|---|---|---|---|
| Name | species | Amino acid Sequences | ID NO: |
| Native | Protein/ | MAGPAYESPMRLMALELLLWHSALWTVEQATPLGPASSLPQSFLLK | 97 |
| hG-CSF | H. sapiens | CLEQVRKIQGDGAALQEKLCATYKLCHPEELVLLGHSLGIPWAPLS | |
| | | SCPSQALQLAGCLSQLHSGLFLYQGLLQALEGISPELGPTLDTLQL | |
| | | DVADFATTIWQQMEELGMAPALQPTQGAMPAFASAFQRRAGGVLVA | |
| | | SHLQSFLEVSYRVLRHLAQP | |
| Modified | Protein/ | TPLGPASSLPQSFLLKCLEQVRKIQGDGAALQEKLCATYKLCHPEE | 98 |
| hG-CSF | Artificial | LVLLGHSLGIPWAPLSSCPSQALQLAGCLSQLHSGLFLYQGLLQAL | |
| (no signal | | EGISPELGPTLDTLQLDVADFATTIWQQMEELGMAPALQPTQGAMP | |
| peptide) | | AFASAFQRRAGGVLVASHLQSFLEVSYRVLRHLAQP | |
| AQB-107 | Protein/ | MFLQNLFLGFLAVVCANATPLGPASSLPQSFLLKCLEQVRKIQGDG | 99 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTHHHHHH | |
| AQB-102 | Protein/ | MFLQNLFLGFLAVVCANATPLGPASSLPQSFLLKCLEQVRKIQGDG | 100 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTHHHHHH | |
| AQB-108 | Protein/ | MFLQNLFLGFLAVVCANATPLGPASSLPQSFLLKCLEQVRKIQGDG | 101 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTGGSGGSNNTGGSGGSNNTGGSGGSNNTGGSGGSNNTGGSG | |
| | | GSNNTHHHHHH | |
| AQB-109 | Protein/ | MFLQNLFLGFLAVVCANATPLGPASSLPQSFLLKCLEQVRKIQGDG | 102 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTGGSGGSNNTGGSGGSNNTGGSGGSNNTGGSGGSNNTGGSG | |
| | | GSNNTGGSGGSNNTGGSGGSNNTGGSGGSNNTGGSGGSNNTGGSGG | |
| | | SNNTHHHHHH | |
| AQB-114 | Protein/ | MFLQNLFLGFLAVVCANATPLGPASSLPQSFLLKCLEQVRKIQGDG | 134 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNT | |
| AQB-115 | Protein/ | MFLQNLFLGFLAVVCANATPLGPASSLPQSFLLKCLEQVRKIQGDG | 135 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNT | |
| AQB-116 | Protein/ | MFLQNLFLGFLAVVCANATPLGPASSLPQSFLLKCLEQVRKIQGDG | 136 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTGGSGGSNNTGGSGGSNNTGGSGGSNNTGGSGGSNNTGGSG | |
| | | GSNNT | |
| AQB-117 | Protein/ | MFLQNLFLGFLAVVCANATPLGPASSLPQSFLLKCLEQVRKIQGDG | 137 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTGGSGGSNNTGGSGGSNNTGGSGGSNNTGGSGGSNNTGGSG | |
| | | GSNNTGGSGGSNNTGGSGGSNNTGGSGGSNNTGGSGGSNNTGGSGG | |
| | | SNNT | |
| AQB-118 | Protein/ | MFLQNLFLGFLAVVCANATPLGPASSLPQSFLLKCLEQVRKIQGDG | 138 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSNNTGGSNNTGGSNNTGGSNNTGGSNNTGGSNNTGG | |
| | | SNNTGGSNNTGGSNNTGGSNNT | |
| AQB-119 | Protein/ | MFLQNLFLGFLAVVCANATPLGPASSLPQSFLLKCLEQVRKIQGDG | 139 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSGGSNNTGGSGGSGGSNNTGGSGGSGGSNNTGG | |
| | | SGGSGGSNNTGGSGGSGGSNNTGGSGGSGGSNNTGGSGGSGGSNNT | |
| | | GGSGGSGGSNNTGGSGGSGGSNNTGGSGGSGGSNNT | |
| AQB-120 | Protein/ | MFLQNLFLGFLAVVCANATPLGPASSLPQSFLLKCLEQVRKIQGDG | 140 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSGGSGGSNNTGGSGGSGGSGGSNNTGGSGGSGG | |
| | | SGGSNNTGGSGGSGGSGGSNNTGGSGGSGGSGGSNNTGGSGGSGGS | |
| | | GGSNNTGGSGGSGGSGGSNNTGGSGGSGGSGGSNNTGGSGGSGGSG | |
| | | GSNNTGGSGGSGGSGGSNNT | |
| hGLP- 1 | Protein/ | HAEGTFTSDVSSYLEGQAAKEFIAWLVKGRG | 103 |
| (7-37) | Homo Sapien | | |
| Modified | Protein/ | HGEGTFTSDVSSYLEGQAAKEFIAWLVKGGG | 104 |
| hGLP-1 | Artificial | | |
| (7-37) | | | |
| AQB-303 | Protein/ | MFLQNLFLGFLAVVCANAHGEGTFTSDVSSYLEGQAAKEFIAWLVK | 105 |
| | Artificial | GGGGGSGGSNNTGGSGGSNNTGGSGGSNNTGGSGGSNNTGGSGGSN | |
| | | NTHHHHHH | |
| AQB-304 | Protein/ | MFLQNLFLGFLAVVCANAHGEGTFTSDVSSYLEGQAAKEFIAWLVK | 106 |
| | Artificial | GGGGGSGGSNNTGGSGGSNNTGGSGGSNNTGGSGGSNNTGGSGGSN | |
| | | NTGGSGGSNNTGGSGGSNNTGGSGGSNNTGGSGGSNNTGGSGGSNN | |
| | | THHHHHH | |
| AQB-301 | Protein/ | MFLQNLFLGFLAVVCANAHGEGTFTSDVSSYLEGQAAKEFIAWLVK | 107 |
| | Artificial | GGGGGSGGSNNTGGSGGSNNTGGSGGSNNTGGSGGSNNTGGSGGSN | |
| | | NTGGSGGSNNTGGSGGSNNTGGSGGSNNTGGSGGSNNTGGSGGSNN | |
| | | TGGSGGSNNTGGSGGSNNTGGSGGSNNTGGSGGSNNTGGSGGSNNT | |
| | | HHHHHH | |
| AQB-305 | Protein/ | MFLQNLFLGFLAVVCANAHGEGTFTSDVSSYLEGQAAKEFIAWLVK | 108 |
| | Artificial | GGGGGSGGSNNTGGSGGSNNTGGSGGSNNTGGSGGSNNTGGSGGSN | |
| | | NTGGSGGSNNTGGSGGSNNTGGSGGSNNTGGSGGSNNTGGSGGSNN | |
| | | TGGSGGSNNTGGSGGSNNTGGSGGSNNTGGSGGSNNTGGSGGSNNT | |
| | | GGSGGSNNTGGSGGSNNTGGSGGSNNTGGSGGSNNTGGSGGSNNTH | |
| | | HHHHH | |
| AQB-307 | Protein/ | MFLQNLFLGFLAVVCANAHGEGTFTSDVSSYLEGQAAKEFIAWLVK | 141 |
| | Artificial | GGGGGSGGSGGSNITGGSGGSGGSNITGGSGGSGGSNITGGSGGSG | |
| | | GSNITGGSGGSGGSNITGGSGGSGGSNITGGSGGSGGSNITGGSGG | |
| | | SGGSNITGGSGGSGGSNITGGSGGSGGSNITGGSGGSGGSNITGGS | |
| | | GGSGGSNITGGSGGSGGSNITGGSGGSGGSNITGGSGGSGGSNITG | |
| | | GSGGSHHHHHH | |
| AQB-308 | Protein/ | MFLQNLFLGFLAVVCANAHGEGTFTSDVSSYLEGQAAKEFIAWLVK | 142 |
| | Artificial | GGGGGSGGSGGSNITGGSGGSGGSNITGGSGGSGGSNITGGSGGSG | |
| | | GSNITGGSGGSGGSNITGGSGGSGGSNITGGSGGSGGSNITGGSGG | |
| | | SGGSNITGGSGGSGGSNITGGSGGSGGSNITGGSGGSGGSNITGGS | |
| | | GGSGGSNITGGSGGSGGSNITGGSGGSGGSNITGGSGGSGGSNITG | |
| | | GSGGSGGSNITGGSGGSGGSNITGGSGGSGGSNITGGSGGSGGSNI | |
| | | TGGSGGSGGSNITGGSGGSHHHHHH | |
| AQB-309 | Protein/ | MFLQNLFLGFLAVVCANAHGEGTFTSDVSSYLEGQAAKEFIAWLVK | 143 |
| | Artificial | GGGGGSGGSGGSNITGGSGGSGGSNITGGSGGSGGSNITGGSGGSG | |
| | | GSNITGGSGGSGGSNITGGSGGSGGSNITGGSGGSGGSNITGGSGG | |
| | | SGGSNITGGSGGSGGSNITGGSGGSGGSNITGGSGGSGGSNITGGS | |
| | | GGSGGSNITGGSGGSGGSNITGGSGGSGGSNITGGSGGSGGSNITG | |
| | | GSGGSGGSNITGGSGGSGGSNITGGSGGSGGSNITGGSGGSGGSNI | |
| | | TGGSGGSGGSNITGGSGGSGGSNITGGSGGSGGSNITGGSGGSGGS | |
| | | NITGGSGGSGGSNITGGSGGSGGSNITGGSGGSHHHHHH | |
| AQB-310 | Protein/ | MFLQNLFLGFLAVVCANAHGEGTFTSDVSSYLEGQAAKEFIAWLVK | 144 |
| | Artificial | GGGGGSGGSGGSGGSNITGGSGGSGGSGGSNITGGSGGSGGSGGSN | |
| | | ITGGSGGSGGSGGSNITGGSGGSGGSGGSNITGGSGGSGGSGGSNI | |
| | | TGGSGGSGGSGGSNITGGSGGSGGSGGSNITGGSGGSGGSGGSNIT | |
| | | GGSGGSGGSGGSNITGGSGGSGGSGGSNITGGSGGSGGSGGSNITG | |
| | | GSGGSGGSGGSNITGGSGGSGGSGGSNITGGSGGSGGSGGSNITGG | |
| | | SGGSHHHHHH | |
| AQB-311 | Protein/ | MFLQNLFLGFLAVVCANAHGEGTFTSDVSSYLEGQAAKEFIAWLVK | 145 |
| | Artificial | GGGGGSGGSGGSGGSGGSNITGGSGGSGGSGGSGGSNITGGSGGSG | |
| | | GSGGSGGSNITGGSGGSGGSGGSGGSNITGGSGGSGGSGGSGGSNI | |
| | | TGGSGGSGGSGGSGGSNITGGSGGSGGSGGSGGSNITGGSGGSGGS | |
| | | GGSGGSNITGGSGGSGGSGGSGGSNITGGSGGSGGSGGSGGSNITG | |
| | | GSGGSGGSGGSGGSNITGGSGGSGGSGGSGGSNITGGSGGSGGSGG | |
| | | SGGSNITGGSGGSGGSGGSGGSNITGGSGGSGGSGGSGGSNITGGS | |
| | | GGSHHHHHH | |
| hEPO | Protein/ | MGVHECPAWLWLLLSLLSLPLGLPVLGAPPRLICDSRVLERYLLEA | 146 |
| | Homo Sapein | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDR | |
| Modified | Protein/ | MGVHECPAWLWLLLSLLSLPLGLPVLGAPPRLICDSRVLERYLLEA | 147 |
| hEPO | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGD | |
| AQB-401 | Protein/ | MGVHECPAWLWLLLSLLSLPLGLPVLGAPPRLICDSRVLERYLLEA | 148 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSNNTGGSGGSGGSNNTGGSGGSGGSNNTGG | |
| | | SGGSGGSNNTGGSGGSGGSNNTGGSGGSGGSNNTGGSGGSGGSNNT | |
| | | GGSGGSGGSNNTGGSGGSGGSNNTGGSGGSGGSNNTGGSGGSHHHH | |
| | | HH | |
| AQB-402 | Protein/ | MGVHECPAWLWLLLSLLSLPLGLPVLGAPPRLICDSRVLERYLLEA | 149 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSNITGGSGGSGGSNITGGSGGSGGSNITGG | |
| | | SGGSGGSNITGGSGGSGGSNITGGSGGSGGSNITGGSGGSGGSNIT | |
| | | GGSGGSGGSNITGGSGGSGGSNITGGSGGSGGSNITGGSGGSHHHH | |
| | | HH | |
| AQB-403 | Protein/ | MFLQNLFLGFLAVVCANAGGSGGSGGSNNTGGSGGSGGSNNTGGSG | 150 |
| | Artificial | GSGGSNNTGGSGGSGGSNNTGGSGGSGGSNNTGGSGGSGGSNNTGG | |
| | | SGGSGGSNNTGGSGGSGGSNNTGGSGGSGGSNNTGGSGGSGGSNNT | |
| | | GGSGGSAPPRLICDSRVLERYLLEAKEAENITTGCAEHCSLNENIT | |
| | | VPDTKVNFYAWKRMEVGQQAVEVWQGLALLSEAVLRGQALLVNSSQ | |
| | | PWEPLQLHVDKAVSGLRSLTTLLRALGAQKEAISPPDAASAAPLRT | |
| | | ITADTFRKLFRVYSNFLRGKLKLYTGEACRTGDHHHHHH | |
| AQB-404 | Protein/ | MGVHECPAWLWLLLSLLSLPLGLPVLGAPPRLICDSRVLERYLLEA | 151 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSGGSNNTGGSGGSGGSGGSNNTGGSGGSGG | |
| | | SGGSNNTGGSGGSGGSGGSNNTGGSGGSGGSGGSNNTGGSGGSGGS | |
| | | GGSNNTGGSGGSGGSGGSNNTGGSGGSGGSGGSNNTGGSGGSGGSG | |
| | | GSNNTGGSGGSGGSGGSNNTGGSGGSHHHHHH | |
| AQB-405 | Protein/ | MGVHECPAWLWLLLSLLSLPLGLPVLGAPPRLICDSRVLERYLLEA | 152 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSGGSGGSNNTGGSGGSGGSGGSGGSNNTGG | |
| | | SGGSGGSGGSGGSNNTGGSGGSGGSGGSGGSNNTGGSGGSGGSGGS | |
| | | GGSNNTGGSGGSGGSGGSGGSNNTGGSGGSGGSGGSGGSNNTGGSG | |
| | | GSGGSGGSGGSNNTGGSGGSGGSGGSGGSNNTGGSGGSGGSGGSGG | |
| | | SNNTGGSGGSHHHHHH | |
| AQB-406 | Protein/ | MGVHECPAWLWLLLSLLSLPLGLPVLGAPPRLICDSRVLERYLLEA | 153 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSGGSGGSGGSNNTGGSGGSGGSGGSGGSGG | |
| | | SNNTGGSGGSGGSGGSGGSGGSNNTGGSGGSGGSGGSGGSGGSNNT | |
| | | GGSGGSGGSGGSGGSGGSNNTGGSGGSGGSGGSGGSGGSNNTGGSG | |
| | | GSGGSGGSGGSGGSNNTGGSGGSGGSGGSGGSGGSNNTGGSGGSGG | |
| | | SGGSGGSGGSNNTGGSGGSGGSGGSGGSGGSNNTGGSGGSHHHHHH | |
| AQB-407 | Protein/ | MGVHECPAWLWLLLSLLSLPLGLPVLGAPPRLICDSRVLERYLLEA | 154 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSNNTGGSGGSGGSNNTGGSGGSGGSNNTGG | |
| | | SGGSGGSNNTGGSGGSGGSNNTGGSGGSHHHHHH | |
| AQB-408 | Protein/ | MGVHECPAWLWLLLSLLSLPLGLPVLGAPPRLICDSRVLERYLLEA | 155 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSNNTGGSGGSGGSNNTGGSGGSGGSNNTGG | |
| | | SGGSGGSNNTGGSGGSGGSNNTGGSGGSGGSNNTGGSGGSGGSNNT | |
| | | GGSGGSGGSNNTGGSGGSGGSNNTGGSGGSGGSNNTGGSGGSGGSN | |
| | | NTGGSGGSGGSNNTGGSGGSGGSNNTGGSGGSGGSNNTGGSGGSGG | |
| | | SNNTGGSGGSHHHHHH | |
| AQB-409 | Protein/ | MGVHECPAWLWLLLSLLSLPLGLPVLGAPPRLICDSRVLERYLLEA | 156 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSGGSNNTGGSGGSGGSNNTGGSGGSGGSNNTGG | |
| | | SGGSGGSNNTGGSGGSGGSNNTGGSGGSGGSNNTGGSGGSGGSNNT | |
| | | GGSGGSGGSNNTGGSGGSGGSNNTGGSGGSGGSNNTGGSGGSGGSN | |
| | | NTGGSGGSGGSNNTGGSGGSGGSNNTGGSGGSGGSNNTGGSGGSGG | |
| | | SNNTGGSGGSGGSNNTGGSGGSGGSNNTGGSGGSGGSNNTGGSGGS | |
| | | GGSNNTGGSGGSGGSNNTGGSGGSHHHHHH | |
| hACTH | Protein/ | SYSMEHFRWGKPVGKKRRPVKVYPNGAEDESAEAFPLEF | 157 |
| | H. sapiens | | |
| AQB-501 | Protein/ | MFLQNLFLGFLAVVCANASYSMEHFRWGKPVGKKRRPVKVYPNGAE | 158 |
| | Artificial | DESAEAFPLEFGGSGGSGGSGGSNNTGGSGGSGGSGGSNNTGGSGG | |
| | | SGGSGGSNNTGGSGGSGGSGGSNNTGGSGGSGGSGGSNNTGGSGGS | |
| | | GGSGGSNNTGGSGGSGGSGGSNNTGGSGGSGGSGGSNNTGGSGGSG | |
| | | GSGGSNNTGGSGGSGGSGGSNNTGGSGGSHHHHHH | |
| AQB-502 | Protein/ | MFLQNLFLGFLAVVCANASYSMEHFRWGKPVGKKRRPVKVYPNGAE | 159 |
| | Artificial | DESAEAFPLEFGGSGGSGGSGGSNNTGGSGGSGGSGGSNNTGGSGG | |
| | | SGGSGGSNNTGGSGGSGGSGGSNNTGGSGGSGGSGGSNNTGGSGGS | |
| | | GGSGGSNNTGGSGGSGGSGGSNNTGGSGGSGGSGGSNNTGGSGGSG | |
| | | GSGGSNNTGGSGGSGGSGGSNNTGGSGGSGGSGGSNNTGGSGGSGG | |
| | | SGGSNNTGGSGGSGGSGGSNNTGGSGGSGGSGGSNNTGGSGGSGGS | |
| | | GGSNNTGGSGGSHHHHHH | |
| AQB-503 | Protein/ | MFLQNLFLGFLAVVCANASYSMEHFRWGKPVGKKRRPVKVYPNGAE | 160 |
| | Artificial | DESAEAFPLEFGGSGGSGGSGGSNNTGGSGGSGGSGGSNNTGGSGG | |
| | | SGGSGGSNNTGGSGGSGGSGGSNNTGGSGGSGGSGGSNNTGGSGGS | |
| | | GGSGGSNNTGGSGGSGGSGGSNNTGGSGGSGGSGGSNNTGGSGGSG | |
| | | GSGGSNNTGGSGGSGGSGGSNNTGGSGGSGGSGGSNNTGGSGGSGG | |
| | | SGGSNNTGGSGGSGGSGGSNNTGGSGGSGGSGGSNNTGGSGGSGGS | |
| | | GGSNNTGGSGGSGGSGGSNNTGGSGGSGGSGGSNNTGGSGGSGGSG | |
| | | GSNNTGGSGGSGGSGGSNNTGGSGGSGGSGGSNNTGGSGGSHHHHH | |
| | | H | |
| AQB-504 | Protein/ | MFLQNLFLGFLAVVCANASYSMEHFRWGKPVGKKRRPVKVYPNGAE | 161 |
| | Artificial | DESAEAFPLEFGGSGGSGGSGGSNNTGGSGGSGGSGGSNNTGGSGG | |
| | | SGGSGGSNNTGGSGGSGGSGGSNNTGGSGGSGGSGGSNNTGGSGGS | |
| | | GGSGGSNNTGGSGGSGGSGGSNNTGGSGGSGGSGGSNNTGGSGGSG | |
| | | GSGGSNNTGGSGGSGGSGGSNNTGGSGGSGGSGGSNNTGGSGGSGG | |
| | | SGGSNNTGGSGGSGGSGGSNNTGGSGGSGGSGGSNNTGGSGGSGGS | |
| | | GGSNNTGGSGGSGGSGGSNNTGGSGGSGGSGGSNNTGGSGGSGGSG | |
| | | GSNNTGGSGGSGGSGGSNNTGGSGGSGGSGGSNNTGGSGGSGGSGG | |
| | | SNNTGGSGGSGGSGGSNNTGGSGGSGGSGGSNNTGGSGGSGGSGGS | |
| | | NNTGGSGGSGGSGGSNNTGGSGGSHHHHHH | |
| AQB-505 | Protein/ | MFLQNLFLGFLAVVCANASYSMEHFRWGKPVGKKRRPVKVYPNGAE | 162 |
| | Artificial | DESAEAFPLEFGGSGGSGGSNNTGGSGGSGGSNNTGGSGGSGGSNN | |
| | | TGGSGGSGGSNNTGGSGGSGGSNNTGGSGGSGGSNNTGGSGGSGGS | |
| | | NNTGGSGGSGGSNNTGGSGGSGGSNNTGGSGGSGGSNNTGGSGGSG | |
| | | GSNNTGGSGGSGGSNNTGGSGGSGGSNNTGGSGGSGGSNNTGGSGG | |
| | | SGGSNNTGGSGGSHHHHHH | |
| AQB-506 | Protein/ | MFLQNLFLGFLAVVCANASYSMEHFRWGKPVGKKRRPVKVYPNGAE | 163 |
| | Artificial | DESAEAFPLEFGGSGGSGGSGGSGGSNNTGGSGGSGGSGGSGGSNN | |
| | | TGGSGGSGGSGGSGGSNNTGGSGGSGGSGGSGGSNNTGGSGGSGGS | |
| | | GGSGGSNNTGGSGGSGGSGGSGGSNNTGGSGGSGGSGGSGGSNNTG | |
| | | GSGGSGGSGGSGGSNNTGGSGGSGGSGGSGGSNNTGGSGGSGGSGG | |
| | | SGGSNNTGGSGGSGGSGGSGGSNNTGGSGGSGGSGGSGGSNNTGGS | |
| | | GGSGGSGGSGGSNNTGGSGGSGGSGGSGGSNNTGGSGGSGGSGGSG | |
| | | GSNNTGGSGGSHHHHHH | |
| pPOMC | Protein/ | SYSMEHFRWGKPVGKKRRPVKVYPNGAEDESAEAFPLEFKRELTGQ | 164 |
| (138-241) | H. sapiens | RLREGDGPDGPADDGAGAQADLEHSLLVAAEKKDEGPYRMEHFRWG | |
| | | SPPKDKRYGGFM | |
| AQB-507 | Protein/ | MFLQNLFLGFLAVVCANASYSMEHFRWGKPVGKKRRPVKVYPNGAE | 165 |
| | Artificial | DESAEAFPLEFKRELTGQRLREGDGPDGPADDGAGAQADLEHSLLV | |
| | | AAEKKDEGPYRMEHFRWGSPPKDKRYGGFMGGSGGSGGSNNTGGSG | |
| | | GSGGSNNTGGSGGSGGSNNTGGSGGSGGSNNTGGSGGSGGSNNTGG | |
| | | SGGSGGSNNTGGSGGSGGSNNTGGSGGSGGSNNTGGSGGSGGSNNT | |
| | | GGSGGSGGSNNTGGSGGSGGSNNTGGSGGSGGSNNTGGSGGSGGSN | |
| | | NTGGSGGSGGSNNTGGSGGSGGSNNTGGSGGSHHHHHH | |
| AQB-508 | Protein/ | MFLQNLFLGFLAVVCANASYSMEHFRWGKPVGKKRRPVKVYPNGAE | 166 |
| | Artificial | DESAEAFPLEFKRELTGQRLREGDGPDGPADDGAGAQADLEHSLLV | |
| | | AAEKKDEGPYRMEHFRWGSPPKDKRYGGFMGGSGGSGGSGGSNNTG | |
| | | GSGGSGGSGGSNNTGGSGGSGGSGGSNNTGGSGGSGGSGGSNNTGG | |
| | | SGGSGGSGGSNNTGGSGGSGGSGGSNNTGGSGGSGGSGGSNNTGGS | |
| | | GGSGGSGGSNNTGGSGGSGGSGGSNNTGGSGGSGGSGGSNNTGGSG | |
| | | GSGGSGGSNNTGGSGGSGGSGGSNNTGGSGGSGGSGGSNNTGGSGG | |
| | | SGGSGGSNNTGGSGGSGGSGGSNNTGGSGGSHHHHHH | |
| AQB-509 | Protein/ | MFLQNLFLGFLAVVCANASYSMEHFRWGKPVGKKRRPVKVYPNGAE | 167 |
| | Artificial | DESAEAFPLEFKRELTGQRLREGDGPDGPADDGAGAQADLEHSLLV | |
| | | AAEKKDEGPYRMEHFRWGSPPKDKRYGGFMGGSGGSGGSGGSGGSN | |
| | | NTGGSGGSGGSGGSGGSNNTGGSGGSGGSGGSGGSNNTGGSGGSGG | |
| | | SGGSGGSNNTGGSGGSGGSGGSGGSNNTGGSGGSGGSGGSGGSNNT | |
| | | GGSGGSGGSGGSGGSNNTGGSGGSGGSGGSGGSNNTGGSGGSGGSG | |
| | | GSGGSNNTGGSGGSGGSGGSGGSNNTGGSGGSGGSGGSGGSNNTGG | |
| | | SGGSGGSGGSGGSNNTGGSGGSGGSGGSGGSNNTGGSGGSGGSGGS | |
| | | GGSNNTGGSGGSGGSGGSGGSNNTGGSGGSHHHHHH | |
| hFIX | Protein/ | MQRVNMIMAESPGLITICLLGYLLSAECTVFLDHENANKILNRPKR | 168 |
| | H. sapiens | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | T | |
| AQB-601 | Protein/ | MQRVNMIMAESPGLITICLLGYLLSAECTVFLDHENANKILNRPKR | 169 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSNITGGSGGSNITGGSGGSNITGGSGGSHHHHHH | |
| AQB-602 | Protein/ | MQRVNMIMAESPGLITICLLGYLLSAECTVFLDHENANKILNRPKR | 170 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSNITGGSGGSNITGGSGGSNITGGSGGSNITGGSGGSNIT | |
| | | GGSGGSNITGGSGGSNITGGSGGSNITGGSGGSNITGGSGGSNITG | |
| | | GSGGSNITGGSGGSNITGGSGGSNITGGSGGSNITGGSGGSNITGG | |
| | | SGGSHHHHHH | |
| AQB-603 | Protein/ | MQRVNMIMAESPGLITICLLGYLLSAECTVFLDHENANKILNRPKR | 171 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSGGSNITGGSGGSGGSNITGGSGGSGGSNITGGSGGSGGS | |
| | | NITGGSGGSGGSNITGGSGGSGGSNITGGSGGSGGSNITGGSGGSG | |
| | | GSNITGGSGGSGGSNITGGSGGSGGSNITGGSGGSGGSNITGGSGG | |
| | | SGGSNITGGSGGSGGSNITGGSGGSGGSNITGGSGGSGGSNITGGS | |
| | | GGSHHHHHH | |
| AQB-604 | Protein/ | MQRVNMIMAESPGLITICLLGYLLSAECTVFLDHENANKILNRPKR | 172 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSGGSGGSNITGGSGGSGGSGGSNITGGSGGSGGSGGSNIT | |
| | | GGSGGSGGSGGSNITGGSGGSGGSGGSNITGGSGGSGGSGGSNITG | |
| | | GSGGSGGSGGSNITGGSGGSGGSGGSNITGGSGGSGGSGGSNITGG | |
| | | SGGSGGSGGSNITGGSGGSGGSGGSNITGGSGGSGGSGGSNITGGS | |
| | | GGSGGSGGSNITGGSGGSGGSGGSNITGGSGGSGGSGGSNITGGSG | |
| | | GSHHHHHH | |
| AQB-605 | Protein/ | MQRVNMIMAESPGLITICLLGYLLSAECTVFLDHENANKILNRPKR | 173 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSGGSGGSGGSNITGGSGGSGGSGGSGGSNITGGSGGSGGS | |
| | | GGSGGSNITGGSGGSGGSGGSGGSNITGGSGGSGGSGGSGGSNITG | |
| | | GSGGSGGSGGSGGSNITGGSGGSGGSGGSGGSNITGGSGGSGGSGG | |
| | | SGGSNITGGSGGSGGSGGSGGSNITGGSGGSGGSGGSGGSNITGGS | |
| | | GGSGGSGGSGGSNITGGSGGSGGSGGSGGSNITGGSGGSGGSGGSG | |
| | | GSNITGGSGGSGGSGGSGGSNITGGSGGSGGSGGSGGSNITGGSGG | |
| | | SHHHHHH | |
| AQB-606 | Protein/ | MQRVNMIMAESPGLITICLLGYLLSAECTVFLDHENANKILNRPKR | 174 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSGGSNITGGSGGSGGSNITGGSGGSGGSNITGGSGGSGGS | |
| | | NITGGSGGSGGSNITGGSGGSHHHHHH | |
| AQB-607 | Protein/ | MQRVNMIMAESPGLITICLLGYLLSAECTVFLDHENANKILNRPKR | 175 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSGGSNITGGSGGSGGSNITGGSGGSGGSNITGGSGGSGGS | |
| | | NITGGSGGSGGSNITGGSGGSGGSNITGGSGGSGGSNITGGSGGSG | |
| | | GSNITGGSGGSGGSNITGGSGGSGGSNITGGSGGSHHHHHH | |
| AQB-608 | Protein/ | MQRVNMIMAESPGLITICLLGYLLSAECTVFLDHENANKILNRPKR | 176 |
| | Artificial | YNSGKLEEFVQGNLERECMEEKCSFEEAREVFENTERTTEFWKQYV | |
| | | DGDQCESNPCLNGGSCKDDINSYECWCPFGFEGKNCELDVTCNIKN | |
| | | GRCEQFCKNSADNKVVCSCTEGYRLAENQKSCEPAVPFPCGRVSVS | |
| | | QTSKLTRAETVFPDVDYVNSTEAETILDNITQSTQSFNDFTRVVGG | |
| | | EDAKPGQFPWQVVLNGKVDAFCGGSIVNEKWIVTAAHCVETGVKIT | |
| | | VVAGEHNIEETEHTEQKRNVIRIIPHHNYNAAINKYNHDIALLELD | |
| | | EPLVLNSYVTPICIADKEYTNIFLKFGSGYVSGWGRVFHKGRSALV | |
| | | LQYLRVPLVDRATCLRSTKFTIYNNMFCAGFHEGGRDSCQGDSGGP | |
| | | HVTEVEGTSFLTGIISWGEECAMKGKYGIYTKVSRYVNWIKEKTKL | |
| | | TGGSGGSGGSNITGGSGGSGGSNITGGSGGSGGSNITGGSGGSGGS | |
| | | NITGGSGGSGGSNITGGSGGSGGSNITGGSGGSGGSNITGGSGGSG | |
| | | GSNITGGSGGSGGSNITGGSGGSGGSNITGGSGGSGGSNITGGSGG | |
| | | SGGSNITGGSGGSGGSNITGGSGGSGGSNITGGSGGSGGSNITGGS | |
| | | GGSGGSNITGGSGGSGGSNITGGSGGSGGSNITGGSGGSGGSNITG | |
| | | GSGGSGGSNITGGSGGSHHHHHH | |
| Native | Protein/ | MPRLFFFHLLGVCLLLNEFSRAVADSWMEEVIKLCGRELVRAQIAI | 109 |
| proRelaxin-2 | H. sapiens | CGMSTWSKRSLSQEDAPQTPRPVAEIVPSFINKDTETINMMSEFVA | |
| | | NLPQELKLTLSEMQPALPQLQQHVPVLKDSSLLFEEFKKLIRNRQS | |
| | | EAADSSPSELKYLGLDTHSRKKRQLYSALANKCCHVGCTKRSLARF | |
| | | C | |
| Modified | Protein/ | MPRLFFFHLLGVCLLLNEFSRAVADSWMEEVIKLCGRELVRAQIAI | 110 |
| proRelaxin-2 | Artificial | CGMSTWSSLSQEDAPQTPRPVAEIVPSFINKDTETINMMSEFVANL | |
| | | PQELKLTLSEMQPALPQLQQHVPVLKDSSLLFEEFKKLIRNRQSEA | |
| | | ADSSPSELKYLGLDTHSQLYSALANKCCHVGCTKRSLARFC | |
| Modified | Protein/ | MPRLFFFHLLGVCLLLNEFSRAVADSWMEEVIKLCGRELVRAQIAI | 111 |
| proRelaxin-2 | Artificial | CGMSTWSSLSQEDHHHHHHAPQTPRPVAEIVPSFINKDTETINMMS | |
| | | EFVANLPQELKLTLSEMQPALPQLQQHVPVLKDSSLLFEEFKKLIR | |
| | | NRQSEAADSSPSELKYLGLDTHSQLYSALANKCCHVGCTKRSLARF | |
| | | C | |
| Partial Fc | Protein/ | DKTHTCPPCPAPELLGGPSVFLFPPKPKDTLMISRTPEVTCVVVDV | 112 |
| fragment of | H. sapiens | SHEDPEVKFNWYVDGVEVHNAKTKPREEQYNSTYRVVSVLTVLHQD | |
| IgG1 | | WLNGKEYKCKVSNKALPAPIEKTISKAKGQPREPQVYTLPPSRDEL | |
| | | TKNQVSLTCLVKGFYPSDIAVEWESNGQPENNYKTTPPVLDSDGSF | |
| | | FLYSKLTVDKSRWQQGNVFSCSVMHEALHNHYTQKSLSLSPGK | |
| AQB-203a | Protein/ | MPRLFFFHLLGVCLLLNEFSRAVADSWMEEVIKLCGRELVRAQIAI | 113 |
| | Artificial | CGMSTWSSLSQEDAPQTPRPVAEIVPSFINKDTETINMMSEFVANL | |
| | | PQELKLTLSEMQPALPQLQQHVPVLKDSSLLFEEFKKLIRNRQSEA | |
| | | ADSSPSELKYLGLDTHSQLYSALANKCCHVGCTKRSLARFCGGSGG | |
| | | SNNTGGSGGSNNTGGSGGSNNTGGSGGSNNTGGSGGSNNTGGSGGS | |
| | | NNTGGSGGSNNTGGSGGSNNTGGSGGSNNTGGSGGSNNTHHHHHH | |
| AQB-236 | Protein/ | MPRLFFFHLLGVCLLLNEFSRAVAHHHHHHGGSGGSNNTGGSGGSN | 114 |
| | Artificial | NTGGSGGSNNTGGSGGSNNTGGSGGSNNTGGGGSGGGGSDSWMEEV | |
| | | IKLCGRELVRAQTATCGMSTWSRRRRSVTGYGSRKKRQLYSALANK | |
| | | CCHVGCTKRSLARFC | |
| AQB-237 | Protein/ | MPRLFFFHLLGVCLLLNEFSRAVAHHHHHHGGSGGSNNTGGSGGSN | 115 |
| | Artificial | NTGGSGGSNNTGGSGGSNNTGGSGGSNNTGGSGGSNNTGGSGGSNN | |
| | | TGGSGGSNNTGGSGGSNNTGGSGGSNNTGGGGSGGGGSDSWMEEVI | |
| | | KLCGRELVRAQIAICGMSTWSRRRRSVTGYGSRKKRQLYSALANKC | |
| | | CHVGCTKRSLARFC | |
| AQB-238 | Protein/ | MPRLFFFHLLGVCLLLNEFSRAVAHHHHHHGGSGGSNNTGGSGGSN | 116 |
| | Artificial | NTGGSGGSNNTGGSGGSNNTGGSGGSNNTGGSGGSNNTGGSGGSNN | |
| | | TGGSGGSNNTGGSGGSNNTGGSGGSNNTGGSGGSNNTGGSGGSNNT | |
| | | GGSGGSNNTGGSGGSNNTGGSGGSNNTGGGGSGGGGSDSWMEEVIK | |
| | | LCGRELVRAQIAICGMSTWSRRRRSVTGYGSRKKRQLYSALANKCC | |
| | | HVGCTKRSLARFC | |
| AQB-238a | Protein/ | MPRLFFFHLLGVCLLLNEFSRAVAHHHHHHGGSGGSNNTGGSGGSN | 117 |
| | Artificial | NTGGSGGSNNTGGSGGSNNTGGSGGSNNTGGSGGSNNTGGSGGSNN | |
| | | TGGSGGSNNTGGSGGSNNTGGSGGSNNTGGSGGSNNTGGSGGSNNT | |
| | | GGSGGSNNTGGSGGSNNTGGSGGSNNTGGSGGSNNTGGSGGSNNTG | |
| | | GSGGSNNTGGSGGSNNTGGSGGSNNTGGGGSGGGGSDSWMEEVIKL | |
| | | CGRELVRAQIAICGMSTWSRRRRSVTGYGSRKKRQLYSALANKCCH | |
| | | VGCTKRSLARFC | |
| AQB-210 | Protein/ | MPRLFFFHLLGVCLLLNEFSRAVAGGSGGSNNTGGSGGSNNTGGSG | 118 |
| | Artificial | GSNNTGGSGGSNNTGGSGGSNNTGGSGGSNNTGGSGGSNNTGGSGG | |
| | | SNNTGGSGGSNNTGGSGGSNNTDSWMEEVIKLCGRELVRAQIAICG | |
| | | MSTWSSLSQEDHHHHHHAPQTPRPVAEIVPSFINKDTETINMMSEF | |
| | | VANLPQELKLTLSEMQPALPQLQQHVPVLKDSSLLFEEFKKLIRNR | |
| | | QSEAADSSPSELKYLGLDTHSQLYSALANKCCHVGCTKRSLARFC | |
| AQB-211 | Protein/ | MPRLFFFHLLGVCLLLNEFSRAVADSWMEEVIKLCGRELVRAQIAI | 119 |
| | Artificial | CGMSTWSHHHHHHGGSGGSNNTGGSGGSNNTGGSGGSNNTGGSGGS | |
| | | NNTGGSGGSNNTGGSGGSNNTGGSGGSNNTGGSGGSNNTGGSGGSN | |
| | | NTGGSGGSNNTGGSGGSQLYSALANKCCHVGCTKRSLARFC | |
| AQB-211a | Protein/ | MPRLFFFHLLGVCLLLNEFSRAVADSWMEEVIKLCGRELVRAQIAI | 120 |
| | Artificial | CGMSTWSSLSQEDHHHHHHAPQTPRPVAEIVPSFINKDTETINMMS | |
| | | EFVANLPQELKLTLSEMQPALPQLQQHVPVLKDSSLLFEEFKKLIR | |
| | | NRQSEAADSSPSELKYLGLDTHSQLYSALANKCCHVGCTKRSLARF | |
| | | CGGSGGSNNTGGSGGSNNTGGSGGSNNTGGSGGSNNTGGSGGSNNT | |
| | | GGSGGSNNTGGSGGSNNTGGSGGSNNTGGSGGSNNTGGSGGSNNT | |
| AQB-110 | Protein/ | MFLQNLFLGFLAVVCANATPLGPASSLPQSFLLKCLEQVRKIQGDG | 121 |
| | Artificial | AALQEKLCATYKLCHPEELVLLGHSLGIPWAPLSSCPSQALQLAGC | |
| | | LSQLHSGLFLYQGLLQALEGISPELGPTLDTLQLDVADFATTIWQQ | |
| | | MEELGMAPALQPTQGAMPAFASAFQRRAGGVLVASHLQSFLEVSYR | |
| | | VLRHLAQPGGSGGSNNTGGSGGSNNTGGSGGSNNTGGGGSGGGGSD | |
| | | KTHTCPPCPAPELLGGPSVFLFPPKPKDTLMISRTPEVTCVVVDVS | |
| | | HEDPEVKFNWYVDGVEVHNAKTKPREEQYNSTYRVVSVLTVLHQDW | |
| | | LNGKEYKCKVSNKALPAPIEKTISKAKGQPREPQVYTLPPSRDELT | |
| | | KNQVSLTCLVKGFYPSDIAVEWESNGQPENNYKTTPPVLDSDGSFF | |
| | | LYSKLTVDKSRWQQGNVFSCSVMHEALHNHYTQKSLSLSPGK | |
| AQB-255 | Protein/ | MPRLFFFHLLGVCLLLNEFSRAVADKTHTCPPCPAPELLGGPSVFL | 122 |
| | Artificial | FPPKPKDTLMISRTPEVTCVVVDVSHEDPEVKFNWYVDGVEVHNAK | |
| | | TKPREEQYNSTYRVVSVLTVLHQDWLNGKEYKCKVSNKALPAPIEK | |
| | | TISKAKGQPREPQVYTLPPSRDELTKNQVSLTCLVKGFYPSDIAVE | |
| | | WESNGQPENNYKTTPPVLDSDGSFFLYSKLTVDKSRWQQGNVFSCS | |
| | | VMHEALHNHYTQKSLSLSPGKGGSGGSNNTGGSGGSNNTGGSGGSN | |
| | | NTGGSGGSNNTGGSGGSNNTGGGGSGGGGSDSWMEEVIKLCGRELV | |
| | | RAQIAICGMSTWSGGGGSGGGGSQLYSALANKCCHVGCTKRSLARF | |
| | | C | |
| AQB-263 | Protein/ | MPRLFFFHLLGVCLLLNEFSRAVAGGSGGSNNTGGSGGSNNTGGSG | 123 |
| | Artificial | GSNNTGGSGGSNNTGGSGGSNNTGGGGSGGGGSGGGGSDKTHTCPP | |
| | | CPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYK | |
| | | CKVSNKALPAPIEKTISKAKGQPREPQVYTLPPSRDELTKNQVSLT | |
| | | CLVKGFYPSDIAVEWESNGQPENNYKTTPPVLDSDGSFFLYSKLTV | |
| | | DKSRWQQGNVFSCSVMHEALHNHYTQKSLSLSPGKGGGGSGGGGSD | |
| | | SWMEEVIKLCGRELVRAQIAICGMSTWSGGGGSGGGGSQLYSALAN | |
| | | KCCHVGCTKRSLARFC | |
| AQB-701 | Protein/ | MFLQNLFLGFLAVVCANAQVQLQQSGAELVRPGSSVKISCKASGYA | 177 |
| | Artificial | FSSYWMNWVKQRPGQGLEWIGQIWPGDGDTNYNGKFKGKATLTADE | |
| | | SSSTAYMQLSSLASEDSAVYFCARRETTTVGRYYYAMDYWGQGTTV | |
| | | TVSSGGGGSGGGGSGGGGSDIQLTQSPASLAVSLGQRATISCKASQ | |
| | | SVDYDGDSYLNWYQQIPGQPPKLLIYDASNLVSGIPPRFSGSGSGT | |
| | | DFTLNIHPVEKVDAATYHCQQSTEDPWTFGGGTKLEIKSGGSGGSN | |
| | | NTGGSGGSNNTGGSGGSNNTGGSGGSNNTGGSGGSNNTGGSGGSDI | |
| | | KLQQSGAELARPGASVKMSCKTSGYTFTRYTMHWVKQRPGQGLEWI | |
| | | GYINPSRGYTNYNQKFKDKATLTTDKSSSTAYMQLSSLTSEDSAVY | |
| | | YCARYYDDHYCLDYWGQGTTLTVSSVEGGSGGSGGSGGSGGVDDIQ | |
| | | LTQSPAIMSASPGEKVTMTCRASSSVSYMNWYQQKSGTSPKRWIYD | |
| | | TSKVASGVPYRFSGSGSGTSYSLTISSMEAEDAATYYCQQWSSNPL | |
| | | TFGAGTKLELKHHHHHH | |
| AQB-702 | Protein/ | MFLQNLFLGFLAVVCANAQVQLQQSGAELVRPGSSVKISCKASGYA | 178 |
| | Artificial | FSSYWMNWVKQRPGQGLEWIGQIWPGDGDTNYNGKFKGKATLTADE | |
| | | SSSTAYMQLSSLASEDSAVYFCARRETTTVGRYYYAMDYWGQGTTV | |
| | | TVSSGGGGSGGGGSGGGGSDIQLTQSPASLAVSLGQRATISCKASQ | |
| | | SVDYDGDSYLNWYQQIPGQPPKLLIYDASNLVSGIPPRFSGSGSGT | |
| | | DFTLNIHPVEKVDAATYHCQQSTEDPWTFGGGTKLEIKSGGSGGSN | |
| | | NTGGSGGSNNTGGSGGSNNTGGSGGSNNTGGSGGSNNTDIKLQQSG | |
| | | AELARPGASVKMSCKTSGYTFTRYTMHWVKQRPGQGLEWIGYINPS | |
| | | RGYTNYNQKFKDKATLTTDKSSSTAYMQLSSLTSEDSAVYYCARYY | |
| | | DDHYCLDYWGQGTTLTVSSVEGGSGGSGGSGGSGGVDDIQLTQSPA | |
| | | IMSASPGEKVTMTCRASSSVSYMNWYQQKSGTSPKRWIYDTSKVAS | |
| | | GVPYRFSGSGSGTSYSLTISSMEAEDAATYYCQQWSSNPLTFGAGT | |
| | | KLELKHHHHHH | |
| AQB-401a | Protein/ | MGVHECPAWLWLLLSLLSLPLGLPVLGAPPRLICDSRVLERYLLEA | 179 |
| | Artificial | KEAENITTGCAEHCSLNENITVPDTKVNFYAWKRMEVGQQAVEVWQ | |
| | | GLALLSEAVLRGQALLVNSSQPWEPLQLHVDKAVSGLRSLTTLLRA | |
| | | LGAQKEAISPPDAASAAPLRTITADTFRKLFRVYSNFLRGKLKLYT | |
| | | GEACRTGDGGSGGSNNTGGSGGSNNTGGSGGSNNTGGSGGSNNTGG | |
| | | SGGSNNTGGSGGSNNTGGSGGSNNTGGSGGSNNTGGSGGSNNTGGS | |
| | | GGSNNTGGSGGSHHHHHH | |
| AQB-501a | Protein/ | MFLQNLFLGFLAVVCANASYSMEHFRWGKPVGKKRRPVKVYPTGAE | 180 |
| | Artificial | DESAEAFPLEFGGSGGSNNTGGSGGSNNTGGSGGSNNTGGSGGSNN | |
| | | TGGSGGSNNTGGSGGSNNTGGSGGSNNTGGSGGSNNTGGSGGSNNT | |
| | | GGSGGSNNTGGSGGSHHHHHH | |

What is claimed is:

1. A glycoprotein conjugate comprising a therapeutically useful polypeptide (TUP) conjugated to one or more GAABCPs, said GAABCPs comprising a polypeptide sequence encoding from 2-100 repeating glycan acceptor motifs separated by spacer motifs.

2. The glycoprotein conjugate of claim 1, wherein said glycan acceptor motifs are all N-glycan acceptor motifs, O-glycan acceptor motifs, or a mixture of N- and O-glycan acceptor motifs.

3. The glycoprotein conjugate of claim 2, wherein said glycan acceptor motifs all comprise the primary structure NX₄(T/S); wherein N, T, and S are asparagine, threonine, and serine, respectively; and X₄ is any amino acid but proline; and (T/S) indicates that either a threonine residue or a serine residue may be present.

4. The glycoprotein conjugate of claim 3, wherein X₄ is selected from the group consisting of asparagine, glycine, alanine, valine, and isoleucine.

5. The glycoprotein conjugate of claim 2, wherein said glycan acceptor motifs are selected from the group consisting of the motifs provided as SEQ ID NOS 24-33.

6. The glycoprotein conjugate of any one of claims 1-5, wherein at least two of said spacer motifs have different amino acid sequence composition.

7. The glycoprotein conjugate of any one of claims 1-6, wherein at least two of said spacer motifs have different amino acid sequence lengths.

8. The glycoprotein conjugate of any one of claims 1-6, wherein all of said spacer motifs comprise an identical number of amino acids.

9. The glycoprotein conjugate of any one of claims 1-5 and 8, wherein all of said spacer motifs comprise identical amino acid sequence compositions.

10. The glycoprotein conjugate of any one of the preceding claims, wherein each spacer motif comprises or consists of greater than 3 amino acids, 6 amino acids, 9 amino acids, or 12 amino acids.

11. The glycoprotein conjugate of any one of the preceding claims, wherein said spacer motifs comprise the primary structure (X₁X₂X₃)ₙ; wherein X₁ and X₂ are selected from the groups consisting of glycine, alanine, valine, isoleucine, and leucine; X₃ is selected from the group consisting of serine, threonine, asparagine, glutamine, glycine, alanine, valine, isoleucine, and leucine; and n is an integer between 1 and 50.

12. The glycoprotein conjugate of claim 11, wherein X₁ is glycine, X₂ is glycine, X₃ is serine, X₁ and X₂ are both glycine, or wherein X₁ and X₂ are glycine and X₃ is serine.

13. The glycoprotein conjugate of claim 11 or 12, wherein n is selected from 2 or at least 2.

14. The glycoprotein conjugate of claim 11 or 12, wherein n is selected from at least 3, 4, 5, 6, or 7.

15. The glycoprotein conjugate of any one of the preceding claims, wherein the spacer sequences are non-native with respect to the TUP to which the GAABCP is conjugated.

16. The glycoprotein conjugate of any one of the preceding claims, further comprising an affinity tag.

17. The glycoprotein conjugate of claim 16, wherein said affinity tag is a 6×His tag.

18. The glycoprotein conjugate of any one of the preceding claims, further comprising a secretion signal peptide.

19. The glycoprotein conjugate of any one of the preceding claims, comprising a GAABCP conjugated to said TUP, N-terminally, C-terminally, or both N- and C-terminally.

20. The glycoprotein conjugate of any one of the preceding claims, comprising a GAABCP conjugated within the internal amino acid sequence of said TUP.

21. The glycoprotein conjugate of any one of the preceding claims, comprising two distinct TUPs conjugated to the N- and C-terminus, respectively, of said GAABCP amino acid sequence.

22. The glycoprotein conjugate of claim 21, wherein the conjugate is a Glyco-BiTE comprising a GAABCP linking two binding domains, wherein one of said binding domains is capable of activating a T cell, and wherein the other of said binding domains targets a tumor associated antigen.

23. The glycoprotein conjugate of claim 1, wherein said repeating glycan acceptor motifs separated by spacer motifs are encoded by repeating units of a peptide motif selected from any one of the sequences provided as SEQ ID NOS: 34-53.

24. The glycoprotein conjugate of claim 23, comprising more than 5 repeating units of said motif.

25. The glycoprotein conjugate of claim 23, comprising 5, 10, 15, or 20 repeating units of said motif.

26. The glycoprotein conjugate of claim 25, comprising any one of the GAABCP sequences provided as SEQ ID NOS: 54-65.

27. The glycoprotein conjugate of any one of the preceding claims, wherein said TUP is a native polypeptide.

28. The glycoprotein conjugate of any one of claims 1-26, wherein said TUP is a fragment or variant of a native polypeptide.

29. The glycoprotein conjugate of claim 28, wherein said variant polypeptide comprises a signal peptide that is identical to its native signal peptide, with respect to amino acid sequence.

30. The glycoprotein conjugate of claim 29, wherein said variant polypeptide comprises a signal peptide that is not its native signal peptide.

31. The glycoprotein conjugate of claim 30, comprising a non-native signal peptide that is the Pho 1 signal peptide provided as SEQ ID NO: 66 or the serum albumin secretion signal peptide provided as SEQ ID NO: 67.

32. The glycoprotein conjugate of claim 1, wherein said TUP is of mammalian origin.

33. The glycoprotein conjugate of claim 32, wherein said TUP is of human origin.

34. The glycoprotein conjugate of any one of the preceding claims, wherein said TUP is selected from the group consisting of a cytokine, a chemokine, a lymphokine, a ligand, a receptor, a hormone, an apoptosis-inducing polypeptide, an enzyme, an antibody, a proteolytic antibody fragment, an antigen specific antibody fragment, a coagulation factor, a complement protein, and fragments or variants thereof.

35. The glycoprotein conjugate of claim 34 wherein said TUP is a cytokine.

36. The glycoprotein conjugate of claim 35, wherein said cytokine is an interferon or a growth factor.

37. The glycoprotein conjugate of claim 36, wherein said growth factor is selected from the group consisting of human growth hormone, granulocyte macrophage colony stimulating factor (GM-CSF), granulocyte colony stimulating factor (G-CSF), macrophage colony stimulating factor (M-CSF), platelet derived growth factor (PDGF), tumor necrosis factor (TNF), epidermal growth factor (EGF), vascular endothelial growth factor (VEGF), fibroblast growth factor, (FGF), nerve growth factor (NGF), and erythropoietin (EPO), and fragments or variants thereof.

38. The glycoprotein conjugate of claim 37, wherein said G-CSF comprises any one of the amino acid sequences provided as SEQ ID NOS: 97 & 98.

39. The glycoprotein conjugate of claim 37, wherein said EPO comprises the amino acid sequence provided as SEQ ID NO: 147.

40. The glycoprotein conjugate of claim 36, wherein said interferon is a beta-interferon or a gamma-interferon.

41. The glycoprotein conjugate of claim 35, wherein said cytokine is selected from the group consisting of interferon gamma inducing factor I (IGIF), RANTES (regulated upon activation, normal T-cell expressed and presumably secreted), macrophage inflammatory protein-1-alpha (MIP-1-α), macrophage inflammatory protein-1-beta (MIP-1-β), glucagon-like peptide 1 (GLP-1), glucagon-like peptide-2 (GLP-2), Leishmania elongation initiating factor (LEIF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), and fragments or variants thereof.

42. The glycoprotein conjugate of claim 41, wherein said GLP-1 comprises any one of the amino acid sequences provided as SEQ ID NOS: 103 & 104.

43. The glycoprotein conjugate of claim 34, wherein said TUP is an antibody, an antigen specific antibody fragment, a proteolytic antibody fragment or domain, a single chain antibody, a genetically or chemically optimized antibody or a proteolytic and antigen-binding fragment thereof, and fragments or variants thereof.

44. The glycoprotein conjugate of claim 34, wherein said TUP is selected from the group consisting of a soluble gp120 glycoprotein, a soluble gp160 glycoprotein, an IL-1 receptor antagonist, a coagulation factor, insulin, relaxin, and fragments or variants thereof.

45. The glycoprotein conjugate of claim 34, wherein said TUP is a receptor, or the extracellular domain of the receptor, selected from the group consisting of tumor necrosis factor (TNF) type I receptor, tumor necrosis factor (TNF) type II receptor, IL-1 receptor type II, IL-4 receptor, and fragments or variants thereof.

46. The glycoprotein conjugate of claim 34, wherein said TUP is a hormone, and fragments or variants thereof.

47. The glycoprotein conjugate of claim 46, wherein said hormone is a relaxin 2 polypeptide, and fragments or variants thereof.

48. The glycoprotein conjugate of claim 47, wherein said relaxin 2 polypeptide comprises any one of the amino acid sequences provided as SEQ ID NOS: 109-111.

49. The glycoprotein conjugate of any one of the preceding claims, wherein one or more flexible linkers connect said TUP to said one or more GAABCPs.

50. The glycoprotein conjugate of claim 49, said flexible linkers comprising two or more amino acid residues selected from the group consisting of Gly (G) and Ser (S).

51. The glycoprotein conjugate of claim 49, said flexible linkers comprising an amino acid sequence identical to any one of the amino acid sequences provided as SEQ ID NOS: 72-96.

52. The glycoprotein conjugate of claim 1, said conjugate comprising an amino acid sequence that is at least about 90%, 95%, 98%, or 99% identical to any one of the sequences provided as SEQ ID NOS: 99-102, 105-108, 113-123, 134-140, 141-145, 148-156, 158-163, 165-167, and 169-180.

53. The glycoprotein conjugate of claim 1, said conjugate comprising an amino acid sequence that identical to any one of the sequences provided as SEQ ID NOS: 99-102, 105-108, 113-123, 134-140, 141-145, 148-156, 158-163, 165-167, and 169-180.

54. The glycoprotein conjugate of any one of the preceding claims, wherein the plasma half-life of said glycoprotein conjugate is increased compared to the plasma half-life of the corresponding unmodified TUP.

55. The glycoprotein conjugate of any one of the preceding claims, further comprising a second TUP, linked to said glycoprotein conjugate via said GAABCP.

56. The glycoprotein conjugate of claim 55, wherein the second TUP, is a different therapeutically useful polypeptide than that which is comprised in the glycoprotein conjugate to which it is linked.

57. The glycoprotein conjugate of any one of the preceding claims, said conjugate comprising at least two GAABCPs.

58. The glycoprotein conjugate of any one of the preceding claims, said conjugate comprising at least one glycan moiety.

59. The glycoprotein conjugate of claim 58, said glycan being an N-glycan.

60. The glycoprotein conjugate of claim 58, comprising a plurality of N-glycan moieties.

61. The glycoprotein conjugate of claim 60, wherein at least one of said N-glycans comprises at least one terminal sialic acid moiety.

62. The glycoprotein conjugate of claim 60, wherein at least 60% of all N-glycan chains are terminally sialyated.

63. A polynucleotide sequence encoding the glycoprotein conjugate of any one of the preceding claims.

64. A vector comprising the polynucleotide sequence of claim 63.

65. The vector of claim 64, wherein said vector is a plasmid.

66. The vector of claim 65, wherein said plasmid comprises a CMV enhancer and an elongation factor 1a promoter, an intron, and a selectable marker.

67. A host cell comprising the polynucleotide sequence of claim 66.

68. A host cell comprising a vector according to any one of claims 64-66.

69. The host cell of claim 67 or 68, said host cell being a mammalian host cell.

70. The host cell of claim 69, wherein said cell is a Chinese hamster ovary (CHO) cell.

71. The host cell of claim 70, wherein said CHO cell is a CHO DG44 cell, a CHOK1 SV GS-KO cell, a CHO-S cell, a CHO-3E7 cell, or a CHO-BRI cell.

72. A method of producing a glycoprotein conjugate according to any one of claims 1-62, said method comprising culturing a host cell according to any one of claims 69-71 under appropriate expression conditions.

73. The method of claim 72, further comprising purifying said glycoprotein conjugate.

74. A method for increasing glycan occupancy on a polypeptide that is a glycoprotein or a glycoprotein conjugate comprising two or more glycan acceptor motifs separated by a spacer motif, said method comprising modifying a construct encoding the glycoprotein or the glycoprotein conjugate by increasing the length of the spacer sequence separating the glycan acceptor sites and expressing the modified glycoprotein or glycoprotein conjugate in a suitable expression system, thereby increasing glycan occupancy on the modified glycoprotein or glycoprotein conjugate as compared to its unmodified form.

75. The method of claim 74, wherein the polypeptide that is to be modified is the GAABCP-TUP of any one of claims 1-62.

76. A method of increasing the plasma half-life of a GAABCP-TUP, comprising increasing the glycan occupancy of the TUP according to the method of claim 74 or claim 75.

77. A method of increasing the plasma half-life of a TUP, comprising conjugating a GAABCP to the TUP.

78. A pharmaceutical composition comprising the glycoprotein conjugate of any one of claims 1-62.

79. A pharmaceutical composition comprising a purified glycoprotein conjugate according to any one of the glycoprotein conjugates of claims 1-62.

80. The pharmaceutical composition of claim 76 or claim 77, wherein said composition comprises a carrier.

81. The pharmaceutical composition of claim 78, wherein said carrier is a pharmaceutically effective carrier.

82. The pharmaceutical composition of any one of claims 76-79, wherein the composition/glycoprotein conjugate is: a) at least about 95% pure; b) less than about 5% aggregated; and c) substantially endotoxin-free.

83. A method of determining whether the glycoprotein conjugate of any one of claims 1-62 exhibits an increased plasma half-life compared to the plasma half-life of the unconjugated TUP comprising: (a) preparing said conjugate according to the method of claim 72 or 73; (b) measuring the plasma half-life of said conjugate; (c) measuring the plasma half-life of the unconjugated therapeutically useful polypeptide; (d) and comparing the half-lives of the native and conjugated polypeptides.
